(19) United States
(12) Patent Application Publication
(10) Pub. No.: US 2025/0387476 A1
(43) Pub. Date: Dec. 25, 2025

(54) Methods of Treating Cancer

(71) Applicant: Tesaro, Inc., Waltham, MA (US)

(72) Inventor: Martin Huber, Boston, MA (US)

(21) Appl. No.: 19/237,504

(22) Filed: Jun. 13, 2025

Related U.S. Application Data
(63) Continuation of application No. 17/273,680, filed on Mar. 4, 2021.
(60) Provisional application No. 62/726,826, filed on Sep. 4, 2018.

(51) Int. Cl. A61K 39/395; A61K 31/4178; A61K 31/454; A61K 31/496; A61K 31/502; A61K 31/5025; A61K 31/519; A61K 31/5377; A61K 31/55; A61P 35/00

(52) U.S. Cl. CPC A61K 39/3955; A61K 31/4178; A61K 31/454; A61K 31/496; A61K 31/502; A61K 31/5025; A61K 31/519; A61K 31/5377; A61K 31/55; A61P 35/00

(57) ABSTRACT

This invention relates to new methods for treating cancer, including cancers characterized by expression of programmed death ligand 1 (PD-L1).

### CROSS-REFERENCE TO RELATED APPLICATIONS

This application is a continuation of U.S. application Ser. No. 17/273,680, filed Mar. 4, 2024, which is a National Stage Entry of filed Sep. 3, 2019, which claims benefit of U.S. Provisional Application No. 62/726,826, filed Sep. 4, 2018, which is hereby incorporated by reference in its entirety.

### SEQUENCE LISTING

This application contains a Sequence Listing that has been submitted electronically as an XML file named 26368-0043002_SL_ST26.xml. The XML file, created on Jun. 11, 2025, is 53,478 bytes in size. The material in the XML file is hereby incorporated by reference in its entirety.

### FIELD OF THE INVENTION

This invention relates to new methods for treating cancer, including cancers characterized by expression of programmed death ligand 1 (PD-L1).

### BACKGROUND

Cancer is a serious public health problem, with about 609,640 people in the United States of America expected to die of cancer in 2018 alone according to the American Cancer Society, Cancer Facts & FIGS. 2018 (https://www.cancer.org/research/cancer-facts-statistics/all-cancer-facts-figures/cancer-facts-figures-2018.html). Accordingly, there continues to be a need for effective therapies to treat cancer patients.

### SUMMARY OF THE INVENTION

In one aspect, the invention features a method of treating a cancer in a subject, the method comprising: measuring a level of PD-L1 expression in a sample obtained from the subject; and administering to the subject based on the level of PD-L1 expression a therapeutically effective dose of a poly (ADP-ribose) polymerase (PARP) inhibitor; and a therapeutically effective dose of an anti-programmed death-1 protein (PD-1) therapy. In embodiments, the subject has not previously received systemic chemotherapy or any previous anti-PD-1 therapy.

In another aspect, the invention features a method of treating a cancer in a subject, the method comprising: selecting a subject based a level of PD-L1 expression in a sample obtained from the subject as compared to a reference level; and administering to the selected subject a therapeutically effective dose of a poly (ADP-ribose) polymerase (PARP) inhibitor; and a therapeutically effective dose of an anti-programmed death-1 protein (PD-1) therapy. In embodiments, the subject has not previously received systemic chemotherapy or any previous anti-PD-1 therapy.

In embodiments, an anti-PD-1 therapy administered intravenously.

In embodiments, an anti-PD-1 therapy administered to the subject is an agent that inhibits PD-1 or PD-L1/L2. In embodiments, an anti-PD-1 therapy administered to the subject is an agent that inhibits PD-1. In embodiments, an anti-PD-1 therapy administered to the subject is an agent that inhibits PD-L1/L2. In embodiments, an anti-PD-1 therapy administered to the subject is an agent that inhibits PD-L1. In embodiments, an anti-PD-1 therapy administered to the subject is an agent that inhibits PD-L2.

In embodiments, an anti-PD-1 therapy administered to the subject is an agent that inhibits PD-1. In embodiments, an agent that inhibits PD-1 is any one of PD-1 Agent Nos. 1-94. In embodiments, an agent that inhibits PD-1 is a small molecule, a nucleic acid, a polypeptide (e.g. an antibody, a carbohydrate, a lipid, a metal, a toxin, or a PD-1 binding agent.

In embodiments, an agent that inhibits PD-1 is a PD-1-binding agent. In embodiments, a PD-1 binding agent is an antibody, an antibody conjugate, or an antigen-binding fragment thereof. In embodiments, a PD-1 binding agent is selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof.

In embodiments, a PD-1 binding agent comprises
a HC—CDR1 defined by an amino acid sequence that is identical in sequence or contains between 1-5 amino acid substitutions as compared to SEQ ID NO: 1;
a HC—CDR2 defined by an amino acid sequence that is identical in sequence or contains between 1-5 amino acid substitutions as compared to SEQ ID NO: 2;
a HC—CDR3 defined by an amino acid sequence that is identical in sequence or contains between 1-5 amino acid substitutions as compared to SEQ ID NO: 3;
a LC—CDR1 defined by an amino acid sequence that is identical in sequence or contains between 1-5 amino acid substitutions as compared to SEQ ID NO: 4;
a LC—CDR2 defined by an amino acid sequence that is identical in sequence or contains between 1-5 amino acid substitutions as compared to SEQ ID NO: 5; and
a LC—CDR3 defined by an amino acid sequence that is identical in sequence or contains between 1-5 amino acid substitutions as compared to SEQ ID NO: 6.

In embodiments, a PD-1 binding agent comprises
a HC—CDR1 defined by SEQ ID NO: 1;
a HC—CDR2 defined by SEQ ID NO: 2;
a HC—CDR3 defined by SEQ ID NO: 3;
a LC—CDR1 defined by SEQ ID NO: 4;
a LC—CDR2 defined by SEQ ID NO: 5; and
a LC—CDR3 defined by SEQ ID NO: 6.

In embodiments, a PD-1 binding agent comprises
a heavy chain variable domain having an amino acid sequence at least 80%, 85%, 90% or 95% identical to SEQ ID NO: 7; and
a light chain variable domain having an amino acid sequence at least 80%, 85%, 90% or 95% identical to SEQ ID NO: 8.

In embodiments, a PD-1 binding agent comprises
a heavy chain variable domain having an amino acid sequence defined by SEQ ID NO: 7; and
a light chain variable domain having an amino acid sequence defined by SEQ ID NO: 8.

In embodiments, a PD-1 binding agent comprises
a heavy chain polypeptide having an amino acid sequence at least 80%, 85%, 90% or 95% identical to SEQ ID NO: 9; and
a light chain polypeptide having an amino acid sequence at least 80%, 85%, 90% or 95% identical to SEQ ID NO: 10.

In embodiments, a PD-1 binding agent comprises
a heavy chain polypeptide having an amino acid sequence defined by SEQ ID NO: 9; and
a light chain polypeptide having an amino acid sequence defined by SEQ ID NO: 10.

In embodiments, a PD-1 binding agent is TSR-042.

In embodiments, a PD-1 binding agent (e.g., TSR-042) is administered intravenously to the patient at a dose that is: a flat dose between about 100-2000 mg; a flat dose about 100 mg; a flat dose about 200 mg; a flat dose about 300 mg; a flat dose about 400 mg; a flat dose about 500 mg; a flat dose about 600 mg; a flat dose about 700 mg; a flat dose about 800 mg; a flat dose about 900 mg; a flat dose about 1000 mg; a flat dose about 1100 mg; a flat dose about 1200 mg; a flat dose about 1300 mg; a flat dose about 1400 mg; a flat dose about 1500 mg; a flat dose about 1600 mg; a flat dose about 1700 mg; a flat dose about 1800 mg; a flat dose about 1900 mg; a flat dose about 2000 mg; about 1 mg/kg; about 3 mg/kg; or about 10 mg/kg.

In embodiments, a dose of the PD-1 binding agent (e.g., TSR-042) is administered to the subject at an administration interval of once a week, once every 2 weeks, once every 3 weeks, once every 4 weeks, once every 5 weeks, once every 6 weeks, or more.

In embodiments, a PD-1 binding agent (e.g., TSR-042) is administered at an administration interval of once every 3 weeks or once every 6 weeks.

In embodiments, a PD-1 binding agent (e.g., TSR-042) is administered to the subject periodically at a dose of about 500 mg or 1000 mg.

In embodiments, a PD-1 binding agent (e.g., TSR-042) is administered intravenously to the patient at a dose of about 500 mg once every about 3 weeks.

In embodiments, a PD-1 binding agent (e.g., TSR-042) is administered intravenously to the patient at a dose of about 1000 mg once every about 6 weeks.

In embodiments, a PD-1 binding agent (e.g., TSR-042) is administered at a first dose and first administration interval for 3, 4, or 5 cycles followed by a second dose and second administration interval for each subsequent cycle.

In embodiments, a PD-1 binding agent (e.g., TSR-042) is administered at a first dose of about 500 mg once every 3 weeks for 3, 4, or 5 cycles followed by a second dose of about 1000 mg once every 6 weeks or more.

In embodiments, a PD-1 binding agent is intravenously administered to the subject at a first dose of about 500 mg once every about 3 weeks for the first four treatment cycles and then at a second dose of about 1000 mg once every about 6 weeks for the fifth and subsequent treatment cycles.

In embodiments, a PD-1 binding agent is pembrolizumab. In embodiments, pembrolizumab is intravenously administered to the patient at a dose of about 200 mg to the patient once every about 3 weeks (Q3W) or about 2 mg/kg to the patient once about every 3 weeks (Q3W). In embodiments, In embodiments, pembrolizumab is intravenously administered to the patient at a dose of about 200 mg to the patient once every about 3 weeks (Q3W). In embodiments, pembrolizumab is intravenously administered to the patient at a dose of about 2 mg/kg to the patient once about every 3 weeks (Q3W).

In embodiments, a PD-1 binding agent is nivolumab. In embodiments, nivolumab is intravenously administered to the patient at a dose of about 200 mg to the patient once every about 3 weeks (Q3W), about 240 mg to the patient once every about 2 weeks (Q2W), about 480 mg to the patient once every about 4 weeks (Q4W), about 1 mg/kg to the patient once every about Q3W, or about 3 mg/kg to the patient once every about Q3W. In embodiments, nivolumab is intravenously administered to the patient at a dose of about 200 mg to the patient once every about 3 weeks (Q3W). In embodiments, nivolumab is intravenously administered to the patient at a dose of about 240 mg to the patient once every about 2 weeks (Q2W). In embodiments, nivolumab is intravenously administered to the patient at a dose of about 480 mg to the patient once every about 4 weeks (Q4W). In embodiments, nivolumab is intravenously administered to the patient at a dose of about 1 mg/kg to the patient once every about Q3W. In embodiments, nivolumab is intravenously administered to the patient at a dose of about 3 mg/kg to the patient once every about Q3W.

In embodiments, a PD-1 binding agent is administered to the patient intravenously over about 30 minutes.

In embodiments, an anti-PD-1 therapy administered to the subject is an anti-PD-L1/L2 agent. In embodiments, an anti-PD-L1/L2 agent is any of PD-L1 Agent Nos. 1-89. In embodiments, an anti-PD-L1/L2 agent is any of PD-L1 Agent Nos. 1-89. In embodiments, an anti-PD-L1/L2 agent is an anti-PD-L1 antibody agent. In embodiments, an anti-PD-L1 antibody agent is atezolizumab, avelumab, CX-072, durvalumab, FAZ053, LY3300054, PD-L1 millamolecule, or derivatives thereof.

In embodiments, a PARP inhibitor is a small molecule, a nucleic acid, a polypeptide (e.g., an antibody), a carbohydrate, a lipid, a metal, or a toxin. In embodiments, a PARP inhibitor is selected from the group consisting of: ABT-767, AZD 2461, BGB-290, BGP 15, CEP 8983, CEP 9722, DR 2313, E7016, E7449, fluzoparib (SHR 3162), IMP 4297, INO1001, JPI 289, JPI 547, monoclonal antibody B3-LysPE40 conjugate, MP 124, niraparib (ZEJULA) (MK-4827), NU 1025, NU 1064, NU 1076, NU1085, olaparib (AZD2281), ON02231, PD 128763, R 503, R554, rucaparib (RUBRACA) (AG-014699, PF-01367338), SBP 101, SC 101914, simmiparib, talazoparib (BMN-673), veliparib (ABT-888), WW 46, 2-(4-(trifluoromethyl)phenyl)-7,8-dihydro-5H-thiopyrano[4,3-d]pyrimidin-4-ol, and salts or derivatives thereof.

In embodiments, a PARP inhibitor is niraparib.

In embodiments, niraparib is orally administered at a daily dose equivalent to about 100 mg of niraparib free base.

In embodiments, niraparib is orally administered at a daily dose equivalent to about 200 mg of niraparib free base.

In embodiments, niraparib is orally administered at a daily dose equivalent to about 300 mg of niraparib free base.

In embodiments, a PARP inhibitor is administered as part of a treatment cycle that is about 3, 4, 5, or 6 weeks. In embodiments, a PARP inhibitor is administered as part of a treatment cycle that is about 3 weeks or about 6 weeks.

In embodiments, a PD-1 therapy administered to the subject is TSR-042 intravenously administered to the patient at a dose of about 500 mg once every about 3 weeks; and a PARP inhibitor is niraparib orally administered at a dose equivalent to about 100 mg, about 200 mg, or about 300 mg of niraparib free base once daily.

In embodiments, a PD-1 therapy administered to the subject is TSR-042 intravenously administered to the patient at a dose of about 500 mg once every about 3 weeks; and a PARP inhibitor is niraparib orally administered at a dose equivalent to about 100 mg, about 200 mg, or about 300 mg of niraparib free base once daily.

In embodiments, a PD-1 therapy administered to the subject is TSR-042 intravenously administered to the patient at a first dose of 500 mg once every about 3 weeks for three, four, or five cycles, and a second dose of about 1000 mg once every about 6 weeks for subsequent cycles; and a PARP inhibitor is niraparib orally administered at a dose equivalent to about 100 mg, about 200 mg, or about 300 mg of niraparib free base once daily.

In embodiments, a PD-1 therapy administered to the subject is pembrolizumab intravenously administered to the patient at a dose of about 200 mg once every about 3 weeks or about 2 mg/kg to the patient once about every 3 weeks (Q3W); and a PARP inhibitor is niraparib orally administered at a dose equivalent to about 100 mg, about 200 mg, or about 300 mg of niraparib free base once daily.

In embodiments, a PD-1 therapy administered to the subject is nivolumab intravenously administered to the patient at a dose of about 200 mg once every about 3 weeks, about 240 mg to the patient once every about 2 weeks, about 480 mg to the patient once every about 4 weeks, about 1 mg/kg to the patient once every about 3 weeks, or about 3 mg/kg to the patient once every about 3 weeks; and a PARP inhibitor is niraparib orally administered at a dose equivalent to about 100 mg, about 200 mg, or about 300 mg of niraparib free base once daily.

In embodiments, a PARP inhibitor is administered at a dose that is less than the FDA-approved dose.

In embodiments, an initial dose of a PARP inhibitor is a dose equivalent to about 200 mg of niraparib free base once daily.

In embodiments, an initial dose of a PARP inhibitor is a dose equivalent to about 300 mg of niraparib free base once daily.

In embodiments, a method comprises at least three treatment cycles.

In embodiments, a dose of the PARP inhibitor is increased if the subject's hemoglobin ≥9 g/dL, platelets ≥100,000/μL and neutrophils ≥1500/μL for all labs performed during one or more treatment cycles.

In embodiments, a dose of the PARP inhibitor is increased after two treatment cycles.

In embodiments, a PARP inhibitor is niraparib, and the dose is increased from a dose equivalent to about 200 mg of niraparib free base once daily to a dose equivalent to about 300 mg of niraparib free base once daily.

In embodiments, an anti-PD-1 therapy and a PARP inhibitor are administered according to a treatment regimen that includes at least one 2-12 week treatment cycle.

In embodiments, an anti-PD-1 therapy and a PARP inhibitor are administered in repeating cycles of 21 days (3 weeks).

In embodiments, an anti-PD-1 therapy and a PARP inhibitor are administered in repeating cycles of 42 days (6 weeks)

In embodiments, an anti-PD-1 therapy is administered on day one of cycle one.

In embodiments, an anti-PD-1 therapy is administered on day one of a subsequent cycle.

In embodiments, an anti-PD-1 therapy is administered between one to three days before or after day one of a subsequent cycle.

In embodiments, a sample obtained from the subject is a skin tissue, liver tissue, kidney tissue, lung tissue, cerebrospinal fluid (CSF), blood, amniotic fluid, sera, urine, feces, epidermal sample, skin sample, cheek swab, sperm, amniotic fluid, cultured cells, bone marrow sample and/or chorionic villi.

In embodiments, a sample obtained from the subject is a tissue sample or blood. In embodiments, a sample obtained from the subject is a tissue sample. In embodiments, a sample obtained from the subject is a blood sample. In embodiments, circulating tumor cells are detected. In embodiments, a sample obtained from the subject is a cancer tissue sample. In embodiments, a sample comprises a tumor cell or a cancer cell.

In embodiments, a level of PD-L1 expression is at least about 1% as measured by an assay. In embodiments, a level of PD-L1 expression is at least about 5% as measured by an assay. In embodiments, a level of PD-L1 expression is at least about 10% as measured by an assay. In embodiments, a level of PD-L1 expression is at least about 25% as measured by an assay. In embodiments, a level of PD-L1 expression is at least about 50% as measured by an assay.

In embodiments, a level of PD-L1 expression is based on PD-L1 expression in tumor cells (TC).

In embodiments, a level of PD-L1 expression is based on PD-L1 expression in tumor infiltrating immune cells (IC).

In embodiments, a level of PD-L1 expression is measured by a tumor proportion score (TPS).

In embodiments, a level of PD-L1 expression is measured by a combined positive score (CPS).

In embodiments, an assay used to determine PD-L1 expression is an immunohistochemical (IHC) assay, flow cytometry, imaging, PET imaging, immunofluorescence, or western blot. In embodiments, an assay used to determine PD-L1 expression is an immunohistochemical (IHC) assay.

In embodiments, a sample obtained from the subject is characterized by ≥1% PD-L1 expression as measured by an assay. In embodiments, a sample obtained from the subject is characterized by ≥5% PD-L1 expression as measured by an assay. In embodiments, a sample obtained from the subject is characterized by 10% PD-L1 expression as measured by an assay. In embodiments, a sample obtained from the subject is characterized by ≥25% PD-L1 expression as measured by an assay. In embodiments, a sample obtained from the subject is characterized by ≥50% PD-L1 expression as measured by an assay. In embodiments, a sample obtained from the subject is characterized by ≥60%, 65%, 70%, 75%, 80%, 85%, or 90% PD-L1 expression as measured by an assay.

In embodiments, a reference level is a tumor proportion score (TPS) of ≥1% as measured by an assay (e.g., immunohistochemical (IHC) assay).

In embodiments, a reference level is a tumor proportion score (TPS) of ≥5% as measured by an assay (e.g., immunohistochemical (IHC) assay).

In embodiments, a reference level is a tumor proportion score (TPS) of ≥10% as measured by an assay (e.g., immunohistochemical (IHC) assay).

In embodiments, a reference level is a tumor proportion score (TPS) of ≥25% as measured by an assay (e.g., immunohistochemical (IHC) assay).

In embodiments, a reference level is a tumor proportion score (TPS) of ≥50% as measured by an assay (e.g., an immunohistochemical (IHC) assay). In embodiments, a sample obtained from the subject is characterized by a TPS≥60%, 65%, 70%, 75%, 80%, 85%, or 90% PD-L1 expression as measured by an assay (e.g., immunohistochemical (IHC) assay).

In embodiments, a sample obtained from the subject is characterized by higher than or equal PD-L1 expression than the reference level.

In embodiments, a sample obtained from the subject is characterized by high PD-L1 expression.

In embodiments, a sample obtained from the subject is characterized by a tumor proportion score (TPS) of at least about 50% as measured by an immunohistochemical (IHC) assay.

In addition to the PD-L1 expression values described, still further exemplary PD-L1 expression thresholds are described herein, including any of those described in Table 1 (including for certain types of cancer).

In another aspect, the invention features a method of treating a cancer in a subject, the method comprising
measuring a level of PD-L1 expression in a sample obtained from the subject;
determining that said sample is characterized by a tumor proportion score (TPS) of at least about 1% (e.g., as measured by an immunohistochemical (IHC) assay); and
administering to the subject a therapeutically effective dose of a poly (ADP-ribose) polymerase (PARP) inhibitor (e.g., niraparib) and a therapeutically effective dose of an anti-PD-1 therapy (e.g., TSR-042, pembrolizumab, or nivolumab).

In embodiments, an anti-PD-1 therapy is: i) an agent that inhibits PD-1; ii) an agent that inhibits PD-L1/L2; iii) a small molecule, a nucleic acid, a polypeptide (e.g. an antibody, a carbohydrate, a lipid, a metal, a toxin, or a PD-1 binding agent that inhibits PD-1; iv) a PD-1 binding agent; v) a PD-1 binding agent that is an antibody, an antibody conjugate, or an antigen-binding fragment thereof; vi) a PD-1 binding agent selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof; vii) any one of PD-1 Agent Nos. 1-94; viii) any of PD-L1 Agent Nos. 1-89; ix) a small molecule, a nucleic acid, a polypeptide (e.g. an antibody, a carbohydrate, a lipid, a metal, a toxin, or a PD-L1 binding agent that inhibits PD-1; x) a PD-L1 binding agent; xi) a PD-L1 binding agent that is an antibody, an antibody conjugate, or an antigen-binding fragment thereof; xii) a PD-L1 agent selected from the group consisting of: atezolizumab, avelumab, CX-072, durvalumab, FAZ053, LY3300054, PD-L1 millamolecule, and derivatives thereof; xiii) TSR-042, pembrolizumab, or nivolumab; or xiv) TSR-042. In embodiments, an anti-PD-1 therapy is TSR-042, pembrolizumab, or nivolumab. In embodiments, an anti-PD-1 therapy is TSR-042. In embodiments, an anti-PD-1 therapy is pembrolizumab. In embodiments, an anti-PD-1 therapy is nivolumab.

In embodiments, a PARP inhibitor is niraparib, and an anti-PD-1 therapy is TSR-042. In embodiments, a PARP inhibitor is niraparib, and an anti-PD-1 therapy is pembrolizumab. In embodiments, a PARP inhibitor is niraparib, and an anti-PD-1 therapy is nivolumab. In embodiments, the TPS is ≥60%, 65%, 70%, 75%, 80%, 85%, or 90%. In embodiments, a TPS is measured by an immunohistochemical (IHC) assay.

In embodiments, an anti-PD-1 therapy is: i) an agent that inhibits PD-1; ii) an agent that inhibits PD-L1/L2; iii) a small molecule, a nucleic acid, a polypeptide (e.g. an antibody, a carbohydrate, a lipid, a metal, a toxin, or a PD-1 binding agent that inhibits PD-1; iv) a PD-1 binding agent; v) a PD-1 binding agent that is an antibody, an antibody conjugate, or an antigen-binding fragment thereof; vi) a PD-1 binding agent selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof; vii) any one of PD-1 Agent Nos. 1-94; viii) any of PD-L1 Agent Nos. 1-89; ix) a small molecule, a nucleic acid, a polypeptide (e.g. an antibody, a carbohydrate, a lipid, a metal, a toxin, or a PD-L1 binding agent that inhibits PD-1; x) a PD-L1 binding agent; xi) a PD-L1 binding agent that is an antibody, an antibody conjugate, or an antigen-binding fragment thereof; xii) a PD-L1 agent selected from the group consisting of: atezolizumab, avelumab, CX-072, durvalumab, FAZ053, LY3300054, PD-L1 millamolecule, and derivatives thereof; xiii) TSR-042, pembrolizumab, or nivolumab; or xiv) TSR-042. In embodiments, an anti-PD-1 therapy is TSR-042, pembrolizumab, or nivolumab. In embodiments, an anti-PD-1 therapy is TSR-042. In embodiments, an anti-PD-1 therapy is pembrolizumab. In embodiments, an anti-PD-1 therapy is nivolumab.

In another aspect, the invention features a method of treating a cancer in a subject, the method comprising
selecting a subject based on a level of PD-L1 expression in a sample obtained from the subject that is equal to or higher as compared to a reference level, wherein the reference level is a tumor proportion score (TPS) of at least about 1% (e.g., as measured by an immunohistochemical (IHC) assay); and
administering to the subject a therapeutically effective dose of a poly (ADP-ribose) polymerase (PARP) inhibitor (e.g., niraparib) and a therapeutically effective dose of an anti-PD-1 therapy (e.g., TSR-042, pembrolizumab, or nivolumab).

In embodiments, an anti-PD-1 therapy is: i) an agent that inhibits PD-1; ii) an agent that inhibits PD-L1/L2; iii) a small molecule, a nucleic acid, a polypeptide (e.g. an antibody, a carbohydrate, a lipid, a metal, a toxin, or a PD-1 binding agent that inhibits PD-1; iv) a PD-1 binding agent; v) a PD-1 binding agent that is an antibody, an antibody conjugate, or an antigen-binding fragment thereof; vi) a PD-1 binding agent selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof; vii) any one of PD-1 Agent Nos. 1-94; viii) any of PD-L1 Agent Nos. 1-89; ix) a small molecule, a nucleic acid, a polypeptide (e.g. an antibody, a carbohydrate, a lipid, a metal, a toxin, or a PD-L1 binding agent that inhibits PD-1; x) a PD-L1 binding agent; xi) a PD-L1 binding agent that is an antibody, an antibody conjugate, or an antigen-binding fragment thereof; xii) a PD-L1 agent selected from the group consisting of: atezolizumab, avelumab, CX-072, durvalumab, FAZ053, LY3300054, PD-L1 millamolecule, and derivatives thereof; xiii) TSR-042, pembrolizumab, or nivolumab; or xiv) TSR-042. In embodiments, an anti-PD-1 therapy is TSR-042, pembrolizumab, or nivolumab. In embodiments, an anti-PD-1 therapy is TSR-042. In embodiments, an anti-PD-1 therapy is pembrolizumab. In embodiments, an anti-PD-1 therapy is nivolumab.

In embodiments, a PARP inhibitor is niraparib, and an anti-PD-1 therapy is TSR-042. In embodiments, a PARP inhibitor is niraparib, and an anti-PD-1 therapy is pembrolizumab. In embodiments, a PARP inhibitor is niraparib, and an anti-PD-1 therapy is nivolumab. In embodiments, the TPS is ≥60%, 65%, 70%, 75%, 80%, 85%, or 90%. In embodiments, a TPS is measured by an immunohistochemical (IHC) assay.

In another aspect, the invention features a method of treating a cancer in a subject, the method comprising
measuring a level of PD-L1 expression in a sample obtained from the subject;
determining that said sample is characterized by a tumor proportion score (TPS) of at least about 50% (e.g., as measured by an immunohistochemical (IHC) assay); and
administering to the subject a therapeutically effective dose of a poly (ADP-ribose) polymerase (PARP) inhibitor (e.g., niraparib) and a therapeutically effective dose of an anti-PD-1 therapy (e.g., TSR-042, pembrolizumab, or nivolumab).

In embodiments, an anti-PD-1 therapy is: i) an agent that inhibits PD-1; ii) an agent that inhibits PD-L1/L2; iii) a small molecule, a nucleic acid, a polypeptide (e.g. an antibody, a carbohydrate, a lipid, a metal, a toxin, or a PD-1 binding agent that inhibits PD-1; iv) a PD-1 binding agent; v) a PD-1 binding agent that is an antibody, an antibody conjugate, or an antigen-binding fragment thereof; vi) a PD-1 binding agent selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof; vii) any one of PD-1 Agent Nos. 1-94; viii) any of PD-L1 Agent Nos. 1-89; ix) a small molecule, a nucleic acid, a polypeptide (e.g. an antibody, a carbohydrate, a lipid, a metal, a toxin, or a PD-L1 binding agent that inhibits PD-1; x) a PD-L1 binding agent; xi) a PD-L1 binding agent that is an antibody, an antibody conjugate, or an antigen-binding fragment thereof; xii) a PD-L1 agent selected from the group consisting of: atezolizumab, avelumab, CX-072, durvalumab, FAZ053, LY3300054, PD-L1 millamolecule, and derivatives thereof; xiii) TSR-042, pembrolizumab, or nivolumab; or xiv) TSR-042. In embodiments, an anti-PD-1 therapy is TSR-042, pembrolizumab, or nivolumab. In embodiments, an anti-PD-1 therapy is TSR-042. In embodiments, an anti-PD-1 therapy is pembrolizumab. In embodiments, an anti-PD-1 therapy is nivolumab.

In embodiments, a PARP inhibitor is niraparib, and an anti-PD-1 therapy is TSR-042. In embodiments, a PARP inhibitor is niraparib, and an anti-PD-1 therapy is pembrolizumab. In embodiments, a PARP inhibitor is niraparib, and an anti-PD-1 therapy is nivolumab. In embodiments, the TPS is ≥60%, 65%, 70%, 75%, 80%, 85%, or 90%. In embodiments, a TPS is measured by an immunohistochemical (IHC) assay.

In another aspect, the invention features a method of treating a cancer in a subject, the method comprising
selecting a subject based on a level of PD-L1 expression in a sample obtained from the subject that is equal to or higher as compared to a reference level, wherein the reference level is a tumor proportion score (TPS) of at least about 50% (e.g., as measured by an immunohistochemical (IHC) assay); and
administering to the subject a therapeutically effective dose of a poly (ADP-ribose) polymerase (PARP) inhibitor (e.g., niraparib) and a therapeutically effective dose of an anti-PD-1 therapy (e.g., TSR-042, pembrolizumab, or nivolumab).

In embodiments, an anti-PD-1 therapy is: i) an agent that inhibits PD-1; ii) an agent that inhibits PD-L1/L2; iii) a small molecule, a nucleic acid, a polypeptide (e.g. an antibody, a carbohydrate, a lipid, a metal, a toxin, or a PD-1 binding agent that inhibits PD-1; iv) a PD-1 binding agent; v) a PD-1 binding agent that is an antibody, an antibody conjugate, or an antigen-binding fragment thereof; vi) a PD-1 binding agent selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof; vii) any one of PD-1 Agent Nos. 1-94; viii) any of PD-L1 Agent Nos. 1-89; ix) a small molecule, a nucleic acid, a polypeptide (e.g. an antibody, a carbohydrate, a lipid, a metal, a toxin, or a PD-L1 binding agent that inhibits PD-1; x) a PD-L1 binding agent; xi) a PD-L1 binding agent that is an antibody, an antibody conjugate, or an antigen-binding fragment thereof; xii) a PD-L1 agent selected from the group consisting of: atezolizumab, avelumab, CX-072, durvalumab, FAZ053, LY3300054, PD-L1 millamolecule, and derivatives thereof; xiii) TSR-042, pembrolizumab, or nivolumab; or xiv) TSR-042. In embodiments, an anti-PD-1 therapy is TSR-042, pembrolizumab, or nivolumab. In embodiments, an anti-PD-1 therapy is TSR-042. In embodiments, an anti-PD-1 therapy is pembrolizumab. In embodiments, an anti-PD-1 therapy is nivolumab.

In embodiments, a PARP inhibitor is niraparib, and an anti-PD-1 therapy is TSR-042. In embodiments, a PARP inhibitor is niraparib, and an anti-PD-1 therapy is pembrolizumab. In embodiments, a PARP inhibitor is niraparib, and an anti-PD-1 therapy is nivolumab. In embodiments, the TPS is ≥60%, 65%, 70%, 75%, 80%, 85%, or 90%. In embodiments, a TPS is measured by an immunohistochemical (IHC) assay.

In embodiments, a PARP inhibitor is niraparib, and an anti-PD-1 therapy is TSR-042. In embodiments, TSR-042 is intravenously administered to the subject at a dose of about 500 mg once every about 3 weeks. In embodiments, niraparib is administered at a dose (e.g. an initial dose) equivalent to about 200 mg of niraparib free base. In embodiments, niraparib is administered at a dose (e.g. an initial dose) equivalent to about 300 mg of niraparib free base.

In embodiments, a PARP inhibitor is niraparib, and an anti-PD-1 therapy is TSR-042. In embodiments, TSR-042 is intravenously administered to the subject at a dose of about 1000 mg once every about 6 weeks. In embodiments, niraparib is administered at a dose (e.g. an initial dose) equivalent to about 200 mg of niraparib free base. In embodiments, niraparib is administered at a dose (e.g. an initial dose) equivalent to about 300 mg of niraparib free base.

In embodiments, a PARP inhibitor is niraparib, and an anti-PD-1 therapy is TSR-042. In embodiments, TSR-042 is intravenously administered to the subject at a first dose of about 5000 mg once every about 3 weeks for 4 treatment cycles and then at a second dose of about 1000 mg once every about 6 weeks for each subsequent treatment cycle. In embodiments, niraparib is administered at a dose (e.g. an initial dose) equivalent to about 200 mg of niraparib free base. In embodiments, niraparib is administered at a dose (e.g. an initial dose) equivalent to about 300 mg of niraparib free base.

In embodiments, a PARP inhibitor is niraparib, and an anti-PD-1 therapy is pembrolizumab. In embodiments, pembrolizumab is intravenously administered to the subject at a dose of about 200 mg once every about 3 weeks or about 2 mg/kg to the patient once about every 3 weeks. In embodiments, niraparib is administered at a dose (e.g. an initial dose) equivalent to about 200 mg of niraparib free base. In embodiments, niraparib is administered at a dose (e.g. an initial dose) equivalent to about 300 mg of niraparib free base.

In embodiments, a PARP inhibitor is niraparib, and an anti-PD-1 therapy is nivolumab. In embodiments, nivolumab is intravenously administered to the subject at a dose of about 200 mg once every about 3 weeks, about 240 mg to the patient once every about 2 weeks, about 480 mg to the patient once every about 4 weeks, about 1 mg/kg to the patient once every about 3 weeks, or about 3 mg/kg to the patient once every about 3 weeks. In embodiments, niraparib is administered at a dose (e.g. an initial dose) equivalent to about 200 mg of niraparib free base. In embodiments, niraparib is administered at a dose (e.g. an initial dose) equivalent to about 300 mg of niraparib free base.

In embodiments, a PARP inhibitor is administered at a dose that is less than the FDA-approved dose.

In embodiments, an initial dose of a PARP inhibitor is a dose equivalent to about 200 mg of niraparib free base once daily.

In embodiments, an initial dose of a PARP inhibitor is a dose equivalent to about 300 mg of niraparib free base once daily.

In embodiments, a method comprises at least three treatment cycles.

In embodiments, a dose of the PARP inhibitor is increased if the subject's hemoglobin ≥9 g/dL, platelets ≥100,000/μL and neutrophils ≥1500/μL for all labs performed during one or more treatment cycles.

In embodiments, a dose of the PARP inhibitor is increased after two treatment cycles.

In embodiments, a PARP inhibitor is niraparib, and the dose is increased from a dose equivalent to about 200 mg of niraparib free base once daily to a dose equivalent to about 300 mg of niraparib free base once daily.

In embodiments, a subject has not previously received systemic chemotherapy. In embodiments, a subject has not previously received platinum-based chemotherapy.

In embodiments, a subject has not previously received any immunotherapy. In embodiments, a subject has not previously received any anti-PD-1 therapy.

In embodiments, a subject has previously been treated with one or more cancer treatment modalities. In embodiments, a subject has previously been treated with surgery or radiotherapy. In embodiments, a subject has previously been treated with chemotherapy or immunotherapy. In embodiments, a subject has been treated with one, two, three, four, or five lines of prior therapy. In embodiments, a subject has been treated with no more than three lines of prior therapy. In embodiments, a subject has been treated with no more than two lines of prior therapy. In embodiments, a subject has been treated with one or two lines of prior therapy. In embodiments, a subject has been treated with one line of prior therapy. In embodiments, a subject has been treated with two lines of prior therapy.

In embodiments, a subject has previously received immunotherapy. In embodiments, a subject has previously received immunotherapy, where the immunotherapy is not an anti-PD-1 therapy. In embodiments, a subject has previously received immunotherapy that is an anti-PD-1 therapy.

In embodiments, a cancer is recurrent cancer and/or advanced cancer.

In embodiments, a cancer is refractory to a previously received cancer treatment (e.g., previously received immunotherapy such as a previously received anti-PD-1 therapy). In embodiments, a cancer was refractory to a previously received cancer treatment at the beginning of treatment. In embodiments, a cancer became refractory to a previously received cancer treatment during the treatment (e.g., a cancer relapsed and stopped responding to a treatment).

In embodiments, a cancer is refractory to a previously received anti-PD-1 therapy. In embodiments, a cancer was refractory to a previously received anti-PD-1 therapy at the beginning of treatment. In embodiments, a cancer became refractory to a previously received anti-PD-1 therapy during the treatment (e.g., a cancer relapsed and stopped responding to a treatment).

In embodiments, a previously received anti-PD-1 therapy is a PD-1 binding agent. In embodiments, a cancer was refractory to a previously received PD-1 binding agent at the beginning of treatment. In embodiments, a cancer became refractory to a previously received PD-1 binding agent during the treatment (e.g., a cancer relapsed and stopped responding to a treatment).

In embodiments, a previously received anti-PD-1 therapy is a PD-L1 binding agent. In embodiments, a cancer was refractory to a previously received PD-L1 binding agent at the beginning of treatment. In embodiments, a cancer became refractory to a previously received PD-L1 binding agent during the treatment (e.g., a cancer relapsed and stopped responding to a treatment).

In embodiments, a subject has previously received chemotherapy. In embodiments, a previously received chemotherapy is platinum-based chemotherapy (e.g., platinum-based doublet chemotherapy). In embodiments, a chemotherapy comprises administration of cisplatin, carboplatin, oxaliplatin, nedaplatin, triplatin tetranitrate, phenanthriplatin, picoplatin, and/or satraplatin. In embodiments, a cancer is recurrent and/or advanced. In embodiments, a cancer is refractory to the previously received chemotherapy. In embodiments, a cancer is refractory to the previously received chemotherapy at the beginning of treatment. In embodiments, a cancer became refractory to the previously received chemotherapy during treatment (also referred to as relapsed cancer).

In embodiments, a method provides a clinical benefit to the subject that is a complete response (“CR”), a partial response (“PR”) or stable disease (“SD”).

In embodiments, a cancer is MSS or MSI-L, is characterized by microsatellite instability, is MSI-H, has high TMB, has high TMB and is MSS or MSI-L, has high TMB and is MSI-H, has a defective DNA mismatch repair system, has a defect in a DNA mismatch repair gene, is a hypermutated cancer, is an HRD or HRR cancer, comprises a mutation in polymerase delta (POLD), or comprises a mutation in polymerase epsilon (POLE).

In embodiments, a cancer is adenocarcinoma, endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anus, squamous cell carcinoma of the penis, squamous cell carcinoma of the cervix, squamous cell carcinoma of the vagina, squamous cell carcinoma of the vulva, soft tissue sarcoma, melanoma, renal cell carcinoma, lung cancer, non-small cell lung cancer, adenocarcinoma of the lung, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, a hematological cancer, multiple myeloma, B-cell lymphoma, T-cell lymphoma, Hodgkin's lymphoma/primary mediastinal B-cell lymphoma, chronic myelogenous leukemia, acute myeloid leukemia, acute lymphoblastic leukemia, non-Hodgkin's lymphoma, neuroblastoma, a CNS tumor, diffuse intrinsic pontine glioma (DIPG), Ewing's sarcoma, embryonal rhabdomyosarcoma, osteosarcoma, or Wilms tumor.

In embodiments, a cancer is melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, endometrial cancer, ovarian cancer, or Merkel cell carcinoma.

In embodiments, a cancer is a solid tumor.

In embodiments, a cancer is lung cancer.

In embodiments, a cancer is a lung cancer (e.g., a solid tumor). In embodiments, a lung cancer is an advanced lung cancer. In embodiments, a lung cancer is a metastatic lung cancer. In embodiments, a lung cancer is squamous cell carcinoma of the lung. In embodiments, a lung cancer is small cell lung cancer (SCLC). In embodiments, a lung cancer is non-small cell lung cancer (NSCLC). In embodiments, a lung cancer is an ALK-translocated lung cancer (e.g., a lung cancer with a known ALK-translocation). In embodiments, a lung cancer is an EGFR-mutant lung cancer (e.g., a lung cancer with a known EGFR mutation). In embodiments, a lung cancer is a MSI-H lung cancer. In embodiments, a lung cancer is a MSS lung cancer. In embodiments, a lung cancer is a POLE-mutant lung cancer. In embodiments, a lung cancer is a POLD-mutant lung cancer. In embodiments, a lung cancer is a high TMB lung cancer. In embodiments, a lung cancer is associated with homologous recombination repair deficiency/homologous repair deficiency (“HRD”) or is characterized by a homologous recombination repair (HRR) gene mutation or deletion.

In embodiments, a lung cancer is non-small cell lung cancer (NSCLC).

In another aspect, the invention features a method of treating non-small cell lung cancer (NSCLC) in a subject, the method comprising: measuring a level of PD-L1 expression in a sample obtained from the subject, wherein the subject has not previously received systemic chemotherapy or any previous anti-PD-1 therapy; determining that said sample is characterized by a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of TSR-042 in an amount that is about 500 mg once every about 3 weeks. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising: selecting a subject based a level of PD-L1 expression in a sample obtained from the subject that is equal to or higher as compared to a reference level, wherein the reference level is a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of TSR-042 in an amount that is about 500 mg once every about 3 weeks; and wherein the subject has not previously received systemic chemotherapy or any previous anti-PD-1 therapy. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising: measuring a level of PD-L1 expression in a sample obtained from the subject, wherein the subject has not previously received systemic chemotherapy or any previous anti-PD-1 therapy; determining that said sample is characterized by a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of TSR-042 in an amount that is about 1000 mg once every about 6 weeks. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising: selecting a subject based a level of PD-L1 expression in a sample obtained from the subject that is equal to or higher as compared to a reference level, wherein the reference level is a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of TSR-042 in an amount that is about 1000 mg once every about 6 weeks; and wherein the subject has not previously received systemic chemotherapy or any previous anti-PD-1 therapy. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising: measuring a level of PD-L1 expression in a sample obtained from the subject, wherein the subject has not previously received systemic chemotherapy or any previous anti-PD-1 therapy; determining that said sample is characterized by a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of TSR-042 as a first dose of about 500 mg TSR-042 once every three weeks for four treatment cycles and then as a second dose of about 1000 mg TSR-042 once every about 6 weeks for each subsequent treatment cycle. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising selecting a subject based a level of PD-L1 expression in a sample obtained from the subject that is equal to or higher as compared to a reference level, wherein the reference level is a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of TSR-042 as a first dose of about 500 mg TSR-042 once every three weeks for four treatment cycles and then as a second dose of about 1000 mg TSR-042 once every about 6 weeks for each subsequent treatment cycle; and wherein the subject has not previously received systemic chemotherapy or any previous anti-PD-1 therapy. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising: measuring a level of PD-L1 expression in a sample obtained from the subject, wherein the subject has not previously received systemic chemotherapy or any previous anti-PD-1 therapy; determining that said sample is characterized by a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of pembrolizumab in an amount that is about 200 mg once every about 3 weeks or about 2 mg/kg to the patient once about every 3 weeks. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising: selecting a subject based a level of PD-L1 expression in a sample obtained from the subject that is equal to or higher as compared to a reference level, wherein the reference level is a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of pembrolizumab in an amount that is about 200 mg once every about 3 weeks or about 2 mg/kg to the patient once about every 3 weeks; and wherein the subject has not previously received systemic chemotherapy or any previous anti-PD-1 therapy. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising measuring a level of PD-L1 expression in a sample obtained from the subject, wherein the subject has not previously received systemic chemotherapy or any previous anti-PD-1 therapy; determining that said sample is characterized by a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of nivolumab in an amount that is about 200 mg once every about 3 weeks, about 240 mg to the patient once every about 2 weeks, about 480 mg to the patient once every about 4 weeks, about 1 mg/kg to the patient once every about 3 weeks, or about 3 mg/kg to the patient once every about 3 weeks. In embodiments, TPS is ≥60%, 65%, 70%, 75%, 80%, 85%, or 90%. In embodiments, the TPS is measured by an immunohistochemical (IHC) assay.

In another aspect, the invention features a method of treating non-small cell lung cancer (NSCLC) in a subject, the method comprising: selecting a subject based a level of PD-L1 expression in a sample obtained from the subject that is equal to or higher as compared to a reference level, wherein the reference level is a tumor proportion score (TPS) of at least about 50%; and orally administering to the subject a therapeutically effective dose of niraparib in an amount that is equivalent to about 200 mg or 300 mg of niraparib free base once daily, and intravenously administering a therapeutically effective dose of nivolumab in an amount that is about 200 mg once every about 3 weeks, about 240 mg to the patient once every about 2 weeks, about 480 mg to the patient once every about 4 weeks, about 1 mg/kg to the patient once every about 3 weeks, or about 3 mg/kg to the patient once every about 3 weeks; and wherein the subject has not previously received systemic chemotherapy or any previous anti-PD-1 therapy. In embodiments, TPS is ≥60%, 65%, 70%, 75%, 80%, 85%, or 90%. In embodiments, the TPS is measured by an immunohistochemical (IHC) assay.

In embodiments, a NSCLC is squamous non-small cell lung cancer (sqNSCLC). In embodiments, a NSCLC is adenocarcinoma. In embodiments, a NSCLC is large-cell carcinoma.

In embodiments, a lung cancer (e.g., NSCLC) is characterized by an ALK translocation.

In embodiments, a lung cancer (e.g., NSCLC) does not have an ALK translocation.

In embodiments, a lung cancer (e.g., NSCLC) is characterized by a ROS-1 translocation.

In embodiments, a lung cancer (e.g., NSCLC) does not have a ROS-1 translocation.

In embodiments, a lung cancer (e.g., NSCLC) is characterized by an EGFR mutation.

In embodiments, a lung cancer (e.g., NSCLC) does not have an EGFR mutation.

In embodiments, a lung cancer (e.g., NSCLC) is characterized by a gene amplification (e.g., in mesenchymal epithelial transition factor (MET)).

In embodiments, a lung cancer (e.g., NSCLC) is not characterized by a gene amplification.

In embodiments, a lung cancer (e.g., NSCLC) is stage III or stage IV. In embodiments, a lung cancer (e.g., NSCLC) is stage III. In embodiments, a lung cancer (e.g., NSCLC) is stage IV.

In embodiments, a lung cancer (e.g., NSCLC) is locally advanced.

In embodiments, a lung cancer (e.g., NSCLC) is metastatic.

In embodiments, a cancer is breast cancer (e.g., triple negative breast cancer). In embodiments, a cancer is ovarian cancer (e.g., epithelial ovarian cancer). In embodiments, a cancer is lung cancer (e.g., non-small cell lung cancer). In embodiments, a cancer is a melanoma. In embodiments, a cancer is acute myeloid leukemia. In embodiments, a cancer is acute lymphoblastic leukemia. In embodiments, a cancer is non-Hodgkin's lymphoma. In embodiments, a cancer is Hodgkin's lymphoma. In embodiments, a cancer is neuroblastoma. In embodiments, a cancer is a CNS tumor. In embodiments, a cancer is diffuse intrinsic pontine glioma (DIPG). In embodiments, a cancer is Ewing's sarcoma. In embodiments, a cancer is embryonal rhabdomyosarcoma. In embodiments, a cancer is osteosarcoma. In embodiments, a cancer is Wilm's tumor. In embodiments, a cancer is a soft tissue sarcoma (e.g., leiomyosarcoma).

In embodiments, a cancer is an advanced cancer. In embodiments, a cancer is a metastatic cancer. In embodiments, a cancer is a MSI-H cancer. In embodiments, a cancer is a MSS cancer. In embodiments, a cancer is a POLE-mutant cancer. In embodiments, a cancer is a POLD-mutant cancer. In embodiments, a cancer is a high TMB cancer. In embodiments, a cancer is associated with homologous recombination repair deficiency/homologous repair deficiency (“HRD”) or is characterized by a homologous recombination repair (HRR) gene mutation or deletion.

In embodiments, a cancer is a solid tumor. In embodiments, a solid tumor is advanced. In embodiments, a solid tumor is a metastatic solid tumor. In embodiments, a solid tumor is a MSI-H solid tumor. In embodiments, a solid tumor is a MSS solid tumor. In embodiments, a solid tumor is a POLE-mutant solid tumor. In embodiments, a solid tumor is a POLD-mutant solid tumor. In embodiments, a solid tumor is a high TMB solid tumor. In embodiments, a solid tumor is associated with homologous recombination repair deficiency/homologous repair deficiency (“HRD”) or is characterized by a homologous recombination repair (HRR) gene mutation or deletion.

In embodiments, a cancer is a non-endometrial cancer (e.g., a non-endometrial solid tumor). In embodiments, a non-endometrial cancer is an advanced cancer. In embodiments, a non-endometrial cancer is a metastatic cancer. In embodiments, a non-endometrial cancer is a MSI-H cancer. In embodiments, a non-endometrial cancer is a MSS cancer. In embodiments, a non-endometrial cancer is a POLE-mutant cancer. In embodiments, a non-endometrial cancer is a solid tumor (e.g., a MSS solid tumor, a MSI-H solid tumor, a POLD mutant solid tumor, or a POLE-mutant solid tumor). In embodiments, a non-endometrial cancer is a high TMB cancer. In embodiments, a non-endometrial cancer is associated with homologous recombination repair deficiency/homologous repair deficiency (“HRD”) or is characterized by a homologous recombination repair (HRR) gene mutation or deletion.

In embodiments, a cancer is endometrial cancer (e.g., a solid tumor). In embodiments, an endometrial cancer is an advanced cancer. In embodiments, an endometrial cancer is a metastatic cancer. In embodiments, an endometrial cancer is a MSI-H endometrial cancer. In embodiments, an endometrial cancer is a MSS endometrial cancer. In embodiments, an endometrial cancer is a POLE-mutant endometrial cancer. In embodiments, an endometrial cancer is a POLD-mutant endometrial cancer. In embodiments, an endometrial cancer is a high TMB endometrial cancer. In embodiments, an endometrial cancer is associated with homologous recombination repair deficiency/homologous repair deficiency (“HRD”) or is characterized by a homologous recombination repair (HRR) gene mutation or deletion.

In embodiments, a cancer is a colorectal (CRC) cancer (e.g., a solid tumor). In embodiments, a colorectal cancer is an advanced colorectal cancer. In embodiments, a colorectal cancer is a metastatic colorectal cancer. In embodiments, a colorectal cancer is a MSI-H colorectal cancer. In embodiments, a colorectal cancer is a MSS colorectal cancer. In embodiments, a colorectal cancer is a POLE-mutant colorectal cancer. In embodiments, a colorectal cancer is a POLD-mutant colorectal cancer. In embodiments, a colorectal cancer is a high TMB colorectal cancer. In embodiments, a colorectal cancer is associated with homologous recombination repair deficiency/homologous repair deficiency (“HRD”) or is characterized by a homologous recombination repair (HRR) gene mutation or deletion.

In embodiments, a cancer is a melanoma. In embodiments, a melanoma is an advanced melanoma. In embodiments, a melanoma is a metastatic melanoma. In embodiments, a melanoma is a MSI-H melanoma. In embodiments, a melanoma is a MSS melanoma. In embodiments, a melanoma is a POLE-mutant melanoma. In embodiments, a melanoma is a POLD-mutant melanoma. In embodiments, a melanoma is a high TMB melanoma. In embodiments, a melanoma is associated with homologous recombination repair deficiency/homologous repair deficiency (“HRD”) or is characterized by a homologous recombination repair (HRR) gene mutation or deletion.

In embodiments, a cancer is squamous cell carcinoma of the anogenital region (e.g., of the anus, penis, cervix, vagina, or vulva). In embodiments, a squamous cell carcinoma of the anogenital region (e.g., of the anus, penis, cervix, vagina, or vulva) is an advanced cancer. In embodiments, a squamous cell carcinoma of the anogenital region (e.g., of the anus, penis, cervix, vagina, or vulva) is a metastatic cancer. In embodiments, a squamous cell carcinoma of the anogenital region (e.g., of the anus, penis, cervix, vagina, or vulva) is MSI-H. In embodiments, a squamous cell carcinoma of the anogenital region (e.g., of the anus, penis, cervix, vagina, or vulva) is MSS. In embodiments, a lung cancer is a POLE-mutant cancer. In embodiments, a squamous cell carcinoma of the anogenital region (e.g., of the anus, penis, cervix, vagina, or vulva) is associated with homologous recombination repair deficiency/homologous repair deficiency (“HRD”) or is characterized by a homologous recombination repair (HRR) gene mutation or deletion.

In embodiments, a cancer is an ovarian cancer. In embodiments, an ovarian cancer is an advanced ovarian cancer. In embodiments, an ovarian cancer is a metastatic ovarian cancer. In embodiments, an ovarian cancer is a MSI-H ovarian cancer. In embodiments, an ovarian cancer is a MSS ovarian cancer. In embodiments, an ovarian cancer is a POLE-mutant ovarian cancer. In embodiments, an ovarian cancer is a POLD-mutant ovarian cancer. In embodiments, an ovarian cancer is a high TMB ovarian cancer. In embodiments, an ovarian cancer is associated with homologous recombination repair deficiency/homologous repair deficiency (“HRD”) or is characterized by a homologous recombination repair (HRR) gene mutation or deletion. In embodiments, an ovarian cancer is a serous cell ovarian cancer. In embodiments, an ovarian cancer is a clear cell ovarian cancer.

In embodiments, a cancer is a fallopian cancer. In embodiments, a fallopian cancer is an advanced fallopian cancer. In embodiments, a fallopian cancer is a metastatic fallopian cancer. In embodiments, a fallopian cancer is a MSI-H fallopian cancer. In embodiments, a fallopian cancer is a MSS fallopian cancer. In embodiments, a fallopian cancer is a POLE-mutant fallopian cancer. In embodiments, a fallopian cancer is a POLD-mutant fallopian cancer. In embodiments, a fallopian cancer is a high TMB fallopian cancer. In embodiments, a fallopian cancer is associated with homologous recombination repair deficiency/homologous repair deficiency (“HRD”) or is characterized by a homologous recombination repair (HRR) gene mutation or deletion. In embodiments, a fallopian cancer is a serous cell fallopian cancer. In embodiments, a fallopian cancer is a clear cell fallopian cancer.

In embodiments, a cancer is a primary peritoneal cancer. In embodiments, a primary peritoneal cancer is an advanced primary peritoneal cancer. In embodiments, a primary peritoneal cancer is a metastatic primary peritoneal cancer. In embodiments, a primary peritoneal cancer is a MSI-H primary peritoneal cancer. In embodiments, a primary peritoneal cancer is a MSS primary peritoneal cancer. In embodiments, a primary peritoneal cancer is a POLE-mutant primary peritoneal cancer. In embodiments, a primary peritoneal cancer is a POLD-mutant primary peritoneal cancer. In embodiments, a primary peritoneal cancer is a high TMB primary peritoneal cancer. In embodiments, a primary peritoneal cancer is associated with homologous recombination repair deficiency/homologous repair deficiency (“HRD”) or is characterized by a homologous recombination repair (HRR) gene mutation or deletion. In embodiments, a primary peritoneal cancer is a serous cell primary peritoneal cancer. In embodiments, a primary peritoneal cancer is a clear cell primary peritoneal cancer.

In embodiments, a cancer is acute lymphoblastic leukemia (“ALL”). In embodiments, acute lymphoblastic leukemia is advanced acute lymphoblastic leukemia. In embodiments, acute lymphoblastic leukemia is metastatic acute lymphoblastic leukemia. In embodiments, acute lymphoblastic leukemia is MSI-H acute lymphoblastic leukemia. In embodiments, acute lymphoblastic leukemia is MSS acute lymphoblastic leukemia. In embodiments, acute lymphoblastic leukemia is POLE-mutant acute lymphoblastic leukemia. In embodiments, acute lymphoblastic leukemia is POLD-mutant acute lymphoblastic leukemia. In embodiments, an acute lymphoblastic leukemia is associated with homologous recombination repair deficiency/homologous repair deficiency (“HRD”) or is characterized by a homologous recombination repair (HRR) gene mutation or deletion.

In embodiments, a cancer is acute myeloid leukemia (“AML”). In embodiments, acute myeloid leukemia is advanced acute myeloid leukemia. In embodiments, acute myeloid leukemia is metastatic acute myeloid leukemia. In embodiments, acute myeloid leukemia is MSI-H acute myeloid leukemia. In embodiments, acute myeloid leukemia is MSS acute myeloid leukemia. In embodiments, acute myeloid leukemia is POLE-mutant acute myeloid leukemia. In embodiments, acute myeloid leukemia is POLD-mutant acute myeloid leukemia. In embodiments, an acute myeloid leukemia is associated with homologous recombination repair deficiency/homologous repair deficiency (“HRD”) or is characterized by a homologous recombination repair (HRR) gene mutation or deletion.

In embodiments, a cancer is non-Hodgkin's lymphoma (NHL). In embodiments, non-Hodgkin's lymphoma is advanced non-Hodgkin's lymphoma. In embodiments, non-Hodgkin's lymphoma is metastatic non-Hodgkin's lymphoma. In embodiments, non-Hodgkin's lymphoma is MSI-H non-Hodgkin's lymphoma. In embodiments, non-Hodgkin's lymphoma is MSS non-Hodgkin's lymphoma In embodiments, non-Hodgkin's lymphoma is POLE-mutant non-Hodgkin's lymphoma. In embodiments, non-Hodgkin's lymphoma is POLD-mutant non-Hodgkin's lymphoma. In embodiments, non-Hodgkin's lymphoma is associated with homologous recombination repair deficiency/homologous repair deficiency (“HRD”) or is characterized by a homologous recombination repair (HRR) gene mutation or deletion.

In embodiments, a cancer is Hodgkin's lymphoma (HL). In embodiments, Hodgkin's lymphoma is advanced Hodgkin's lymphoma. In embodiments, Hodgkin's lymphoma is metastatic Hodgkin's lymphoma. In embodiments, Hodgkin's lymphoma is MSI-H Hodgkin's lymphoma. In embodiments, Hodgkin's lymphoma is MSS Hodgkin's lymphoma In embodiments, Hodgkin's lymphoma is POLE-mutant Hodgkin's lymphoma. In embodiments, Hodgkin's lymphoma is POLD-mutant Hodgkin's lymphoma. In embodiments, Hodgkin's lymphoma is associated with homologous recombination repair deficiency/homologous repair deficiency (“HRD”) or is characterized by a homologous recombination repair (HRR) gene mutation or deletion.

In embodiments, a cancer is a neuroblastoma (NB). In embodiments, a neuroblastoma is an advanced neuroblastoma. In embodiments, a neuroblastoma is a metastatic neuroblastoma. In embodiments, neuroblastoma is a MSI-H neuroblastoma. In embodiments, a neuroblastoma is a MSS neuroblastoma. In embodiments, a neuroblastoma is a POLE-mutant neuroblastoma. In embodiments, a neuroblastoma is a POLD-mutant neuroblastoma. In embodiments, a neuroblastoma is a high TMB neuroblastoma. In embodiments, a neuroblastoma is associated with homologous recombination repair deficiency/homologous repair deficiency (“HRD”) or is characterized by a homologous recombination repair (HRR) gene mutation or deletion.

In embodiments, a cancer is a CNS tumor. In embodiments, a CNS tumor is advanced. In embodiments, a CNS tumor is a metastatic CNS tumor. In embodiments, a CNS tumor is a MSI-H CNS tumor. In embodiments, a CNS tumor is a MSS CNS tumor. In embodiments, a CNS tumor is a POLE-mutant CNS tumor. In embodiments, a CNS tumor is a POLD-mutant CNS tumor. In embodiments, a CNS tumor is a high TMB CNS tumor. In embodiments, a CNS tumor is associated with homologous recombination repair deficiency/homologous repair deficiency (“HRD”) or is characterized by a homologous recombination repair (HRR) gene mutation or deletion.

In embodiments, a cancer is diffuse intrinsic pontine glioma (DIPG). In embodiments, a DIPG is an advanced DIPG. In embodiments, a DIPG is a metastatic DIPG. In embodiments, DIPG is a MSI-H DIPG. In embodiments, a DIPG is a MSS DIPG. In embodiments, a DIPG is a POLE-mutant DIPG. In embodiments, a DIPG is a POLD-mutant DIPG. In embodiments, a DIPG is a high TMB DIPG. In embodiments, a DIPG is associated with homologous recombination repair deficiency/homologous repair deficiency (“HRD”) or is characterized by a homologous recombination repair (HRR) gene mutation or deletion.

In embodiments, a cancer is Ewing's sarcoma. In embodiments, Ewing's sarcoma is an advanced Ewing's sarcoma. In embodiments, Ewing's sarcoma is a metastatic Ewing's sarcoma. In embodiments, Ewing's sarcoma is a MSI-H Ewing's sarcoma. In embodiments, Ewing's sarcoma is a MSS Ewing's sarcoma. In embodiments, Ewing's sarcoma is a POLE-mutant Ewing's sarcoma. In embodiments, Ewing's sarcoma is a POLD-mutant Ewing's sarcoma. In embodiments, Ewing's sarcoma is a high TMB Ewing's sarcoma. In embodiments, Ewing's sarcoma is associated with homologous recombination repair deficiency/homologous repair deficiency (“HRD”) or is characterized by a homologous recombination repair (HRR) gene mutation or deletion.

In embodiments, a cancer is an embryonal rhabdomyosarcoma (ERS). In embodiments, an embryonal rhabdomyosarcoma is an advanced embryonal rhabdomyosarcoma. In embodiments, an embryonal rhabdomyosarcoma is a metastatic embryonal rhabdomyosarcoma. In embodiments, an embryonal rhabdomyosarcoma is a MSI-H embryonal rhabdomyosarcoma. In embodiments, an embryonal rhabdomyosarcoma is a MSS embryonal rhabdomyosarcoma. In embodiments, an embryonal rhabdomyosarcoma is a POLE-mutant embryonal rhabdomyosarcoma. In embodiments, an embryonal rhabdomyosarcoma is a POLD-mutant embryonal rhabdomyosarcoma. In embodiments, an embryonal rhabdomyosarcoma is a high TMB embryonal rhabdomyosarcoma. In embodiments, an embryonal rhabdomyosarcoma is associated with homologous recombination repair deficiency/homologous repair deficiency (“HRD”) or is characterized by a homologous recombination repair (HRR) gene mutation or deletion.

In embodiments, a cancer is an osteosarcoma (OS). In embodiments, an osteosarcoma is an advanced osteosarcoma. In embodiments, an osteosarcoma is a metastatic osteosarcoma. In embodiments, an osteosarcoma is a MSI-H osteosarcoma. In embodiments, an osteosarcoma is a MSS osteosarcoma. In embodiments, an osteosarcoma is a POLE-mutant osteosarcoma. In embodiments, an osteosarcoma is a POLD-mutant osteosarcoma. In embodiments, an osteosarcoma is a high TMB osteosarcoma. In embodiments, an osteosarcoma is associated with homologous recombination repair deficiency/homologous repair deficiency (“HRD”) or is characterized by a homologous recombination repair (HRR) gene mutation or deletion.

In embodiments, a cancer is a soft tissue sarcoma. In embodiments, a soft tissue sarcoma is an advanced soft tissue sarcoma. In embodiments, a soft tissue sarcoma is a metastatic soft tissue sarcoma. In embodiments, a soft tissue sarcoma is a MSI-H soft tissue sarcoma. In embodiments, a soft tissue sarcoma is a MSS soft tissue sarcoma. In embodiments, a soft tissue sarcoma is a POLE-mutant soft tissue sarcoma. In embodiments, a soft tissue sarcoma is a POLD-mutant soft tissue sarcoma. In embodiments, a soft tissue sarcoma is a high TMB soft tissue sarcoma. In embodiments, a soft tissue sarcoma is associated with homologous recombination repair deficiency/homologous repair deficiency (“HRD”) or is characterized by a homologous recombination repair (HRR) gene mutation or deletion. In embodiments, a soft tissue sarcoma is leiomyosarcoma.

In embodiments, a cancer is Wilms tumor. In embodiments, Wilms tumor is an advanced Wilms tumor. In embodiments, Wilms tumor is a metastatic Wilms tumor. In embodiments, Wilms tumor is a MSI-H Wilms tumor. In embodiments, Wilms tumor is a MSS Wilms tumor. In embodiments, Wilms tumor is a POLE-mutant Wilms tumor. In embodiments, Wilms tumor is a POLD-mutant Wilms tumor. In embodiments, Wilms tumor is a high TMB Wilms tumor. In embodiments, Wilms tumor is associated with homologous recombination repair deficiency/homologous repair deficiency (“HRD”) or is characterized by a homologous recombination repair (HRR) gene mutation or deletion.

In embodiments, a method inhibits tumor growth or reduces tumor size.

In embodiments, a method further comprises administering another therapeutic agent or treatment.

In embodiments, a method further comprises administering one or more of surgery, a radiotherapy, a chemotherapy, an immunotherapy, an anti-angiogenic agent, or an anti-inflammatory agent.

In embodiments, a method further comprises administering an immune checkpoint inhibitor. In embodiments, a method comprises further administering one, two, or three immune checkpoint inhibitors. In embodiments, an immune checkpoint inhibitor is an inhibitor of PD-1, TIM-3, LAG-3, CTLA-4, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R. In embodiments, an immune checkpoint inhibitor is an agent that inhibits programmed death-1 protein (PD-1) signaling, T cell immunoglobulin and mucin protein 3 (TIM-3), lymphocyte activation gene-3 (LAG-3), cytotoxic T-lymphocyte-associated protein 4 (CTLA-4), T cell immunoglobulin and ITIM domain (TIGIT), indoleamine 2,3-dioxygenase (IDO), or colony-stimulating factor 1 receptor (CSF1R).

In embodiments, a method comprises administering an anti-TIM-3 therapy (e.g., an agent that inhibits T cell immunoglobulin and mucin protein 3 (TIM-3)).

In embodiments, an anti-TIM-3 therapy is any one of TIM-3 Agent Nos. 1-21 (FIG. 1D).

In embodiments, an anti-TIM-3 therapy is an agent that inhibits TIM-3.

In embodiments, an anti-TIM-3 therapy is a small molecule, a nucleic acid, a polypeptide (e.g. an antibody), a carbohydrate, a lipid, a metal, a toxin or a TIM-3 binding agent.

In embodiments, an anti-TIM-3 therapy is a TIM-3 binding agent.

In embodiments, a TIM-3 binding agent is an antibody, an antibody conjugate, or an antigen-binding fragment thereof. In embodiments, a TIM-3 binding agent is MBG453, LY3321367, Sym023, TSR-022 or a derivative thereof. In embodiments, a TIM-3 binding agent is TSR-022 or a derivative thereof.

In embodiments, a TIM-3 binding agent comprises:
a HC—CDR1 defined by an amino acid sequence that is identical in sequence or contains between 1-5 amino acid substitutions as compared to SEQ ID NO: 11;
a HC—CDR2 defined by an amino acid sequence that is identical in sequence or contains between 1-5 amino acid substitutions as compared to SEQ ID NO: 12;
a HC—CDR3 defined by an amino acid sequence that is identical in sequence or contains between 1-5 amino acid substitutions as compared to SEQ ID NO: 13;
a LC—CDR1 defined by an amino acid sequence that is identical in sequence or contains between 1-5 amino acid substitutions as compared to SEQ ID NO: 14;
a LC—CDR2 defined by an amino acid sequence that is identical in sequence or contains between 1-5 amino acid substitutions as compared to SEQ ID NO: 15; and
a LC—CDR3 defined by an amino acid sequence that is identical in sequence or contains between 1-5 amino acid substitutions as compared to SEQ ID NO: 16.

In embodiments, a TIM-3 binding agent comprises:
a HC—CDR1 defined by SEQ ID NO: 11;
a HC—CDR2 defined by SEQ ID NO: 12;
a HC—CDR3 defined by SEQ ID NO: 13;
a LC—CDR1 defined by SEQ ID NO: 14;
a LC—CDR2 defined by SEQ ID NO: 15; and
a LC—CDR3 defined by SEQ ID NO: 16.

In embodiments, a TIM-3 binding agent comprises:
a heavy chain variable domain having an amino acid sequence at least 80%, 85%, 90% or 95% identical to SEQ ID NO: 17 or 18; and
a light chain variable domain having an amino acid sequence at least 80%, 85%, 90% or 95% identical to SEQ ID NO: 19 or 20.

In embodiments, a TIM-3 binding agent comprises:
a heavy chain variable domain having an amino acid sequence defined by SEQ ID NO: 17 or 18; and
a light chain variable domain having an amino acid sequence defined by SEQ ID NO: 19 or 20.

In embodiments, a TIM-3 binding agent comprises:
a heavy chain polypeptide having an amino acid sequence at least 80%, 85%, 90% or 95% identical to SEQ ID NO: 21; and
a light chain polypeptide having an amino acid sequence at least 80%, 85%, 90% or 95% identical to SEQ ID NO: 22.

In embodiments, a TIM-3 binding agent comprises:
a heavy chain polypeptide having an amino acid sequence defined by SEQ ID NO: 21; and
a light chain polypeptide having an amino acid sequence defined by SEQ ID NO: 22.

In embodiments, a therapeutically effective dose of the anti-TIM-3 therapy (e.g., a TIM-3 binding agent) is a flat dose of about 100 mg, about 300 mg, about 500 mg, about 900 mg, or about 1200 mg or a weight-based dose of about 1 mg/kg, about 3 mg/kg, or about 10 mg/kg.

In embodiments, a therapeutically effective dose of the anti-TIM-3 therapy is a flat dose of about 100 mg. In embodiments, an anti-TIM-3 therapy is a TIM-3 binding agent (e.g., TSR-022).

In embodiments, a therapeutically effective dose of the anti-TIM-3 therapy is a flat dose of about 300 mg. In embodiments, an anti-TIM-3 therapy is a TIM-3 binding agent (e.g., TSR-022).

In embodiments, a therapeutically effective dose of the anti-TIM-3 therapy is a flat dose of about 900 mg. In embodiments, an anti-TIM-3 therapy is a TIM-3 binding agent (e.g., TSR-022).

In embodiments, an anti-TIM-3 therapy is administered intravenously once every three weeks. In embodiments, an anti-TIM-3 therapy is a TIM-3 binding agent (e.g., TSR-022).

In embodiments, a method comprises administering an anti-LAG-3 therapy (e.g., an agent that inhibits lymphocyte activation gene-3 (LAG-3)). In embodiments, an anti-LAG-3 therapy is an agent that inhibits LAG-3.

In embodiments, an agent that inhibits LAG-3 is any one of LAG-3 Agent Nos. 1-24.

In embodiments, an agent that inhibits LAG-3 is a small molecule, a nucleic acid, a polypeptide (e.g. an antibody, a carbohydrate, a lipid, a metal, a toxin, or a LAG-3 binding agent.

In embodiments, an agent that inhibits LAG-3 is a LAG-3-binding agent.

In embodiments, a LAG-3 binding agent is an antibody, an antibody conjugate, or an antigen-binding fragment thereof.

In embodiments, a LAG-3 binding agent is IMP321, relatlimab (BMS-986016), BI 754111, GSK2831781 (IMP-731), Novartis LAG525 (IMP701), REGN3767, MK-4280, MGD-013, GSK-2831781, FS-118, XmAb22841, INCAGN-2385, FS-18, ENUM-006, AVA-017, AM-0003, Avacta PD-L1/LAG-3 bispecific affamer, iOnctura anti-LAG-3 antibody, Arcus anti-LAG-3 antibody, or Sym022, and derivatives thereof.

In embodiments, a LAG-3 binding agent is TSR-033 or a derivative thereof.

In embodiments, a LAG-3 binding agent comprises:
a HC—CDR1 defined by an amino acid sequence that is identical in sequence or contains between 1-5 amino acid substitutions as compared to SEQ ID NO: 23;
a HC—CDR2 defined by an amino acid sequence that is identical in sequence or contains between 1-5 amino acid substitutions as compared to SEQ ID NO: 24;
a HC—CDR3 defined by an amino acid sequence that is identical in sequence or contains between 1-5 amino acid substitutions as compared to SEQ ID NO: 25;
a LC—CDR1 defined by an amino acid sequence that is identical in sequence or contains between 1-5 amino acid substitutions as compared to SEQ ID NO: 26;
a LC—CDR2 defined by an amino acid sequence that is identical in sequence or contains between 1-5 amino acid substitutions as compared to SEQ ID NO: 27; and
a LC—CDR3 defined by an amino acid sequence that is identical in sequence or contains between 1-5 amino acid substitutions as compared to SEQ ID NO: 28.

In embodiments, a LAG-3 binding agent comprises:
a HC—CDR1 defined by SEQ ID NO: 23;
a HC—CDR2 defined by SEQ ID NO: 24;
a HC—CDR3 defined by SEQ ID NO: 25;
a LC—CDR1 defined by SEQ ID NO: 26;
a LC—CDR2 defined by SEQ ID NO: 27; and
a LC—CDR3 defined by SEQ ID NO: 28.

In embodiments, a LAG-3 binding agent comprises:
a heavy chain variable domain having an amino acid sequence at least 80%, 85%, 90% or 95% identical to SEQ ID NO: 29; and
a light chain variable domain having an amino acid sequence at least 80%, 85%, 90% or 95% identical to SEQ ID NO: 30.

In embodiments, a LAG-3 binding agent comprises: a heavy chain variable domain having an amino acid sequence defined by SEQ ID NO: 29; and
a light chain variable domain having an amino acid sequence defined by SEQ ID NO: 30.

In embodiments, a LAG-3 binding agent comprises:
a heavy chain polypeptide having an amino acid sequence at least 80%, 85%, 90% or 95% identical to SEQ ID NO: 31; and
a light chain polypeptide having an amino acid sequence at least 80%, 85%, 90% or 95% identical to SEQ ID NO: 32.

In embodiments, a LAG-3 binding agent comprises:
a heavy chain polypeptide having an amino acid sequence defined by SEQ ID NO: 31; and
a light chain polypeptide having an amino acid sequence defined by SEQ ID NO: 32.

In embodiments, an anti-LAG-3 therapy is administered as a flat dose of about 240 mg once every two weeks (Q2W), a flat dose of about 500 mg once every two weeks (Q2W), a flat dose of about 720 mg once every two weeks (Q2W), a flat dose of about 900 mg once every two weeks (Q2W), a flat dose of about 1000 mg once every two weeks (Q2W), a flat dose of about 1500 mg once every two weeks (Q2W), a weight-based dose of about 3 mg/kg once every two weeks (Q2W), a weight-based dose of about 10 mg/kg once every two weeks (Q2W), a weight-based dose of about 12 mg/kg once every two weeks (Q2W), a weight-based dose of about 15 mg/kg once every two weeks (Q2W), a flat dose of about 500 mg once every three weeks (Q3W), a flat dose of about 720 mg once every three weeks (Q3W), a flat dose of about 900 mg once every three weeks (Q3W), a flat dose of about 1000 mg once every three weeks (Q3W), a flat dose of about 1500 mg once every three weeks (Q3W), a flat dose of about 1800 mg once every three weeks (Q3W), a flat dose of about 2100 mg once every three weeks (Q3W), a flat dose of about 2200 mg once every three weeks (Q3W), a flat dose of about 2500 mg once every three weeks (Q3W), a weight-based dose of about 10 mg/kg once every three weeks (Q3W), a weight-based dose of about 12 mg/kg once every three weeks (Q3W), a weight-based dose of about 15 mg/kg once every three weeks (Q3W), a weight-based dose of about 20 mg/kg once every three weeks (Q3W), or a weight-based dose of about 25 mg/kg once every three weeks (Q3W).

In another aspect, the invention features a poly (ADP-ribose) polymerase (PARP) inhibitor and an anti-programmed death-1 protein (PD-1) inhibitor for simultaneous or sequential use in the treatment of cancer; where the human has at least one solid tumour and has not previously received systemic chemotherapy or any previous anti-PD-1 therapy; and where the PD-L1 expression level in said solid tumour is high.

In another aspect, the invention features a use of a poly (ADP-ribose) polymerase (PARP) inhibitor in the manufacture of a medicament for use in treating cancer in a human patient; where the PARP inhibitor is to be administered to said human in combination, simultaneously or sequentially in any order, with an anti-programmed death-1 protein (PD-1) inhibitor; where the human has at least one solid tumour and has not previously received systemic chemotherapy or any previous anti-PD-1 therapy; and where the PD-L1 expression level in said solid tumour is high.

In another aspect, the invention features a use of an anti-programmed death-1 protein (PD-1) inhibitor in the manufacture of a medicament for use in treating cancer in a human patient; where the anti-PD-1 inhibitor is to be administered to said human in combination, simultaneously or sequentially in any order, with a poly (ADP-ribose) polymerase (PARP) inhibitor; where the human has at least one solid tumour and has not previously received systemic chemotherapy or any previous anti-PD-1 therapy; and where the PD-L1 expression level in said solid tumour is high.

### BRIEF DESCRIPTION OF THE DRAWING

The Drawing included herein, which is composed of the following Figures, is for illustration purposes only and not for limitation.

FIGS. 1A-1D describe exemplary immune checkpoint inhibitors suitable for use in methods described herein. FIG. 1A describes exemplary PD-1 agents. FIG. 1B describes exemplary PD-L1 agents. FIG. 1C describes exemplary LAG-3 agents. FIG. 1D describes exemplary TIM-3 agents.

FIG. 2 depicts percent tumor shrinkage observed in the patients of Cohort 1, showing the nine patients having partial response (PR) with tumor shrinkage of 30% or more.

FIG. 3 depicts the duration of treatment and tumor response as assessed by RECIST v1.1 in the patients of Cohort 1 who received at least one dose of treatment.

### CERTAIN DEFINITIONS

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. Examples of routes of administration include parenteral, e.g., intravenous, intradermal, subcutaneous, oral (e.g., inhalation), transdermal (i.e., topical), transmucosal, and rectal administration. For example, in some embodiments, administration may be ocular, oral, parenteral, topical, etc. In embodiments, administration is parenteral (e.g., intravenous administration). In embodiments, intravenous administration is intravenous infusion. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc), or other pendant group (e.g., poly-ethylene glycol, etc).

Antibodies include antibody fragments. Antibodies also include, but are not limited to, polyclonal monoclonal, chimeric dAb (domain antibody), single chain, F_{ab}, F_{ab′}, F_{(ab′)2} fragments, scFvs, and F_{ab} expression libraries. An antibody may be a whole antibody, or immunoglobulin, or an antibody fragment.

As detailed above, whole antibodies consist of two pairs of a “light chain” (LC) and a “heavy chain” (HC) (such light chain (LC)/heavy chain pairs are abbreviated herein as LC/HC). The light chains and heavy chains of such antibodies are polypeptides consisting of several domains. In a whole antibody, each heavy chain comprises a heavy chain variable region (abbreviated herein as HCVR or VH) and a heavy chain constant region. The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain VL and a light chain constant domain CL. The variable domains VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (Janeway, C. A., Jr, et al, (2001). Immunobiology., 5th ed., Garland Publishing; and Woof, J., Burton, D., Nat Rev Immunol 4 (2004) 89-99). The two pairs of heavy chain and light chain (HC/LC) are capable of specifically binding to the same antigen. Thus said whole antibody is a bivalent, monospecific antibody. Such “antibodies” include e.g., mouse antibodies, human antibodies, chimeric antibodies, humanized antibodies and genetically engineered antibodies (variant or mutant antibodies) as long as their characteristic properties are retained. In some embodiments, antibodies or binding agents are humanized antibodies, especially as recombinant human or humanized antibodies.

In some embodiments, the antibody or binding agent can be “symmetrical.” By “symmetrical” is meant that the antibody or binding agent has the same kind of Fv regions (e.g., the antibody has two Fab regions). In some embodiments, the antibody or binding agent can be “asymmetrical.” By “asymmetrical” is meant that the antibody or binding agent has at least two different kinds of Fv regions (e.g., the antibody has: Fab and scFv regions, Fab and scFv2 regions, or Fab-VHH regions). Various asymmetrical antibody or binding agent architectures are known in the art (Brinkman and Kontermann et al. 2017 Mabs (9)(2):182-212).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments, an antibody agent is or comprises a polypeptide whose amino acid sequence includes at least one CDR (e.g., at least one heavy chain CDR and/or at least one light chain CDR) that is substantially identical to one found in a reference antibody. In some embodiments, an included CDR is substantially identical to a reference CDR in that it is either identical in sequence or contains between 1-5 amino acid substitutions as compared with the reference CDR. In some embodiments, an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 95%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89.

For example, in some instances the following six groups each contain amino acids that are conservative substitutions for one another: 1) Serine, Threonine; 2) Aspartic Acid, Glutamic Acid; 3) Asparagine, Glutamine; 4) Arginine, Lysine; 5) Isoleucine, Leucine, Methionine, Alanine, Valine, and 6) Phenylalanine, Tyrosine, Tryptophan. Other appropriate substitutions are known to the person of ordinary skill in the art in addition to the non-limiting examples described herein.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). In some embodiments, “binding” refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473).

Binding agent: In general, the term “binding agent” is used herein to refer to any entity that binds to a target of interest as described herein. In many embodiments, a binding agent of interest is one that binds specifically with its target in that it discriminates its target from other potential binding partners in a particular interaction context. In general, a binding agent may be or comprise an entity of any chemical class (e.g., polymer, non-polymer, small molecule, polypeptide, carbohydrate, lipid, nucleic acid, etc.). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding moiety partner. In some embodiments, binding agents are or comprise polypeptides (including, e.g., antibodies or antibody fragments). In some embodiments, binding agents are or comprise small molecules. In some embodiments, binding agents are or comprise nucleic acids. In some embodiments, binding agents are aptamers. In some embodiments, binding agents are polymers; in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins. In some embodiments, binding agents are or comprise peptidomimetics. In some embodiments, binding agents are or comprise scaffold proteins. In some embodiments, binding agents are or comprise mimeotopes. In some embodiments, binding agents are or comprise nucleic acids, such as DNA or RNA. In embodiments, a binding agent is an isolated polypeptide as described herein. In embodiments, a binding agent is an antibody, an antibody conjugate, or an antigen-binding fragment thereof. In embodiments, a binding agent is an antibody.

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure identifies certain cancers to which its teachings may be relevant. In some embodiments, a relevant cancer may be characterized as a solid tumor. In some embodiments, a relevant cancer may be characterized as a hematologic tumor. In embodiments, a cancer is adenocarcinoma, adenocarcinoma of the lung, acute myeloid leukemia (“AML”), acute lymphoblastic leukemia (“ALL”), adrenocortical carcinoma, anal cancer (e.g., squamous cell carcinoma of the anus), appendiceal cancer, B-cell derived leukemia, B-cell derived lymphoma, bladder cancer, brain cancer, breast cancer (e.g., triple negative breast cancer (TNBC)), cancer of the fallopian tube(s), cancer of the testes, cerebral cancer, cervical cancer (e.g., squamous cell carcinoma of the cervix), cholagiocarcinoma, choriocarcinoma, chronic myelogenous leukemia, a CNS tumor, colon cancer or colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, esophageal cancer (e.g., squamous cell carcinoma of the esophagus), Ewing's sarcoma, eye cancer (e.g., uveal melanoma), follicular lymphoma (“FL”), gallbladder cancer, gastric cancer, gastrointestinal cancer, glioma, head and neck cancer (e.g., squamous cell carcinoma of the head and neck (SCHNC)), a hematological cancer, hepatocellular cancer, Hodgkin's lymphoma (HL)/primary mediastinal B-cell lymphoma, kidney cancer, kidney clear cell cancer, laryngeal cancer, leukemia, liver cancer, lung cancer (e.g., non-small cell lung cancer (NSCLC), small cell lung cancer, lung adenocarcinoma, or squamous cell carcinoma of the lung), lymphoma, melanoma, Merkel cell carcinoma, mesothelioma, monocytic leukemia, multiple myeloma, myeloma, a neuroblastic-derived CNS tumor (e.g., neuroblastoma (NB)), non-Hodgkin's lymphoma (NHL), oral cancer, osteosarcoma, ovarian cancer, ovarian carcinoma, pancreatic cancer, peritoneal cancer, primary peritoneal cancer, prostate cancer, relapsed or refractory classic Hodgkin's Lymphoma (cHL), renal cancer (e.g., renal cell carcinoma), rectal cancer, salivary gland cancer (e.g., a salivary gland tumor), sarcoma, skin cancer, small intestine cancer, stomach cancer, squamous cell carcinoma, squamous cell carcinoma of the penis, stomach cancer, T-cell derived leukemia, T-cell derived lymphoma, thymic cancer, a thymoma, thyroid cancer, uveal melanoma, urothelial cell carcinoma, uterine cancer (e.g., uterine endometrial cancer or uterine sarcoma), vaginal cancer (e.g., squamous cell carcinoma of the vagina), vulvar cancer (e.g., squamous cell carcinoma of the vulva), or Wilms tumor.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components. In some embodiments, the carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, it may be desirable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

CDR: The term “CDR” as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Boundaries of CDRs have been defined differently depending on the system, of which several are known in the art (e.g., Kabat, Chothia, etc.).

Combination therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Compound and Agent: The terms “compound” and “agent” are used herein interchangeably. They refer to any naturally occurring or non-naturally occurring (i.e., synthetic or recombinant) molecule, such as a biological macromolecule (e.g., nucleic acid, polypeptide or protein), organic or inorganic molecule, or an extract made from biological materials such as bacteria, plants, fungi, or animal (e.g., mammalian, including human) cells or tissues. The compound may be a single molecule or a mixture or complex of at least two molecules.

Comparable: The term “comparable” as used herein, refers to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Epitope: As used herein, the term “epitope” includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Framework or framework region: As used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Glycan: as used herein, “glycan” refers to a sugar polymer (moiety) component (e.g., such as of a glycoprotein). The term “glycan” can encompass free glycans, including glycans that have been cleaved or otherwise released from a glycoprotein. The term “glycoform” used herein can refer to a particular form of a glycoprotein. That is, when a glycoprotein includes a particular polypeptide that has the potential to be linked to different glycans or sets of glycans, then each different version of the glycoprotein (i.e., where the polypeptide is linked to a particular glycan or set of glycans) can be referred to as a “glycoform.”

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna. CMP matrix.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Human antibody: As used herein, is intended to include antibodies having variable and constant regions generated (or assembled) from human immunoglobulin sequences. In some embodiments, antibodies (or antibody components) may be considered to be “human” even though their amino acid sequences include residues or elements not encoded by human germline immunoglobulin sequences (e.g., include sequence variations, for example that may (originally) have been introduced by random or site-specific mutagenesis in vitro or by somatic mutation in vivo), for example in one or more CDRs and in particular CDR3.

Humanized: As is known in the art, the term “humanized” is commonly used to refer to antibodies (or antibody components) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a CH₁, hinge, CH₂, CH₃, and, optionally, a CH₄ region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical or at least 80%, 85%, 90%, 95%, or 99% identical. In some embodiments, a nucleic acid sequence or amino acid sequence is substantially identical to a reference sequence in that it is either identical in sequence or contains between 1-5 substitutions as compared with the reference sequence. For example, in some embodiments, an amino acid sequence is substantially identical to a reference amino acid sequence in that it is either identical in sequence or contains between 1-5 amino acid substitutions as compared with the reference sequence. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna. CMP matrix.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same type and approximately the same severity of a disease, disorder, or condition as the individual being treated, who is about the same age as the individual being treated (to ensure that the stages of the disease in the treated individual and the control individual(s) are comparable).

Isolated: as used herein, refers to a substance and/or entity (e.g. a nucleic acid or a polypeptide) that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

K_{D}: as used herein, refers to the dissociation constant of a binding agent (e.g., an antibody or binding component thereof) from a complex with its partner (e.g., the epitope to which the antibody or binding component thereof binds).

K_{off}: as used herein, refers to the off rate constant for dissociation of a binding agent (e.g., an antibody or binding component thereof) from a complex with its partner (e.g., the epitope to which the antibody or binding component thereof binds).

Kₒₙ: as used herein, refers to the on rate constant for association of a binding agent (e.g., an antibody or binding component thereof) with its partner (e.g., the epitope to which the antibody or binding component thereof binds).

Kit: As used herein, the term “kit” refers to any delivery system for delivering materials. Such delivery systems may include systems that allow for the storage, transport, or delivery of various diagnostic or therapeutic reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes, cartridges, bottles, ampoules, etc.) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a subportion of the total kit components are included in the term “fragmented kit.” In contrast, a “combined kit” refers to a delivery system containing all of the components in a single container (e.g., in a single box housing each of the desired components). The term “kit” includes both fragmented and combined kits.

Normal: As used herein, the term “normal,” when used to modify the term “individual” or “subject” refers to an individual or group of individuals who does not have a particular disease or condition and is also not a carrier of the disease or condition. The term “normal” is also used herein to qualify a biological specimen or sample isolated from a normal or wild-type individual or subject, for example, a “normal biological sample.”

Nucleic acid: as used herein, the term “nucleic acid” refers to a polymer of at least three nucleotides. In some embodiments, a nucleic acid comprises DNA. In some embodiments comprises RNA. In some embodiments, a nucleic acid is single stranded. In some embodiments, a nucleic acid is double stranded. In some embodiments, a nucleic acid can contain non-natural or altered nucleotides. The terms “nucleic acid” and “polynucleotide” as used herein can refer to a polymeric form of nucleotides of any length, either ribonucleotides (RNA) or deoxyribonucleotides (DNA). These terms can refer to the primary structure of the molecule, and thus include double- and single-stranded DNA, and double- and single-stranded RNA. The terms can include, as equivalents, analogs of either RNA or DNA made from nucleotide analogs and modified polynucleotides such as, though not limited to, methylated and/or capped polynucleotides. Nucleic acids can be linked via phosphate bonds to form nucleic acid sequences or polynucleotides, though many other linkages are known in the art (e.g., phosphorothioates, boranophosphates, and the like).

Patient or subject: As used herein, the term “patient” or “subject” refers to any organism to which provided compound or compounds described herein are administered in accordance with the present invention e.g., for experimental, diagnostic, prophylactic, and/or therapeutic purposes. Typical subjects include animals. The term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone. In embodiments, animals are mammals such as mice, rats, rabbits, non-human primates, and humans; insects; worms; etc. In embodiments, a subject is a human. In some embodiments, a subject may be suffering from, and/or susceptible to a disease, disorder, and/or condition (e.g., cancer). As used herein, a “patient population” or “population of subjects” refers to a plurality of patients or subjects.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Refractory: As used herein, the term “refractory” can be used interchangeably with the term “resistant” and is used to refer to a cancer that does not respond to treatment generally and/or to a particular treatment. In embodiments, a refractory cancer may be resistant at the beginning of a treatment (e.g., a cancer that never responds to a treatment such an immunotherapy such as anti-PD-1 therapy or to chemotherapy). In embodiments, a refractory cancer can become resistant during a treatment (e.g., a cancer that initially responds to a treatment such an immunotherapy such as anti-PD-1 therapy or to chemotherapy but then stops responding to the treatment, also known as relapsed cancer). Thus, in embodiments, a cancer can be refractory to one or more previously received lines of therapy. In embodiments, a cancer that is refractory to a previously received immunotherapy that is an anti-PD-1 therapy may be referred to interchangeably as “PD-1-refractory” or “PD-1-resistant.” In embodiments, a cancer that is refractory to previously received chemotherapy may be referred to interchangeable as “chemotherapy-refractory” or “chemotherapy-resistant.”

Sample: As used herein, the term “sample” encompasses any sample obtained from a biological source. The terms “biological sample” and “sample” are used interchangeably. A biological sample can, by way of non-limiting example, include skin tissue, liver tissue, kidney tissue, lung tissue, cerebrospinal fluid (CSF), blood, amniotic fluid, sera, urine, feces, epidermal sample, skin sample, cheek swab, sperm, amniotic fluid, cultured cells, bone marrow sample and/or chorionic villi. Cell cultures of any biological samples can also be used as biological samples. A biological sample can also be, e.g., a sample obtained from any organ or tissue (including a biopsy or autopsy specimen), can comprise cells (whether primary cells or cultured cells), medium conditioned by any cell, tissue or organ, tissue culture. In some embodiments, biological samples suitable for the invention are samples which have been processed to release or otherwise make available a nucleic acid for detection as described herein. Fixed or frozen tissues also may be used.

Solid Tumor: As used herein, the term “solid tumor” refers to an abnormal mass of tissue that usually does not contain cysts or liquid areas. In some embodiments, a solid tumor may be benign; in some embodiments, a solid tumor may be malignant. Those skilled in the art will appreciate that different types of solid tumors are typically named for the type of cells that form them. Examples of solid tumors are carcinomas, lymphomas, and sarcomas. In some embodiments, solid tumors may be or comprise adrenal, bile duct, bladder, bone, brain, breast, cervix, colon, endometrium, esophagum, eye, gall bladder, gastrointestinal tract, kidney, larynx, liver, lung, nasal cavity, nasopharynx, oral cavity, ovary, penis, pituitary, prostate, retina, salivary gland, skin, small intestine, stomach, testis, thymus, thyroid, uterine, vaginal, and/or vulval tumors.

Suffering from: An individual who is “suffering from” a disease, disorder, and/or condition (e.g., any cancer described herein) has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, and/or condition; (5) a family history of the disease, disorder, and/or condition; (6) reaction to certain bacteria or viruses; (7) exposure to certain chemicals. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically Effective Amount: As used herein, a “therapeutically effective amount” or “effective amount” is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, and/or delays progression of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen. In embodiments, a therapeutically effective dose may be a reduced dose e.g., as compared to a dosage amount, form, or frequency that has been approved by a regulatory agency such as the Food and Drug Administration (e.g., reduced compared to an amount of a therapeutic agent in an FDA-approved dosage form) or, for combination therapy, as compared to a dosage amount, form, or frequency suitable for monotherapy (e.g., reduced compared to an amount of a therapeutic agent in an FDA-approved dosage form used in monotherapy).

Treatment: As used herein, the term “treatment” (also “treat” or “treating”) refers to any administration of a therapeutic molecule (e.g., any compound described herein) that partially or completely alleviates, ameliorates, relieves, inhibits, delays onset of, delays progression of, reduces severity of and/or reduces incidence of one or more symptoms or features of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### DETAILED DESCRIPTION OF CERTAIN EMBODIMENTS

Exemplary methods for treating cancer in a subject are described herein.

The present disclosure also encompasses the recognition that a combination therapy with an agent that inhibits programmed death-1 protein (PD-1) signaling and an agent that inhibits poly [ADP-ribose]polymerase (PARP) is useful for treating certain cancers, including cancers characterized by the expression of programmed death ligand 1 (PD-L1). In particular, a possible synergistic interaction between immune checkpoint inhibitors (e.g., anti-PD-1 therapies such as pembrolizumab and TSR-042) and PARP inhibitors (e.g., niraparib) can result in the methods described herein having particular benefit for PD-1- and PARP-sensitive patient populations, including treatment-naïve population (e.g., patients having lung cancer such as NSCLC) and/or patients with cancers expressing PD-L1.

For example, methods described herein can be useful for the treatment of subjects as a first-line therapy for a cancer characterized by PD-L1 expression (including a cancer characterized by high PD-L1 expression as described herein). Methods described herein can also be particularly useful for the treatment of a subject having cancer who has not previously received immunotherapy or chemotherapy for treatment of the cancer. In particular, methods described herein therefore can result in clinical benefit to a patient such as stable disease (SD), partial response (PR), or complete response (CR).

In some embodiments, methods described herein administering one or both of a therapy that inhibits programmed death-1 protein (PD-1) signaling (“anti-PD-1 therapy”) and a therapy that inhibits poly [ADP-ribose]polymerase (PARP) (“anti-PARP therapy”) to a subject so that the subject receives treatment with both therapies.

In another aspect, the invention features a poly (ADP-ribose) polymerase (PARP) inhibitor and an anti-programmed death-1 protein (PD-1) inhibitor for simultaneous or sequential use in the treatment of cancer; where the human has at least one solid tumour and has not previously received systemic chemotherapy or any previous anti-PD-1 therapy; and where the PD-L1 expression level in said solid tumour is high.

In another aspect, the invention features a use of a poly (ADP-ribose) polymerase (PARP) inhibitor in the manufacture of a medicament for use in treating cancer in a human patient; where the PARP inhibitor is to be administered to said human in combination, simultaneously or sequentially in any order, with an anti-programmed death-1 protein (PD-1) inhibitor; where the human has at least one solid tumour and has not previously received systemic chemotherapy or any previous anti-PD-1 therapy; and where the PD-L1 expression level in said solid tumour is high.

In another aspect, the invention features a use of an anti-programmed death-1 protein (PD-1) inhibitor in the manufacture of a medicament for use in treating cancer in a human patient; where the anti-PD-1 inhibitor is to be administered to said human in combination, simultaneously or sequentially in any order, with a poly (ADP-ribose) polymerase (PARP) inhibitor; where the human has at least one solid tumour and has not previously received systemic chemotherapy or any previous anti-PD-1 therapy; and where the PD-L1 expression level in said solid tumour is high.

### PD-L1 Expression

Methods described herein can be particularly beneficial for the treatment of cancers characterized by the expression of programmed death ligand 1 (PD-L1).

Programmed death ligand 1 (PD-L1) is a protein that interacts with programmed cell death protein 1 (PD-1) and is expressed on, e.g., immune cells and tumor cells (see, e.g., Kim et al., Sci. Rep. 6, 36956; doi:10.1038/srep36956 (2016). In particular, expression of PD-L1 on tumors provides a mechanism of cancer-induced immune suppression, and targeting this pathway can be effective for treating certain cancers (Shukuya et al., Journal of Thoracic Oncology, 11(7):976-988, 2016.

In embodiments, a subject has a cancer characterized by PD-L1 expression.

In embodiments, a method comprises measuring a level of PD-L1 expression in a sample obtained from a subject.

In embodiments, the measured PD-L1 expression of a sample obtained from a subject is compared to a reference level.

In embodiments, a subject is selected for treatment based on the measured PD-L1 expression of a sample as compared to a reference level.

In embodiments, a method further comprises a step of identifying a treatment regimen for the subject.

In embodiments, a sample is obtained from cerebrospinal fluid (CSF), cells, tissue, whole blood, mouthwash, plasma, serum, urine, stool, saliva, cord blood, chorionic villus sample, chorionic villus sample culture, amniotic fluid, amniotic fluid culture, transcervical lavage fluid, and combinations thereof.

In embodiments, a sample obtained from a subject is a tissue sample (e.g., a cancer tissue sample).

In embodiments, a sample obtained from a subject is a tumor sample.

In embodiments, a sample is obtained from a subject who has not been previously treated with an immunotherapy. In embodiments, a sample is obtained from a subject who has previously been treated with an immunotherapy. In embodiments, an immunotherapy is an anti-PD-1 therapy (e.g., a PD-1 binding agent). In embodiments, a sample is obtained before treatment with an immunotherapy (e.g., an anti-PD-1 therapy such as a PD-1 binding agent). In embodiments, a sample is obtained during treatment with an immunotherapy (e.g., an anti-PD-1 therapy such as a PD-1 binding agent). In embodiments, a sample is obtained after treatment with an immunotherapy (e.g., an anti-PD-1 therapy such as a PD-1 binding agent).

In embodiments, a sample is obtained from a subject who has not previously been treated with a line of therapy against cancer. In embodiments, a sample is obtained from a subject who has previously been treated with one or more lines of therapy against cancer. In embodiments, a sample is obtained from a subject who has previously been treated with one line of therapy against cancer. In embodiments, a sample is obtained from a subject who has previously been treated with two lines of therapy against cancer. In embodiments, a sample is obtained from a subject who has previously been treated with two or more lines of therapy against cancer. In embodiments, a line of therapy is one or more of surgery, a radiotherapy, a chemotherapy, an immunotherapy, an anti-angiogenic agent, or an anti-inflammatory agent.

PD-L1 expression can be evaluated by various methods known in the art. Exemplary methods are described in, e.g., Udall et al., Diagnostic Pathology, 13:12 (2018). In some embodiments, PD-L1 tumor status is determined by the presence or absence of PD-L1 expression. Exemplary methods for determining the presence or absence of PD-L1 are described in, e.g., U.S. Patent Publication US20150071910A1. In some embodiments, the percentage of PD-L1 expressed compared to a reference level is determined. In some embodiments, presence and/or expression level/amount of PD-L1 is determined using a method comprising: (a) performing gene expression profiling, PCR (such as rtPCR or qRT-PCR), RNA-seq, microarray analysis, SAGE, MassARRAY technique, or FISH on a sample (such as a subject cancer sample); and b) determining presence and/or expression level/amount of a PD-L1 in the sample. In some embodiments, the microarray method comprises the use of a microarray chip having one or more nucleic acid molecules that can hybridize under stringent conditions to a nucleic acid molecule encoding PD-L1 or having one or more polypeptides (such as peptides or antibodies) that can bind to PD-L1. In one embodiment, the PCR method is qRT-PCR. In one embodiment, the PCR method is multiplex-PCR. In some embodiments, gene expression is measured by microarray. In some embodiments, gene expression is measured by qRT-PCR. In some embodiments, expression is measured by multiplex-PCR.

In some embodiments, PD-L1 expression in the sample obtained from the patient is compared to a control sample characterized by the absence of detectable levels of PD-L1. In some embodiments, the control sample is a healthy individual.

In embodiments, PD-L1 expression is determined using immunohistochemistry (IHC), flow cytometry, PET imaging, immunofluorescence, and/or western blotting. See, e.g., Rom-Jurek et al., Int. J. Mol. Sci., 19:563, 2018. In embodiments, PD-L1 expression is determined using immunohistochemistry (IHC). In embodiments, PD-L1 expression is determined using flow cytometry. In embodiments, PD-L1 expression is determined using PET imaging. In embodiments, PD-L1 expression is determined using immunofluorescence. In embodiments, PD-L1 expression is determined using western blotting. In embodiments, determination of PD-L1 expression comprises the use of a PD-L1 binding agent (e.g., a diagnostic antibody or antibody fragment).

In embodiments, PD-L1 expression is determined using immunohistochemistry (IHC). In embodiments, PD-L1 expression is determined using an IHC assay approved by the FDA. In embodiments, an IHC assay comprises the use of an anti-PD-L1 antibody that is 22C3, 22-8, SP142, SP263, and/or E1L3N. In embodiments, an IHC assay comprises the use of an anti-PD-L1 antibody that is 22C3.

In embodiments, PD-L1 expression is determined using a sample that is formalin fixed. In embodiments, PD-L1 expression is determined using a sample that is formalin fixed and paraffin-embedded (FFPE).

In embodiments, a sample is determined to have positive PD-L1 expression.

In embodiments, a sample (e.g., a tumor sample from a subject) is determined to have high PD-L1 expression.

In embodiments, PD-L1 expression in a sample (e.g., a tumor sample from a subject) is determined using the Tumor Proportion Score (TPS).

In embodiments, PD-L1 expression in a sample is determined by using Combined Positive Score (CPS).

In embodiments, threshold values of PD-L1 expression can vary for different types of cancer.

Table 1 provides a summary of exemplary companion diagnostic devices that can be useful to measure PD-L1 expression, as well as exemplary thresholds of PD-L1 expression that can be used to identify specific cancers that can particularly benefit from an anti-PD-1 therapy (e.g., an inhibitor of PD-1 or of PD-L1). Such exemplary values in Table 1 can also be used to identify patients that can particularly benefit from the methods described herein, including threshold values of PD-L1 expression.

TABLE
FDA-Approved Devices for Determining PD-L1 Expression

| | Intended Use/Indications for Use | | |
|---|---|---|---|
| Device Name/ | | Measure (if | PD-L1 Expression |
| Description | Cancer | specified) | Threshold |
| Dako PD-L1 IHC 22C3 | NSCLC | TPS | ≥1%: cancer expresses |
| pharmDx Qualitative | | | PD-L1 ≥50%: high |
| IHC assay using | | | PD-L1 cancer |
| monoclonal mouse | | | |
| anti-PD-L1: Clone | | % TC | ≥1% |
| 22C3 in FFPE samples | Head and neck squamous | TPS | ≥1% |
| | cell carcinoma | % IC | >1%, >50% |
| | (HNSCC) | or % TC | |
| | Melanoma gastric or | CPS | ≥1% |
| | gastroesophageal | | ≥1%: cancer |
| | junction (GEJ) | | expresses PD-L1 |
| | adenocarcinoma | | |
| | cervical cancer | | |
| | urothelial | CPS | ≥10%: cancer |
| | carcinoma (UC) | | expresses PD-L1 |
| Dako PD-L1 IHC 28-8 | Non-squamous NSCLC | % TC | ≥1%, 5%, or 10% |
| pharmDx: Qualitative | HNSCC | % TC | >1% or >5% |
| IHC using monoclonal | UC | % TC | >1% or >5% |
| rabbit anti-PD-L1: | squamous NSCLC | % TC | ≥5% |
| Clone 22-8 in FFPE | melanoma | % TC | ≥1% or 5% |
| samples | | | |
| Dako PD-L1 IHC 73-10 | NSCLC | % TC | ≥1%, 50%, or 80% |
| Assay: Clone 73-10 | UC | % IC | ≥5% IC |
| Ventana PD-L1 | NSCLC | % IC | ≥50% TC or ≥10% IC |
| (SP142) qualitative | | or % TC | |
| immunohistochemical | HNSCC | % IC | ≥5% |
| assay using rabbit | | or % TC | |
| monoclonal anti-PD-L1 | | | |
| clone SP142 in FFPE | | | |
| samples | | | |
| VENTANA PD-L1 | NSCLC | % TC | ≥25% |
| (SP263) Rabbit | HNSCC | % TC | ≥25% |
| monoclonal | UC | % IC | ≥25% |
| | | or % TC | |
| | Bladder cancer | | ≥25% |

TPS (Tumor Proportion Score): percentage of viable tumor cells showing partial or complete membrane staining at any intensity
CPS (Combined Positive Score): number of PD-L1 staining cells (tumor cells, lymphocytes, macrophages) divided by the total number of viable tumor cells
% IC: the proportion of tumor area occupied by PD-L1 expressing tumor-infiltrating immune cells (% IC) of any intensity
% TC: the percentage of PD-L1 expressing tumor cells (% TC) of any intensity

Accordingly, in some embodiments, a cancer suitable for treatment according to methods described herein is characterized by a PD-L1 expression of 11% (e.g., as determined by an immunohistochemical assay (IHC) such as an FDA-approved IHC assay or an IHC described herein).

In some embodiments, a cancer suitable for treatment according to methods described herein is characterized by a PD-L1 expression of 55% (e.g., as determined by an immunohistochemical assay (IHC) such as an FDA-approved IHC assay or an IHC described herein). In some embodiments, a cancer suitable for treatment according to methods described herein is characterized by a PD-L1 expression of ≥10% (e.g., as determined by an immunohistochemical assay (IHC) such as an FDA-approved IHC assay or an IHC described herein). In some embodiments, a cancer suitable for treatment according to methods described herein is characterized by a PD-L1 expression of ≥25% (e.g., as determined by an immunohistochemical assay (IHC) such as an FDA-approved IHC assay or an IHC described herein). In some embodiments, a cancer suitable for treatment according to methods described herein is characterized by a PD-L1 expression of ≥50% (e.g., as determined by an immunohistochemical assay (IHC) such as an FDA-approved IHC assay or an IHC described herein). In some embodiments, a cancer suitable for treatment according to methods described herein is characterized by a PD-L1 expression of ≥60% (e.g., as determined by an immunohistochemical assay (IHC) such as an FDA-approved IHC assay or an IHC described herein). In some embodiments, a cancer suitable for treatment according to methods described herein is characterized by a PD-L1 expression of ≥70% (e.g., as determined by an immunohistochemical assay (IHC) such as an FDA-approved IHC assay or an IHC described herein). In some embodiments, a cancer suitable for treatment according to methods described herein is characterized by a PD-L1 expression of ≥80% (e.g., as determined by an immunohistochemical assay (IHC) such as an FDA-approved IHC assay or an IHC described herein). In some embodiments, a cancer suitable for treatment according to methods described herein is characterized by a PD-L1 expression of ≥90% (e.g., as determined by an immunohistochemical assay (IHC) such as an FDA-approved IHC assay or an IHC described herein).

### Tumor Proportion Score (TPS)

In embodiments, PD-L1 expression is expressed as Tumor Proportion Score (TPS).

The Tumor Proportion Score (TPS) of a sample can be determined by the percentage of viable tumor cells showing partial or complete membrane staining at any intensity. In embodiments, the TPS of a sample is determined using IHC.

In embodiments, a positive PD-L1 expression is characterized by a TPS of at least about 1% (i.e., a TPS≥1%). In embodiments, a positive PD-L1 expression is characterized by a TPS of about 1% to 49%.

In embodiments, a sample that expresses PD-L1 has a TPS of at least about 1% (i.e., a TPS≥1%).

In embodiments, a sample that expresses PD-L1 has a TPS of at least about 5% (i.e., a TPS≥5%).

In embodiments, a sample that expresses PD-L1 has a TPS of at least about 10% (i.e., a TPS≥10%).

In embodiments, a sample that expresses PD-L1 has a TPS of at least about 25% (i.e., a TPS≥25%).

In embodiments, a sample that expresses PD-L1 has a TPS of about 1% to 49%.

In embodiments, a sample that expresses PD-L1 has a TPS of at least about 50% (i.e., a TPS≥50%).

In embodiments, a sample that expresses PD-L1 has a TPS of at least about 60% (i.e., a TPS≥60%).

In embodiments, a sample that expresses PD-L1 has a TPS of at least about 70% (i.e., a TPS≥70%).

In embodiments, a sample that expresses PD-L1 has a TPS of at least about 80% (i.e., a TPS≥80%).

In embodiments, a sample that expresses PD-L1 has a TPS of at least about 90% (i.e., a TPS≥90%).

In embodiments, a high PD-L1 expression is characterized by a TPS of at least about 20% (i.e., a TPS≥20%). In embodiments, a high PD-L1 expression is characterized by a TPS of at least about 30% (i.e., a TPS≥30%). In embodiments, a high PD-L1 expression is characterized by a TPS of at least about 40% (i.e., a TPS≥40%). In embodiments, a high PD-L1 expression is characterized by a TPS of at least about 50% (i.e., a TPS≥50%). In embodiments, a high PD-L1 expression is characterized by a TPS of at least about 55% (i.e., a TPS≥55%). In embodiments, a high PD-L1 expression is characterized by a TPS of at least about 60% (i.e., a TPS≥60%).

In embodiments, a Tumor Proportion Score (TPS) of a sample is compared to a reference TPS. In embodiments, a subject is selected for treatment based on the TPS of a sample as compared to a reference TPS.

In embodiments, a reference level is a TPS of 0%.

In embodiments, a sample does not express PD-L1 and the TPS of the sample is 0%. In embodiments, a subject is selected due to a measured TPS of a sample from the subject that is 0%.

In embodiments, a reference level is a TPS of 1%.

In embodiments, the TPS of a sample from a selected subject is at least about 1% (i.e., a TPS≥1%). In embodiments, a subject is selected due to a measured TPS of a sample from the subject that is at least about 1% (i.e., a TPS≥1%).

In embodiments, the TPS of a sample from a selected subject is no more than about 1% (i.e., a TPS<1%). In embodiments, a subject is selected due to a measured TPS of a sample from the subject that is no more than about 1% (i.e., a TPS<1%).

In embodiments, a reference level is a TPS of 5%.

In embodiments, the TPS of a sample from a selected subject is at least about 5% (i.e., a TPS≥1%). In embodiments, a subject is selected due to a measured TPS of a sample from the subject that is at least about 5% (i.e., a TPS≥5%).

In embodiments, the TPS of a sample from a selected subject is no more than about 5% (i.e., a TPS<5%). In embodiments, a subject is selected due to a measured TPS of a sample from the subject that is no more than about 5% (i.e., a TPS<5%).

In embodiments, a reference level is a TPS of 10%.

In embodiments, the TPS of a sample from a selected subject is at least about 10% (i.e., a TPS≥10%). In embodiments, a subject is selected due to a measured TPS of a sample from the subject that is at least about 10% (i.e., a TPS≥10%).

In embodiments, the TPS of a sample from a selected subject is no more than about 10% (i.e., a TPS<10%). In embodiments, a subject is selected due to a measured TPS of a sample from the subject that is no more than about 10% (i.e., a TPS<10%).

In embodiments, a reference level is a TPS of 25%.

In embodiments, the TPS of a sample from a selected subject is no more than about 25% (i.e., a TPS<25%). In embodiments, a subject is selected due to a measured TPS of a sample from the subject that is no more than about 25% (i.e., a TPS<25%).

In embodiments, a reference level is a TPS of 50%.

In embodiments, the TPS of a sample from a selected subject is at least about 50% (i.e., a TPS≥50%). In embodiments, a subject is selected due to a measured TPS of a sample from the subject that is at least about 50% (i.e., a TPS≥50%).

In embodiments, the TPS of a sample from a selected subject is no more than about 50% (i.e., a TPS<50%). In embodiments, the TPS of a sample from a selected subject is at least about 1% and less than or equal to 49%. In embodiments, a subject is selected due to a measured TPS of a sample from the subject that is at no more than about 50% (i.e., a TPS<50%). In embodiments, a subject is selected due to a measured TPS of a sample from the subject that is at least about 1% and less than or equal to 49%.

In embodiments, a sample is a tumor sample from a patient having lung cancer (e.g., NSCLC).

### Combined Positive Score (CPS)

In embodiments, PD-L1 expression is expressed as Combined Positive Score (CPS).

The Combined Positive Score (CPS) of a sample can be determined by the number of PD-L1 staining cells (tumor cells, lymphocytes, and macrophages) divided by the total number of viable tumor cells and then multiplied by 100. In embodiments, the TPS of a sample is determined using IHC.

In embodiments, a sample that expresses PD-L1 has a CPS of at least about 1 (i.e., a CPS≥1).

In embodiments, a positive PD-L1 expression is characterized by a CPS of at least about 1% (i.e., a CPS≥1%). In embodiments, a positive PD-L1 expression is characterized by a CPS of about 1% to 49%. In embodiments, a sample that expresses PD-L1 has a CPS of at least about 1% (i.e., a CPS≥1%). In embodiments, a sample that expresses PD-L1 has a CPS of at least about 5% (i.e., a CPS≥5%). In embodiments, a sample that expresses PD-L1 has a CPS of at least about 10% (i.e., a CPS≥10%). In embodiments, a sample that expresses PD-L1 has a CPS of about 1% to 49%. In embodiments, a sample that expresses PD-L1 has a CPS of at least about 50% (i.e., a CPS≥50%). In embodiments, a sample that expresses PD-L1 has a CPS of at least about 60% (i.e., a CPS≥60%). In embodiments, a sample that expresses PD-L1 has a CPS of at least about 70% (i.e., a CPS≥70%). In embodiments, a sample that expresses PD-L1 has a CPS of at least about 80% (i.e., a CPS≥80%). In embodiments, a sample that expresses PD-L1 has a CPS of at least about 90% (i.e., a CPS≥90%).

In embodiments, a high PD-L1 expression is characterized by a CPS of at least about 20% (i.e., a CPS≥20%). In embodiments, a high PD-L1 expression is characterized by a CPS of at least about 30% (i.e., a CPS≥30%). In embodiments, a high PD-L1 expression is characterized by a CPS of at least about 40% (i.e., a CPS≥40%). In embodiments, a high PD-L1 expression is characterized by a CPS of at least about 50% (i.e., a CPS≥50%). In embodiments, a high PD-L1 expression is characterized by a CPS of at least about 55% (i.e., a CPS≥55%). In embodiments, a high PD-L1 expression is characterized by a CPS of at least about 60% (i.e., a CPS≥60%).

In embodiments, a Combined Positive Score (CPS) of a sample is compared to a reference CPS. In embodiments, a subject is selected for treatment based on the CPS of a sample as compared to a reference CPS.

In embodiments, a reference level is a CPS of 0%. In embodiments, a sample does not express PD-L1 and the CPS of the sample is 0%. In embodiments, a subject is selected due to a measured CPS of a sample from the subject that is 0%. In embodiments, a reference level is a CPS of 1%. In embodiments, the CPS of a sample from a selected subject is at least about 1% (i.e., a CPS≥1%). In embodiments, a subject is selected due to a measured CPS of a sample from the subject that is at least about 1% (i.e., a CPS≥1%). In embodiments, the CPS of a sample from a selected subject is no more than about 1% (i.e., a CPS<1%). In embodiments, a subject is selected due to a measured CPS of a sample from the subject that is no more than about 1% (i.e., a CPS<1%).

In embodiments, a reference level is a CPS of 5%. In embodiments, the CPS of a sample from a selected subject is at least about 5% (i.e., a CPS≥1%). In embodiments, a subject is selected due to a measured CPS of a sample from the subject that is at least about 5% (i.e., a CPS≥5%). In embodiments, the CPS of a sample from a selected subject is no more than about 5% (i.e., a CPS<5%). In embodiments, a subject is selected due to a measured CPS of a sample from the subject that is no more than about 5% (i.e., a CPS<5%). In embodiments, a reference level is a CPS of 10%. In embodiments, the CPS of a sample from a selected subject is at least about 10% (i.e., a CPS≥10%). In embodiments, a subject is selected due to a measured CPS of a sample from the subject that is at least about 10% (i.e., a CPS≥10%). In embodiments, the CPS of a sample from a selected subject is no more than about 10% (i.e., a CPS<10%). In embodiments, a subject is selected due to a measured CPS of a sample from the subject that is no more than about 10% (i.e., a CPS<10%).

In embodiments, a reference level is a CPS of 25%. In embodiments, the CPS of a sample from a selected subject is no more than about 25% (i.e., a CPS<25%). In embodiments, a subject is selected due to a measured CPS of a sample from the subject that is no more than about 25% (i.e., a CPS<25%).

In embodiments, a reference level is a CPS of 50%. In embodiments, the CPS of a sample from a selected subject is at least about 50% (i.e., a CPS≥50%). In embodiments, a subject is selected due to a measured CPS of a sample from the subject that is at least about 50% (i.e., a CPS≥50%). In embodiments, the CPS of a sample from a selected subject is no more than about 50% (i.e., a CPS<50%).

In embodiments, the CPS of a sample from a selected subject is at least about 1% and less than or equal to 49%. In embodiments, a subject is selected due to a measured CPS of a sample from the subject that is at no more than about 50% (i.e., a CPS<50%). In embodiments, a subject is selected due to a measured CPS of a sample from the subject that is at least about 1% and less than or equal to 49%.

In embodiments, a sample is a tumor sample from a patient having lung cancer (e.g., NSCLC).

### Proportion of Tumor Area Occupied by PD-L1 Expressing Tumor-Infiltrating Immune Cells (% IC) of any Intensity

In embodiments, PD-L1 expression is expressed as the proportion of tumor area occupied by PD-L1 expressing tumor-infiltrating immune cells (% IC) of any intensity.

In embodiments, a positive PD-L1 expression is characterized by a % IC of at least about 1% (i.e., a % IC≥1%). In embodiments, a positive PD-L1 expression is characterized by a % IC of about 1% to 49%. In embodiments, a sample that expresses PD-L1 has a % IC of at least about 1% (i.e., a % IC≥1%). In embodiments, a sample that expresses PD-L1 has a % IC of at least about 5% (i.e., a % IC≥5%). In embodiments, a sample that expresses PD-L1 has a % IC of at least about 10% (i.e., a % IC≥10%). In embodiments, a sample that expresses PD-L1 has a % IC of about 1% to 49%. In embodiments, a sample that expresses PD-L1 has a % IC of at least about 50% (i.e., a % IC≥50%). In embodiments, a sample that expresses PD-L1 has a % IC of at least about 60% (i.e., a % IC≥60%). In embodiments, a sample that expresses PD-L1 has a % IC of at least about 70% (i.e., a % IC≥70%). In embodiments, a sample that expresses PD-L1 has a % IC of at least about 80% (i.e., a % IC≥80%). In embodiments, a sample that expresses PD-L1 has a % IC of at least about 90% (i.e., a % IC≥90%).

In embodiments, a high PD-L1 expression is characterized by a % IC of at least about 20% (i.e., a % IC≥20%). In embodiments, a high PD-L1 expression is characterized by a % IC of at least about 30% (i.e., a % IC≥30%). In embodiments, a high PD-L1 expression is characterized by a % IC of at least about 40% (i.e., a % IC≥40%). In embodiments, a high PD-L1 expression is characterized by a % IC of at least about 50% (i.e., a % IC≥50%). In embodiments, a high PD-L1 expression is characterized by a % IC of at least about 55% (i.e., a % IC≥55%). In embodiments, a high PD-L1 expression is characterized by a % IC of at least about 60% (i.e., a % IC≥60%).

In embodiments, a % IC of a sample is compared to a reference % IC. In embodiments, a subject is selected for treatment based on the % IC of a sample as compared to a reference % IC.

In embodiments, a reference level is a % IC of 0%. In embodiments, a sample does not express PD-L1 and the % IC of the sample is 0%. In embodiments, a subject is selected due to a measured % IC of a sample from the subject that is 0%. In embodiments, a reference level is a % IC of 1%. In embodiments, the % IC of a sample from a selected subject is at least about 1% (i.e., a % IC≥1%). In embodiments, a subject is selected due to a measured % IC of a sample from the subject that is at least about 1% (i.e., a % IC≥1%). In embodiments, the % IC of a sample from a selected subject is no more than about 1% (i.e., a % IC<1%). In embodiments, a subject is selected due to a measured % IC of a sample from the subject that is no more than about 1% (i.e., a % IC<1%).

In embodiments, a reference level is a % IC of 5%. In embodiments, the % IC of a sample from a selected subject is at least about 5% (i.e., a % IC≥1%). In embodiments, a subject is selected due to a measured % IC of a sample from the subject that is at least about 5% (i.e., a % IC≥5%). In embodiments, the % IC of a sample from a selected subject is no more than about 5% (i.e., a % IC<5%). In embodiments, a subject is selected due to a measured % IC of a sample from the subject that is no more than about 5% (i.e., a % IC<5%). In embodiments, a reference level is a % IC of 10%. In embodiments, the % IC of a sample from a selected subject is at least about 10% (i.e., a % IC≥10%). In embodiments, a subject is selected due to a measured % IC of a sample from the subject that is at least about 10% (i.e., a % IC≥10%). In embodiments, the % IC of a sample from a selected subject is no more than about 10% (i.e., a % IC<10%). In embodiments, a subject is selected due to a measured % IC of a sample from the subject that is no more than about 10% (i.e., a % IC<10%).

In embodiments, a reference level is a % IC of 25%. In embodiments, the % IC of a sample from a selected subject is no more than about 25% (i.e., a % IC<25%). In embodiments, a subject is selected due to a measured % IC of a sample from the subject that is no more than about 25% (i.e., a % IC<25%).

In embodiments, a reference level is a % IC of 50%. In embodiments, the % IC of a sample from a selected subject is at least about 50% (i.e., a % IC≥50%). In embodiments, a subject is selected due to a measured % IC of a sample from the subject that is at least about 50% (i.e., a % IC≥50%). In embodiments, the % IC of a sample from a selected subject is no more than about 50% (i.e., a % IC<50%).

In embodiments, the % IC of a sample from a selected subject is at least about 1% and less than or equal to 49%. In embodiments, a subject is selected due to a measured % IC of a sample from the subject that is at no more than about 50% (i.e., a % IC<50%). In embodiments, a subject is selected due to a measured % IC of a sample from the subject that is at least about 1% and less than or equal to 49%.

In embodiments, a sample is a tumor sample from a patient having lung cancer (e.g., NSCLC).

### Percentage of PD-L1 Expressing Tumor Cells (% TC) of any Intensity

In embodiments, PD-L1 expression is expressed as the percentage of PD-L1 expressing tumor cells (% TC) of any intensity

In embodiments, a positive PD-L1 expression is characterized by a % TC of at least about 1% (i.e., a % TC≥1%). In embodiments, a positive PD-L1 expression is characterized by a % TC of about 1% to 49%. In embodiments, a sample that expresses PD-L1 has a % TC of at least about 1% (i.e., a % TC≥1%). In embodiments, a sample that expresses PD-L1 has a % TC of at least about 5% (i.e., a % TC≥5%). In embodiments, a sample that expresses PD-L1 has a % TC of at least about 10% (i.e., a % TC≥10%). In embodiments, a sample that expresses PD-L1 has a % TC of about 1% to 49%. In embodiments, a sample that expresses PD-L1 has a % TC of at least about 50% (i.e., a % TC≥50%). In embodiments, a sample that expresses PD-L1 has a % TC of at least about 60% (i.e., a % TC≥60%). In embodiments, a sample that expresses PD-L1 has a % TC of at least about 70% (i.e., a % TC≥70%). In embodiments, a sample that expresses PD-L1 has a % TC of at least about 80% (i.e., a % TC≥80%). In embodiments, a sample that expresses PD-L1 has a % TC of at least about 90% (i.e., a % TC≥90%).

In embodiments, a high PD-L1 expression is characterized by a % TC of at least about 20% (i.e., a % TC≥20%). In embodiments, a high PD-L1 expression is characterized by a % TC of at least about 30% (i.e., a % TC≥30%). In embodiments, a high PD-L1 expression is characterized by a % TC of at least about 40% (i.e., a % TC≥40%). In embodiments, a high PD-L1 expression is characterized by a % TC of at least about 50% (i.e., a % TC≥50%). In embodiments, a high PD-L1 expression is characterized by a % TC of at least about 55% (i.e., a % TC≥55%). In embodiments, a high PD-L1 expression is characterized by a % TC of at least about 60% (i.e., a % TC≥60%).

In embodiments, a % TC of a sample is compared to a reference % TC. In embodiments, a subject is selected for treatment based on the % TC of a sample as compared to a reference % TC.

In embodiments, a reference level is a % TC of 0%. In embodiments, a sample does not express PD-L1 and the % TC of the sample is 0%. In embodiments, a subject is selected due to a measured % TC of a sample from the subject that is 0%. In embodiments, a reference level is a % TC of 1%. In embodiments, the % TC of a sample from a selected subject is at least about 1% (i.e., a % TC≥1%). In embodiments, a subject is selected due to a measured % TC of a sample from the subject that is at least about 1% (i.e., a % TC≥1%). In embodiments, the % TC of a sample from a selected subject is no more than about 1% (i.e., a % TC<1%). In embodiments, a subject is selected due to a measured % TC of a sample from the subject that is no more than about 1% (i.e., a % TC<1%).

In embodiments, a reference level is a % TC of 5%. In embodiments, the % TC of a sample from a selected subject is at least about 5% (i.e., a % TC≥1%). In embodiments, a subject is selected due to a measured % TC of a sample from the subject that is at least about 5% (i.e., a % TC≥5%). In embodiments, the % TC of a sample from a selected subject is no more than about 5% (i.e., a % TC<5%). In embodiments, a subject is selected due to a measured % TC of a sample from the subject that is no more than about 5% (i.e., a % TC<5%). In embodiments, a reference level is a % TC of 10%. In embodiments, the % TC of a sample from a selected subject is at least about 10% (i.e., a % TC≥10%). In embodiments, a subject is selected due to a measured % TC of a sample from the subject that is at least about 10% (i.e., a % TC≥10%). In embodiments, the % TC of a sample from a selected subject is no more than about 10% (i.e., a % TC<10%). In embodiments, a subject is selected due to a measured % TC of a sample from the subject that is no more than about 10% (i.e., a % TC<10%).

In embodiments, a reference level is a % TC of 25%. In embodiments, the % TC of a sample from a selected subject is no more than about 25% (i.e., a % TC<25%). In embodiments, a subject is selected due to a measured % TC of a sample from the subject that is no more than about 25% (i.e., a % TC<25%).

In embodiments, a reference level is a % TC of 50%. In embodiments, the % TC of a sample from a selected subject is at least about 50% (i.e., a % TC≥50%). In embodiments, a subject is selected due to a measured % TC of a sample from the subject that is at least about 50% (i.e., a % TC≥50%). In embodiments, the % TC of a sample from a selected subject is no more than about 50% (i.e., a % TC<50%).

In embodiments, the % TC of a sample from a selected subject is at least about 1% and less than or equal to 49%. In embodiments, a subject is selected due to a measured % TC of a sample from the subject that is at no more than about 50% (i.e., a % TC<50%). In embodiments, a subject is selected due to a measured % TC of a sample from the subject that is at least about 1% and less than or equal to 49%.

In embodiments, a sample is a tumor sample from a patient having lung cancer (e.g., NSCLC).

### PD-L1 Negative Cancer

In another aspect, the invention relates to methods for treating cancer that is not characterized by the expression of programmed death ligand 1 (PD-L1). In some embodiments, a patient has a cancer that does not express PD-L1 (i.e., a PD-L1 negative cancer).

### Therapeutic Methods

Methods described comprise the administration of a combination of therapeutic agents to a subject with cancer. In particular, the present disclosure provides a method of treating cancer in a subject, comprising administering a therapy that inhibits PD-1 signaling (“anti-PD-1 therapy”) and a therapy that inhibits PARP (“anti-PARP therapy”) to a subject so that the subject receives treatment with both therapies. In another aspect, the invention features a poly (ADP-ribose) polymerase (PARP) inhibitor and an anti-programmed death-1 protein (PD-1) inhibitor for simultaneous or sequential use in the treatment of cancer. Methods described herein can be particularly beneficial to a subject having cancer characterized by PD-L1 expression (including cancers characterized by high PD-L1 expression such as ≥50% PD-L1 expression).

In embodiments, a method described herein results in a therapeutic effect (e.g., a desired pharmacologic and/or physiologic effect). A therapeutic effect can encompass partially or completely curing a disease, relieving one or more adverse symptoms attributable to the disease, and/or delaying progression of the disease. To this end, the inventive method comprises administering a therapeutically effective amount of a therapeutic agent. A therapeutically effective amount can be an amount effective, at dosages and for periods of time necessary, to achieve a desired therapeutic result. A therapeutically effective amount may vary according to factors such as the disease state, age, sex, and weight of the individual, and the ability of the binding agent to elicit a desired response in the individual.

As used herein, the terms “treatment,” “treating,” and the like can refer to obtaining a desired pharmacologic and/or physiologic effect. In some embodiments, the effect can be therapeutic, i.e., the effect partially or completely cures a disease and/or adverse symptom attributable to the disease. To this end, the disclosed method can comprise administering a “therapeutically effective amount” of an immune checkpoint inhibitor. A “therapeutically effective amount” can refer to an amount effective, at dosages and for periods of time necessary, to achieve a desired therapeutic result. The therapeutically effective amount may vary according to factors such as the disease state, age, sex, and weight of the individual, and the ability of an immune checkpoint inhibitor to elicit a desired response in the individual.

Alternatively, the pharmacologic and/or physiologic effect may be prophylactic, i.e., the effect of completely or partially prevents a disease or symptom thereof (e.g., delaying onset or slowing progression of a disease or symptom thereof). In this respect, the inventive method comprises administering a “prophylactically effective amount” of the binding agent. A “prophylactically effective amount” refers to an amount effective, at dosages and for periods of time necessary, to achieve a desired prophylactic result. Therapeutic or prophylactic efficacy can be monitored by periodic assessment of treated patients. For repeated administrations over several days or longer, depending on the condition, the treatment can be repeated until a desired suppression of disease symptoms occurs, or alternatively, the treatment can be continued for the lifetime of the patient. However, other dosage regimens may be useful and can be within the scope of the disclosure. The desired dosage can be delivered by a single bolus administration of the composition, by multiple bolus administrations of the composition, or by continuous infusion administration of the composition.

In one aspect, the invention features a method of inducing an immune response in a subject, the method comprising: measuring a level of PD-L1 expression in a sample obtained from the subject; and administering to the subject based on the level of PD-L1 expression a therapeutically effective dose of an immune checkpoint inhibitor and a therapeutically effective dose of a PARP inhibitor. In embodiments, an immune checkpoint inhibitor is an anti-PD-1 agent (e.g., a PD-1 binding agent such as TSR-042).

In another aspect, the invention features a method of inducing an immune response in a subject, the method comprising: selecting a subject based a level of PD-L1 expression in a sample obtained from the subject as compared to a reference level; and administering to the selected subject a therapeutically effective dose of an immune checkpoint inhibitor and a therapeutically effective dose of a PARP inhibitor. In embodiments, an immune checkpoint inhibitor is an anti-PD-1 agent (e.g., a PD-1 binding agent such as TSR-042).

In embodiments, a mammal has a disorder that is characterized by PD-L1 expression. In some embodiments, such a method comprises administering an effective amount of a first immune checkpoint inhibitor. In some embodiments, such a method comprises administering an effective amount of a first immune checkpoint inhibitor and a second immune checkpoint inhibitor. In some embodiments, such a method comprises administering an effective amount of a first immune checkpoint inhibitor, a second immune checkpoint inhibitor, and a third immune checkpoint inhibitor. In some embodiments, such a method comprises administering an effective amount of an immune checkpoint inhibitor that is a polypeptide. In some embodiments, such a method comprises administering an effective amount of an isolated nucleic acid encoding polypeptide that is an immune checkpoint inhibitor. In some embodiments, such a method comprises administering an effective amount of a vector that encodes an immune checkpoint inhibitor that is a polypeptide. In some embodiments, such a method comprises administering an effective amount of an isolated cell comprising a nucleic acid or a vector encoding an immune checkpoint inhibitor that is a polypeptide. In some embodiments, such a method comprises administering an effective amount of a composition comprising a polypeptide, nucleic acid, vector or cell as described herein. In some embodiments, upon administration of a polypeptide, nucleic acid, vector, cell or composition of the present disclosure, an immune response is induced in the mammal.

In one aspect, the invention features a method of enhancing an immune response or increasing the activity of an immune cell in a subject, the method comprising: measuring a level of PD-L1 expression in a sample obtained from the subject; and administering to the subject based on the level of PD-L1 expression a therapeutically effective dose of an immune checkpoint inhibitor (e.g., an anti-PD-1 agent) and a PARP inhibitor. In embodiments, an immune checkpoint inhibitor is an anti-PD-1 agent (e.g., a PD-1 binding agent such as TSR-042).

In another aspect, the invention features a method of enhancing an immune response or increasing the activity of an immune cell in a subject, the method comprising: selecting a subject based a level of PD-L1 expression in a sample obtained from the subject as compared to a reference level; and administering to the selected subject a therapeutically effective dose of an immune checkpoint inhibitor and a PARP inhibitor. In embodiments, an immune checkpoint inhibitor is an anti-PD-1 agent (e.g., a PD-1 binding agent such as TSR-042).

In embodiments, a mammal has a disorder that is responsive to immune checkpoint inhibition and characterized by PD-L1 expression. In some embodiments, such a method comprises administering an effective amount of a first immune checkpoint inhibitor. In some embodiments, such a method comprises administering an effective amount of a first immune checkpoint inhibitor and a second immune checkpoint inhibitor. In some embodiments, such a method comprises administering an effective amount of a first immune checkpoint inhibitor, a second immune checkpoint inhibitor, and a third immune checkpoint inhibitor. In some embodiments, such a method comprises administering an effective amount of an immune checkpoint inhibitor that is a polypeptide. In some embodiments, such a method comprises administering an effective amount of an isolated nucleic acid encoding polypeptide that is an immune checkpoint inhibitor. In some embodiments, such a method comprises administering an effective amount of a vector that encodes an immune checkpoint inhibitor that is a polypeptide. In some embodiments, such a method comprises administering an effective amount of an isolated cell comprising a nucleic acid or a vector encoding an immune checkpoint inhibitor that is a polypeptide. In some embodiments, such a method comprises administering an effective amount of a composition comprising a polypeptide, nucleic acid, vector or cell as described herein. In some embodiments, upon administration of a polypeptide, nucleic acid, vector, cell or composition of the present disclosure, an immune response is induced in the mammal. In some embodiments, the immune response is a humoral or cell mediated immune response. In some embodiments, the immune response is a CD4 or CD8 T cell response. In some embodiments, the immune response is a B cell response.

In another aspect, the invention features a poly (ADP-ribose) polymerase (PARP) inhibitor and an anti-programmed death-1 protein (PD-1) inhibitor for simultaneous or sequential use in the treatment of cancer. In embodiments, the human has at least one solid tumour. In embodiments, the human has not previously received systemic chemotherapy and/or any previous anti-PD-1 therapy. In embodiments, the PD-L1 expression level in a solid tumour is high.

Thus, the present disclosure further provides a method of treating cancer in a subject. The method can comprise administering the aforementioned composition to a subject, whereupon the disorder is treated in the mammal.

### PARP Inhibitors

In embodiments, an additional therapy is a poly (ADP-ribose) polymerase (PARP) inhibitor.

In embodiments, the invention features a use of a poly (ADP-ribose) polymerase (PARP) inhibitor in the manufacture of a medicament for use in treating cancer in a human patient; where the PARP inhibitor is to be administered to said human in combination, simultaneously or sequentially in any order, with an anti-programmed death-1 protein (PD-1) inhibitor. In embodiments, the human has at least one solid tumour. In embodiments, the human has not previously received systemic chemotherapy and/or any previous anti-PD-1 therapy. In embodiments, the PD-L1 expression level in a solid tumour is high.

### Role of Poly(ADP-Ribose) Polymerases (PARPs)

Poly(ADP-ribose) polymerases (PARPs) are a family of enzymes that cleave NAD+, releasing nicotinamide, and successively add ADP-ribose units to form ADP-ribose polymers. Accordingly, activation of PARP enzymes can lead to depletion of cellular NAD+ levels (e.g., PARPs as NAD+ consumers) and mediates cellular signaling through ADP-ribosylation of downstream targets. PARP-1 is a zinc-finger DNA-binding enzyme that is activated by binding to DNA double or single strand breaks. It was known that anti-alkylating agents could deplete the NAD+ content of tumor cells, and the discovery of PARPs explained this phenomenon. (Parp Inhibitors and Cancer Therapy. Curtin N. in Poly ADP Ribosylation. ed. Alexander Burke, Lands Bioscience and Springer Bioscience, 2006: 218-233). Anti-alkylating agents induce DNA strand breaks, which activates of PARP-1, which is part of the DNA repair pathway. Poly ADP-ribosylation of nuclear proteins by PARP-1 converts DNA damage into intracellular signals that can either activate DNA repair (e.g. by the base excision repair (BER) pathway); or trigger cell death in the presence of DNA damage that is too extensive and cannot be efficiently repaired.

PARP-2 contains a catalytic domain and is capable of catalyzing a poly(ADP-ribosyl)ation reaction. PARP-2 displays auto-modification properties similar to PARP-1. The protein is localized in the nucleus in vivo and may account for the residual poly(ADP-ribose) synthesis observed in PARP-1-deficient cells, treated with alkylating agents or hydrogen peroxide. Some agents that inhibit PARP (e.g., agents primarily aimed at inhibiting PARP-1) may also inhibit PARP-2 (e.g., niraparib).

The role of PARP enzymes in DNA damage response (e.g. repair of DNA in response to genotoxic stress) has led to the compelling suggestion that PARP inhibitors may be useful anti-cancer agents. PARP inhibitors may be particularly effective in treating cancers resulting from germ line or sporadic deficiency in the homologous recombination DNA repair pathway, such as BRCA-1 and/or BRCA-2 deficient cancers.

Pre-clinical ex vivo and in vivo experiments suggest that PARP inhibitors are selectively cytotoxic for tumors with homozygous inactivation of BRCA-1 and/or BRCA-2 genes, which are known to be important in the homologous recombination (HR) DNA repair pathway. The biological basis for the use of PARP inhibitors as single agents in cancers with defects in BRCA-1 and/or BRCA-2 is the requirement of PARP-1 and PARP-2 for base excision repair (BER) of the damaged DNA. Upon formation of single-strand DNA breaks, PARP-1 and PARP-2 bind at sites of lesions, become activated, and catalyze the addition of long polymers of ADP-ribose (PAR chains) on several proteins associated with chromatin, including histones, PARP itself, and various DNA repair proteins. This results in chromatin relaxation and fast recruitment of DNA repair factors that access and repair DNA breaks. Normal cells repair up to 10,000 DNA defects daily and single strand breaks are the most common form of DNA damage. Cells with defects in the BER pathway enter S phase with unrepaired single strand breaks. Pre-existing single strand breaks are converted to double strand breaks as the replication machinery passes through the break. Double strand breaks present during S phase are preferentially repaired by the error-free HR pathway. Cells with inactivation of genes required for HR, such as BRCA-1 and/or BRCA-2, accumulate stalled replication forks during S phase and may use error-prone non-homologous end joining (NHEJ) to repair damaged DNA. Both the inability to complete S phase (because of stalled replication forks) and error-prone repair by NHEJ, are thought to contribute to cell death.

Without wishing to be bound by theory, it is hypothesized that treatment with PARP inhibitors may selectively kill a subset of cancer cells with deficiencies in DNA repair pathways (e.g., inactivation of BRCA-1 and/or BRCA-2). For example, a tumor arising in a patient with a germline BRCA mutation has a defective homologous recombination DNA repair pathway and would be increasingly dependent on BER, a pathway blocked by PARP inhibitors, for maintenance of genomic integrity. This concept of inducing death by use of PARP inhibitors to block one DNA repair pathway in tumors with pre-existing deficiencies in a complementary DNA repair pathways is called synthetic lethality.

The therapeutic potential of PARP inhibitors is further expanded by the observation that PARP inhibitors not only have monotherapy activity in HR-deficient tumors, but are also effective in preclinical models in combination with other agents such as cisplatin, carboplatin, alkylating and methylating agents, radiation therapy, and topoisomerase I inhibitors. In contrast to the rationale for monotherapy in which PARP inhibition alone is sufficient for cell death in HR-deficient cancers (due to endogenous DNA damage), PARP is required for repair of DNA damage induced by standard cytotoxic chemotherapy. In some cases, the specific role of PARP is not known, but PARP is known to be required to release trapped topoisomerase I/irinotecan complexes from DNA. Temozolomide-induced DNA damage is repaired by the BER pathway, which requires PARP to recruit repair proteins. Combination therapies that enhance or synergize the cancer therapy without significantly increasing toxicity would provide substantial benefit to cancer patients, including ovarian cancer patients.

### PARP Inhibitors

Without wishing to be bound by theory, treatment with PARP inhibitors (e.g., PARP-1/2 inhibitors) may selectively kill a subset of cancer cell types by exploiting their deficiencies in DNA repair. Human cancers exhibit genomic instability and an increased mutation rate due to underlying defects in DNA repair. These deficiencies render cancer cells more dependent on the remaining DNA repair pathways and targeting these pathways is expected to have a much greater impact on the survival of the tumor cells than on normal cells.

In embodiments, a PARP inhibitor inhibits PARP-1 and/or PARP-2. In some embodiments, the agent is a small molecule, a nucleic acid, a polypeptide (e.g., an antibody), a carbohydrate, a lipid, a metal, or a toxin. In related embodiments, the agent is ABT-767, AZD 2461, BGB-290, BGP 15, CEP 8983, CEP 9722, DR 2313, E7016, E7449, fluzoparib (SHR 3162), IMP 4297, INO1001, JPI 289, JPI 547, monoclonal antibody B3-LysPE40 conjugate, MP 124, niraparib (ZEJULA) (MK-4827), NU 1025, NU 1064, NU 1076, NU1085, olaparib (AZD2281), ON02231, PD 128763, R 503, R554, rucaparib (RUBRACA) (AG-014699, PF-01367338), SBP 101, SC 101914, simmiparib, talazoparib (BMN-673), veliparib (ABT-888), WW 46, 2-(4-(trifluoromethyl)phenyl)-7,8-dihydro-5H-thiopyrano[4,3-d]pyrimidin-4-ol, and salts or derivatives thereof. In some embodiments, an agent that inhibits PARP is a small molecule. In some embodiments, an agent that inhibits PARP is an antibody agent. In some embodiments, an agent that inhibits PARP is a combination of agents. In some certain embodiments, a PARP inhibitor is niraparib, olaparib, rucaparib, talazoparib, veliparib, or any combination thereof. In some embodiments, a PARP inhibitor can be prepared as a pharmaceutically acceptable salt. In some related embodiments, an agent is niraparib, olaparib, rucaparib, talazoparib, veliparib, or salts or derivatives thereof. In certain embodiments, an agent is niraparib or a salt or derivative thereof. In certain embodiments, an agent is olaparib or a salt or derivative thereof. In certain embodiments, an agent is rucaparib or a salt or derivative thereof. In certain embodiments, an agent is talazoparib or a salt or derivative thereof. In certain embodiments, an agent is veliparib or a salt or derivative thereof. One of skill in the art will appreciate that such salt forms can exist as solvated or hydrated polymorphic forms.

Target engagement has also been demonstrated by measuring PARP activity in tumor homogenates from tumor xenograft studies. Niraparib has been shown to induce cell cycle arrest, particularly arrest in the G2/M phase of the cell cycle. Accordingly, in some embodiments, the present invention provides a method of inducing cell cycle arrest of a tumor cell, the method comprising administering niraparib to a patient in need thereof. In some embodiments, the present invention provides a method of inducing arrest of the G2/M phase of the cell cycle of a tumor cell, the method comprising administering niraparib to a patient in need thereof. In some embodiments, the present invention provides a method of inducing arrest in the G2/M phase of the cell cycle of BRCA-1 and/or BRCA-2-deficient cells, the method comprising administering niraparib to a patient in need thereof.

### Niraparib

Niraparib, (3S)-3-[4-{7-(aminocarbonyl)-2H-indazol-2-yl}phenyl]piperidine, is an orally available, potent, poly (adenosine diphosphate [ADP]-ribose) polymerase (PARP)-1 and -2 inhibitor. See WO 2008/084261 (published on Jul. 17, 2008) and WO 2009/087381 (published Jul. 16, 2009), the entirety of each of which is hereby incorporated by reference. Niraparib can be prepared according to Scheme 1 of WO 2008/084261.

As used herein, the term “niraparib” means any of the free base compound ((3S)-3-[4-{7-(aminocarbonyl)-2H-indazol-2-yl}phenyl]piperidine), a salt form, including pharmaceutically acceptable salts, of (3S)-3-[4-{7-(aminocarbonyl)-2H-indazol-2-yl}phenyl]piperidine (e.g., (3S)-3-[4-{7-(aminocarbonyl)-2H-indazol-2-yl}phenyl]piperidine tosylate), or a solvated or hydrated form thereof (e.g., (3S)-3-[4-{7-(aminocarbonyl)-2H-indazol-2-yl}phenyl]piperidine tosylate monohydrate). In some embodiments, such forms may be individually referred to as “niraparib free base”, “niraparib tosylate” and “niraparib tosylate monohydrate”, respectively. Unless otherwise specified, the term “niraparib” includes all forms of the compound (3S)-3-[4-{7-(aminocarbonyl)-2H-indazol-2-yl}phenyl]piperidine.

In some embodiments, niraparib can be prepared as a pharmaceutically acceptable salt. One of skill in the art will appreciate that such salt forms can exist as solvated or hydrated polymorphic forms. In some embodiments, niraparib is prepared in the form of a hydrate.

In certain embodiments, niraparib is prepared in the form of a tosylate salt. In some embodiments, niraparib is prepared in the form of a tosylate monohydrate. The molecular structure of the tosylate monohydrate salt of niraparib is shown below:

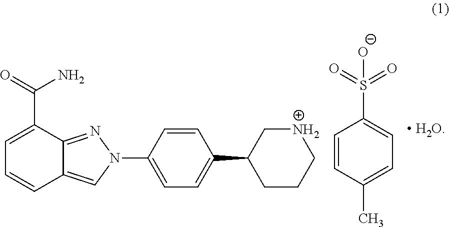

The crystalline tosylate monohydrate salt of niraparib is being developed as a monotherapy agent for tumors with defects in the homologous recombination (HR) deoxyribonucleic acid (DNA) repair pathway and as a sensitizing agent in combination with cytotoxic agents and radiotherapy.

Niraparib is a potent and selective PARP-1 and PARP-2 inhibitor with inhibitory concentration at 50% of control (IC₅₀)=3.8 and 2.1 nM, respectively, and is at least 100-fold selective over other PARP-family members. Niraparib inhibits PARP activity, stimulated as a result of DNA damage caused by addition of hydrogen peroxide, in various cell lines with an IC₅₀ and an inhibitory concentration at 90% of control (IC₉₀) of about 4 and 50 nM, respectively.

Niraparib demonstrates selective anti-proliferative activity for cancer cell lines that have been silenced for BRCA-1 or BRCA-2, or carry BRCA-1 or BRCA-2 mutations compared to their wild type counterparts. The antiproliferative activity of niraparib on BRCA-defective cells is a consequence of a cell cycle arrest in G2/M followed by apoptosis. Niraparib is also selectively cytotoxic for selected Ewing's sarcoma, acute lymphocytic leukemia (ALL), non-small cell lung cancer (NSCLC), and small cell lung cancer (SCLC) cell lines, as well as for tumor cell lines carrying homozygous inactivation of the ATM gene. Niraparib demonstrates weak activity on normal human cells. In vivo studies demonstrated strong antitumor activity with BRCA-1 mutant breast cancer (MDA-MB-436), BRCA-2 mutant pancreatic cancer (CAPAN-1), ATM-mutant mantle cell lymphoma (GRANTA-519), serous ovarian cancer (OVCAR3), colorectal cancer (HT29 and DLD-1), patient derived Ewing's sarcoma, and TNBC xenograft models in mice.

In embodiments, niraparib is administered at a dose equivalent to about 100 mg of niraparib free base (e.g., a pharmaceutically acceptable salt of niraparib such as niraparib tosylate monohydrate is administered at a dose equivalent to about 100 mg of niraparib free base). In embodiments, niraparib is administered at a dose equivalent to about 200 mg of niraparib free base (e.g., a pharmaceutically acceptable salt of niraparib such as niraparib tosylate monohydrate is administered at a dose equivalent to about 200 mg of niraparib free base In embodiments, niraparib is administered at a dose equivalent to about 300 mg of niraparib free base (e.g., a pharmaceutically acceptable salt of niraparib such as niraparib tosylate monohydrate is administered at a dose equivalent to about 300 mg of niraparib free base).

Agents that Inhibit PD-1 Signaling

Programmed Death 1 (PD-1) (also known as Programmed Cell Death 1) (encoded by the gene Pdcd1) is a type I transmembrane protein of 268 amino acids originally identified by subtractive hybridization of a mouse T cell line undergoing apoptosis (Ishida et al., Embo J., 11: 3887-95 (1992)). The normal function of PD-1, expressed on the cell surface of activated T cells under healthy conditions, is to down-modulate unwanted or excessive immune responses, including autoimmune reactions.

PD-1 is a member of the CD28/CTLA-4 family of T-cell regulators, and is expressed on activated T-cells, B-cells, and myeloid lineage cells (Greenwald et al., Annu. Rev. Immunol., 23: 515-548 (2005); and Sharpe et al., Nat. Immunol., 8: 239-245 (2007)). PD-1 is an inhibitory member of the CD28 family of receptors that also includes CD28, CTLA-4, ICOS and BTLA. PD-1 is expressed on activated B cells, T cells, and myeloid cells (Agata et al., supra; Okazaki et al. (2002) Curr. Opin. Immunol 14:391779-82; Bennett et al. (2003) J. Immunol. 170:711-8).

Two ligands for PD-1 have been identified, PD ligand 1 (PD-L1) and PD ligand 2 (PD-L2), both of which belong to the B7 protein superfamily (Greenwald et al, supra). PD-1 has been shown to negatively regulate antigen receptor signaling upon engagement of its ligands (PD-L1 and/or PD-L2).

Favorable response rates have been observed with certain PD-1/L1 checkpoint inhibitors in the clinic, however, there remains a considerable unmet need for alternative treatments in patients who exhibit primary resistance or suffer a relapse due to acquired or adaptive immune resistance. (Sharma et al., Cell, 2017; 168(4):707-723).

In one aspect, the invention features a method of inducing an immune response in a subject, the method comprising: measuring a level of PD-L1 expression in a sample obtained from the subject; and administering to the subject based on the level of PD-L1 expression a therapeutically effective dose of an immune checkpoint inhibitor. In another aspect, the invention features a method of enhancing an immune response or increasing the activity of an immune cell in a subject, the method comprising: measuring a level of PD-L1 expression in a sample obtained from the subject; and administering to the subject based on the level of PD-L1 expression a therapeutically effective dose of an immune checkpoint inhibitor. In still another aspect, the invention features a method of treating a subject, the method comprising: measuring a level of PD-L1 expression in a sample obtained from the subject; and administering to the subject based on the level of PD-L1 expression a therapeutically effective dose of an immune checkpoint inhibitor.

In another aspect, the invention features a method of inducing an immune response in a subject, the method comprising: selecting a subject based a level of PD-L1 expression in a sample obtained from the subject as compared to a reference level; and administering to the selected subject a therapeutically effective dose of an immune checkpoint inhibitor. In another aspect, the invention features a method of enhancing an immune response or increasing the activity of an immune cell in a subject, the method comprising: selecting a subject based a level of PD-L1 expression in a sample obtained from the subject as compared to a reference level; and administering to the selected subject a therapeutically effective dose of an immune checkpoint inhibitor. In still another aspect, the invention features a method of treating a subject, the method comprising: selecting a subject based a level of PD-L1 expression in a sample obtained from the subject as compared to a reference level; and administering to the selected subject a therapeutically effective dose of an immune checkpoint inhibitor.

In embodiments, a mammal has a disorder that is responsive to Programmed death-1 protein (PD-1) inhibition. In embodiments, a mammal has a disorder that is responsive to Programmed death-1 protein (PD-1) inhibition and characterized by PD-L1 expression. In some embodiments, such a method comprises administering an effective amount of an agent that is capable of inhibiting Programmed death-1 protein (PD-1) signaling (PD-1 agent). In some embodiments, such a method comprises administering an effective amount of an agent that is capable of inhibiting Programmed death-1 protein (PD-1) signaling (PD-1 agent) and an effective amount of a second immune checkpoint inhibitor (e.g., an effective amount of an agent that is capable of Lymphocyte Activation Gene-3 (LAG-3) signaling (LAG-3 agent) or an effective amount of an agent that is capable of inhibiting T cell immunoglobulin and mucin protein 3 (TIM-3) signaling (TIM-3 agent)). In some embodiments, such a method comprises administering an effective amount of an agent that is capable of inhibiting programmed death-1 protein (PD-1) signaling (PD-1 agent) and an effective amount of an agent capable of inhibiting Lymphocyte Activation Gene-3 (LAG-3) signaling (LAG-3 agent). In some embodiments, such a method comprises administering an effective amount of an agent that is capable of inhibiting programmed death-1 protein (PD-1) signaling (PD-1 agent) and an effective amount of an agent that is capable of inhibiting T cell immunoglobulin and mucin protein 3 (TIM-3) signaling (TIM-3 agent). In some embodiments, such a method comprises administering an effective amount of an agent that is capable of inhibiting programmed death-1 protein (PD-1) signaling (PD-1 agent), an effective amount of an agent that is capable of inhibiting Lymphocyte Activation Gene-3 (LAG-3) signaling (LAG-3 agent), and an effective amount of an agent that is capable of inhibiting T cell immunoglobulin and mucin protein 3 (TIM-3) signaling (TIM-3 agent). In some embodiments, such a method comprises administering an effective amount of a polypeptide that is capable of binding PD-1. In some embodiments, such a method comprises administering an effective amount of an isolated nucleic acid encoding polypeptide that is capable of binding PD-1. In some embodiments, such a method comprises administering an effective amount of a vector that encodes a polypeptide that is capable of binding PD-1. In some embodiments, such a method comprises administering an effective amount of an isolated cell comprising a nucleic acid or a vector encoding polypeptide that is capable of binding PD-1. In some embodiments, such a method comprises administering an effective amount of a composition comprising a polypeptide, nucleic acid, vector or cell as described herein. In some embodiments, upon administration of a polypeptide, nucleic acid, vector, cell or composition of the present disclosure, an immune response is induced in the mammal. In some embodiments, the immune response is a humoral or cell mediated immune response. In some embodiments, the immune response is a CD4 or CD8 T cell response. In some embodiments, the immune response is a B cell response. In embodiments, a LAG-3 agent is TSR-033. In embodiments, a PD-1 agent is TSR-042. In embodiments, a TIM-3 agent is TSR-022. In embodiments, a disorder is cancer.

In another aspect, the invention features a use of an anti-programmed death-1 protein (PD-1) inhibitor in the manufacture of a medicament for use in treating cancer in a human patient; where the anti-PD-1 inhibitor is to be administered to said human in combination, simultaneously or sequentially in any order, with a poly (ADP-ribose) polymerase (PARP) inhibitor. In embodiments, the human has at least one solid tumour. In embodiments, the human has not previously received systemic chemotherapy and/or any previous anti-PD-1 therapy. In embodiments, the PD-L1 expression level in a solid tumour is high.

Agents that inhibit PD-1 signaling for use in therapies of the present disclosure include those that bind to and block PD-1 receptors on T cells without triggering inhibitory signal transduction, agents that bind to PD-1 ligands to prevent their binding to PD-1, agents that do both, and agents that prevent expression of genes that encode either PD-1 or natural ligands of PD-1. Compounds that bind to natural ligands of PD-1 include PD-1 itself, as well as active fragments of PD-1, and in the case of the B7-H1 ligand, B7.1 proteins and fragments. Such antagonists include proteins, antibodies, anti-sense molecules and small organics.

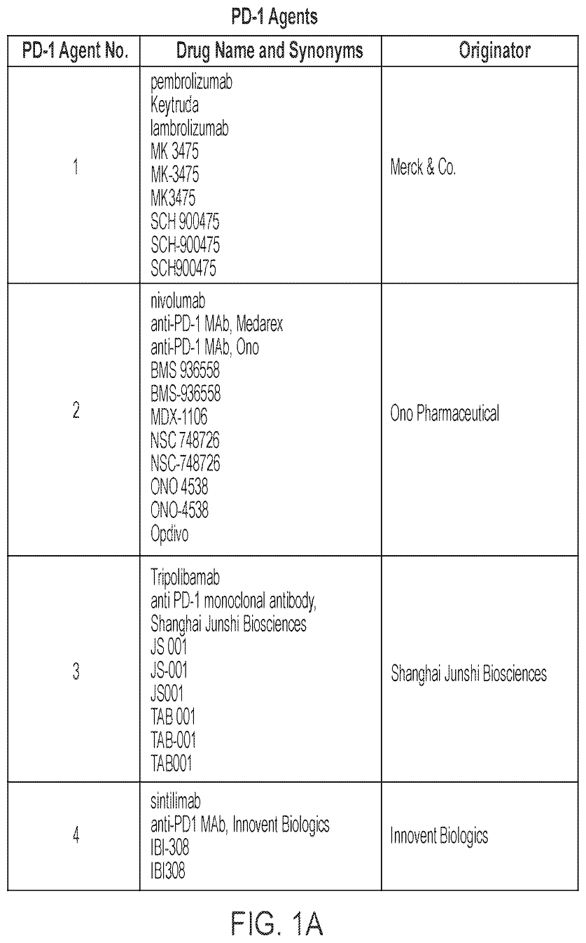

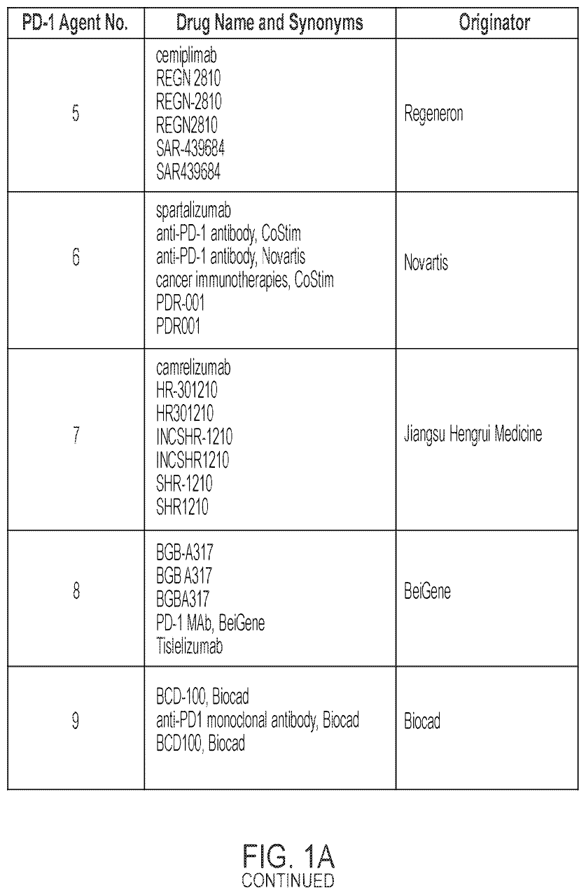

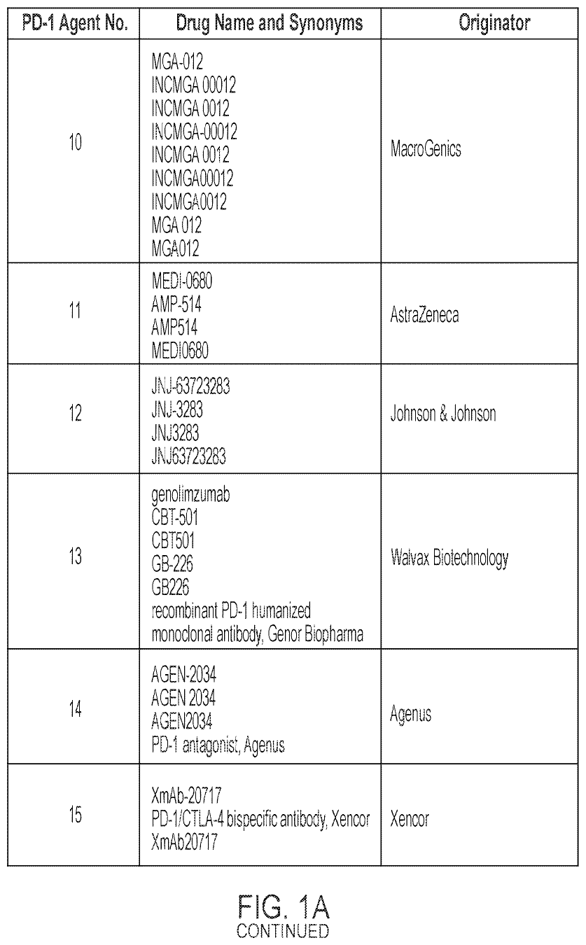

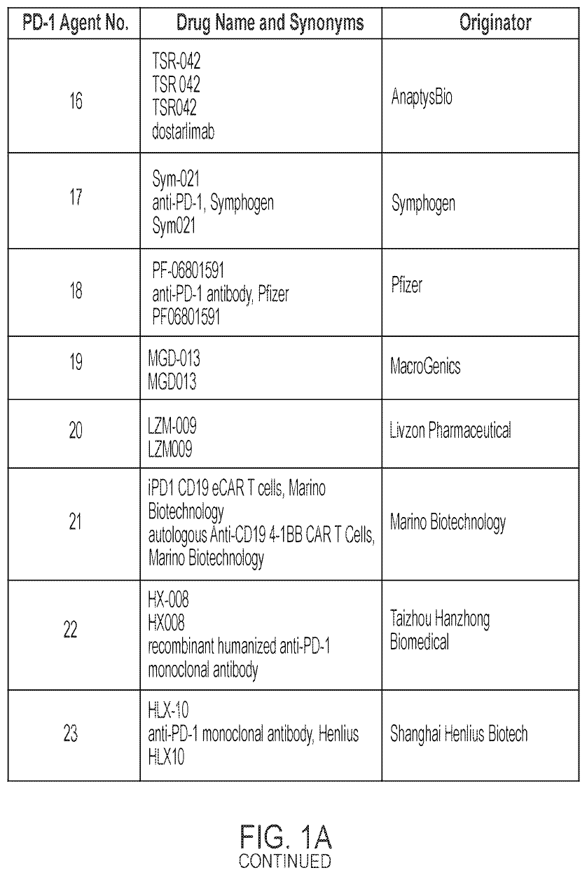

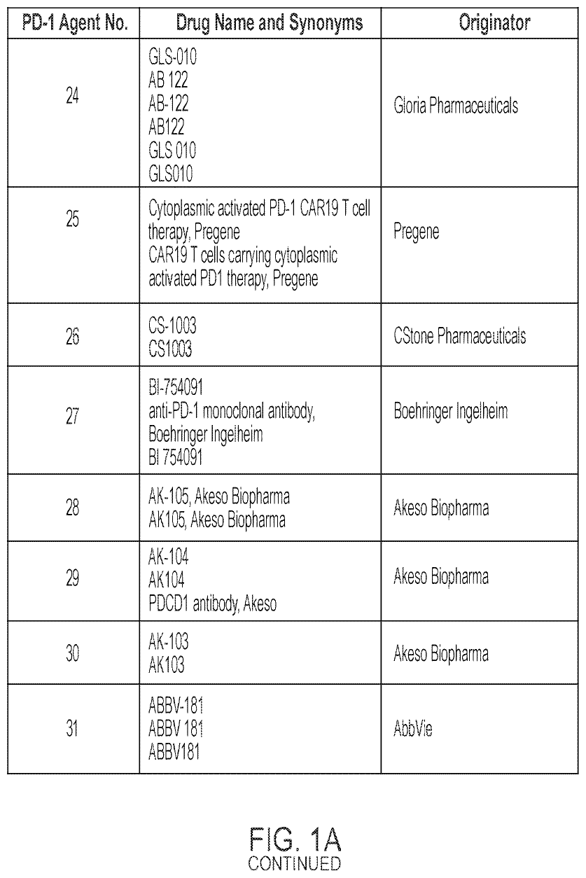

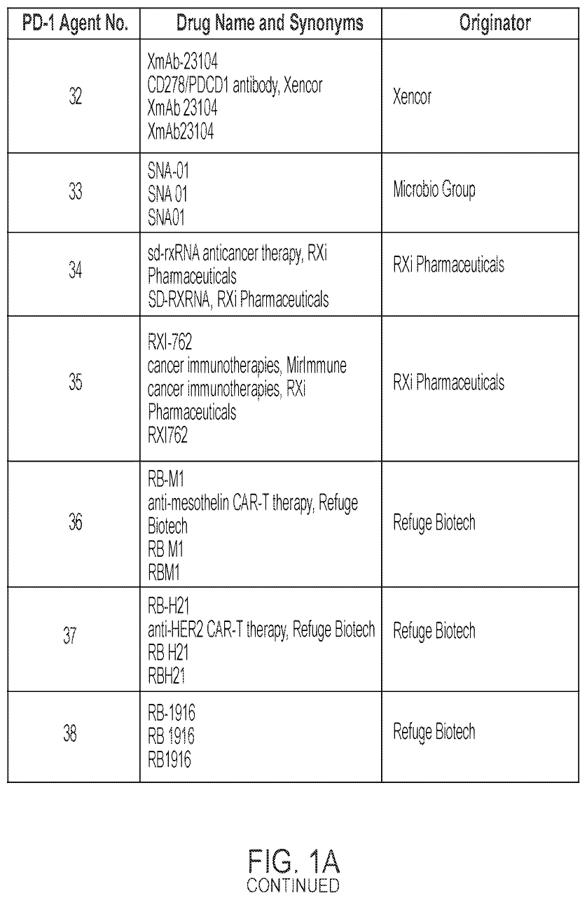

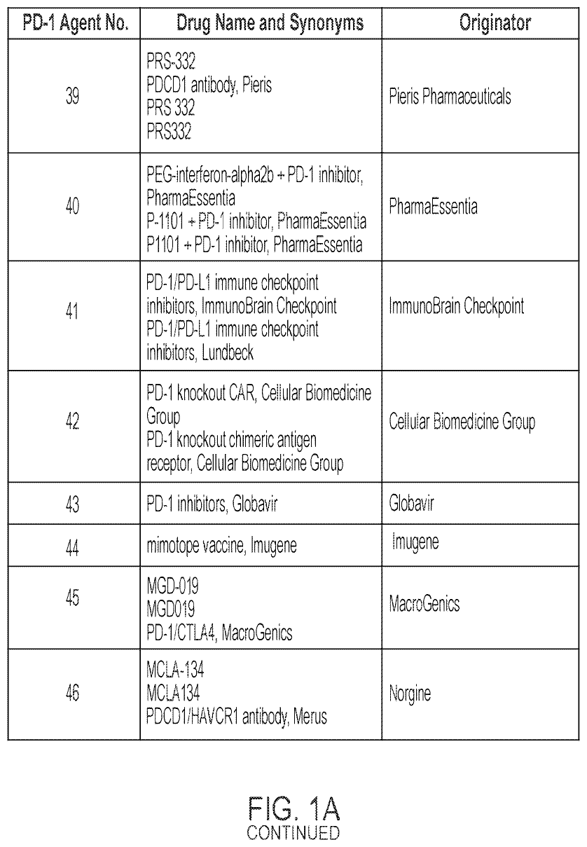

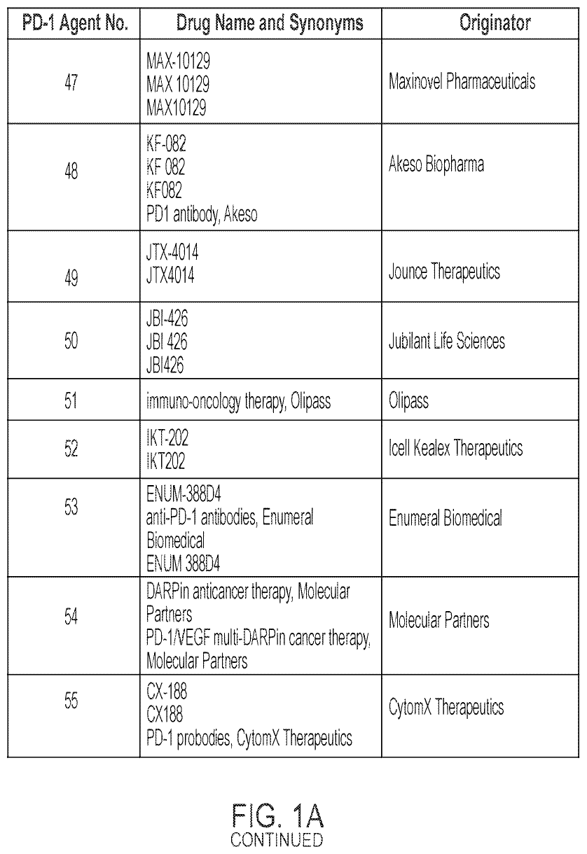

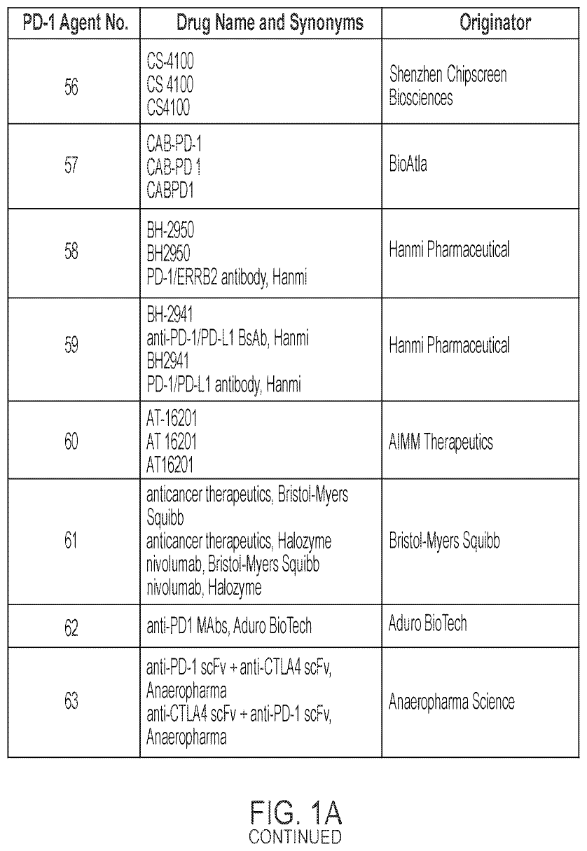

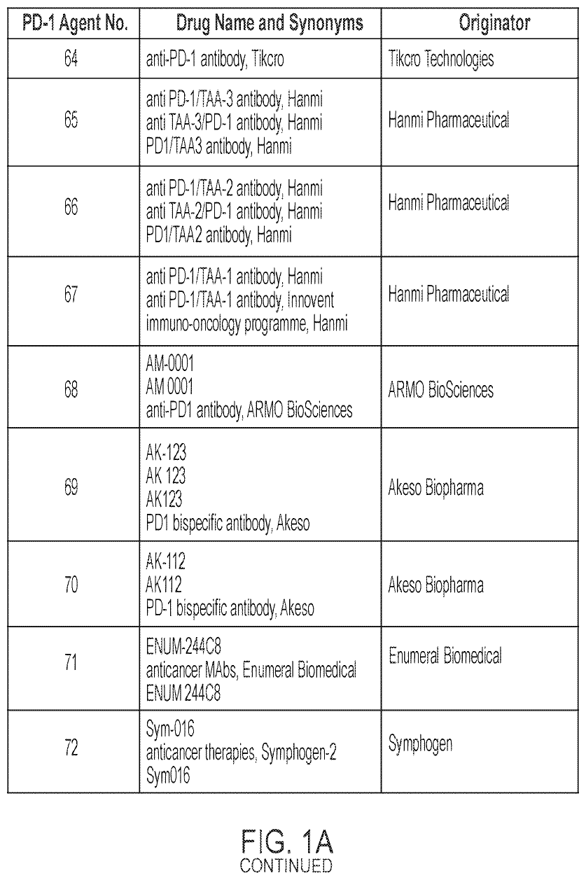

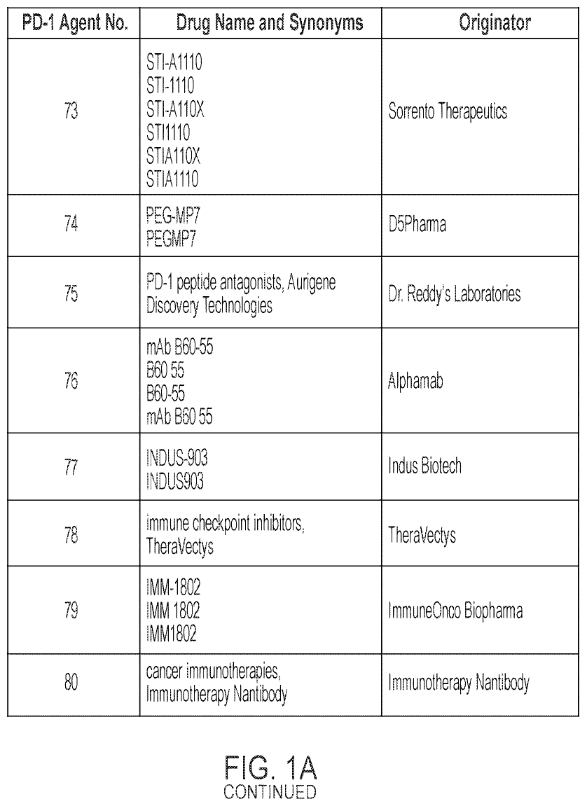

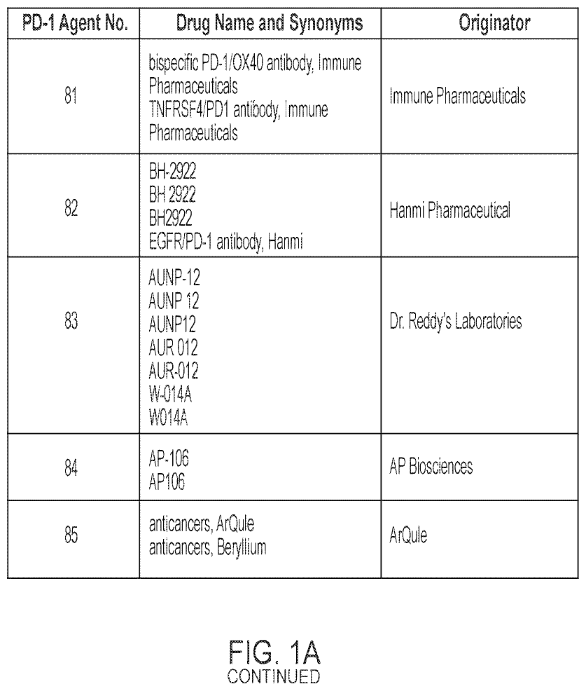

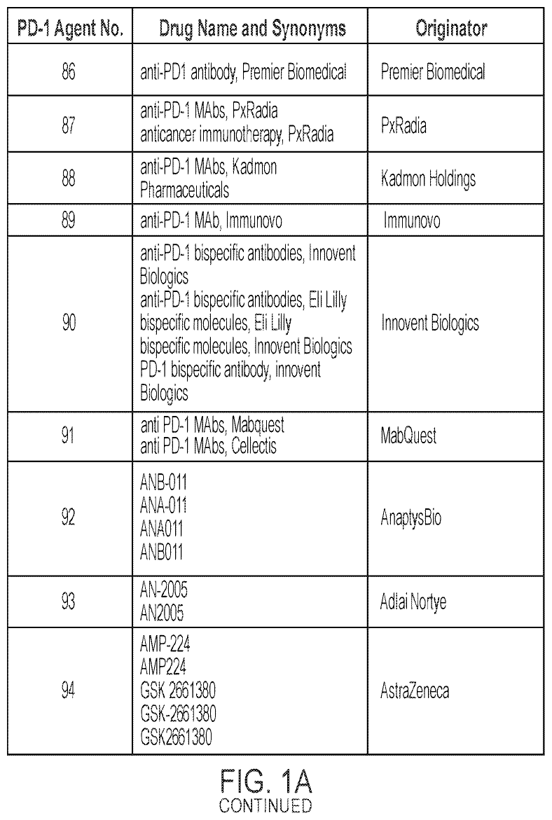

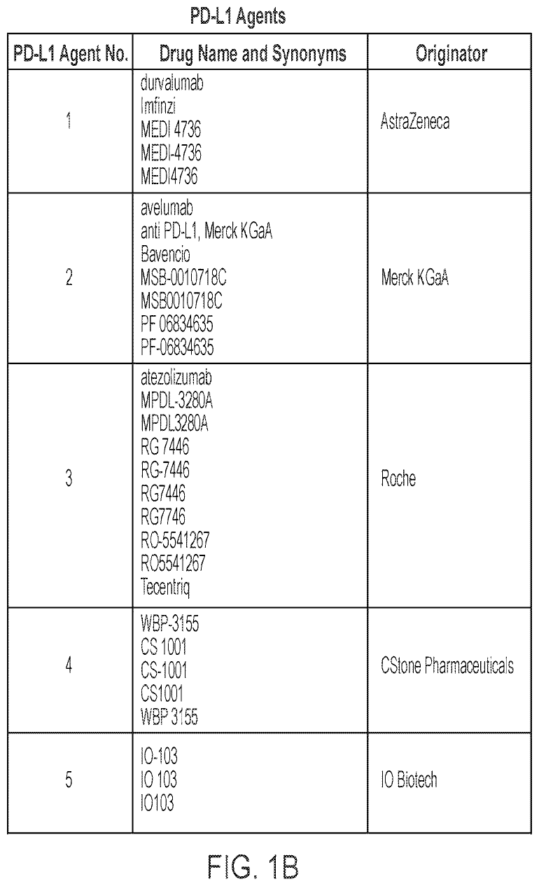

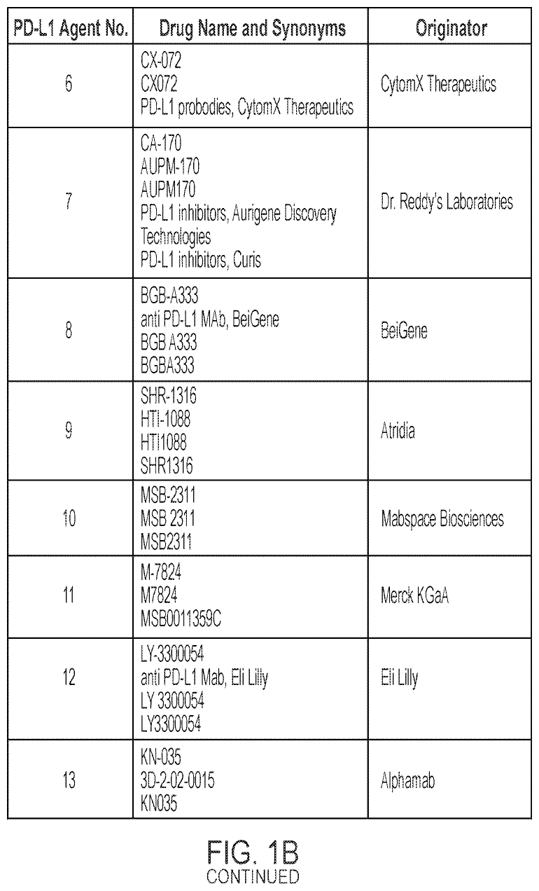

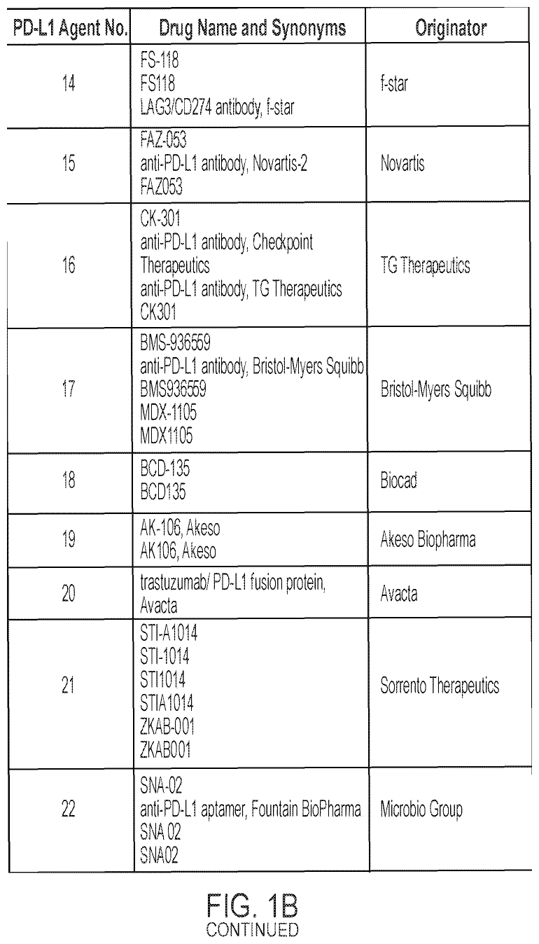

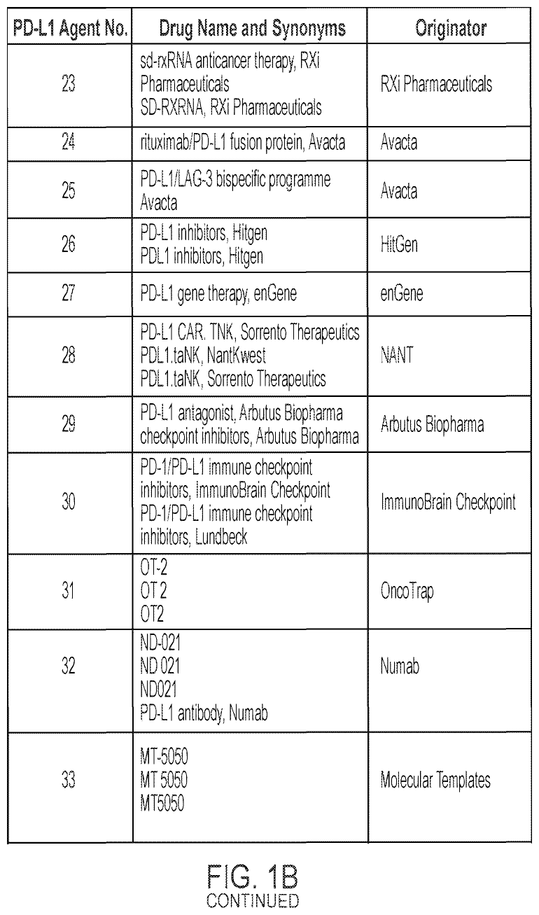

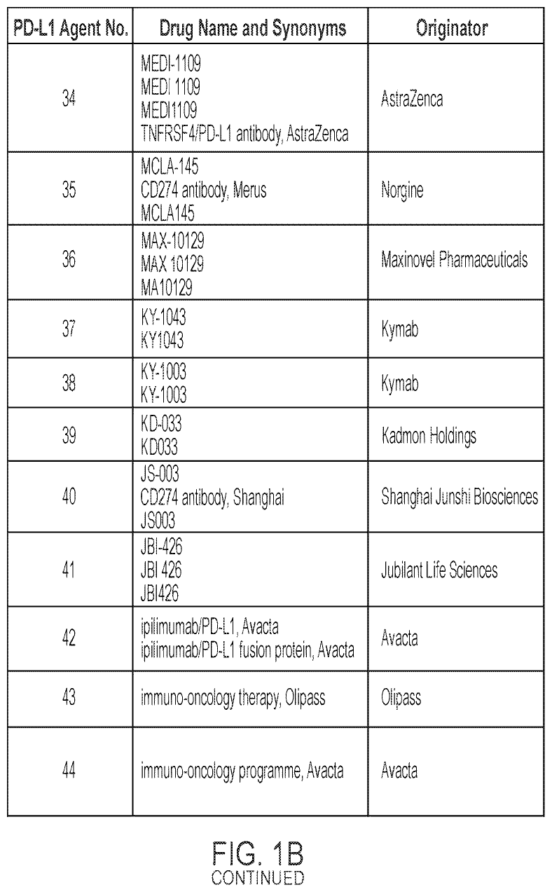

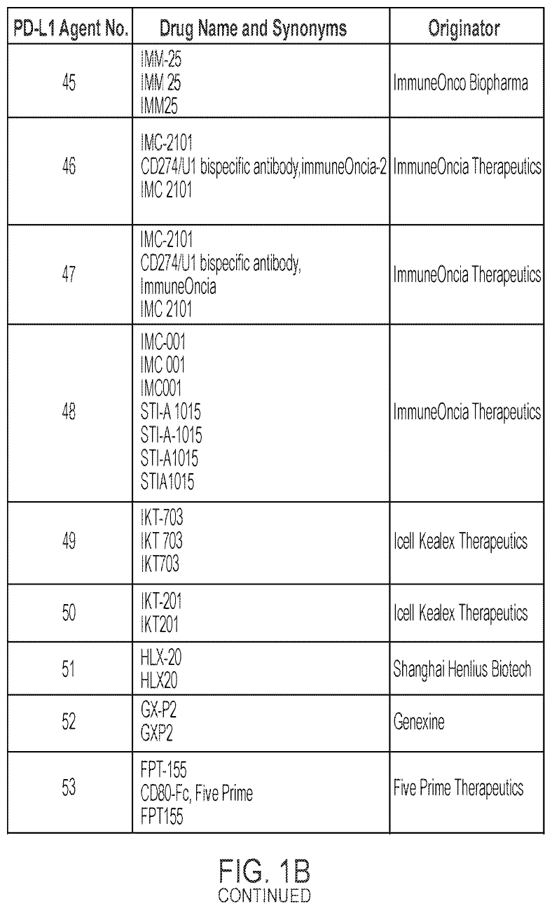

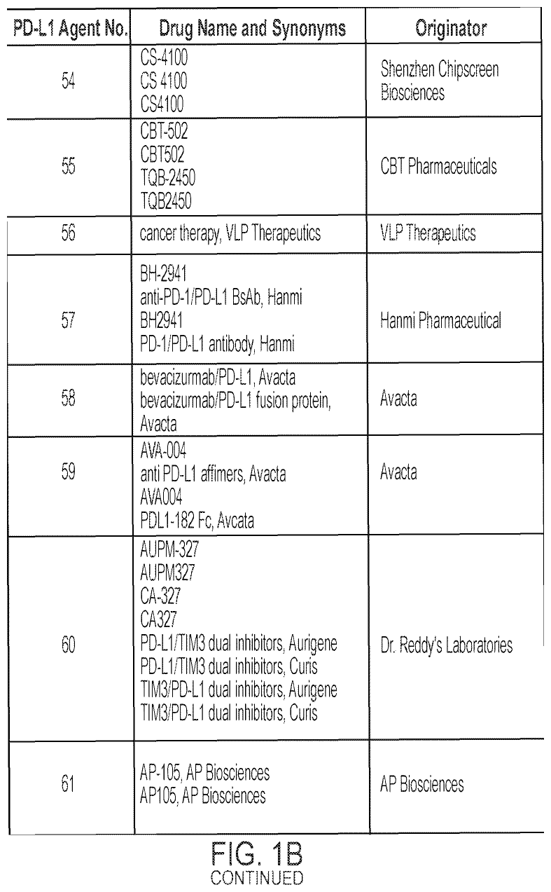

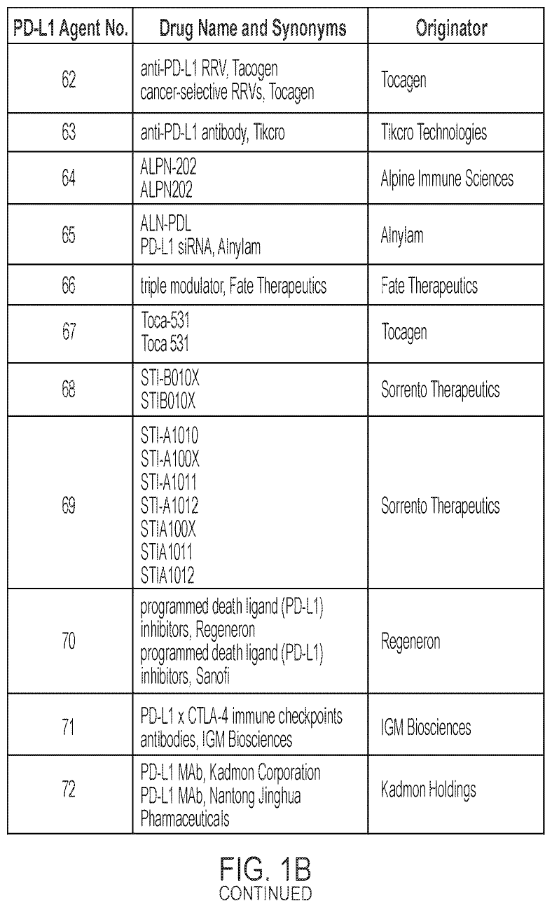

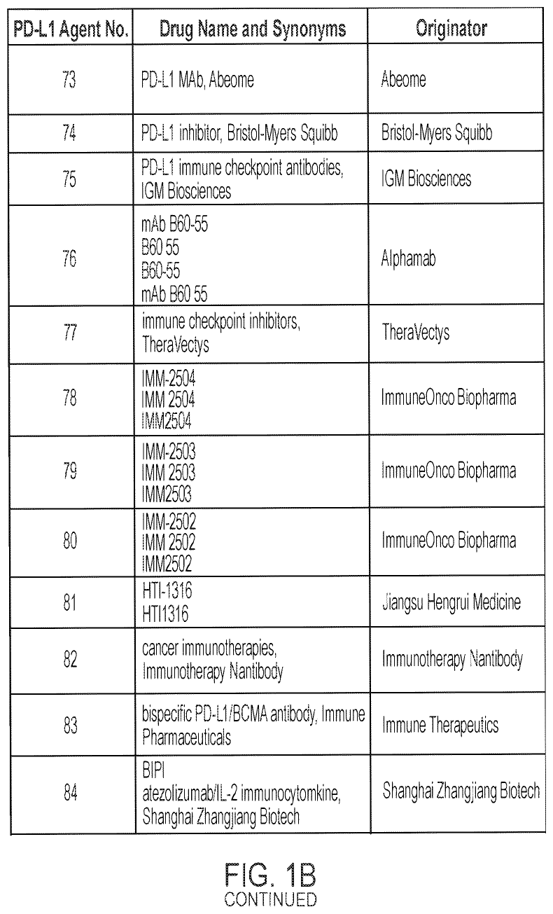

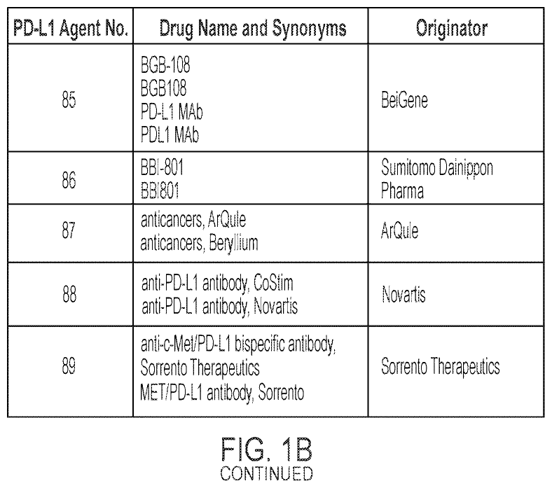

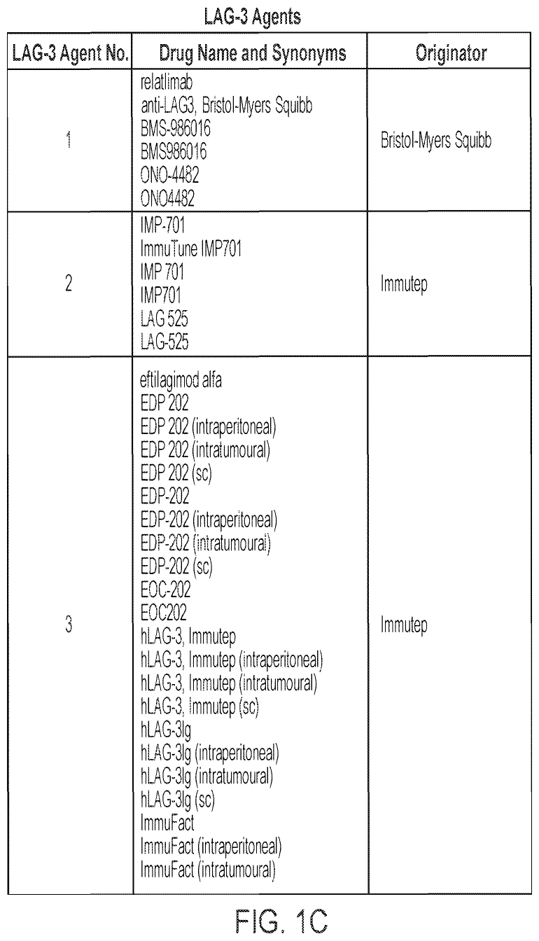

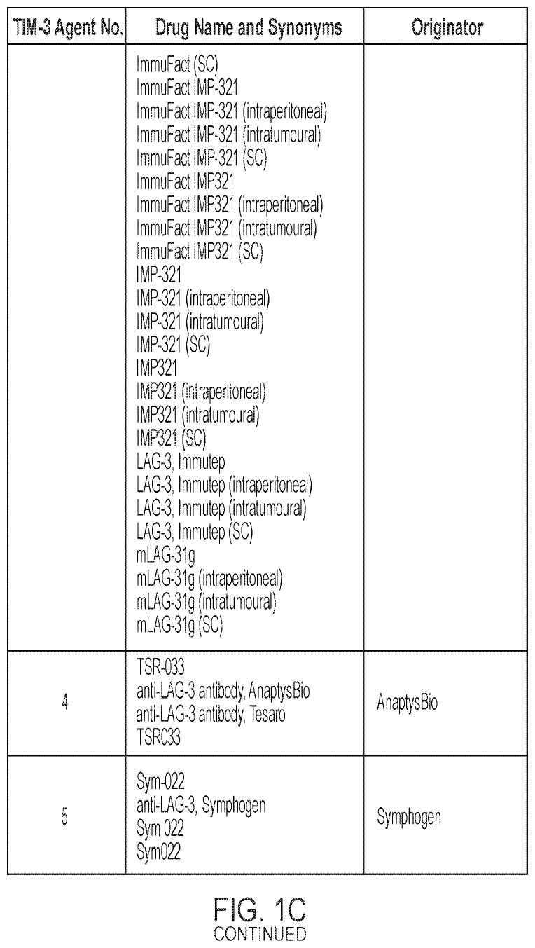

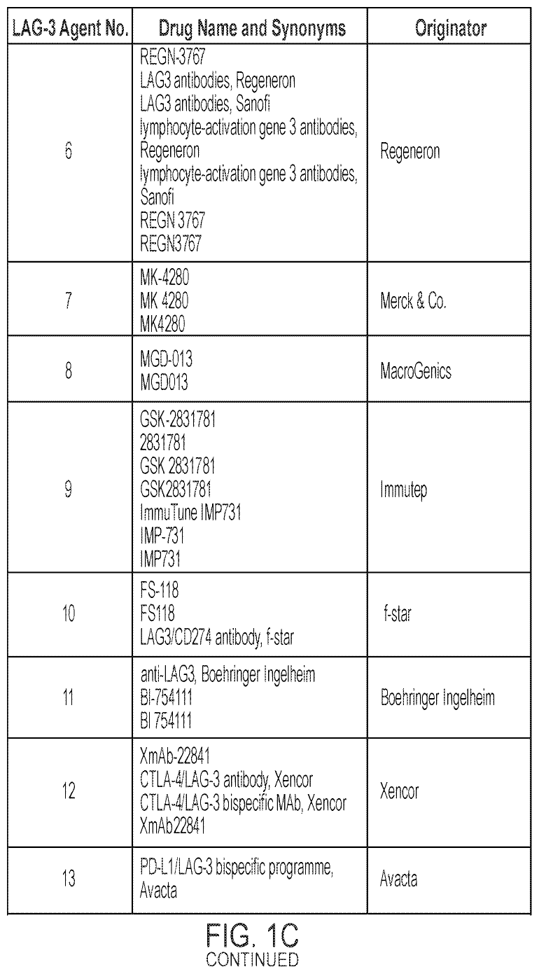

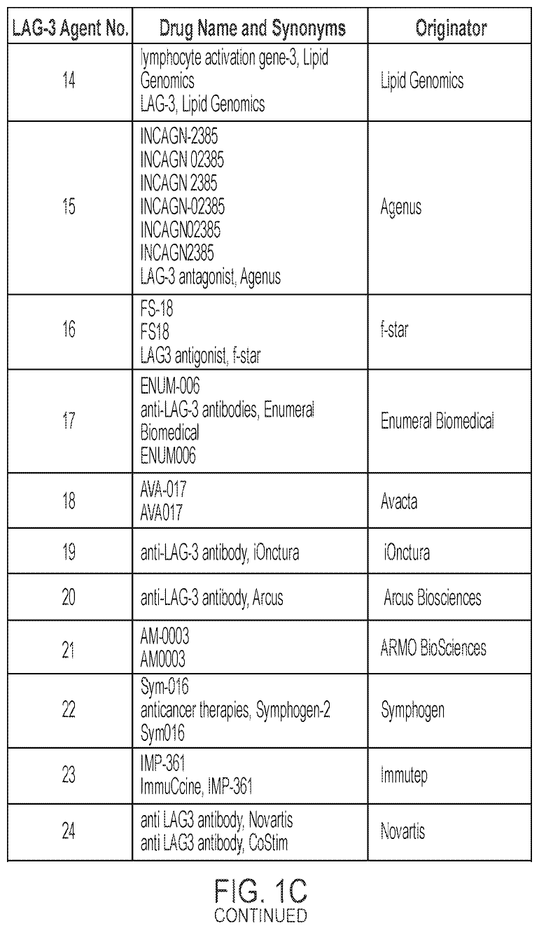

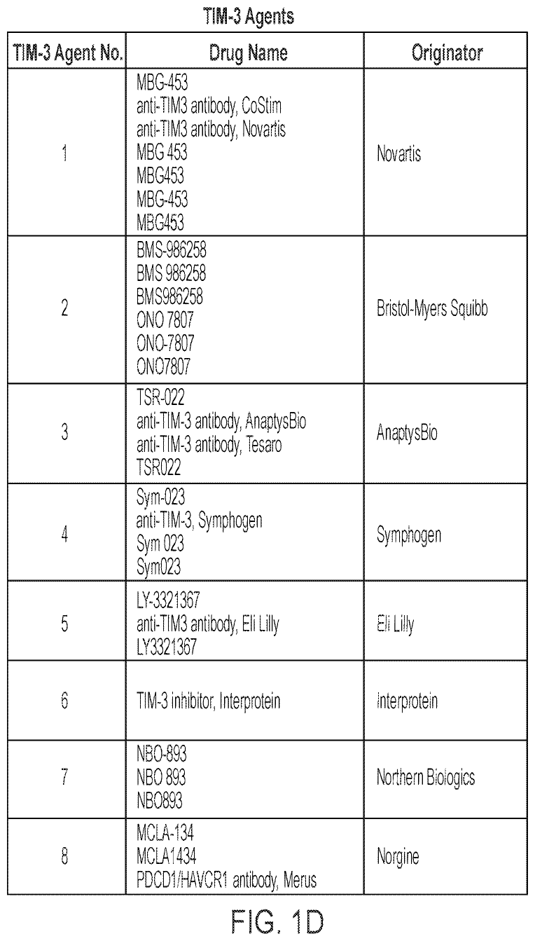

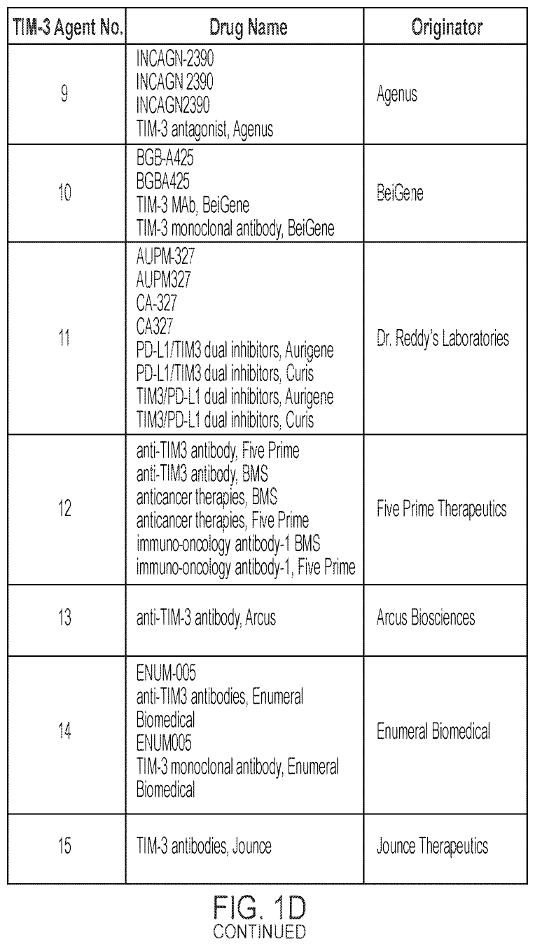

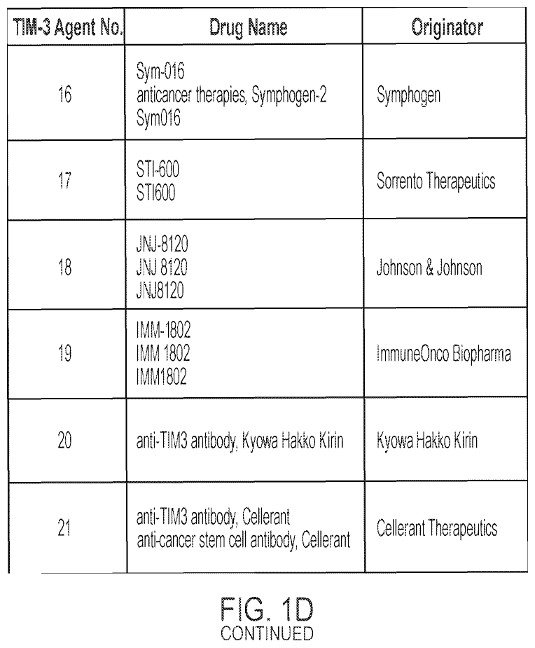

Exemplary PD-1 agents are described in FIG. 1A.

In embodiments, a PD-1 agent is any of PD-1 agent nos. 1-94 of FIG. 1A.

In some embodiments, an agent that inhibits PD-1 signaling binds to human PD-1. In some embodiments, an agent that inhibits PD-1 signaling binds to human PD-L1.

Exemplary PD-L1 agents are described in FIG. 1B.

In embodiments, a PD-L1 agent is any of PD-L1 agent nos. 1-89 of FIG. 1B.

In some embodiments, an agent that inhibits PD-1 signaling for use in combination therapies of the present disclosure is an antibody agent. In some embodiments, a PD-1 antibody agent binds an epitope of PD-1 which blocks the binding of PD-1 to any one or more of its putative ligands. In some embodiments, a PD-1 antibody agent binds an epitope of PD-1 which blocks the binding of PD-1 to two or more of its putative ligands. In embodiments, a PD-1 antibody agent binds an epitope of a PD-1 protein which blocks the binding of PD-1 to PD-L1 and/or PD-L2. PD-1 antibody agents of the present disclosure may comprise a heavy chain constant region (F_{c}) of any suitable class. In some embodiments, a PD-1 antibody agent comprises a heavy chain constant region that is based upon wild-type IgG1, IgG2, or IgG4 antibodies, or variants thereof.

In some embodiments, an agent that inhibits PD-1 signaling is a monoclonal antibody, or a fragment thereof. In some embodiments, an antibody agent that inhibits PD-1 signaling is a PD-1 antibody or fragment thereof. Monoclonal antibodies that target PD-1 that have been tested in clinical studies and/or received marketing approval in the United. Examples of antibody agents that target PD-1 signaling include, for example, any of the antibody agents listed in the following Table 2:

TABLE

| Antibody Agent | |
|---|---|
| Target (Format) | Developer |
| Opdivo Nivolumab | Bristol-Myers |
| PD-1 (Human IgG4) | Squibb ONO |
| Keytruda Pembrolizumab | Merck |
| PD-1 (Humanized IgG4) | |
| Tecentriq Atezolizumab | Roche |
| PD-L1 (Human IgG1) | |
| Imfinzi Durvalumab | Astra Zeneca |
| PD-L1 (Human IgG1) | |
| Bavencio Avelumab | Merck KGaA/Pfizer |
| PD-L1 (Human IgG1) | |
| PDR001 | Novartis |
| PD-1 (Humanized IgG4) | |
| REGN2810 (SAR-439684) | Sanofi, Regeneron |
| PD-1 (fully human IgG4) | |
| BGB-A317 | BeiGene |
| PD-1 (Humanized IgG4) | |
| engineered to not bind FcγRI | |
| LY3300054 PD-L1 | Eli Lilly |
| BI 754091 (anti-PD-1) | Boehringer Ingelheim |
| IBI308 (anti-PD-1) | Innovent Biologics |
| | (Eli Lilly) |
| INCSHR-1210 (anti-PD-1) | Incyte |
| JNJ-63723283 (anti-PD-1) | Janssen Research & |
| | Development, LLC |
| JS-001 (anti-PD-1) | Shanghai Junshi |
| | Bioscience Co., Ltd. |
| MEDI0680 (AMP-514) | MedImmune Inc |
| anti-PD-1 (Humanized IgG4) | |
| MGA-012 (anti-PD-1) | MacroGenics |
| PF-06801591 (anti-PD-1) | Pfizer |
| REGN-2810 (anti-PD-1) | Regeneron |
| TSR-042 anti-PD-1 | TESARO |
| (Humanized IgG4) | |
| CX-072 anti-PD-L1 | CytomX Therapeutics |
| FAZ053 anti-PD-L1 | Novartis |
| PD-L1 millamolecule | Bristol-Myers Squibb |

In some embodiments, an antibody agent that inhibits PD-1 signaling is atezolizumab, avelumab, BGB-A317, BI 754091, CX-072, durvalumab, FAZ053, IBI308, INCSHR-1210, JNJ-63723283, JS-001, MEDI-0680, MGA-012, nivolumab, PDR001, pembrolizumab, PF-06801591, REGN-2810, TSR-042, any of the antibodies disclosed in WO2014/179664, or derivatives thereof. In some embodiments, an antibody agent that inhibits PD-1 signaling is a PD-1 antibody selected from the group consisting of BGB-A317, BI 754091, CX-072, FAZ053, IBI308, INCSHR-1210, JNJ-63723283, JS-001, LY3300054, MEDI-0680, MGA-012, nivolumab, PD-L1 millamolecule, PDR001, pembrolizumab, PF-06801591, REGN-2810, and TSR-042. In some embodiments, an antibody agent that inhibits PD-1 signaling is a PD-1 antibody selected from the group consisting of nivolumab, pembrolizumab, and TSR-042.

In some embodiments, a PD-1 binding agent is TSR-042, nivolumab, pembrolizumab, atezolizumab, durvalumab, avelumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, BGB-A333, AMP-514 (MEDI-0680), AGEN-2034, CS1001, Sym-021, SHR-1316, PF-06801591, LZM009, KN-035, AB122, genolimzumab (CBT-501), FAZ-053, CK-301, AK 104, or GLS-010, or any of the PD-1 antibodies disclosed in WO2014/179664. In embodiments, an immune checkpoint inhibitor is a PD-1 inhibitor. In embodiments, a PD-1 inhibitor is a PD-1 binding agent (e.g., an antibody, an antibody conjugate, or an antigen-binding fragment thereof). In embodiments, a PD-1 inhibitor is a PD-L1 or PD-L2 binding agent is durvalumab, atezolizumab, avelumab, BGB-A333, SHR-1316, FAZ-053, CK-301, or, PD-L1 millamolecule, or derivatives thereof.

### TSR-042

In some embodiments, a PD-1 antibody agent is as disclosed in International Patent Application Publication WO2014/179664, the entirety of which is incorporated herein. In some embodiments, a PD-1 antibody agent comprises one or more CDR sequences as disclosed in International Patent Application Publication WO2014/179664, the entirety of which is incorporated herein. In some embodiments, a PD-1 antibody agent comprises one or more CDR sequences as disclosed in International Patent Application Publication WO2014/179664, the entirety of which is incorporated herein. In some embodiments, a PD-1 antibody agent comprises a light chain variable domain as disclosed in International Patent Application Publication WO2014/179664, the entirety of which is incorporated herein. In some embodiments, a PD-1 antibody agent comprises a heavy chain variable domain as disclosed in International Patent Application Publication WO2014/179664, the entirety of which is incorporated herein. In some embodiments, a PD-1 antibody agent comprises a light chain polypeptide as disclosed in International Patent Application Publication WO2014/179664, the entirety of which is incorporated herein. In some embodiments, a PD-1 antibody agent comprises a heavy chain polypeptide as disclosed in International Patent Application Publication WO2014/179664, the entirety of which is incorporated herein.

In embodiments, a PD-1 antibody agent is as disclosed in International Patent Application Publication. WO 2018/085468, the entirety of which is incorporated herein. In some embodiments, a PD-1 antibody agent comprises one or more CDR sequences as disclosed in International Patent Application Publication. WO 2018/085468, the entirety of which is incorporated herein. In some embodiments, a PD-1 antibody agent comprises a light chain variable domain as disclosed in International Patent Application Publication. WO 2018/085468, the entirety of which is incorporated herein. In some embodiments, a PD-1 antibody agent comprises a heavy chain variable domain as disclosed in International Patent Application Publication. WO 2018/085468, the entirety of which is incorporated herein. In some embodiments, a PD-1 antibody agent comprises a light chain polypeptide as disclosed in International Patent Application Publication. WO 2018/085468, the entirety of which is incorporated herein. In some embodiments, a PD-1 antibody agent comprises a heavy chain polypeptide as disclosed in International Patent Application Publication. WO 2018/085468, the entirety of which is incorporated herein.

In embodiments, a PD-1 antibody agent is as disclosed in International Patent Application No. PCT/US18/13029, the entirety of which is incorporated herein. In some embodiments, a PD-1 antibody agent comprises one or more CDR sequences as disclosed in International Patent Application No. PCT/US18/13029, the entirety of which is incorporated herein. In some embodiments, a PD-1 antibody agent comprises a light chain variable domain as disclosed in International Patent Application No. PCT/US18/13029, the entirety of which is incorporated herein. In some embodiments, a PD-1 antibody agent comprises a heavy chain variable domain as disclosed in International Patent Application No. PCT/US18/13029, the entirety of which is incorporated herein. In some embodiments, a PD-1 antibody agent comprises a light chain polypeptide as disclosed in International Patent Application No. PCT/US18/13029, the entirety of which is incorporated herein. In some embodiments, a PD-1 antibody agent comprises a heavy chain polypeptide as disclosed in International Patent Application No. PCT/US18/13029, the entirety of which is incorporated herein.

In embodiments, a PD-1 inhibitor is TSR-042.

In some embodiments, a PD-1 antibody agent comprises one or more CDR sequences that are 90%, 95%, 97%, 98%, 99% or 100% identical to SEQ ID NOs: 1-6.

In some embodiments, a PD-1 antibody agent comprises one, two or three heavy chain CDR sequences that are 90%, 95%, 97%, 98%, 99% or 100% identical to CDR sequences of SEQ ID NOs: 1-3.

In some embodiments, a PD-1 antibody agent comprises one, two or three light chain CDR sequences that are 90%, 95%, 97%, 98%, 99% or 100% identical to CDR sequences of SEQ ID NOs: 4-6.

In some embodiments, a PD-1 antibody agent comprises one, two or three heavy chain CDR sequences that is 90%, 95%, 97%, 98%, 99% or 100% identical to CDR sequences of SEQ ID NOs: 1-3 and one, two or three light chain CDR sequences that is 90%, 95%, 97%, 98%, 99% or 100% identical to CDR sequences of SEQ ID NOs: 4-6.

In some embodiments, a PD-1 antibody agent comprises six CDR sequences of SEQ ID NOs: 1-6.

In some embodiments, a PD-1 antibody agent comprises a heavy chain variable domain that is 90%, 95%, 97%, 98%, 99% or 100% identical to SEQ ID NO:8.

In some embodiments, a PD-1 antibody agent comprises a light chain variable domain that is 90%, 95%, 97%, 98%, 99% or 100% identical to SEQ ID NO:7.

In some embodiments, a PD-1 antibody agent comprises a heavy chain variable domain that is 90%, 95%, 97%, 98%, 99% or 100% identical to SEQ ID NO:8 and a light chain variable domain that is 90%, 95%, 97%, 98%, 99% or 100% identical to SEQ ID NO:7.

In some embodiments, a PD-1 antibody agent comprises a heavy chain polypeptide that is 90%, 95%, 97%, 98%, 99% or 100% identical to SEQ ID NO:9.

In some embodiments, a PD-1 antibody agent comprises a light chain polypeptide that is 90%, 95%, 97%, 98%, 99% or 100% identical to SEQ ID NO:10.

In some embodiments, a PD-1 antibody agent comprises a heavy chain polypeptide that is 90%, 95%, 97%, 98%, 99% or 100% identical to SEQ ID NO:9, and a light chain polypeptide that is 90%, 95%, 97%, 98%, 99% or 100% identical to SEQ ID NO:10.

SEQ ID NOs: 9 and 10 describe an exemplary humanized monoclonal anti-PD-1 antibody (TSR-042) utilizing a human IGHG4*01 heavy chain gene, and a human IGKC*01 kappa light chain gene, as scaffolds. There is a single Ser to Pro point mutation in the hinge region of the IgG4 heavy chain. This mutation is at the canonical S228 position. Without wishing to be bound by theory, it is envisioned that this point mutation serves to stabilize the hinge of the antibody heavy chain.

Table 3 shows the expected residues involved in disulfide linkages of an exemplary anti-PD-1 antibody agent heavy chain having an amino acid sequence as set forth in SEQ ID NO: 9. Table 4 shows the expected residues involved in disulfide linkages of an exemplary anti-PD-1 antibody agent light chain having an amino acid sequence as set forth in SEQ ID NO: 10.

TABLE

| Cysteine | anti-PD-1 mAb |
|---|---|
| residue | HC Residue |
| ID after | (position in |
| Edelman^{a} | SEQ ID NO: 9) |
| I | 22 |
| II | 96 |
| III | 130 |
| IV | 143 |
| V | 199 |
| VI | 222 |
| VII | 225 |
| VIII | 257 |
| IX | 317 |
| X | 363 |
| XI | 421 |

TABLE

| Cysteine | anti-PD-1 mAb |
|---|---|
| residue | LC Residue |
| ID after | (position in |
| Edelman^{a} | SEQ ID NO: 10) |
| I | 23 |
| II | 88 |
| III | 134 |
| IV | 194 |
| V | 214 |

This exemplary anti-PD-1 antibody exhibits an occupied N-glycosylation site at asparagine residue 293 in the CH2 domain of each heavy chain in the mature protein sequence (SEQ ID NO:9). The expressed N-glycosylation at this site is a mixture of oligosaccharide species typically observed on IgGs expressed in mammalian cell culture, for example, shown below is the relative abundance of glycan species from a preparation of this exemplary anti-PD-1 antibody cultured in Chinese Hamster Ovary (CHO) cells (Table 5).

TABLE
Glycan Analysis of an anti-PD-1 antibody binding agent TSR-042

| | Abundance | |
|---|---|---|
| | (% of total | |
| Species | oligosaccharide) | Description of Glycan |
| G0 | <0.1% | Nonfucosylated agalactobiantennary |
| | | complex-type oligosaccharide |
| G0F | 19.5% | Core fucosylated agalactobiantennary |
| | | complex type oligosaccharide |
| G1 | 0.1% | Nonfucosylated monogalactosylated |
| | | biantennary complex type oligosaccharide |
| G1F | 45.6% | Core fucosylated monogalactosylated |
| | | biantennary complex type oligosaccharide |
| G2F | 27.4% | Core fucosylated galactosylated biantennary |
| | | complex type oligosaccharide |
| M5 | 0.5% | Oligomannosidic N-glycan, Man₅GlcNAc₂ |

### Pembrolizumab

In some embodiments, a PD-1 antibody is pembrolizumab.

Pembrolizumab is an anti-PD-1 monoclonal antibody (“mAb”) (also known as MK-3475, SCH 9000475, Keytruda). Pembrolizumab is an immunoglobulin G4/kappa isotype humanized mAb. The mechanism of pembrolizumab consists of the mAb binding to the PD-1 receptor of lymphocytes to block the interaction of PD-1 with PD-L1 and PD-L2 ligands produced by other cells in the body, including tumor cells of certain cancers.

In some embodiments, a PD-1 antibody agent comprises a heavy chain that is 90%, 95%, 97%, 98%, 99% or 100% identical to SEQ ID NO:33, or a fragment thereof. In some embodiments, a PD-1 antibody agent comprises a light chain variable domain that is 90%, 95%, 97%, 98%, 99% or 100% identical to SEQ ID NO:34, or a fragment thereof. In some embodiments, a PD-1 antibody agent comprises a heavy chain variable domain that is 90%, 95%, 97%, 98%, 99% or 100% identical to SEQ ID NO:33 and a light chain variable domain that is 90%, 95%, 97%, 98%, 99% or 100% identical to SEQ ID NO:34.

In embodiments, pembrolizumab can be intravenously administered to a subject at a dose of about 200 mg once every about 3 weeks or about 2 mg/kg to the patient once about every Q3W.

### Nivolumab

Similarly to pembrolizumab, nivolumab (also known as BMS-936558, Opdivo) was first approved by the FDA in 2014 to treat melanoma that cannot be surgically removed or has metastasized following treatment with ipilimumab and a BRAF inhibitor where appropriate.

In embodiments, nivolumab can be intravenously administered to a subject at a dose of about 200 mg once every about 3 weeks, about 240 mg to the patient once every about 2 weeks (Q2W), about 480 mg to the patient once every about 4 weeks (Q4W), about 1 mg/kg to the patient once every about Q3W, or about 3 mg/kg to the patient once every about Q3W.

### Exemplary Dosage Regimens

In embodiments, a dose of an anti-PD-1 therapy (e.g., an PD-1-binding agent that is anti-PD-1 antibody such as TSR-042 or pembrolizumab) is administered once every about two weeks (Q2W or a 14-day treatment cycle), once every about three weeks (Q3W or a 21-day treatment cycle), once every about four weeks (Q4W or a 28-day treatment cycle), once every about five weeks (Q5W or a 35-day treatment cycle), or once every about six weeks (Q6W or a 42-day treatment cycle). In embodiments, an anti-PD-1 therapy is administered on about the first day of a treatment cycle, optionally with a permissible window of administration of ±3 days: that is, an anti-PD-1 therapy can be administered in a period spanning from about three days before the first day of a treatment cycle to about three days after the first day of a treatment cycle.

For an anti-PD-1 therapy administered intravenously (e.g., via infusion), administration may occur over a time period of about 10 minutes to about 60 minutes. In embodiments, a targeted time period for administration can be identified, optionally with permitted variation such as a range of about 15 minutes or a range of about 20 minutes. For example, a targeted time period for administration (e.g., a targeted time period of 30 minutes) can vary from about −5 minutes to about +10 minutes or from about −5 minutes to about +15 minutes). In embodiments, a targeted time period for administration is about 30 minutes, with variance of about −5 minutes to about +10 minutes: administration can therefore last from about 25 minutes to about 40 minutes. In other embodiments, a targeted time period for administration is about 30 minutes, with variance of about −5 minutes to about +15 minutes: administration can therefore last from about 25 minutes to about 45 minutes.

In some embodiments, a PD-1-binding agent (e.g., an anti-PD-1 antibody such as TSR-042 or pembrolizumab) is administered at a dose of about 1, 3 or 10 mg/kg.

In some embodiments, a PD-1-binding agent (e.g., an anti-PD-1 antibody such as TSR-042 or pembrolizumab) is administered according to a regimen that includes a dose of about 1, 3 or 10 mg/kg every two weeks (Q2W). In embodiments, a PD-1-binding agent (e.g., TSR-042 or pembrolizumab) is administered according to a regimen that includes a dose of about 1 mg/kg every two weeks (Q2W). In embodiments, a PD-1-binding agent (e.g., TSR-042 or pembrolizumab) is administered according to a regimen that includes a dose of about 3 mg/kg every two weeks (Q2W). In embodiments, a PD-1-binding agent (e.g., TSR-042 or pembrolizumab) is administered according to a regimen that includes a dose of about 10 mg/kg every two weeks (Q2W).

In some embodiments, a PD-1-binding agent (e.g., an anti-PD-1 antibody such as TSR-042 or pembrolizumab) is administered according to a regimen that includes a dose of about 1, 3 or 10 mg/kg every three weeks (Q3W). In embodiments, a PD-1-binding agent (e.g., TSR-042 or pembrolizumab) is administered according to a regimen that includes a dose of about 1 mg/kg every three weeks (Q3W). In embodiments, a PD-1-binding agent (e.g., TSR-042 or pembrolizumab) is administered according to a regimen that includes a dose of about 3 mg/kg every three weeks (Q3W). In embodiments, a PD-1-binding agent (e.g., TSR-042 or pembrolizumab) is administered according to a regimen that includes a dose of about 10 mg/kg every three weeks (Q3W).

In some embodiments, a PD-1-binding agent (e.g., an anti-PD-1 antibody such as TSR-042 or pembrolizumab) is administered according to a regimen that includes a dose of about 1, 3 or 10 mg/kg every four weeks (Q4W). In embodiments, a PD-1-binding agent (e.g., TSR-042 or pembrolizumab) is administered according to a regimen that includes a dose of about 1 mg/kg every four weeks (Q4W). In embodiments, a PD-1-binding agent (e.g., TSR-042 or pembrolizumab) is administered according to a regimen that includes a dose of about 3 mg/kg every four weeks (Q4W). In embodiments, a PD-1-binding agent (e.g., TSR-04 or pembrolizumab 2) is administered according to a regimen that includes a dose of about 10 mg/kg every four weeks (Q4W). In embodiments, a PD-1 binding agent is TSR-042.

In some embodiments, a PD-1-binding agent (e.g., TSR-042 or pembrolizumab) is administered according to a regimen that includes a flat dose of about 100 mg to about 1500 mg. In embodiments, a PD-1 binding agent is TSR-042. In embodiments, a PD-1 binding agent is pembrolizumab.

In embodiments, a flat dose of PD-1-binding agent (e.g., an anti-PD-1 antibody such as TSR-042 or pembrolizumab) is administered once every two weeks (Q2W), once every three weeks (Q3W), once every four weeks (Q4W), once every five weeks (Q5W), or once every six weeks (Q6W). In embodiments, a PD-1 binding agent is TSR-042. In embodiments, a PD-1 binding agent is pembrolizumab.

In embodiments, a PD-1-binding agent (e.g., TSR-042 or pembrolizumab) is administered according to a regimen that includes a flat dose of about 200 mg. In embodiments, a PD-1-binding agent (e.g., TSR-042 or pembrolizumab) is administered according to a regimen that includes a flat dose of about 300 mg. In embodiments, a PD-1-binding agent (e.g., TSR-042 or pembrolizumab) is administered according to a regimen that includes a flat dose of about 400 mg. In embodiments, a PD-1-binding agent (e.g., TSR-042 or pembrolizumab) is administered according to a regimen that includes a flat dose of about 500 mg. In embodiments, a PD-1-binding agent (e.g., TSR-042 or pembrolizumab) is administered according to a regimen that includes a flat dose of about 600 mg. In embodiments, a PD-1-binding agent (e.g., TSR-042 or pembrolizumab) is administered according to a regimen that includes a flat dose of about 700 mg. In embodiments, a PD-1-binding agent (e.g., TSR-042 or pembrolizumab) is administered according to a regimen that includes a flat dose of about 800 mg. In embodiments, a PD-1-binding agent (e.g., TSR-042 or pembrolizumab) is administered according to a regimen that includes a flat dose of about 900 mg. In embodiments, a PD-1-binding agent (e.g., TSR-042 or pembrolizumab) is administered according to a regimen that includes a flat dose of about 1000 mg. In embodiments, a PD-1-binding agent (e.g., TSR-042 or pembrolizumab) is administered according to a regimen that includes a flat dose of about 1100 mg. In embodiments, a PD-1-binding agent (e.g., TSR-042 or pembrolizumab) is administered according to a regimen that includes a flat dose of about 1200 mg. In embodiments, a PD-1 binding agent is TSR-042. In embodiments, a PD-1 binding agent is pembrolizumab.

In some embodiments, a PD-1-binding agent (e.g., pembrolizumab) is administered according to a regimen that includes a flat dose of about 200 mg administered once about every 1-6 weeks. In embodiments, a PD-1-binding agent (e.g., pembrolizumab) is administered according to a regimen that includes a flat dose of about 200 mg every about two weeks (Q2W). In embodiments, a PD-1-binding agent (e.g., pembrolizumab) is administered according to a regimen that includes a flat dose of about 200 mg every about three weeks (Q3W). In embodiments, a PD-1-binding agent (e.g., pembrolizumab) is administered according to a regimen that includes a flat dose of about 200 mg every about four weeks (Q4W). In embodiments, a PD-1-binding agent (e.g., pembrolizumab) is administered according to a regimen that includes a flat dose of about 200 mg every about five weeks (Q5W). In embodiments, a PD-1-binding agent (e.g., pembrolizumab) is administered according to a regimen that includes a flat dose of about 200 mg every about six weeks (Q6W).

In embodiments, a PD-1 binding agent administered in combination with a PARP inhibitor (e.g., niraparib) is pembrolizumab. In embodiments, pembrolizumab is administered according to a regimen that includes a flat dose of about 200 mg every about three weeks (Q3W), which also can be referred to as a 21-day treatment cycle. In embodiments, pembrolizumab is administered on about the first day of a treatment cycle, optionally with a permissible window of administration of ±3 days: that is, pembrolizumab can be administered in a period spanning from about three days before the first day of a treatment cycle to about three days after the first day of a treatment cycle.

In embodiments, pembrolizumab is administered intravenously (e.g., via infusion). In embodiments, pembrolizumab is administered intravenously (e.g., via infusion) over a time period of about 15 minutes to about 45 minutes. In embodiments, pembrolizumab is administered intravenously (e.g., via infusion) over a targeted time period of about 30 minutes, with an optionally permitted window between about −5 minutes and about +10 minutes: that, is pembrolizumab is administered intravenously (e.g., via infusion) over a time period of about 25 minutes to about 40 minutes.

In some embodiments, a PD-1-binding agent (e.g., TSR-042) is administered according to a regimen that includes a flat dose of about 500 mg. In embodiments, a PD-1-binding agent (e.g., TSR-042) is administered according to a regimen that includes a flat dose of about 500 mg every about two weeks (Q2W). In embodiments, a PD-1-binding agent (e.g., TSR-042) is administered according to a regimen that includes a flat dose of about 500 mg every about three weeks (Q3W). In embodiments, a PD-1-binding agent (e.g., TSR-042) is administered according to a regimen that includes a flat dose of about 500 mg every about four weeks (Q4W). In embodiments, a PD-1-binding agent (e.g., TSR-042) is administered according to a regimen that includes a flat dose of about 500 mg every about five weeks (Q5W). In embodiments, a PD-1-binding agent (e.g., TSR-042) is administered according to a regimen that includes a flat dose of about 500 mg every about six weeks (Q6W). In embodiments, a PD-1 binding agent is TSR-042.

In some embodiments, a PD-1-binding agent (e.g., TSR-042) is administered according to a regimen that includes a flat dose of about 1000 mg. In embodiments, a PD-1-binding agent (e.g., TSR-042) is administered according to a regimen that includes a flat dose of about 1000 mg every about two weeks (Q2W). In embodiments, a PD-1-binding agent (e.g., TSR-042) is administered according to a regimen that includes a flat dose of about 1000 mg every about three weeks (Q3W). In embodiments, a PD-1-binding agent (e.g., TSR-042) is administered according to a regimen that includes a flat dose of about 1000 mg every four weeks (Q4W). In embodiments, a PD-1-binding agent (e.g., TSR-042) is administered according to a regimen that includes a flat dose of about 1000 mg every about five weeks (Q5W). In embodiments, a PD-1-binding agent (e.g., TSR-042) is administered according to a regimen that includes a flat dose of about 1000 mg every about six weeks (Q6W). In embodiments, a PD-1 binding agent is TSR-042.

In some embodiments, a PD-1-binding agent (e.g., an anti-PD-1 antibody such as TSR-042) is administered according to a regimen that includes a first dose of about 500 mg every three weeks (Q3W) for the first 2-6 (e.g., the first 2, 3, 4, 5, or 6) dosage cycles and a second dose of about 1000 mg every six weeks (Q6W) until treatment is discontinued (e.g., due to disease progression, adverse effects, or as determined by a physician). In embodiments, a PD-1 binding agent is TSR-042.

In some embodiments, a PD-1-binding agent (e.g., an anti-PD-1 antibody such as TSR-042) is administered according to a regimen that includes a first dose of about 500 mg every three weeks (Q3W) for the first four dosage cycles and a second dose of about 1000 mg every six weeks (Q6W) until treatment is discontinued (e.g., due to disease progression, adverse effects, or as determined by a physician). In embodiments, a PD-1 binding agent is TSR-042.

In embodiments, a PD-1 binding agent is TSR-042. In embodiments, TSR-042 is administered according to a regimen that includes a flat dose of about 500 mg every about three weeks (Q3W), which also can be referred to as a 21-day treatment cycle. In embodiments, TSR-042 is administered on about the first day of a treatment cycle, optionally with a permissible window of administration of ±3 days: that is, TSR-042 can be administered in a period spanning from about three days before the first day of a treatment cycle to about three days after the first day of a treatment cycle.

In embodiments, TSR-042 is administered intravenously (e.g., via infusion). In embodiments, TSR-042 is administered intravenously (e.g., via infusion) over a time period of about 15 minutes to about 45 minutes. In embodiments, TSR-042 is administered intravenously (e.g., via infusion) over a targeted time period of about 30 minutes, with an optionally permitted window between about −5 minutes and about +15 minutes: that, is TSR-042 is administered intravenously (e.g., via infusion) over a time period of about 25 minutes to about 45 minutes.

In certain methods, an anti-PD-1 antibody agent can be administered prior to (e.g., 5 minutes, 15 minutes, 30 minutes, 45 minutes, 1 hour, 2 hours, 4 hours, 6 hours, 12 hours, 24 hours, 48, hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of another therapeutic agent to a subject in need thereof.

General Protocols for Dosing an Anti-PD-1 Therapy in Combination with a PARP Inhibitor

Exemplary dosing regiments have been described herein for PARP inhibitors (e.g., niraparib) and anti-PD-1 therapies (e.g., TSR-042 or pembrolizumab). Accordingly, any exemplary dose or dosing regimen described herein for a PARP inhibitor (e.g., niraparib) can be used in combination with any exemplary dose or dosing regimen described herein for an anti-PD-1 therapy (e.g., TSR-042 or pembrolizumab). Still further exemplary, general protocols for combination therapy of a PARP inhibitor and an anti-PD-1 therapy are described herein.

As described herein, provided methods comprise administering a therapy that inhibits PARP and an anti-PD-1 therapy (e.g., a therapy that inhibits PD-1 signaling) in combination to a patient, a subject, or a population of subjects according to a regimen that achieves a clinical benefit (e.g., any one of or combination of: prolonged progression free survival; reduced hazard ratio for disease progression or death; and/or prolonged overall survival or a positive overall response rate).

In some embodiments, an agent that inhibits PARP (e.g., niraparib) is administered in combination (e.g., simultaneously or sequentially) with an anti-PD-1 therapy (e.g., TSR-042 or pembrolizumab). In some embodiments, an anti-PD-1 therapy is an agent that inhibits PD-1 signaling (e.g., a protein, antibody, anti-sense molecule or small organic molecule inhibitor of PD-1 signaling). In some embodiments, an agent that inhibits PD-1 signaling binds to PD-1. In some embodiments, an agent that inhibits PD-1 signaling is a PD-1 antibody agent (e.g., pembrolizumab or TSR-042).

In some embodiments, an agent that inhibits PARP (e.g., niraparib) is administered in combination (e.g., simultaneously or sequentially) with an immunotherapy (e.g. a PD-1 antibody agent). In some embodiments, the immunotherapy is or comprises administration of an agent that targets a specific antigen (e.g. PD-1); in some embodiments, immunotherapy is or comprises administration of an antibody agent that targets PD-1 (e.g., pembrolizumab or TSR-042).

In some embodiments, one or more doses of an agent that inhibits PARP (e.g., niraparib) is administered before, during, or after administration of one or more doses of an agent that inhibits PD-1 signaling (e.g., pembrolizumab or TSR-042). In some embodiments, an agent that inhibits PARP (e.g., niraparib) and an agent that inhibits PD-1 signaling (e.g., pembrolizumab or TSR-042) are administered in overlapping regimens. In some embodiments, at least one cycle of an agent that inhibits PARP (e.g., niraparib) is administered prior to initiation of therapy with an agent that inhibits PD-1 signaling (e.g., pembrolizumab or TSR-042). In some embodiments, administration “in combination” includes administration of an agent that inhibits PARP (e.g., niraparib) and simultaneously or sequentially administering an agent that inhibits PD-1 signaling (e.g., an antibody agent such as pembrolizumab or TSR-042).

In some embodiments, administration of a particular dose or cycle of an agent that inhibits PARP (e.g., niraparib) is separated in time from a particular dose or cycle of an agent that inhibits PD-1 signaling (e.g., pembrolizumab or TSR-042) by a time period having a length that may be, for example, 1 minute, 5 minutes, 30 minutes, 1 hour, 2 hours, 5 hours, 10 hours, 12 hours, 24 hours, 48 hours, 72 hours, 96 hours, 1 week, 2 weeks, or more. In some embodiments, the range may be bounded by a lower limit and an upper limit, the upper limit being larger than the lower limit. In some embodiments, the lower limit may be about 1 minute, about 5 minutes, about 15 minutes, about 30 minutes, about 45 minutes, about 1 hour, about 2 hours, about 4 hours, about 6 hours, about 12 hours, about 24 hours, about 48, hours, about 72 hours, about 96 hours, or about 1 week. In some embodiments, the upper limit may be about 2 weeks, about 3 weeks, about 4 weeks, about 5 weeks, about 6 weeks, about 8 weeks, or about 12 weeks. In some embodiments, the administration of a particular dose of an agent that inhibits PARP (e.g., niraparib) is separated in time from a particular dose of an agent that inhibits PD-1 signaling (e.g., pembrolizumab or TSR-042) by a time period within the range of about 1 minute to about 12 weeks. In some embodiments, the range may be about 1 minute to about 8 weeks. In some embodiments, the range may be about 1 minute to about 6 weeks. In some embodiments, the range may be about 1 minute to about 4 weeks. In some embodiments, the range may be about 1 minute to about 2 weeks. In some embodiments, the range may be about 1 minute to about 1 week. In some embodiments, the range may be about 1 minute to about 96 hours. In some embodiments, the range may be about 1 minute to about 72 hours. In some embodiments, the range may be about 1 minute to about 48 hours. In some embodiments, the range may be about 1 minute to about 24 hours. In some embodiments, the range may be about 1 minute to about 12 hours. In some embodiments, the range may be about 1 minute to about 8 hours. In some embodiments, the range may be about 1 minute to about 4 hours. In some embodiments, the range may be about 1 minute to about 2 hours. In some embodiments, the range may be about 1 minute to about 1 hour. In some embodiments, the range may be about 1 minute to about 11 minute.

In some embodiments, the regimen comprises at least one oral dose of an agent that inhibits PARP (e.g., niraparib). In some embodiments, the regimen comprises a plurality of oral doses. In some embodiments, the regimen comprises once daily (QD) dosing. In some embodiments, an agent that inhibits PARP (e.g., niraparib) is administered on the first day of a 21-day cycle upon completion of infusion with an agent that inhibits PD-1 signaling (e.g., pembrolizumab or TSR-042). In some embodiments, an agent that inhibits PARP (e.g., niraparib) is administered daily throughout the regimen cycle at the same time every day. In some embodiments the same time every day is preferably in the morning.

In some embodiments, the regimen comprises of one infusion of an agent that inhibits PD-1 signaling (e.g., pembrolizumab or TSR-042) per regimen cycle. In some embodiments, the regimen comprises of one, 30-minute infusion of an agent that inhibits PD-1 signaling (e.g., pembrolizumab or TSR-042) per regimen cycle. In some embodiments, the regimen comprises of one, 30-minute infusion of an agent that inhibits PD-1 signaling (e.g., pembrolizumab or TSR-042) on the first day of each regimen cycle.

In some embodiments, the regimen comprises at least one 2 week-8 week cycle. In some embodiments, the regimen comprises a plurality of 2 week-8 week cycles. In some embodiments, the regimen comprises one 2 week-8 week cycle. In some embodiments, the regimen comprises two 2 week-8 week cycles. In some embodiments, the regimen comprises three or more 2 week-8 week cycles. In some embodiments, the regimen comprises continuous 2 week-8 week cycles.

In some embodiments, the regimen comprises at least one 28 day cycle. In some embodiments, the regimen comprises a plurality of 28 day cycles. In some embodiments, the regimen comprises one 28 day cycle. In some embodiments, the regimen comprises two 28 day cycles. In some embodiments, the regimen comprises three or more 28 day cycles. In some embodiments, the regimen comprises continuous 28 day cycles.

In some embodiments, the regimen comprises at least one 21 day cycle. In some embodiments, the regimen comprises a plurality of 21 day cycles. In some embodiments, the regimen comprises one 21 day cycle. In some embodiments, the regimen comprises two 21 day cycles. In some embodiments, the regimen comprises three or more 21 day cycles. In some embodiments, the regimen comprises continuous 21 day cycles.

In some embodiments, the regimen comprises administration of an effective dose of an agent that inhibits PARP (e.g., niraparib) daily until disease progression or unacceptable toxicity occurs. In some embodiments, the regimen comprises a daily dose of 100 mg, 200 mg, 300 mg or more of a PARP inhibitor (e.g., niraparib) per day dosed until disease progression or unacceptable toxicity occurs. In some embodiments, the range is bounded by a lower limit and an upper limit, the upper limit being larger than the lower limit. In some embodiments, the lower limit may be about 10 mg, about 25 mg, about 50 mg, or about 100 mg. In some embodiments, the upper limit may be about 150 mg, about 200 mg, about 250 mg, about 300 mg, about 350 mg, about 400 mg or about 500 mg. In some embodiments, the oral dose is an amount of a PARP inhibitor (e.g., niraparib) within a range of about 10 mg to about 500 mg. In some embodiments, the dose is within a range of about 25 mg to about 400 mg. In some embodiments, the dose is within a range of about 50 mg to about 300 mg. In some embodiments, the dose is within a range of about 150 mg to about 350 mg. In some embodiments, the dose is within a range of about 50 mg to about 250 mg. In some embodiments, the dose is within a range of about 50 mg to about 200 mg. In some embodiments, the dose is within a range of about 50 mg to about 100 mg. In some embodiments, the dose is within a range of about 100 mg to about 300 mg.

In some embodiments, the oral dose of niraparib is administered in one or more unit dosage forms. In some embodiments, the one or more unit dosage forms are capsules. In some embodiments, each unit dosage form comprises about 100 mg of PARP inhibitor (e.g., niraparib). It is understood that any combination of unit dosage forms can be combined to form a once daily (QD) dose. For example, three 100 mg unit dosage forms can be taken once daily such that 300 mg of PARP inhibitor (e.g., niraparib) is administered once daily. In some embodiments, two 100 mg unit dosage forms can be taken once daily such that 200 mg of PARP inhibitor (e.g., niraparib) is administered once daily In some embodiments, one 100 mg unit dosage forms can be taken once daily such that 100 mg of PARP inhibitor (e.g., niraparib) is administered once daily.

In some embodiments, the regimen comprises a single infusion of at least 200 mg of an agent that inhibits PD-1 signaling (e.g., about 200 mg of pembrolizumab or about 500 mg of TSR-042). In some embodiments, the regimen comprises a single infusion of an agent that inhibits PD-1 signaling (e.g., pembrolizumab or TSR-042) over a time period of at least 25 minutes, 30 minutes, 35 minutes, 40 minutes, 45 minutes, or more. In some embodiments, the range may be bounded by a lower limit and an upper limit, the upper limit being larger than the lower limit. In some embodiments, the lower limit may be about 25 minutes, or about 30 minutes. In some embodiments, the upper limit may be about 35 minutes, about 40 minutes, or about 45 minutes. In some embodiments, the range may be about 25 minutes to about 45 minutes. In some embodiments, the range may be about 25 minutes to about 40 minutes. In some embodiments, the range may be about 25 minutes to about 35 minutes. In some embodiments, the range may be about 25 minutes to about 30 minutes. In some embodiments an agent that inhibits PD-1 signaling (e.g., pembrolizumab or TSR-042) is administered through intravenous (IV) infusion. In some embodiments an intravenous dose of an agent that inhibits PD-1 signaling (e.g., pembrolizumab or TSR-042) is administered in one or more unit dosage forms.

### Treatment of Cancer

Accordingly, in one aspect, the invention provides methods for preventing, treating or alleviating a cell proliferative disease or disorder or a symptom of said disease or disorder in a subject (e.g., a subject having a cancer or a cell proliferative disease or disorder or a subject at risk of a cancer or a cell proliferative disease or disorder). Subjects at risk for cell proliferation-related diseases or disorders include patients who have a family history of cancer or a subject exposed to a known or suspected cancer-causing agent. Administration of a prophylactic agent can occur prior to the manifestation of the disease or disorder such that it is prevented or, alternatively, delayed in its progression.

The inventive methods can be used to treat any type of cancer known in the art.

In embodiments, a cancer is a refractory cancer, which can also be interchangeably referred to as a resistant cancer. In embodiments, a cancer is refractory or resistant to all treatment. In embodiments, a cancer is refractory or resistant to a particular treatment. In embodiments, a cancer never responds to a treatment: the cancer is refractory or resistant from the beginning of treatment. In embodiments, a cancer initially responds to a treatment but then stops responding: the cancer becomes refractory or resistant during the treatment (also known as relapse). A cancer can be refractory or resistant to one or more previously received lines of therapy (e.g., an immunotherapy such as anti-PD-1 therapy and/or a chemotherapy including cytotoxic chemotherapy such as platinum-based chemotherapy).

In embodiments, a cancer is refractory or resistant to a previously-received immunotherapy. In embodiments, a cancer is refractory or resistant to a previously-received immunotherapy from the beginning of treatment with the immunotherapy. In embodiments, a cancer becomes refractory or resistant to a previously-received immunotherapy during a treatment with the immunotherapy (also referred to as a relapsed cancer). In embodiments, a cancer is refractory or resistant to a previously-received anti-PD-1 therapy. In embodiments, a cancer is refractory or resistant to a previously-received anti-PD-1 therapy from the beginning of treatment with the anti-PD-1 therapy. In embodiments, a cancer becomes refractory or resistant to a previously-received anti-PD-1 therapy during a treatment with the anti-PD-1 therapy (also referred to as a relapsed cancer).

In embodiments, a cancer is refractory or resistant to a previously-received chemotherapy (e.g., a cytotoxic chemotherapy). In embodiments, a cancer is refractory or resistant to a previously-received chemotherapy (e.g., a cytotoxic chemotherapy) from the beginning of treatment with the chemotherapy (e.g., a cytotoxic chemotherapy). In embodiments, a cancer becomes refractory or resistant to a previously-received chemotherapy (e.g., a cytotoxic chemotherapy) during a treatment with the chemotherapy (e.g., a cytotoxic chemotherapy) and also may be referred to as a relapsed cancer. In embodiments, a cancer is refractory or resistant to a previously-received platinum-based chemotherapy. In embodiments, a cancer is refractory or resistant to a previously-received platinum-based chemotherapy from the beginning of treatment with the platinum-based chemotherapy. In embodiments, a cancer becomes refractory or resistant to a previously-received platinum-based chemotherapy during a treatment with the platinum-based chemotherapy.

In embodiments, a cancer is an advanced cancer. In some embodiments, a cancer is a stage II, stage III or stage IV cancer. In some embodiments, a cancer is a stage II cancer. In some embodiments, a cancer is a stage III cancer. In some embodiments, a cancer is a stage IV cancer.

In embodiments, a cancer is a locally advanced cancer.

In embodiments, a cancer is a metastatic cancer.

In embodiments, methods described herein are useful for reducing tumors or inhibiting the growth of tumor cells in a subject.

In embodiments, a cancer is a recurrent cancer.

Cancers that can be treated with methods described herein also include cancers associated with a high tumor mutation burden (TMB), cancers that microsatellite stable (MSS), cancers that are characterized by microsatellite instability, cancers that have a high microsatellite instability status (MSI-H), cancers that have low microsatellite instability status (MSI-L), cancers associated with high TMB and MSI-H, cancers associated with high TMB and MSI-L or MSS), cancers having a defective DNA mismatch repair system, cancers having a defect in a DNA mismatch repair gene, hypermutated cancers, cancers having homologous recombination repair deficiency/homologous repair deficiency (“HRD”), cancers comprising a mutation in polymerase delta (POLD), and cancers comprising a mutation in polymerase epsilon (POLE). In embodiments, a cancer is a cancer is characterized by a homologous recombination repair (HRR) gene deletion, a mutation in the DNA damage repair (DDR) pathway, BRCA deficiency, isocitrate dehydrogenase (IDH) mutation, and/or a chromosomal translocation. In embodiments, a cancer is a hypermutant cancer, a MSI-H cancer, a MSI-L cancer, or a MSS cancer. In embodiments, a cancer is characterized by one or more of these characteristics.

In embodiments, immune-related gene expression signatures can be predictive of a response to an anti-PD-1 therapy for cancer as described herein. For example, a gene panel that includes genes associated with IFN-γ signaling can be useful in identifying cancer patients who would benefit from anti-PD-1 therapy. Exemplary gene panels are described in Ayers et al., J. Clin. Invest., 127(8):2930-2940, 2017. In embodiments, a cancer patient has a cancer that is breast cancer (e.g., TNBC) or ovarian cancer. In embodiments, a cancer patient has a cancer that is bladder cancer, gastric cancer, bilary cancer, esophageal cancer, or head and neck squamous cell carcinoma (HNSCC). In embodiments, a cancer patient has a cancer that is anal cancer or colorectal cancer.

In embodiments, a patient is treatment-naïve (e.g., has not previously received any line of treatment for the cancer to be treated according to methods described herein).

In embodiments, a patient has not been previously treated with an immunotherapy (e.g., a patient has not been previously treated with an anti-PD-1 therapy (e.g., an anti-PD-1 agent or an anti-PD-L1/L2 agent), anti-CTLA-4, anti-TIM-3, and/or anti-LAG-3 therapy). In embodiments, a patient has not been previously treated with an anti-PD-1 immunotherapy. In embodiments, a patient has not been previously treated with an anti-PD-L1 immunotherapy. In embodiments, a patient has not been previously treated with an anti-CTLA-4 immunotherapy. In embodiments, a patient has not been previously treated with an anti-TIM-3 immunotherapy. In embodiments, a patient has not been previously treated with an anti-LAG-3 immunotherapy. In embodiments, a patient who has not been previously treated with an immunotherapy has received at least one other line of treatment (LOT) as described herein. In embodiments, a patient who has not been previously treated with an immunotherapy has received one, two, three, four, or five prior LOT (e.g., any LOT as described herein).

In embodiments, a patient has not been previously treated with chemotherapy (e.g., cytotoxic chemotherapy such as platinum-based chemotherapy).

In some embodiments, a patient has previously been treated with one or more different cancer treatment modalities. In some embodiments, at least some of the patients in the cancer patient population have previously been treated with one or more of surgery, radiotherapy, chemotherapy or immunotherapy. In some embodiments, at least some of the patients in the cancer patient population have previously been treated with chemotherapy (e.g., platinum-based chemotherapy). For example, a patient who has received two lines of cancer treatment can be identified as a 2L cancer patient (e.g., a 2L NSCLC patient). In embodiments, a patient has received two lines or more lines of cancer treatment (e.g., a 2L+ cancer patient such as a 2L+ endometrial cancer patient). In embodiments, a patient has not been previously treated with an anti-PD-1 therapy. In embodiments, a patient previously received at least one line of cancer treatment (e.g., a patient previously received at least one line or at least two lines of cancer treatment). In embodiments, a patient previously received at least one line of treatment for metastatic cancer (e.g., a patient previously received one or two lines of treatment for metastatic cancer).

In embodiments, a subject is resistant to treatment with an agent that inhibits PD-1. In embodiments, a subject is refractory to treatment with an agent that inhibits PD-1. In embodiments, a method described herein sensitizes the subject to treatment with an agent that inhibits PD-1.

In embodiments, tumors with high levels of the tumor infiltrating lymphocytes (lymphoid index) tumor infiltrating myeloid cells (myeloid index) tumor mutational burden (TMB), tumor inflammation, homologous recombination deficiency (HRD or HRR gene mutations) and Th1 (Th1 index) or Th2 (Th2) index cytokines are more likely to respond to PD-1 and LAG-3 blockade.

In embodiments, the subject has a cancer or an infectious disease that has a Th2 cytokine profile. In embodiments, cancers with a high Th2 index include large B-cell lymphoma, lung adenocarcinoma, head and neck squamous cell carcinoma, pancreatic cancer, esophageal cancer, cervical cancer, gastric cancer, lung squamous carcinoma, thyroid cancer, bladder cancer, triple negative breast cancer and colorectal cancer.

In embodiments, cancers that have high lymphoid index include Large B-cell lymphoma, thymoma, acute myeloid leukemia, testicular tumors, lung adenocarcinoma, kidney clear cell, triple negative breast cancer, gastric cancer, lung squamous carcinoma and mesothelioma.

In embodiments, cancers that have high lymphoid, tumor mutational burden and tumor inflammation indices include, Large B-cell lymphoma, lung adenocarcinoma, lung squamous carcinoma, gastric cancer, melanoma, renal cell carcinoma, triple negative breast cancer, head and neck cancer, cervical cancer, colorectal cancer and esophageal cancer.

In embodiments, cancers that are characterized by high lymphoid index and high myeloid index include: Large b-cell lymphoma, acute myeloid leukemia, kidney clear cell, lung adenocarcinoma, thymoma, testicular tumors, breast-TNBC, mesothelioma, pancreatic cancer and lung squamous cell.

In embodiments, cancers that have high lymphoid, myeloid indices and tumor mutational burden include lung adenocarcinoma, Large-B-cell lymphoma, lung squamous cell, breast-TNBC, kidney clear cell, head and neck cancer, gastric cancer, pancreatic cancer, cervical cancer and mesothelioma.

In embodiments, cancers that have high levels of lymphoid, myeloid, interferon/cytokine indices include lung adenocarcinoma, lung squamous cell, breast-TNBC, gastric cancer, head and neck cancer, Large B-cell lymphoma, esophageal, pancreatic cancer, cervical cancer, kidney clear cell, mesothelioma, melanoma, bladder cancer, and colon adenocarcinoma.

In embodiments, a cancer is characterized by microsatellite instability. Microsatellite instability (“MSI”) is or comprises a change that in the DNA of certain cells (such as tumor cells) in which the number of repeats of microsatellites (short, repeated sequences of DNA) is different than the number of repeats that was contained in the DNA from which it was inherited. Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load. It has been demonstrated that some tumors characterized by MSI-H have improved responses to certain anti-PD-1 agents (Le et al., (2015) N. Engl. J. Med. 372(26):2509-2520; Westdorp et al., (2016) Cancer Immunol. Immunother. 65(10):1249-1259). In some embodiments, a cancer has a microsatellite instability of high microsatellite instability (e.g., MSI-H status). In some embodiments, a cancer has a microsatellite instability status of low microsatellite instability (e.g., MSI-Low or MSI-L). In some embodiments, a cancer has a microsatellite instability status of microsatellite stable (e.g., MSS status). In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR. About 15% of sporadic colorectal cancers (CRC) harbor widespread alterations in the length of microsatellite (MS) sequences, known as microsatellite instability (MSI) (Boland and Goel, 2010). Sporadic MSI CRC tumors display unique clinicopathological features including near-diploid karyotype, higher frequency in older populations and in females, and a better prognosis (de la Chapelle and Hampel, 2010; Popat et al., 2005). MSI is also present in other tumors, such as in endometrial cancer (EC) of the uterus, the most common gynecological malignancy (Duggan et al., 1994). The same reference Bethesda panel originally developed to screen an inherited genetic disorder (Lynch syndrome) (Umar et al., 2004) is currently applied to test MSI for CRCs and ECs.

In embodiments, a cancer has a low microsatellite instability status (MSI-L).

In embodiments, a cancer has a high microsatellite instability status (MSI-H). In embodiments, a MSI-H cancer is MSI-H endometrial cancer. In embodiments, a MSI-H cancer is a solid tumor. In embodiments, a MSI-H cancer is a metastatic tumor. In embodiments, a MSI-H cancer is endometrial cancer. In embodiments, a MSI-H cancer is a non-endometrial cancer. In embodiments, a MSI-H cancer is colorectal cancer.

In embodiments, a cancer is microsatellite stable (MSS). In embodiments, a MSS cancer is MSS endometrial cancer.

In embodiments, a cancer has a defective DNA mismatch repair system (e.g., is a a mismatch repair deficient (MMRd) cancer).

In embodiments, a cancer has a defect in a DNA mismatch repair gene.

In embodiments, a cancer is a hypermutated cancer.

In embodiments, a cancer comprises a mutation in polymerase delta (POLD) (i.e., a cancer is a POLD-mutant cancer). In embodiments, a POLD mutation is a mutation in the exonuclease domain. In embodiments, a POLD mutation is a somatic mutation. In embodiments, a POLD mutation is a germline mutation. In embodiments, a POLD-mutant cancer is identified using sequencing. In embodiments, a POLD-mutant cancer is endometrial cancer. In embodiments, a POLD-mutant cancer is colorectal cancer. In embodiments, a POLD-mutant cancer is brain cancer.

In embodiments, a cancer comprises a mutation in polymerase epsilon (POLE) (i.e., a cancer is a POLE-mutant cancer). In embodiments, a POLE mutation is a mutation in the exonuclease domain. In embodiments, a POLE mutation is a germline mutation. In embodiments, a POLE mutation is a sporadic mutation. In embodiments, a MSI cancer also is associated with a POLE mutation. In embodiments, a cancer is a MSI-H comprising a POLE mutation. In embodiments, a MSS cancer also is associated with a POLE mutation. In embodiments, a POLE mutation is identified using sequencing. In embodiments, a POLE-mutant cancer is endometrial cancer. In embodiments, a POLE-mutant cancer is colon cancer. In embodiments, a POLE-mutant cancer is pancreatic cancer, ovarian cancer, or cancer of the small intestine.

In embodiments, a cancer has homologous recombination repair deficiency/homologous repair deficiency (“HRD”).

In embodiments, immune-related gene expression signatures can be predictive of a response to an anti-PD-1 therapy for cancer as described herein. For example, a gene panel that includes genes associated with IFN-γ signaling can be useful in identifying cancer patients who would benefit from anti-PD-1 therapy. Exemplary gene panels are described in Ayers et al., J. Clin. Invest., 127(8):2930-2940, 2017. In embodiments, a cancer patient has a cancer that is breast cancer (e.g., TNBC) or ovarian cancer. In embodiments, a cancer patient has a cancer that is bladder cancer, gastric cancer, bilary cancer, esophageal cancer, or head and neck squamous cell carcinoma (HNSCC). In embodiments, a cancer patient has a cancer that is anal cancer or colorectal cancer.

In embodiments, a patient has a cancer with elevated expression of tumor-infiltrating lymphocytes (TILs), i.e., a patient has a high-TIL cancer. In embodiments, a high-TIL cancer is breast cancer (e.g., triple negative breast cancer (TNBC) or HER2-positive breast cancer). In embodiments, a high-TIL cancer is a metastatic cancer (e.g., a metastatic breast cancer).

In some embodiments, a patient has a tumor that expresses PD-L1. In some embodiments, PD-L1 status is evaluated in a patient or patient population. In some embodiments, mutational load and baseline gene expression profiles in archival or fresh pre-treatment biopsies are evaluated before, during and/or after treatment with an anti-PD-1 antibody agent. In some embodiments, the status and/or expression of TIM-3 and/or LAG-3 are evaluated in patients.

In embodiments, a cancer is associated with a high tumor mutation burden (TMB) (i.e., a cancer is a high TMB cancer). In embodiments, a cancer is associated with high TMB and MSI-H. In embodiments, a cancer is associated with high TMB and MSI-L. In embodiments, a cancer is associated with high TMB and MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS. In embodiments, a high TMB cancer is colorectal cancer. In embodiments, a high TMB cancer is lung cancer (e.g., small cell lung cancer (SCLC) or non-small cell lung cancer (NSCLC) such as squamous NSCLC or non-squamous NSCLC). In embodiments, a high TMB cancer is melanoma. In embodiments, a high TMB cancer is urothelial cancer.

Cancers can include, for example, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, endometrial cancer, ovarian cancer, or Merkel cell carcinoma (see, e.g., Bhatia et al., Curr. Oncol. Rep., 13(6): 488-497 (2011)).

In embodiments, a cancer is adenocarcinoma, adenocarcinoma of the lung, acute myeloid leukemia (“AML”), acute lymphoblastic leukemia (“ALL”), adrenocortical carcinoma, anal cancer (e.g., squamous cell carcinoma of the anus), appendiceal cancer, B-cell derived leukemia, B-cell derived lymphoma, bladder cancer, brain cancer, breast cancer (e.g., triple negative breast cancer (TNBC)), cancer of the fallopian tube(s), cancer of the testes, cerebral cancer, cervical cancer (e.g., squamous cell carcinoma of the cervix), cholagiocarcinoma, choriocarcinoma, chronic myelogenous leukemia, a CNS tumor, colon cancer or colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, esophageal cancer (e.g., squamous cell carcinoma of the esophagus), Ewing's sarcoma, eye cancer (e.g., uveal melanoma), follicular lymphoma (“FL”), gallbladder cancer, gastric cancer, gastrointestinal cancer, glioma, head and neck cancer (e.g., squamous cell carcinoma of the head and neck (SCHNC)), a hematological cancer, hepatocellular cancer, Hodgkin's lymphoma (HL)/primary mediastinal B-cell lymphoma, kidney cancer, kidney clear cell cancer, laryngeal cancer, leukemia, liver cancer, lung cancer (e.g., non-small cell lung cancer (NSCLC), small cell lung cancer, lung adenocarcinoma, or squamous cell carcinoma of the lung), lymphoma, melanoma, Merkel cell carcinoma, mesothelioma, monocytic leukemia, multiple myeloma, myeloma, a neuroblastic-derived CNS tumor (e.g., neuroblastoma (NB)), non-Hodgkin's lymphoma (NHL), oral cancer, osteosarcoma, ovarian cancer, ovarian carcinoma, pancreatic cancer, peritoneal cancer, primary peritoneal cancer, prostate cancer, relapsed or refractory classic Hodgkin's Lymphoma (cHL), renal cancer (e.g., renal cell carcinoma), rectal cancer, salivary gland cancer (e.g., a salivary gland tumor), sarcoma, skin cancer, small intestine cancer, stomach cancer, squamous cell carcinoma, squamous cell carcinoma of the penis, stomach cancer, T-cell derived leukemia, T-cell derived lymphoma, thymic cancer, a thymoma, thyroid cancer, uveal melanoma, urothelial cell carcinoma, uterine cancer (e.g., uterine endometrial cancer or uterine sarcoma), vaginal cancer (e.g., squamous cell carcinoma of the vagina), vulvar cancer (e.g., squamous cell carcinoma of the vulva), or Wilms tumor.

In embodiments, a cancer is adenocarcinoma, endometrial cancer, breast cancer, ovarian cancer, cervical cancer, fallopian tube cancer, testicular cancer, primary peritoneal cancer, colon cancer, colorectal cancer, stomach cancer, small intestine cancer, squamous cell carcinoma of the anus, squamous cell carcinoma of the penis, squamous cell carcinoma of the cervix, squamous cell carcinoma of the vagina, squamous cell carcinoma of the vulva, soft tissue sarcoma, melanoma, renal cell carcinoma, lung cancer, non-small cell lung cancer, adenocarcinoma of the lung, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, a hematological cancer, multiple myeloma, B-cell lymphoma, T-cell lymphoma, Hodgkin's lymphoma/primary mediastinal B-cell lymphoma, chronic myelogenous leukemia, acute myeloid leukemia, acute lymphoblastic leukemia, non-Hodgkin's lymphoma, neuroblastoma, a CNS tumor, diffuse intrinsic pontine glioma (DIPG), Ewing's sarcoma, embryonal rhabdomyosarcoma, osteosarcoma, or Wilms tumor. In embodiments, the cancer is MSS or MSI-L, is characterized by microsatellite instability, is MSI-H, has high TMB, has high TMB and is MSS or MSI-L, has high TMB and is MSI-H, has a defective DNA mismatch repair system, has a defect in a DNA mismatch repair gene, is a hypermutated cancer, is an HRD cancer, comprises a mutation in polymerase delta (POLD), or comprises a mutation in polymerase epsilon (POLE).

In embodiments, a cancer is bladder cancer, breast cancer (e.g., triple negative breast cancer (TNBC)), cancer of the fallopian tube(s), cholagiocarcinoma, colon adenocarcinoma, endometrial cancer, esophageal cancer, Ewing's sarcoma, gastric cancer, kidney clear cell cancer, lung cancer (e.g., lung adenocarcinoma or lung squamous cell cancer), mesothelioma, ovarian cancer, pancreatic cancer, peritoneal cancer, prostate cancer, uterine endometrial cancer, or uveal melanoma. In embodiments, a cancer is ovarian cancer, cancer of the fallopian tube(s), or peritoneal cancer. In embodiments, a cancer is breast cancer (e.g., TNBC). In embodiments, a cancer is lung cancer (e.g., non-small cell lung cancer). In embodiments, a cancer is prostate cancer.

In embodiments, a cancer is a solid tumor. In embodiments, a cancer is a solid tumor such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non-small cell lung cancer (NSCLC), small cell lung carcinoma, bladder carcinoma, lung cancer, epithelial carcinoma, skin cancer, melanoma, neuroblastoma (NB), or retinoblastoma. In embodiments, a solid tumor is advanced. In embodiments, a solid tumor is a metastatic solid tumor. In embodiments, a solid tumor is a MSI-H solid tumor. In embodiments, a solid tumor is a MSS solid tumor. In embodiments, a solid tumor is a POLE-mutant solid tumor. In embodiments, a solid tumor is a POLD-mutant solid tumor. In embodiments, a solid tumor is a high TMB solid tumor. In embodiments, a solid tumor is associated with HRD.

In other embodiments, a cancer is melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma (see, e.g., Bhatia et al., Curr. Oncol. Rep., 13(6): 488-497 (2011)).

In embodiments a cancer is a lymphoma such as Hodgkin's disease, non-Hodgkin's Lymphoma, multiple myeloma, Waldenstrom's macroglobulinemia, heavy chain disease and polycythemia vera.

In some embodiments, a cancer is a gynecologic cancer (e.g., breast cancer or a cancer of the female reproductive system such as ovarian cancer, fallopian tube cancer, cervical cancer, vaginal cancer, vulvar cancer, uterine cancer, or primary peritoneal cancer). In some embodiments, cancers of the female reproductive system include, but are not limited to, ovarian cancer, cancer of the fallopian tube(s), peritoneal cancer, and breast cancer.

In embodiments, a cancer is an ovarian cancer. In embodiments, an ovarian cancer is an advanced ovarian cancer. In embodiments, an ovarian cancer is a metastatic ovarian cancer. In embodiments, an ovarian cancer is a MSI-H ovarian cancer. In embodiments, an ovarian cancer is a MSS ovarian cancer. In embodiments, an ovarian cancer is a POLE-mutant ovarian cancer. In embodiments, an ovarian cancer is a POLD-mutant ovarian cancer. In embodiments, an ovarian cancer is a high TMB ovarian cancer. In embodiments, an ovarian cancer is associated with homologous recombination repair deficiency/homologous repair deficiency (“HRD”). In embodiments, an ovarian cancer is an ovarian adenocarcinoma. In embodiments, an ovarian cancer is a serous cell ovarian cancer. In embodiments, an ovarian cancer is a clear cell ovarian cancer. In embodiments, an ovarian cancer is epithelial ovarian cancer.

The term ‘ovarian cancer’ is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, a cancer is or comprises a stromal tumor. Stromal tumors develop in the connective tissue cells that hold the ovaries together, which sometimes is the tissue that makes female hormones called estrogen. In some embodiments, a cancer is or comprises a granulosa cell tumor. Granulosa cell tumors may secrete estrogen resulting in unusual vaginal bleeding at the time of diagnosis. In some embodiments, a gynecologic cancer is associated with homologous recombination repair deficiency/homologous repair deficiency (“HRD”) and/or BRCA1/2 mutation(s). In some embodiments, a gynecologic cancer is platinum-sensitive. In some embodiments, a gynecologic cancer has responded to a platinum-based therapy. In some embodiments, a gynecologic cancer has developed resistance to a platinum-based therapy. In some embodiments, a gynecologic cancer has at one time shown a partial or complete response to platinum-based therapy (e.g., a partial or complete response to the last platinum-based therapy or to the penultimate platinum-based therapy). In some embodiments, a gynecologic cancer is now resistant to platinum-based therapy.

In embodiments, a cancer is a fallopian cancer. In embodiments, a fallopian cancer is an advanced fallopian cancer. In embodiments, a fallopian cancer is a metastatic fallopian cancer. In embodiments, a fallopian cancer is a MSI-H fallopian cancer. In embodiments, a fallopian cancer is a MSS fallopian cancer. In embodiments, a fallopian cancer is a POLE-mutant fallopian cancer. In embodiments, a fallopian cancer is a POLD-mutant fallopian cancer. In embodiments, a fallopian cancer is a high TMB fallopian cancer. In embodiments, a fallopian cancer is associated with homologous recombination repair deficiency/homologous repair deficiency (“HRD”). In embodiments, a fallopian cancer is a serous cell fallopian cancer. In embodiments, a fallopian cancer is a clear cell fallopian cancer.

In embodiments, a cancer is a primary peritoneal cancer. In embodiments, a primary peritoneal cancer is an advanced primary peritoneal cancer. In embodiments, a primary peritoneal cancer is a metastatic primary peritoneal cancer. In embodiments, a primary peritoneal cancer is a MSI-H primary peritoneal cancer. In embodiments, a primary peritoneal cancer is a MSS primary peritoneal cancer. In embodiments, a primary peritoneal cancer is a POLE-mutant primary peritoneal cancer. In embodiments, a primary peritoneal cancer is a POLD-mutant primary peritoneal cancer. In embodiments, a primary peritoneal cancer is a high TMB primary peritoneal cancer. In embodiments, a primary peritoneal cancer is associated with homologous recombination repair deficiency/homologous repair deficiency (“HRD”). In embodiments, a primary peritoneal cancer is a serous cell primary peritoneal cancer. In embodiments, a primary peritoneal cancer is a clear cell primary peritoneal cancer.

In embodiments, a cancer is a breast cancer. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative. In embodiments, a breast cancer is an advanced breast cancer. In some embodiments, a cancer is a stage II, stage III or stage IV breast cancer. In some embodiments, a cancer is a stage IV breast cancer. In embodiments, a breast cancer is a metastatic breast cancer. In embodiments, a breast cancer is a MSI-H breast cancer. In embodiments, a breast cancer is a MSS breast cancer. In embodiments, a breast cancer is a POLE-mutant breast cancer. In embodiments, a breast cancer is a POLD-mutant breast cancer. In embodiments, a breast cancer is a high TMB breast cancer. In embodiments, a breast cancer is associated with homologous recombination repair deficiency/homologous repair deficiency (“HRD”). In embodiments, a cancer is ER-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or triple negative breast cancer (TNBC). In embodiments, a cancer is triple negative breast cancer (TNBC).

In embodiments, a cancer is endometrial cancer (“EC”). From the pathogenetic point of view, EC falls into two different types, so-called types I and II. Type I tumors are low-grade and estrogen-related endometrioid carcinomas (EEC) while type II are non-endometrioid (NEEC) (mainly serous and clear cell) carcinomas. The World Health Organization has recently updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, lowgrade EEC (EEC 1-2) contains tubular glands, somewhat resembling the proliferative endometrium, with architectural complexity with fusion of the glands and cribriform pattern. High-grade EEC shows solid pattern of growth. In contrast, SC occurs in postmenopausal patients in absence of hyperestrogenism. At the microscope, SC shows thick, fibrotic or edematous papillae with prominent stratification of tumor cells, cellular budding, and anaplastic cells with large, eosinophilic cytoplasms. The vast majority of EEC are low grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC 3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively. Endometrial cancers can also be classified into four molecular subgroups: (1) ultramutated/POLE-mutant; (2) hypermutated MSI+ (e.g., MSI-H or MSI-L); (3) copy number low/microsatellite stable (MSS); and (4) copy number high/serous-like. Approximately 28% of cases are MSI-high. (Murali, Lancet Oncol. (2014). In some embodiments, a patient has a mismatch repair deficient subset of 2L endometrial cancer. In embodiments, an endometrial cancer is an advanced cancer. In embodiments, an endometrial cancer is a metastatic cancer. In embodiments, an endometrial cancer is a MSI-H endometrial cancer. In embodiments, an endometrial cancer is a MSI-L endometrial cancer. In embodiments, an endometrial cancer is a MSS endometrial cancer. In embodiments, an endometrial cancer is a POLE-mutant endometrial cancer (e.g., a MSI-H endometrial cancer comprising a POLE mutation). In embodiments, an endometrial cancer is a POLD-mutant endometrial cancer (e.g., MSI-H endometrial cancer comprising a POLD mutation). In embodiments, an endometrial cancer is a high TMB endometrial cancer. In embodiments, an endometrial cancer is associated with homologous recombination repair deficiency/homologous repair deficiency (“HRD”).

In embodiments a cancer is a gonadal tumor.

In embodiments, a cancer is a non-endometrial cancer (e.g., a non-endometrial solid tumor). In embodiments, a non-endometrial cancer is an advanced cancer. In embodiments, a non-endometrial cancer is a metastatic cancer. In embodiments, a non-endometrial cancer is a MSI-H cancer. In embodiments, a non-endometrial cancer is a MSI-L endometrial cancer. In embodiments, a non-endometrial cancer is a MSS cancer. In embodiments, a non-endometrial cancer is a POLE-mutant cancer (e.g., a MSI-H non-endometrial cancer comprising a POLE mutation). In embodiments, a non-endometrial cancer is a POLD-mutant cancer (e.g., a MSI-H non-endometrial cancer comprising a POLD mutation). In embodiments, a non-endometrial cancer is a solid tumor (e.g., a MSS solid tumor, a MSI-H solid tumor, a POLD mutant solid tumor, or a POLE-mutant solid tumor). In embodiments, a non-endometrial cancer is a high TMB cancer. In embodiments, a non-endometrial cancer is associated with homologous recombination repair deficiency/homologous repair deficiency (“HRD”).

In some embodiments, a patient or population of patients has a hematological cancer. In some embodiments, the patient has a hematological cancer such as diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), acute lymphoblastic leukemia (“ALL”), or Multiple myeloma (“MM”). In embodiments, a cancer is a blood-borne cancer such as acute lymphoblastic leukemia (“ALL”), acute lymphoblastic B-cell leukemia, acute lymphoblastic T-cell leukemia, acute myeloblastic leukemia (“AML”), acute lymphoblastic leukemia (“ALL”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CCML”), chronic lymphocytic leukemia (“CLL”), hairy cell leukemia and multiple myeloma; acute and chronic leukemias such as lymphoblastic, myelogenous, lymphocytic, and myelocytic leukemias. In embodiments, a hematological cancer is a lymphoma (e.g., Hodgkin's lymphoma (e.g., relapsed or refractory classic Hodgkin's Lymphoma (cHL), non-Hodgkin's lymphoma, diffuse large B-cell lymphoma, or precursor T-lymphoblastic lymphoma), lymphoepithelial carcinoma, or malignant histiocytosis

In embodiments, a cancer is diffuse large B cell lymphoma (“DLBCL”). In embodiments, diffuse large B cell lymphoma is advanced diffuse large B cell lymphoma. In embodiments, diffuse large B cell lymphoma is metastatic diffuse large B cell lymphoma. In embodiments, diffuse large B cell lymphoma is MSI-H diffuse large B cell lymphoma. In embodiments, diffuse large B cell lymphoma is MSS diffuse large B cell lymphoma. In embodiments, diffuse large B cell lymphoma is POLE-mutant diffuse large B cell lymphoma. In embodiments, diffuse large B cell lymphoma is POLD-mutant diffuse large B cell lymphoma. In embodiments, a diffuse large B cell lymphoma is associated with homologous recombination repair deficiency/homologous repair deficiency (“HRD”).

In embodiments, a cancer is acute lymphoblastic leukemia (“ALL”). In embodiments, acute lymphoblastic leukemia is advanced acute lymphoblastic leukemia. In embodiments, acute lymphoblastic leukemia is metastatic acute lymphoblastic leukemia. In embodiments, acute lymphoblastic leukemia is MSI-H acute lymphoblastic leukemia. In embodiments, acute lymphoblastic leukemia is MSS acute lymphoblastic leukemia. In embodiments, acute lymphoblastic leukemia is POLE-mutant acute lymphoblastic leukemia. In embodiments, acute lymphoblastic leukemia is POLD-mutant acute lymphoblastic leukemia. In embodiments, an acute lymphoblastic leukemia is associated with homologous recombination repair deficiency/homologous repair deficiency (“HRD”).

In embodiments, a cancer is acute myeloid leukemia (“AML”). In embodiments, acute myeloid leukemia is advanced acute myeloid leukemia. In embodiments, acute myeloid leukemia is metastatic acute myeloid leukemia. In embodiments, acute myeloid leukemia is MSI-H acute myeloid leukemia. In embodiments, acute myeloid leukemia is MSS acute myeloid leukemia. In embodiments, acute myeloid leukemia is POLE-mutant acute myeloid leukemia. In embodiments, acute myeloid leukemia is POLD-mutant acute myeloid leukemia. In embodiments, an acute myeloid leukemia is associated with homologous recombination repair deficiency/homologous repair deficiency (“HRD”).

In embodiments, a cancer is non-Hodgkin's lymphoma (NHL). In embodiments, non-Hodgkin's lymphoma is advanced non-Hodgkin's lymphoma. In embodiments, non-Hodgkin's lymphoma is metastatic non-Hodgkin's lymphoma. In embodiments, non-Hodgkin's lymphoma is MSI-H non-Hodgkin's lymphoma. In embodiments, non-Hodgkin's lymphoma is MSS non-Hodgkin's lymphoma In embodiments, non-Hodgkin's lymphoma is POLE-mutant non-Hodgkin's lymphoma. In embodiments, non-Hodgkin's lymphoma is POLD-mutant non-Hodgkin's lymphoma. In embodiments, non-Hodgkin's lymphoma is associated with homologous recombination repair deficiency/homologous repair deficiency (“HRD”).

In embodiments, a cancer is Hodgkin's lymphoma (HL). In embodiments, Hodgkin's lymphoma is advanced Hodgkin's lymphoma. In embodiments, Hodgkin's lymphoma is metastatic Hodgkin's lymphoma. In embodiments, Hodgkin's lymphoma is MSI-H Hodgkin's lymphoma. In embodiments, Hodgkin's lymphoma is MSS Hodgkin's lymphoma In embodiments, Hodgkin's lymphoma is POLE-mutant Hodgkin's lymphoma. In embodiments, Hodgkin's lymphoma is POLD-mutant Hodgkin's lymphoma. In embodiments, Hodgkin's lymphoma is associated with homologous recombination repair deficiency/homologous repair deficiency (“HRD”).

In embodiments, a cancer is a non-CNS cancer (e.g., a non-CNS solid tumor). In embodiments, a cancer is neuroblastoma, hepatoblastoma, hepatocellular carcinoma, Wilms tumor, renal cell carcinoma, melanoma, adrenocortical carcinoma, adenocarcinoma of the colon, myoepithelial carcinoma, thymic cell carcinoma, nasopharyngeal carcinoma, squamous cell carcinoma, mesothelioma, or clivus chordoma. In embodiments, a cancer is extracranial embryonal neuroblastoma.

In embodiments, a cancer is a CNS cancer (e.g., a primary CNS malignancy) such as brain cancer. In embodiments, a cancer is ependymoma. In embodiments, a cancer is a brain cancer (e.g., glioblastoma multiforme, gliosarcoma, astrocytoma, glioblastoma, medulloblastoma, glioma, supratentorial primitive neuroectodermal tumor, atypical teratoid rhabdoid tumor, choroid plexus carcinoma, malignant ganglioma, gliomatosis cerebri, meningioma, or paraganglioma). In embodiments, a cancer is high-grade astrocytoma, low-grade astrocytoma, anaplastic astrocytoma, fibrillary astrocytoma, pilocytic astrocytoma, a high-grade glioma, low-grade glioma, diffuse intrinsic pontine glioma (DIPG), or anaplastic mixed glioma. In embodiments, a cancer is neuroblastoma (NB), glioma, diffuse intrinsic pontine glioma (DIPG), pilocytic astrocytoma, astrocytoma, anaplastic astrocytoma, glioblastoma multiforme, medulloblastoma, craniopharyngioma, ependymoma, pinealoma, hemangioblastoma, acoustic neuroma, oligodendroglioma, meningioma, vestibular schwannoma, adenoma, metastatic brain tumor, meningioma, spinal tumor, or medulloblastoma.

In embodiments, a cancer is a CNS tumor. In embodiments, a CNS tumor is advanced. In embodiments, a CNS tumor is a metastatic CNS tumor. In embodiments, a CNS tumor is a MSI-H CNS tumor. In embodiments, a CNS tumor is a MSS CNS tumor. In embodiments, a CNS tumor is a POLE-mutant CNS tumor. In embodiments, a CNS tumor is a POLD-mutant CNS tumor. In embodiments, a CNS tumor is a high TMB CNS tumor. In embodiments, a CNS tumor is associated with homologous recombination repair deficiency/homologous repair deficiency (“HRD”). In embodiments, a CNS tumor is an advanced well-differentiated neuroendocrine tumor.

In embodiments, a cancer is a neuroblastoma (NB). In embodiments, a neuroblastoma is an advanced neuroblastoma. In embodiments, a neuroblastoma is a metastatic neuroblastoma. In embodiments, neuroblastoma is a MSI-H neuroblastoma. In embodiments, a neuroblastoma is a MSS neuroblastoma. In embodiments, a neuroblastoma is a POLE-mutant neuroblastoma. In embodiments, a neuroblastoma is a POLD-mutant neuroblastoma. In embodiments, a neuroblastoma is a high TMB neuroblastoma. In embodiments, a neuroblastoma is associated with homologous recombination repair deficiency/homologous repair deficiency (“HRD”).

In embodiments, a cancer is diffuse intrinsic pontine glioma (DIPG). In embodiments, a DIPG is an advanced DIPG. In embodiments, a DIPG is a metastatic DIPG. In embodiments, DIPG is a MSI-H DIPG. In embodiments, a DIPG is a MSS DIPG. In embodiments, a DIPG is a POLE-mutant DIPG. In embodiments, a DIPG is a POLD-mutant DIPG. In embodiments, a DIPG is a high TMB DIPG. In embodiments, a DIPG is associated with homologous recombination repair deficiency/homologous repair deficiency (“HRD”).

In embodiments, a cancer is a sarcoma.

In embodiments, a sarcoma is Ewings sarcoma, osteosarcoma, rhabdomyosarcoma, embryonal rhabdomyosarcoma, synovial sarcoma, alveolar rhabdomyosarcoma, alveolar soft part sarcoma, spindle cell sarcoma, angiosarcoma, epithelialoid sarcoma, inflammatory myofibroblastic tumor, or malignant rhadoid tumor.

In embodiments, a sarcoma is an advanced sarcoma. In embodiments, a sarcoma is a metastatic sarcoma. In embodiments, a sarcoma is a MSI-H sarcoma. In embodiments, a sarcoma is a MSS sarcoma. In embodiments, a sarcoma is a POLE-mutant sarcoma. In embodiments, a sarcoma is a POLD-mutant sarcoma. In embodiments, a sarcoma is a high TMB sarcoma. In embodiments, a sarcoma is associated with homologous recombination repair deficiency/homologous repair deficiency (“HRD”).

In embodiments, a cancer is Ewing's sarcoma. In embodiments, Ewing's sarcoma is an advanced Ewing's sarcoma. In embodiments, Ewing's sarcoma is a metastatic Ewing's sarcoma. In embodiments, Ewing's sarcoma is a MSI-H Ewing's sarcoma. In embodiments, Ewing's sarcoma is a MSS Ewing's sarcoma. In embodiments, Ewing's sarcoma is a POLE-mutant Ewing's sarcoma. In embodiments, Ewing's sarcoma is a POLD-mutant Ewing's sarcoma. In embodiments, Ewing's sarcoma is a high TMB Ewing's sarcoma. In embodiments, Ewing's sarcoma is associated with homologous recombination repair deficiency/homologous repair deficiency (“HRD”).

In embodiments, a cancer is an embryonal rhabdomyosarcoma (ERS). In embodiments, an embryonal rhabdomyosarcoma is an advanced embryonal rhabdomyosarcoma. In embodiments, an embryonal rhabdomyosarcoma is a metastatic embryonal rhabdomyosarcoma. In embodiments, an embryonal rhabdomyosarcoma is a MSI-H embryonal rhabdomyosarcoma. In embodiments, an embryonal rhabdomyosarcoma is a MSS embryonal rhabdomyosarcoma. In embodiments, an embryonal rhabdomyosarcoma is a POLE-mutant embryonal rhabdomyosarcoma. In embodiments, an embryonal rhabdomyosarcoma is a POLD-mutant embryonal rhabdomyosarcoma. In embodiments, an embryonal rhabdomyosarcoma is a high TMB embryonal rhabdomyosarcoma. In embodiments, an embryonal rhabdomyosarcoma is associated with homologous recombination repair deficiency/homologous repair deficiency (“HRD”).

In embodiments, a cancer is an osteosarcoma (OS). In embodiments, an osteosarcoma is an advanced osteosarcoma. In embodiments, an osteosarcoma is a metastatic osteosarcoma. In embodiments, an osteosarcoma is a MSI-H osteosarcoma. In embodiments, an osteosarcoma is a MSS osteosarcoma. In embodiments, an osteosarcoma is a POLE-mutant osteosarcoma. In embodiments, an osteosarcoma is a POLD-mutant osteosarcoma. In embodiments, an osteosarcoma is a high TMB osteosarcoma. In embodiments, an osteosarcoma is associated with homologous recombination repair deficiency/homologous repair deficiency (“HRD”).

In embodiments, a cancer is a soft tissue sarcoma. In embodiments, a soft tissue sarcoma is an advanced soft tissue sarcoma. In embodiments, a soft tissue sarcoma is a metastatic soft tissue sarcoma. In embodiments, a soft tissue sarcoma is a MSI-H soft tissue sarcoma. In embodiments, a soft tissue sarcoma is a MSS soft tissue sarcoma. In embodiments, a soft tissue sarcoma is a POLE-mutant soft tissue sarcoma. In embodiments, a soft tissue sarcoma is a POLD-mutant soft tissue sarcoma. In embodiments, a soft tissue sarcoma is a high TMB soft tissue sarcoma. In embodiments, a soft tissue sarcoma is associated with homologous recombination repair deficiency/homologous repair deficiency (“HRD”). In embodiments, a soft tissue sarcoma is leiomyosarcoma.

In embodiments, a cancer is a lung cancer. In embodiments, a lung cancer is a squamous cell carcinoma of the lung. In embodiments, a lung cancer is a MSI-H lung cancer. In embodiments, a lung cancer is a MSS lung cancer. In embodiments, a lung cancer is a POLE-mutant lung cancer. In embodiments, a lung cancer is a POLD-mutant lung cancer. In embodiments, a lung cancer is a high TMB lung cancer. In embodiments, a lung cancer is associated with homologous recombination repair deficiency/homologous repair deficiency (“HRD”). In embodiments, a lung cancer is small cell lung cancer (SCLC). In embodiments, a lung cancer is non-small cell lung cancer (NSCLC) such as squamous NSCLC. In embodiments, a lung cancer is an ALK-translocated lung cancer (e.g., ALK-translocated NSCLC). In embodiments, a cancer is NSCLC with an identified ALK translocation. In embodiments, a lung cancer is an EGFR-mutant lung cancer (e.g., EGFR-mutant NSCLC). In embodiments, a cancer is NSCLC with an identified EGFR mutation. In embodiments, a lung cancer is mesothelioma.

In embodiments, a cancer is a melanoma. In embodiments, a melanoma is an advanced melanoma. In embodiments, a melanoma is a metastatic melanoma. In embodiments, a melanoma is a MSI-H melanoma. In embodiments, a melanoma is a MSS melanoma. In embodiments, a melanoma is a POLE-mutant melanoma. In embodiments, a melanoma is a POLD-mutant melanoma. In embodiments, a melanoma is a high TMB melanoma.

In embodiments, a cancer is a carcinoma. In embodiments, a carcinoma is an advanced carcinoma. In embodiments, a carcinoma is a metastatic carcinoma. In embodiments, a carcinoma is a MSI-H carcinoma. In embodiments, a carcinoma is a MSS carcinoma. In embodiments, a carcinoma is a POLE-mutant carcinoma. In embodiments, a carcinoma is a POLD-mutant carcinoma. In embodiments, a carcinoma is a high TMB carcinoma. In embodiments, a carcinoma is renal cell carcinoma (RCC).

In embodiments, a cancer is a squamous cell carcinoma. In embodiments, a squamous cell carcinoma is an advanced cancer. In embodiments, a squamous cell carcinoma is a metastatic cancer. In embodiments, a squamous cell carcinoma is MSI-H. In embodiments, a squamous cell carcinoma is MSS. In embodiments, a squamous cell carcinoma is a POLE-mutant cancer. In embodiments, squamous cell carcinoma is associated with homologous recombination repair deficiency/homologous repair deficiency (“HRD”). In embodiments, a cancer is squamous cell carcinoma of the lung. In embodiments, a cancer is squamous cell carcinoma of the esophagus. In embodiments, a cancer is squamous cell carcinoma of the anus, penis, cervix, vagina, or vulva. In embodiments, a cancer is head and neck squamous cell carcinoma (HNSCC).

In embodiments, a cancer is an adenocarcinoma. In embodiments, an adenocarcinoma is an advanced adenocarcinoma. In embodiments, an adenocarcinoma is a metastatic adenocarcinoma. In embodiments, an adenocarcinoma is a MSI-H adenocarcinoma. In embodiments, an adenocarcinoma is a MSS adenocarcinoma. In embodiments, an adenocarcinoma is a POLE-mutant adenocarcinoma. In embodiments, an adenocarcinoma is a POLD-mutant adenocarcinoma. In embodiments, an adenocarcinoma is a high TMB adenocarcinoma. In embodiments, an adenocarcinoma is gastric adenocarcinoma. In embodiments, an adenocarcinoma is esophageal adenocarcinoma. In embodiments, an adenocarcinoma is prostate adenocarcinoma (e.g., castration resistant prostate adenocarcinoma). In embodiments, an adenocarcinoma is an ovarian adenocarcinoma.

In embodiments, a cancer is Wilms tumor. In embodiments, Wilms tumor is an advanced Wilms tumor. In embodiments, Wilms tumor is a metastatic Wilms tumor. In embodiments, Wilms tumor is a MSI-H Wilms tumor. In embodiments, Wilms tumor is a MSS Wilms tumor. In embodiments, Wilms tumor is a POLE-mutant Wilms tumor. In embodiments, Wilms tumor is a POLD-mutant Wilms tumor. In embodiments, Wilms tumor is a high TMB Wilms tumor. In embodiments, Wilms tumor is associated with homologous recombination repair deficiency/homologous repair deficiency (“HRD”).

In embodiments, a cancer is a colorectal (CRC) cancer (e.g., a solid tumor). In embodiments, a colorectal cancer is an advanced colorectal cancer. In embodiments, a colorectal cancer is a metastatic colorectal cancer. In embodiments, a colorectal cancer is a MSI-H colorectal cancer. In embodiments, a colorectal cancer is a MSS colorectal cancer. In embodiments, a colorectal cancer is a POLE-mutant colorectal cancer. In embodiments, a colorectal cancer is a POLD-mutant colorectal cancer. In embodiments, a colorectal cancer is a high TMB colorectal cancer. In embodiments, a colorectal cancer is associated with homologous recombination repair deficiency/homologous repair deficiency (“HRD”).

### Lung Cancer

In embodiments, a cancer is a lung cancer.

Lung cancer is the most common cause of cancer mortality globally and the second most common cancer in both men and women. About 14% of all new cancers are lung cancers. In the United States (US), there are projected to be 222,500 new cases of lung cancer (116,990 in men and 105,510 in women) and 155,870 deaths from lung cancer (84,590 in men and 71,280 in women) in 2017.

The two major forms of lung cancer are non-small cell lung cancer (NSCLC) and small cell lung cancer. NSCLC is a heterogeneous disease that consists of adenocarcinoma, large-cell carcinoma, and squamous cell carcinoma (sqNSCLC), and comprises approximately 80% to 85% of all lung cancers. Squamous cell carcinoma of the lung accounts for 20% to 30% of NSCLC. Despite advances in early detection and standard treatment, NSCLC is often diagnosed at an advanced stage, has poor prognosis, and is the leading cause of cancer deaths worldwide.

Platinum-based doublet therapy, maintenance chemotherapy, and anti-angiogenic agents in combination with chemotherapy have contributed to improved patient outcomes in advanced NSCLC. The identification of point mutations (epidermal growth factor receptor [EGFR], BRAF), gene fusions due to chromosomal translocations (anaplastic lymphoma kinase [ALK], ROS-1), and gene amplifications (mesenchymal epithelial transition factor [MET]) can serve as oncogenic drivers in providing treatment to the cancer patient. For most NSCLC patients without targetable oncogene drivers, first-line platinum-based chemotherapy was until recently the only standard treatment approach.

In embodiments, a lung cancer is an advanced lung cancer. In embodiments, a lung cancer is a metastatic lung cancer. In embodiments, a lung cancer is squamous cell carcinoma of the lung. In embodiments, a lung cancer is small cell lung cancer (SCLC). In embodiments, a lung cancer is non-small cell lung cancer (NSCLC). In embodiments, a lung cancer is an ALK-translocated lung cancer (e.g., a lung cancer with a known ALK-translocation). In embodiments, a lung cancer is an EGFR-mutant lung cancer (e.g., a lung cancer with a known EGFR mutation). In embodiments, a lung cancer is a MSI-H lung cancer. In embodiments, a lung cancer is a MSS lung cancer. In embodiments, a lung cancer is a POLE-mutant lung cancer. In embodiments, a lung cancer is a POLD-mutant lung cancer. In embodiments, a lung cancer is a high TMB lung cancer. In embodiments, a lung cancer is associated with homologous recombination repair deficiency/homologous repair deficiency (“HRD”) or is characterized by a homologous recombination repair (HRR) gene mutation or deletion.

In embodiments, an advanced lung cancer (e.g., advanced NSCLC) is stage III cancer or stage IV cancer. In embodiments, an advanced lung cancer (e.g., advanced NSCLC) is stage III cancer. In embodiments, an advanced lung cancer (e.g., advanced NSCLC) is stage IV cancer. In embodiments, an advanced lung cancer (e.g., advanced NSCLC) is locally advanced. In embodiments, an advanced lung cancer (e.g., advanced NSCLC) is metastatic.

In embodiments, a subject having lung cancer (e.g., NSCLC such as advanced NSCLC) is treatment-naïve for the lung cancer. In embodiments, a subject having lung cancer (e.g., NSCLC such as advanced NSCLC) is treatment-naïve for the lung cancer and has not previously received immunotherapy (e.g., anti-PD-1 therapy) nor chemotherapy. In embodiments, a subject having lung cancer (e.g., NSCLC such as advanced NSCLC) is treatment-naïve for the lung cancer and has not previously received immunotherapy. In embodiments, a subject having lung cancer (e.g., NSCLC such as advanced NSCLC) is treatment-naïve for the lung cancer and has not previously received an anti-PD-1 therapy (“PD-1-naïve”). In embodiments, a subject having lung cancer (e.g., NSCLC such as advanced NSCLC) is treatment-naïve for the lung cancer and has not previously received chemotherapy (“chemotherapy-naïve”). In embodiments, a subject having lung cancer (e.g., NSCLC such as advanced NSCLC) is treatment-naïve for the lung cancer and has not previously received chemotherapy such as platinum-based chemotherapy or chemotherapy comprising an inhibitor of EGFR, ALK, ROS-1, and/or MET.

In embodiments, a lung cancer (e.g., NSCLC such as advanced NSCLC) does not express PD-L1.

In embodiments, a lung cancer (e.g., NSCLC such as advanced NSCLC) expresses PD-L1 (e.g., as determined by an assay such as an immunohistochemical (IHC) assay). In embodiments, a lung cancer (e.g., NSCLC such as advanced NSCLC) expresses ≥1% PD-L1 (e.g., as determined by an assay such as an immunohistochemical (IHC) assay). In embodiments, a lung cancer (e.g., NSCLC such as advanced NSCLC) expresses ≥50% PD-L1 (e.g., as determined by an assay such as an immunohistochemical (IHC) assay). In embodiments, a lung cancer (e.g., NSCLC such as advanced NSCLC) is a high PD-L1 cancer (e.g., a cancer that expresses ≥50% PD-L1 (e.g., as determined by an assay such as an immunohistochemical (IHC) assay)).

In embodiments, a lung cancer is small cell lung cancer (SCLC).

In embodiments, a lung cancer is non-small cell lung cancer (NSCLC) such as adenocarcinoma, large-cell carcinoma, or squamous cell carcinoma (sqNSCLC). In embodiments, a NSCLC is lung adenocarcinoma. In embodiments, a NSCLC is large cell carcinoma of the lung. In embodiments, a NSCLC is squamous cell carcinoma of the lung (sqNSCLC).

In embodiments, a lung cancer is an ALK-translocated lung cancer (e.g., ALK-translocated NSCLC). In embodiments, a cancer is NSCLC (e.g., advanced NSCLC) with an identified ALK translocation.

In embodiments, a lung cancer (e.g., NSCLC such as advanced NSCLC) does not have an ALK-translocation. In embodiments, a cancer is NSCLC (e.g., advanced NSCLC) without ALK translocation.

In embodiments, a lung cancer (e.g., NSCLC such as advanced NSCLC) is an EGFR-mutant lung cancer (e.g., EGFR-mutant NSCLC). In embodiments, a cancer is NSCLC (e.g., advanced NSCLC) with an identified EGFR mutation.

In embodiments, a lung cancer (e.g., NSCLC such as advanced NSCLC) does not have an EGFR mutation. In embodiments, a cancer is NSCLC (e.g., advanced NSCLC) without an EGFR mutation.

In embodiments, a lung cancer (e.g., NSCLC such as advanced NSCLC) is an ROS-1-translocated lung cancer (e.g., ROS-1-translocated NSCLC). In embodiments, a cancer is NSCLC (e.g., advanced NSCLC) with an identified ROS-1 translocation.

In embodiments, a lung cancer (e.g., NSCLC such as advanced NSCLC) does not have an ROS-1-translocation. In embodiments, a cancer is NSCLC (e.g., advanced NSCLC) without ROS-1 translocation.

In embodiments, a lung cancer (e.g., NSCLC such as advanced NSCLC) is characterized by a gene amplification (e.g., in mesenchymal epithelial transition factor (MET)). In embodiments, a cancer is NSCLC (e.g., advanced NSCLC) characterized by a MET amplification.

In embodiments, a lung cancer (e.g., NSCLC such as advanced NSCLC) is characterized by an EGFR mutation, an ALK translocation, a ROS-1 translocation, and/or a gene amplification in mesenchymal epithelial transition factor (MET).

In embodiments, a lung cancer (e.g., NSCLC such as advanced NSCLC) does not have an EGFR mutation, an ALK translocation, a ROS-1 translocation, nor a gene amplification in mesenchymal epithelial transition factor (MET).

In embodiments, a lung cancer (e.g., NSCLC such as advanced NSCLC) is not characterized by a gene amplification. In embodiments, a cancer is NSCLC (e.g., advanced NSCLC) that is not characterized by a gene amplification. In embodiments, a cancer is NSCLC (e.g., advanced NSCLC) that is not characterized by a gene amplification in mesenchymal epithelial transition factor (MET).

In embodiments, a subject is treatment-naïve (e.g., chemotherapy-naïve and/or PD-1-naïve). In embodiments, a treatment-naïve subject has not previously received chemotherapy (e.g., chemotherapy that is platinum-based chemotherapy and/or an inhibitor of any of EGFR, ALK, ROS-1, and MET) nor a previous anti-PD-1 therapy (e.g., anti-PD-1 therapy that is an inhibitor of PD-1 and/or PD-L1/L2). In embodiments, a lung cancer (e.g., NSCLC such as advanced NSCLC) is advanced. In embodiments, an advanced lung cancer (e.g., advanced NSCLC) is locally advanced. In embodiments, an advanced lung cancer (e.g., advanced NSCLC) is metastatic. In embodiments, a lung cancer (e.g., NSCLC such as advanced NSCLC) expresses PD-L1. In embodiments, a lung cancer (e.g., NSCLC such as advanced NSCLC) is high PD-L1 (e.g., TPS≥50%). In embodiments, PD-L1 expression is determined using a immunohistochemical (IHC) assay.

### Measuring Tumor Response

In embodiments, methods described herein can provide a clinical benefit to a subject.

In some embodiments, a clinical benefit is a complete response (“CR”), a partial response (“PR”) or a stable disease (“SD”). In some embodiments, a clinical benefit corresponds to at least SD. In some embodiments, a clinical benefit corresponds to at least a PR. In some embodiments, a clinical benefit corresponds to a CR. In some embodiments, at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90% or 95% of patients achieve a clinical benefit. In some embodiments, at least 5% of patients achieve a clinical benefit. In some embodiments, at least 5% of patients achieve SD. In some embodiments, at least 5% of patients achieve at least a PR. In some embodiments, at least 5% of patients achieve CR. In some embodiments, at least 20% of patients achieve a clinical benefit. In some embodiments, at least 20% of patients achieve SD.

In some embodiments, the clinical benefit (e.g., SD, PR and/or CR) is determined in accordance with Response Evaluation Criteria in Solid Tumors (RECIST). In some embodiments, the clinical benefit (e.g., SD, PR and/or CR) is determined in accordance RECIST guidelines.

In some embodiments, tumor response can be measured by, for example, the RECIST v 1.1 guidelines. The guidelines are provided by E. A. Eisenhauer, et al., “New response evaluation criteria in solid tumors: Revised RECIST guideline (version 1.1.),” Eur. J. of Cancer, 45: 228-247 (2009), which is incorporated by reference in its entirety. The guidelines require, first, estimation of the overall tumor burden at baseline, which is used as a comparator for subsequent measurements. Tumors can be measured via use of any imaging system known in the art, for example, by a CT scan, or an X-ray. Measurable disease is defined by the presence of at least one measurable lesion. In studies where the primary endpoint is tumor progression (either time to progression or proportion with progression at a fixed date), the protocol must specify if entry is restricted to those with measurable disease or whether patients having non-measurable disease only are also eligible.

When more than one measurable lesion is present at baseline, all lesions up to a maximum of five lesions total (and a maximum of two lesions per organ) representative of all involved organs should be identified as target lesions and will be recorded and measured at baseline (this means in instances where patients have only one or two organ sites involved a maximum of two and four lesions respectively will be recorded).

Target lesions should be selected on the basis of their size (lesions with the longest diameter), be representative of all involved organs, but in addition should be those that lend themselves to reproducible repeated measurements.

Lymph nodes merit special mention since they are normal anatomical structures which may be visible by imaging even if not involved by tumor. Pathological nodes which are defined as measurable and may be identified as target lesions must meet the criterion of a short axis of P15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT scan this is almost always the axial plane; for MRI the plane of acquisition may be axial, sagittal or coronal). The smaller of these measures is the short axis.

For example, an abdominal node which is reported as being 20 mm-30 mm has a short axis of 20 mm and qualifies as a malignant, measurable node. In this example, 20 mm should be recorded as the node measurement. All other pathological nodes (those with short axis P10 mm but <15 mm) should be considered non-target lesions. Nodes that have a short axis <10 mm are considered non-pathological and should not be recorded or followed.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterize any objective tumor regression in the measurable dimension of the disease.

All other lesions (or sites of disease) including pathological lymph nodes should be identified as non-target lesions and should also be recorded at baseline. Measurements are not required and these lesions should be followed as ‘present’, ‘absent’, or in rare cases ‘unequivocal progression.’ In addition, it is possible to record multiple nontarget lesions involving the same organ as a single item on the case record form (e.g., ‘multiple enlarged pelvic lymph nodes’ or ‘multiple liver metastases’).

In some embodiments, tumor response can be measured by, for example, the immune-related RECIST (irRECIST) guidelines, which include immune related Response Criteria (irRC). In irRC, measurable lesions are measured that have at least one dimension with a minimum size of 10 mm (in the longest diameter by CT or MRI scan) for nonnodal lesions and greater than or equal to 15 mm for nodal lesions, or at least 20 mm by chest X-ray.

In some embodiments, Immune Related Response Criteria include CR (complete disappearance of all lesions (measurable or not, and no new lesions)); PR (decrease in tumor burden by 50% or more relative to baseline); SD (not meeting criteria for CR or PR in the absence of PD); or PD (an increase in tumor burden of at 25% or more relative to nadir). Detailed description of irRECIST can be found at Bohnsack et al., (2014) ESMO, ABSTRACT 4958 and Nishino et al., (2013) Clin. Cancer Res. 19(14): 3936-43.

In some embodiments, tumor response can be assessed by either irRECIST or RECIST version 1.1. In some embodiments, tumor response can be assessed by both irRECIST and RECIST version 1.1.

### Enhancement of Immune Response and Treatment of Immune Disorders

In an embodiment, the invention provides a method of enhancing an immune response in a mammal, or treating or preventing a disease or disorder in a mammal that is responsive to immune checkpoint inhibition, which method comprises administering to a mammal in need thereof one or more immune checkpoint inhibitors or pharmaceutical composition described herein, whereupon an immune response in the mammal is enhanced, or the disease or disorder is treated in the mammal. The immune response is augmented for example by augmenting antigen specific T effector function. The antigen can be a viral (e.g., HIV), bacterial, parasitic or tumor antigen (e.g., any antigen described herein). In embodiments, an immune response is a natural immune response. By natural immune response is meant an immune response that is a result of an infection. In embodiments, an infection is a chronic infection. In embodiments, an infection is an acute infection.

Increasing or enhancing an immune response to an antigen can be measured by a number of methods known in the art. For example, an immune response can be measured by measuring any one of the following: T cell activity, T cell proliferation, T cell activation, production of effector cytokines, and T cell transcriptional profile. In embodiments, an immune response is a response induced due to a vaccination. Accordingly, in another aspect the invention provides a method of increasing vaccine efficiency by administering to the subject a monoclonal antibody or scFv antibody of the invention and a vaccine. The antibody and the vaccine are administered sequentially or concurrently. The vaccine is a tumor vaccine a bacterial vaccine or a viral vaccine.

In embodiments, methods described herein are useful for increasing T cell activation or T cell effector function in a subject.

In embodiments, methods described herein are useful for inducing an immune response in a subject.

In embodiments, methods described herein are useful for enhancing an immune response or increasing the activity of an immune cell in a subject.

In embodiments, methods described herein are useful for treating T-cell dysfunctional disorders (e.g., cancer).

In embodiments, methods described herein are useful for reducing tumors or inhibiting the growth of tumor cells in a subject.

Thus, the inventive method can be used to treat any type of infectious disease (i.e., a disease or disorder caused by a bacterium, a virus, a fungus, or a parasite). Examples of infectious diseases that can be treated by the inventive method include, but are not limited to, diseases caused by a human immunodeficiency virus (HIV), a respiratory syncytial virus (RSV), an influenza virus, a dengue virus, a hepatitis B virus (HBV, or a hepatitis C virus (HCV)). When an inventive method treats an infectious disease, an antibody agent can be administered in combination with at least one anti-bacterial agent or at least one anti-viral agent. In this respect, the anti-bacterial agent can be any suitable antibiotic known in the art. The anti-viral agent can be any vaccine of any suitable type that specifically targets a particular virus (e.g., live-attenuated vaccines, subunit vaccines, recombinant vector vaccines, and small molecule anti-viral therapies (e.g., viral replication inhibitors and nucleoside analogs).

In embodiments, the inventive methods can be used to treat any type of autoimmune disease (i.e., as disease or disorder caused by immune system over-activity in which the body attacks and damages its own tissues), such as those described in, for example, MacKay I. R. and Rose N. R., eds., The Autoimmune Diseases, Fifth Edition, Academic Press, Waltham, MA (2014). Examples of autoimmune diseases that can be treated by the inventive method include, but are not limited to, multiple sclerosis, type 1 diabetes mellitus, rheumatoid arthritis, scleroderma, Crohn's disease, psoriasis, systemic lupus erythematosus (SLE), and ulcerative colitis. When the inventive method treats an autoimmune disease, an antibody agent described herein can be used in combination with an anti-inflammatory agent including, for example, corticosteroids (e.g., prednisone and fluticasone) and non-steroidal anti-inflammatory drugs (NSAIDs) (e.g., aspirin, ibuprofen, and naproxen).

### Combination Therapies

Provided herein are methods that comprise administering a further therapeutic agent (e.g., an immune checkpoint inhibitor).

### Checkpoint Inhibitors

Combination treatments that simultaneously target two or more of these immune checkpoint pathways have demonstrated improved and potentially synergistic antitumor activity (see, e.g., Sakuishi et al., J. Exp. Med., 207: 2187-2194 (2010); Ngiow et al., Cancer Res., 71: 3540-3551 (2011); and Woo et al., Cancer Res., 72: 917-927 (2012)).

In embodiments, a checkpoint inhibitor is an agent capable of inhibiting any of the following: PD-1 (e.g., inhibition via anti-PD-1, anti-PD-L1, or anti-PD-L2 therapies), CTLA-4, TIM-3, TIGIT, LAGs (e.g., LAG-3), CEACAM (e.g., CEACAM-1, -3 and/or -5), VISTA, BTLA, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, TGFR (e.g., TGFR beta), B7-H1, B7-H4 (VTCN1), OX-40, CD137, CD40, IDO, or CSF-1R. In embodiments, a checkpoint inhibitor is a small molecule, a nucleic acid, a polypeptide (e.g., an antibody), a carbohydrate, a lipid, a metal, or a toxin. In embodiments, a checkpoint inhibitor is an antibody, an antibody conjugate, or an antigen-binding fragment thereof.

In embodiments, an immune checkpoint inhibitor is an agent that inhibits programmed death-1 protein (PD-1) signaling, cytotoxic T-lymphocyte-associated protein 4 (CTLA-4), T-cell immunoglobulin domain and mucin domain 3 protein (TIM-3) T cell immunoglobulin, lymphocyte activation gene-3 (LAG-3), and ITIM domain (TIGIT), indoleamine 2,3-dioxygenase (IDO), or colony stimulating factor 1 receptor (CSF1R). In some embodiments, methods are provided for treating or preventing cancer, infection diseases, or autoimmune disease in a mammal, comprising administering (i) an antibody agent that binds to a LAG-3 protein and (ii) an agent that inhibits PD-1 signaling and/or an agent that inhibits T-cell immunoglobulin and mucin-domain-containing 3 (TIM-3).

A typical dose of an immune checkpoint inhibitor can be, for example, in the range of 1 pg/kg to 20 mg/kg of animal or human body weight; however, doses below or above this exemplary range can be within the scope of the disclosure. The daily parenteral dose can be about 0.00001 μg/kg to about 20 mg/kg of total body weight (e.g., about 0.001 μg/kg, about 0.1 μg/kg, about 1 μg/kg, about 5 μg/kg, about 10 μg/kg, about 100 μg/kg, about 500 μg/kg, about 1 mg/kg, about 5 mg/kg, about 10 mg/kg, or a range defined by any two of the foregoing values), from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), or from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values).

Agents that Inhibit CTLA-4

In embodiments, an immune checkpoint inhibitor is a CTLA-4 inhibitor (e.g., an antibody, an antibody conjugate, or an antigen-binding fragment thereof). In embodiments, a CTLA-4 inhibitor is a small molecule, a nucleic acid, a polypeptide (e.g., an antibody), a carbohydrate, a lipid, a metal, or a toxin. In embodiments, a CTLA-4 inhibitor is a small molecule. In embodiments, a CTLA-4 inhibitor is a CTLA-4 binding agent. In embodiments, a CTLA-4 inhibitor is an antibody, an antibody conjugate, or an antigen-binding fragment thereof. In embodiments, a CTLA-4 inhibitor is ipilimumab (Yervoy), AGEN1884, or tremelimumab.

Agents that Inhibit TIGIT

In embodiments, an immune checkpoint inhibitor is a TIGIT inhibitor (e.g., an antibody, an antibody conjugate, or an antigen-binding fragment thereof). In embodiments, a TIGIT inhibitor is a small molecule, a nucleic acid, a polypeptide (e.g., an antibody), a carbohydrate, a lipid, a metal, or a toxin. In embodiments, a TIGIT inhibitor is small molecule. In embodiments, a TIGIT inhibitor is a TIGIT binding agent. In embodiments, a TIGIT inhibitor is an antibody, an antibody conjugate, or an antigen-binding fragment thereof. In embodiments, a TIGIT inhibitor is MTIG7192A, BMS-986207, or OMP-31M32.

Agents that Inhibit IDO

In embodiments, an immune checkpoint inhibitor is an IDO inhibitor. In embodiments, an IDO inhibitor is a small molecule, a nucleic acid, a polypeptide (e.g., an antibody), a carbohydrate, a lipid, a metal, or a toxin. In embodiments, an IDO inhibitor is small molecule. In embodiments, an IDO inhibitor is an IDO binding agent. In embodiments, an IDO inhibitor is an antibody, an antibody conjugate, or an antigen-binding fragment thereof.

Agents that Inhibit CSF1R

In embodiments, an immune checkpoint inhibitor is a CSF1R inhibitor. In embodiments, a CSF1R inhibitor is a small molecule, a nucleic acid, a polypeptide (e.g., an antibody), a carbohydrate, a lipid, a metal, or a toxin. In embodiments, a CSF1R inhibitor is small molecule. In embodiments, a CSF1R inhibitor is a CSF1R binding agent. In embodiments, a CSF1R inhibitor is an antibody, an antibody conjugate, or an antigen-binding fragment thereof.

Agents that Inhibit PD-1

In embodiments, an immune checkpoint inhibitor is a PD-1 inhibitor (e.g., as described herein). In embodiments, a PD-1 inhibitor is an agent described in FIG. 1A or FIG. 1B. In embodiments, a PD-1 inhibitor is a small molecule, a nucleic acid, a polypeptide (e.g., an antibody, an antibody conjugate, or an antigen-binding fragment thereof), a carbohydrate, a lipid, a metal, or a toxin. In embodiments, a PD-1 inhibitor is a PD-1 binding agent (e.g., an antibody, an antibody conjugate, or an antigen-binding fragment thereof). In embodiments, a PD-1 binding agent is an antibody, an antibody conjugate, or an antigen-binding fragment thereof. In embodiments, a PD-1 binding agent is TSR-042, nivolumab, pembrolizumab, atezolizumab, durvalumab, avelumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, BGB-A333, AMP-514 (MEDI-0680), AGEN-2034, CS1001, Sym-021, SHR-1316, PF-06801591, LZM009, KN-035, AB122, genolimzumab (CBT-501), FAZ-053, CK-301, AK 104, or GLS-010, or any of the PD-1 antibodies disclosed in WO2014/179664. In embodiments, an anti-PD-1 agent is TSR-042. In embodiments, a PD-1 inhibitor is a PD-L1 or PD-L2 binding agent such as durvalumab, atezolizumab, avelumab, BGB-A333, SHR-1316, FAZ-053, CK-301, or, PD-L1 millamolecule, or derivatives thereof.

Agents that Inhibit TIM-3

In embodiments, an immune checkpoint inhibitor is a TIM-3 inhibitor (e.g., as described herein).

TIM-3 has been proposed to play a role in T-cell exhaustion and limiting the antitumor immune response and is targeted to treat cancer, infectious disease, or autoimmune disease.

TIM-3 is a 60 kDa type 1 transmembrane protein comprised of three domains: an N-terminal Ig variable (IgV)-like domain, a central Ser/Thr-rich mucin domain, and a transmembrane domain with a short intracellular tail (see, e.g., Kane, L. P., Journal of Immunology, 184(6): 2743-2749 (2010)). TIM-3 was initially identified on terminally differentiated Th1 cells, and negatively regulates the T-cell response by inducing T-cell apoptosis (see, e.g., Hastings et al., Eur. J. Immunol., 39(9): 2492-2501 (2009)). TIM-3 also is expressed on activated Th17 and Tc1 cells, and dysregulation of Tim-3 expression on CD4+ T-cells and CD8+ T-cells is associated with several autoimmune diseases, viral infections, and cancer (see, e.g., Liberal et al., Hepatology, 56(2): 677-686 (2012); Wu et al., Eur. J. Immunol., 42(5): 1180-1191 (2012); Anderson, A. C., Curr. Opin. Immunol., 24(2): 213-216 (2012); and Han et al., Frontiers in Immunology, 4: 449 (2013)).

Putative ligands for TIM-3 include phosphatidylserine (Nakayama et al., Blood, 113: 3821-3830 (2009)), galectin-9 (Zhu et al., Nat. Immunol., 6: 1245-1252 (2005)), high-mobility group protein 1 (HMGB1) (Chiba et al., Nature Immunology, 13: 832-842 (2012)), and carcinoembryonic antigen cell adhesion molecule 1 (CEACAM1) (Huang et al., Nature, 517(7534): 386-90 (2015)).

TIM-3 functions to regulate various aspects of the immune response. The interaction of TIM-3 and galectin-9 (Gal-9) induces cell death and in vivo blockade of this interaction exacerbates autoimmunity and abrogates tolerance in experimental models, strongly suggesting that TIM-3 is a negative regulatory molecule. In contrast to its effect on T-cells, the TIM-3-Gal-9 interaction exhibits antimicrobial effects by promoting macrophage clearance of intracellular pathogens (see, e.g., Sakuishi et al., Trends in Immunology, 32(8): 345-349 (2011)). In vivo, suppression of TIM-3 has been shown to enhance the pathological severity of experimental autoimmune encephalomyelitis (Monney et al., supra; and Anderson, A. C. and Anderson, D. E., Curr. Opin. Immunol., 18: 665-669 (2006)). Studies also suggest that dysregulation of the TIM-3-galectin-9 pathway could play a role in chronic autoimmune diseases, such as multiple sclerosis (Anderson and Anderson, supra). TIM-3 promotes clearance of apoptotic cells by binding phosphatidyl serine through its unique binding cleft (see, e.g., DeKruyff et al., J. Immunol., 184(4):1918-1930 (2010)).

In one aspect, the invention features a method of inducing an immune response in a subject, the method comprising: measuring a level of PD-L1 expression in a sample obtained from the subject; and administering to the subject based on the level of PD-L1 expression a therapeutically effective dose of an immune checkpoint inhibitor. In another aspect, the invention features a method of enhancing an immune response or increasing the activity of an immune cell in a subject, the method comprising: measuring a level of PD-L1 expression in a sample obtained from the subject; and administering to the subject based on the level of PD-L1 expression a therapeutically effective dose of an immune checkpoint inhibitor. In still another aspect, the invention features a method of treating a subject, the method comprising: measuring a level of PD-L1 expression in a sample obtained from the subject; and administering to the subject based on the level of PD-L1 expression a therapeutically effective dose of an immune checkpoint inhibitor.

In another aspect, the invention features a method of inducing an immune response in a subject, the method comprising: selecting a subject based a level of PD-L1 expression in a sample obtained from the subject as compared to a reference level; and administering to the selected subject a therapeutically effective dose of an immune checkpoint inhibitor. In another aspect, the invention features a method of enhancing an immune response or increasing the activity of an immune cell in a subject, the method comprising: selecting a subject based a level of PD-L1 expression in a sample obtained from the subject as compared to a reference level; and administering to the selected subject a therapeutically effective dose of an immune checkpoint inhibitor. In still another aspect, the invention features a method of treating a subject, the method comprising: selecting a subject based a level of PD-L1 expression in a sample obtained from the subject as compared to a reference level; and administering to the selected subject a therapeutically effective dose of an immune checkpoint inhibitor.

In embodiments, a mammal has a disorder that is responsive to T cell immunoglobulin and mucin protein 3 (TIM-3) inhibition. In embodiments, a mammal has a disorder that is responsive to T cell immunoglobulin and mucin protein 3 (TIM-3) inhibition and characterized by PD-L1 expression. In some embodiments, such a method comprises administering an effective amount of an agent that is capable of inhibiting T cell immunoglobulin and mucin protein 3 (TIM-3) signaling (TIM-3 agent). In some embodiments, such a method comprises administering an effective amount of an agent that is capable of inhibiting T cell immunoglobulin and mucin protein 3 (TIM-3) signaling (TIM-3 agent) and an effective amount of a second immune checkpoint inhibitor (e.g., an effective amount of an agent that is capable of Lymphocyte Activation Gene-3 (LAG-3) signaling (LAG-3 agent) or an effective amount of an agent that is capable of inhibiting programmed death-1 protein (PD-1) signaling (PD-1 agent)). In some embodiments, such a method comprises administering an effective amount of an agent that is capable of inhibiting T cell immunoglobulin and mucin protein 3 (TIM-3) signaling (TIM-3 agent) and an effective amount of an agent capable of inhibiting Lymphocyte Activation Gene-3 (LAG-3) signaling (LAG-3 agent). In some embodiments, such a method comprises administering an effective amount of an agent that is capable of inhibiting T cell immunoglobulin and mucin protein 3 (TIM-3) signaling (TIM-3 agent) and an effective amount of an agent that is capable of inhibiting programmed death-1 protein (PD-1) signaling (PD-1 agent). In some embodiments, such a method comprises administering an effective amount of an agent that is capable of inhibiting programmed death-1 protein (PD-1) signaling (PD-1 agent), an effective amount of an agent that is capable of inhibiting Lymphocyte Activation Gene-3 (LAG-3) signaling (LAG-3 agent), and an effective amount of an agent that is capable of inhibiting T cell immunoglobulin and mucin protein 3 (TIM-3) signaling (TIM-3 agent). In some embodiments, such a method comprises administering an effective amount of a polypeptide that is capable of binding TIM-3. In some embodiments, such a method comprises administering an effective amount of an isolated nucleic acid encoding polypeptide that is capable of binding TIM-3. In some embodiments, such a method comprises administering an effective amount of a vector that encodes a polypeptide that is capable of binding TIM-3. In some embodiments, such a method comprises administering an effective amount of an isolated cell comprising a nucleic acid or a vector encoding polypeptide that is capable of binding TIM-3. In some embodiments, such a method comprises administering an effective amount of a composition comprising a polypeptide, nucleic acid, vector or cell as described herein. In some embodiments, upon administration of a polypeptide, nucleic acid, vector, cell or composition of the present disclosure, an immune response is induced in the mammal. In some embodiments, the immune response is a humoral or cell mediated immune response. In some embodiments, the immune response is a CD4 or CD8 T cell response. In some embodiments, the immune response is a B cell response. In embodiments, a LAG-3 agent is TSR-033. In embodiments, a PD-1 agent is TSR-042. In embodiments, a TIM-3 agent is TSR-022. In embodiments, a disorder is cancer.

Inhibition of TIM-3 activity, such as through use of monoclonal antibodies, is currently under investigation as an immunotherapy for tumors based on preclinical studies (see, e.g., Ngiow et al., Cancer Res., 71(21): 1-5 (2011); Guo et al., Journal of Translational Medicine, 11: 215 (2013); and Ngiow et al., Cancer Res., 71(21): 6567-6571 (2011)).

Exemplary TIM-3 agents are described in FIG. 1D.

In embodiments, a TIM-3 agent is any of TIM-3 agent nos. 1-21 of FIG. 1D.

In some embodiments, an agent that inhibits TIM-3 signaling is administered to a subject.

In some embodiments, an agent that inhibits TIM-3 signaling for use in therapies of the present disclosure is an antibody agent. In some embodiments, a TIM-3 binding agent binds an epitope of TIM-3 which blocks the binding of TIM-3 to any one or more of its putative ligands. TIM-3 antibody agents of the present disclosure may comprise a heavy chain constant region (F_{c}) of any suitable class. In some embodiments, a TIM-3 antibody agent comprises a heavy chain constant region that is based upon wild-type IgG1, IgG2, or IgG4 antibodies, or variants thereof.

In some embodiments, an agent that inhibits TIM-3 signaling is a monoclonal antibody, or a fragment thereof. In some embodiments, an antibody agent that inhibits TIM-3 signaling is a TIM-3 antibody or fragment thereof. Monoclonal antibodies that target TIM-3 that have been tested in clinical studies and/or received marketing approval in the United States.

In some embodiments, a TIM-3 antibody agent is MBG453, LY3321367, Sym023, or a derivative thereof. In some embodiments, a TIM-3 antibody agent is as disclosed in International Patent Application Publication WO2016/161270, the entirety of which is incorporated herein.

In some embodiments, a TIM-3 antibody agent is as disclosed in International Patent Application Publication WO2016/161270, the entirety of which is incorporated herein. In some embodiments, a TIM-3 antibody agent comprises one or more CDR sequences as disclosed in International Patent Application Publication WO2016/161270, the entirety of which is incorporated herein. In some embodiments, a TIM-3 antibody agent comprises one or more CDR sequences as disclosed in International Patent Application Publication WO2016/161270, the entirety of which is incorporated herein. In some embodiments, a TIM-3 antibody agent comprises a light chain variable domain as disclosed in International Patent Application Publication WO2016/161270, the entirety of which is incorporated herein. In some embodiments, a TIM-3 antibody agent comprises a heavy chain variable domain as disclosed in International Patent Application Publication WO2016/161270, the entirety of which is incorporated herein. In some embodiments, a TIM-3 antibody agent comprises a light chain polypeptide as disclosed in International Patent Application Publication WO2016/161270, the entirety of which is incorporated herein. In some embodiments, a TIM-3 antibody agent comprises a heavy chain polypeptide as disclosed in International Patent Application Publication WO2016/161270, the entirety of which is incorporated herein.

In embodiments, a TIM-3 antibody agent is as disclosed in International Patent Application Publication WO2018/085469, the entirety of which is incorporated herein. In some embodiments, a TIM-3 antibody agent comprises one or more CDR sequences as disclosed in International Patent Application Publication WO2018/085469, the entirety of which is incorporated herein. In some embodiments, a TIM-3 antibody agent comprises a light chain variable domain as disclosed in International Patent Application Publication WO2018/085469, the entirety of which is incorporated herein. In some embodiments, a TIM-3 antibody agent comprises a heavy chain variable domain as disclosed in International Patent Application Publication WO2018/085469, the entirety of which is incorporated herein. In some embodiments, a TIM-3 antibody agent comprises a light chain polypeptide as disclosed in International Patent Application Publication WO2018/085469, the entirety of which is incorporated herein. In some embodiments, a TIM-3 antibody agent comprises a heavy chain polypeptide as disclosed in International Patent Application Publication WO2018/085469, the entirety of which is incorporated herein.

In embodiments, a TIM-3 antibody agent is as disclosed in International Patent Application No. PCT/US18/13021, the entirety of which is incorporated herein. In some embodiments, a TIM-3 antibody agent comprises one or more CDR sequences as disclosed in International Patent Application No. PCT/US18/13021, the entirety of which is incorporated herein. In some embodiments, a TIM-3 antibody agent comprises a light chain variable domain as disclosed in International Patent Application No. PCT/US18/13021, the entirety of which is incorporated herein. In some embodiments, a TIM-3 antibody agent comprises a heavy chain variable domain as disclosed in International Patent Application No. PCT/US18/13021, the entirety of which is incorporated herein. In some embodiments, a TIM-3 antibody agent comprises a light chain polypeptide as disclosed in International Patent Application No. PCT/US18/13021, the entirety of which is incorporated herein. In some embodiments, a TIM-3 antibody agent comprises a heavy chain polypeptide as disclosed in International Patent Application No. PCT/US18/13021, the entirety of which is incorporated herein.

In embodiments, a TIM-3 inhibitor is TSR-022.

In some embodiments, a TIM-3 antibody agent comprises one or more CDR sequences that is 90%, 95%, 97%, 98%, 99% or 100% identical to SEQ ID NOs: 11-16.

In some embodiments, a TIM-3 antibody agent comprises one, two or three heavy chain CDR sequences that is 90%, 95%, 97%, 98%, 99% or 100% identical to CDR sequences of SEQ ID NOs: 11-13.

In some embodiments, a TIM-3 antibody agent comprises one, two or three light chain CDR sequences that is 90%, 95%, 97%, 98%, 99% or 100% identical to CDR sequences of SEQ ID NOs: 14-16.

In some embodiments, a TIM-3 antibody agent comprises one, two or three heavy chain CDR sequences that is 90%, 95%, 97%, 98%, 99% or 100% identical to CDR sequences of SEQ ID NOs: 11-13 and one, two or three light chain CDR sequences that is 90%, 95%, 97%, 98%, 99% or 100% identical to CDR sequences of SEQ ID NOs: 14-16.

In some embodiments, a TIM-3 antibody agent comprises six CDR sequences of SEQ ID NOs: 11-16.

In some embodiments, a TIM-3 antibody agent comprises a heavy chain variable domain that is 90%, 95%, 97%, 98%, 99% or 100% identical to SEQ ID NO:17.

In some embodiments, a TIM-3 antibody agent comprises a heavy chain variable domain that is 90%, 95%, 97%, 98%, 99% or 100% identical to SEQ ID NO:18.

In some embodiments, a TIM-3 antibody agent comprises a light chain variable domain that is 90%, 95%, 97%, 98%, 99% or 100% identical to SEQ ID NO:19.

In some embodiments, a TIM-3 antibody agent comprises a light chain variable domain that is 90%, 95%, 97%, 98%, 99% or 100% identical to SEQ ID NO:20.

In some embodiments, a TIM-3 antibody agent comprises a heavy chain variable domain that is 90%, 95%, 97%, 98%, 99% or 100% identical to SEQ ID NO:17 or 18 and a light chain variable domain that is 90%, 95%, 97%, 98%, 99% or 100% identical to SEQ ID NO:19 or 20.

In some embodiments, a TIM-3 antibody agent comprises a heavy chain polypeptide that is 90%, 95%, 97%, 98%, 99% or 100% identical to SEQ ID NO:21.

In some embodiments, a TIM-3 antibody agent comprises a light chain polypeptide that is 90%, 95%, 97%, 98%, 99% or 100% identical to SEQ ID NO:22.

In some embodiments, a TIM-3 antibody agent comprises a heavy chain polypeptide that is 90%, 95%, 97%, 98%, 99% or 100% identical to SEQ ID NO:21, and a light chain polypeptide that is 90%, 95%, 97%, 98%, 99% or 100% identical to SEQ ID NO:22.

TSR-022 comprises a humanized monoclonal anti-TIM-3 antibody comprising a heavy chain whose amino acid sequence comprises SEQ ID NO: 21 and a light chain whose amino acid sequence comprises SEQ ID NO:22. This anti-TIM-3 antibody utilizes a human IGHG4*01 heavy chain gene, and a human IGKC*01 kappa light chain gene, as scaffolds. Further, there is a single Ser to Pro point mutation in the hinge region of the IgG4 heavy chain at the canonical S228 position. Without wishing to be bound by theory, it is envisioned that this point mutation serves to stabilize the hinge of the antibody heavy chain.

Additional biophysical and biochemical characterization of this exemplary humanized monoclonal anti-TIM-3 antibody is also provided regarding observed disulfide linkages and glycosylation. Lys-C and trypsin digested peptides were well separated and detected by on-line LC-MS analysis. The disulfide bond linkages were confirmed by comparison of total ion chromatograms in the non-reduced (NR) condition with the reduced condition. Disulfide linkages are consistent with the expected disulfide linkage pattern for an IgG4 molecule. The residues involved in the expected inter- and intrachain disulfide linkages are tabulated below (Tables 6, 7, and 8).

TABLE
Expected residues involved in disulfide linkages of an
exemplary anti-TIM-3 antibody agent heavy chain having
an amino acid sequence as set forth in SEQ ID NO: 21.

| | anti-TIM-3 mAb |
|---|---|
| Cysteine | HC Residue |
| residue | (position in |
| ID | SEQ ID NO: 21) |
| I | 22 |
| II | 96 |
| III | 127 |
| IV | 140 |
| V | 196 |
| VI | 219 |
| VII | 222 |
| VIII | 254 |
| IX | 314 |
| X | 360 |
| XI | 418 |

TABLE
Expected residues involved in disulfide linkages of an
exemplary anti-TIM-3 antibody agent light chain having
an amino acid sequence as set forth in SEQ ID NO: 22.

| | anti-TIM-3 mAb |
|---|---|
| Cysteine | LC Residue |
| residue | (position in |
| ID | SEQ ID NO: 22) |
| I | 23 |
| II | 88 |
| III | 134 |
| IV | 194 |
| V | 214 |

TABLE
Exemplary disulfide bond assignments for an anti-TIM-3 antibody TSR-022

| | | | Linkage site on | Linkage site on |
|---|---|---|---|---|
| Disulfide | | SEQ ID | HC (position in | LC (position in |
| bond NO. | Disulfide-containing peptides | NOS | SEQ ID NO: 21) | SEQ ID NO: 22) |
| DS1 | VTITCR=FSGSGSGTDFTLTISSLQPEDF | 35 & 36 | | 23 |
| | AVYYCQQSHSAPLTFGGGTK | | | 88 |
| DS2 | SGTASVVCLLNNFYPR=VYACEVTHQGL | 37 & 38 | | 134 |
| | SSPVTK | | | 194 |
| DS3 | SFNRGEC=GPSVFPLAPCSR | 39, 40 | 127 | 214 |
| | GEC=GPSVFPLAPCSR | & 41 | | |
| DS4 | LSCAAASGFTFSSYDMSWVR=AEDTA | 42 & 43 | 22 | |
| | VYYCASMDYWGQGTTVTVSSASTK | | 97 | |
| DS5 | STSESTAALGCLVK=TYTCNVDHK | 44, 45 | 140 | |
| | STSESTAALGCLVK=TYTCNVDHKPSNTK | & 46 | 196 | |
| DS6 | YGPPCPPCPAPEFLGGPSVFLFPPK=YGPP | 47, 48 | 219 | |
| | CPPCPAPEFLGGPSVFLFPPK | & 49 | 222 | |
| | YGPPCPPCPAPEFLGGPSVFLFPPK=YGPP | | | |
| | CPPCPAPEFLGGPSVFLFPPKPK | | | |
| DS7 | TPEVTCVVVDVSQEDPEVQFNWYVDGV | 50 | 254 | |
| | EVHNAK=CK | | 314 | |
| DS8 | NQVSLTCLVK=WQEGNVFSCSVMHEAL | 51 & 52 | 360 | |
| | HNHYTQK | | 418 | |

LC: light chain; HC: heavy chain

This exemplary anti-TIM-3 antibody exhibits an occupied N-glycosylation site at asparagine residue 290 in the CH2 domain of each heavy chain in the mature protein sequence (SEQ ID NO:31). The expressed N-glycosylation at this site is a mixture of oligosaccharide species typically observed on IgGs expressed in mammalian cell culture, for example, shown below is the relative abundance of glycan species from a preparation of this exemplary anti-TIM-3 antibody cultured in Chinese Hamster Ovary (CHO) cells (Table 9).

TABLE
Glycan Analysis of an anti- TIM-3 antibody binding agent

| | Abundance | |
|---|---|---|
| | (% of total | |
| Species | oligosaccharide) | Description of Glycan |
| G0F | 20.1% | Core fucosylated agalactobiantennary |
| | | complex-type oligosaccharide |
| G1F | 41.9% | Core fucosylated monogalactosylated |
| | | biantennary complex type oligosaccharide |
| G2F | 29.0% | Core-fucosylated galactosylated biantennary |
| | | complex type oligosaccharide |
| G2FS1 | 3.2% | Monosialylated core fucosylated |
| | | galactosylated biantennary complex |
| | | type oligosaccharide |
| G2FS2 | 1.2% | Disialylated core fucosylated galactosylated |
| | | biantennary complex type oligosaccharide |
| M5 | 0.4% | Oligomannosidic N-linked oligosaccharide, |
| | | Man₅GlcNAc₂ |

### Exemplary Dosage Regimens

For example, a TIM-3 inhibitor (e.g., TSR-022) can be administered in a dose of about 1, 3 or 10 mg/kg (e.g., about 1 mg/kg; about 3 mg/kg; or about 10 mg/kg) or a flat dose between about 100-1500 mg (e.g., a flat dose about 100 mg; a flat dose about 200 mg; a flat dose about 300 mg; a flat dose about 400 mg; a flat dose about 500 mg; a flat dose about 600 mg; a flat dose about 700 mg; a flat dose about 800 mg; a flat dose about 900 mg; a flat dose about 1000 mg; a flat dose about 1100 mg; a flat dose about 1200 mg; a flat dose about 1300 mg; a flat dose about 1400 mg; or a flat dose about 1500 mg).

In some embodiments, a TIM-3 binding agent (e.g., an anti-TIM-3 antibody) is administered at a dose of 0.1, 1, 3 or 10 mg/kg. In some embodiments, a TIM-3 binding agent is administered according to a regimen that includes a dose of 0.1, 1, 3 or 10 mg/kg every two weeks. In some embodiments, a TIM-3 binding agent is administered according to a regimen that includes a dose of 1, 3 or 10 mg/kg every three weeks.

In some embodiments, a TIM-3 binding agent is administered according to a regimen that includes a dose of 1, 3 or 10 mg/kg every four weeks. In some embodiments, a TIM-3 binding agent at a fixed dose within a range of 200 mg to 1,500 mg. In some embodiments, a TIM-3 binding agent at a fixed dose within a range of 100 mg to 1,000 mg such as 300 mg to 1,000 mg. In some embodiments, a TIM-3 binding agent is administered according to a regimen that includes a fixed dose every two weeks. In some embodiments, a TIM-3 binding agent is administered according to a regimen that includes a fixed dose every three weeks. In some embodiments, a TIM-3 binding agent is administered according to a regimen that includes a fixed dose every four weeks.

In some embodiments, a TIM-3 binding agent (e.g., TSR-022) is administered at a dose of 0.1, 1, 3 or 10 mg/kg.

In some embodiments, a TIM-3 binding agent (e.g., TSR-022) is administered according to a regimen that includes a dose of 0.1, 1, 3 or 10 mg/kg every two weeks. In some embodiments, a TIM-3 binding agent (e.g., TSR-022) is administered according to a regimen that includes a dose of about 1 mg/kg every two weeks. In some embodiments, a TIM-3 binding agent (e.g., TSR-022) is administered according to a regimen that includes a dose of about 3 mg/kg every two weeks. In some embodiments, a TIM-3 binding agent (e.g., TSR-022) is administered according to a regimen that includes a dose of about 10 mg/kg every two weeks.

In some embodiments, a TIM-3 binding agent (e.g., TSR-022) is administered according to a regimen that includes a dose of 1, 3 or 10 mg/kg every three weeks. In some embodiments, a TIM-3 binding agent (e.g., TSR-022) is administered according to a regimen that includes a dose of about 1 mg/kg every three weeks. In some embodiments, a TIM-3 binding agent (e.g., TSR-022) is administered according to a regimen that includes a dose of about 3 mg/kg every three weeks. In some embodiments, a TIM-3 binding agent (e.g., TSR-022) is administered according to a regimen that includes a dose of about 10 mg/kg every three weeks.

In some embodiments, a TIM-3-binding agent (e.g., TSR-022) is administered according to a regimen that includes a dose of 1, 3 or 10 mg/kg every four weeks. In some embodiments, a TIM-3 binding agent (e.g., TSR-022) is administered according to a regimen that includes a dose of about 1 mg/kg every four weeks. In some embodiments, a TIM-3 binding agent (e.g., TSR-022) is administered according to a regimen that includes a dose of about 3 mg/kg every four weeks. In some embodiments, a TIM-3 binding agent (e.g., TSR-022) is administered according to a regimen that includes a dose of about 10 mg/kg every four weeks.

In some embodiments, a TIM-3 binding agent (e.g., TSR-022) is administered at a fixed dose within a range of 200 mg to 1,500 mg. In some embodiments, a TIM-3 binding agent (e.g., TSR-022) is administered at a fixed dose within a range of about 100 mg to about 1000 mg such as about 300 mg to about 1,000 mg. In embodiments, a TIM-3 binding agent is TSR-022. In some embodiments, a TIM-3 binding agent (e.g., TSR-022) is administered according to a regimen that includes a fixed dose every two weeks (Q2W). In some embodiments, a TIM-3 binding agent (e.g., TSR-022) is administered according to a regimen that includes a fixed dose every three weeks (Q3W). In some embodiments, a TIM-3 binding agent (e.g., TSR-022) is administered according to a regimen that includes a fixed dose every four weeks (Q4W). In embodiments, a TIM-3 binding agent is TSR-022.

In embodiments, a fixed dose of 100 mg, 200 mg, 300 mg, 500 mg, 800 mg, 900 mg, 1000 mg, or 1200 mg of a TIM-3 binding agent (e.g., TSR-022) is administered once every two weeks (Q2W). In embodiments, a fixed dose of 100 mg of a TIM-3 binding agent (e.g., TSR-022) is administered once every two weeks (Q2W). In embodiments, a fixed dose of 300 mg of a TIM-3 binding agent (e.g., TSR-022) is administered once every two weeks (Q2W). In embodiments, a fixed dose of 500 mg of a TIM-3 binding agent (e.g., TSR-022) is administered once every two weeks (Q2W). In embodiments, a fixed dose of 800 mg of a TIM-3 binding agent (e.g., TSR-022) is administered once every two weeks (Q2W). In embodiments, a fixed dose of 900 mg of a TIM-3 binding agent (e.g., TSR-022) is administered once every two weeks (Q2W). In embodiments, a fixed dose of 1000 mg of a TIM-3 binding agent (e.g., TSR-022) is administered once every two weeks (Q2W). In embodiments, a fixed dose of 1200 mg of a TIM-3 binding agent (e.g., TSR-022) is administered once every two weeks (Q2W). In embodiments, a TIM-3 binding agent is TSR-022.

In embodiments, a fixed dose of 100 mg, 200 mg, 300 mg, 500 mg, 800 mg, 900 mg, 1000 mg, or 1200 mg of a TIM-3 binding agent (e.g., TSR-022) is administered once every three weeks (Q3W). In embodiments, a fixed dose of 100 mg of a TIM-3 binding agent (e.g., TSR-022) is administered once every three weeks (Q3W). In embodiments, a fixed dose of 300 mg of a TIM-3 binding agent (e.g., TSR-022) is administered once every three weeks (Q3W). In embodiments, a fixed dose of 500 mg of a TIM-3 binding agent (e.g., TSR-022) is administered once every three weeks (Q3W). In embodiments, a fixed dose of 800 mg of a TIM-3 binding agent (e.g., TSR-022) is administered once every three weeks (Q3W). In embodiments, a fixed dose of 900 mg of a TIM-3 binding agent (e.g., TSR-022) is administered once every three weeks (Q3W). In embodiments, a fixed dose of 1000 mg of a TIM-3 binding agent (e.g., TSR-022) is administered once every three weeks (Q3W). In embodiments, a fixed dose of 1200 mg of a TIM-3 binding agent (e.g., TSR-022) is administered once every three weeks (Q3W). In embodiments, a TIM-3 binding agent is TSR-022.

In embodiments, a fixed dose of 100 mg, 200 mg, 300 mg, 500 mg, 800 mg, 900 mg, 1000 mg, or 1200 mg of a TIM-3 binding agent (e.g., TSR-022) is administered once every four weeks (Q4W). In embodiments, a fixed dose of 100 mg of a TIM-3 binding agent (e.g., TSR-022) is administered once every four weeks (Q4W). In embodiments, a fixed dose of 300 mg of a TIM-3 binding agent (e.g., TSR-022) is administered once every four weeks (Q4W). In embodiments, a fixed dose of 500 mg of a TIM-3 binding agent (e.g., TSR-022) is administered once every four weeks (Q4W). In embodiments, a fixed dose of 800 mg of a TIM-3 binding agent (e.g., TSR-022) is administered once every four weeks (Q4W). In embodiments, a fixed dose of 900 mg of a TIM-3 binding agent (e.g., TSR-022) is administered once every four weeks (Q4W). In embodiments, a fixed dose of 1000 mg of a TIM-3 binding agent (e.g., TSR-022) is administered once every four weeks (Q4W). In embodiments, a fixed dose of 1200 mg of a TIM-3 binding agent (e.g., TSR-022) is administered once every four weeks (Q4W). In embodiments, a TIM-3 binding agent is TSR-022.

In embodiments, a TIM-3 binding agent is TSR-022. In embodiments, TSR-022 is administered according to a regimen that includes a flat dose of about 100 mg every about three weeks (Q3W), which also can be referred to as a 21-day treatment cycle. In embodiments, TSR-022 is administered according to a regimen that includes a flat dose of about 300 mg every about three weeks (Q3W), which also can be referred to as a 21-day treatment cycle. In embodiments, TSR-022 is administered on about the first day of a treatment cycle, optionally with a permissible window of administration of ±3 days: that is, TSR-022 can be administered in a period spanning from about three days before the first day of a treatment cycle to about three days after the first day of a treatment cycle.

In embodiments, TSR-022 is administered intravenously (e.g., via infusion). In embodiments, TSR-022 is administered intravenously (e.g., via infusion) over a time period of about 15 minutes to about 45 minutes. In embodiments, TSR-022 is administered intravenously (e.g., via infusion) over a targeted time period of about 30 minutes, with an optionally permitted window between about −5 minutes and about +15 minutes: that, is TSR-022 is administered intravenously (e.g., via infusion) over a time period of about 25 minutes to about 45 minutes.

In certain methods, a TIM-3 binding agent can be administered prior to (e.g., 5 minutes, 15 minutes, 30 minutes, 45 minutes, 1 hour, 2 hours, 4 hours, 6 hours, 12 hours, 24 hours, 48, hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a LAG-3 binding agent to a subject in need thereof.

Agents that Inhibit LAG-3

Lymphocyte Activation Gene-3 (LAG-3), which is also known as cluster of differentiation 223 (CD223), is a member of the immunoglobulin supergene family and is structurally and genetically related to CD4. LAG-3 is expressed on T-cells, B cells, natural killer (NK) cells and plasmacytoid dendritic cells (pDCs). Like CD4, LAG-3 ectodomain is composed of four Ig-like domains (D1-D4) and LAG-3 has been demonstrated to interact with MHC Class II molecules (Baixeras et al., J. Exp. Med., 176: 327-337 (1992)), but binds at a distinct site (Huard et al., Proc. Nail Acad. Sci. USA, 94(11): 5744-5749 (1997)). For example, a soluble LAG-3 immunoglobulin fusion protein (sLAG-3Ig) directly and specifically binds via LAG-3 to MHC class II on the cell surface (Huard et al., Eur. J. Immunol., 26: 1180-1186 (1996)).

LAG-3 is upregulated following T-cell activation, and modulates T-cell function as well as T-cell homeostasis (Sierro et al., Expert Opin. Ther. Targets, 15(1): 91-101(2011)). The LAG-3/MHC class II interaction may play a role in down-regulating antigen-dependent stimulation of CD4+ T lymphocytes, as demonstrated in in vitro studies of antigen-specific T-cell proliferation, higher expression of activation antigens such as CD25, and higher concentrations of cytokines such as interferon-gamma and interleukin-4 (Huard et al., Eur. J. Immunol., 24: 3216-3221 (1994)). CD4+CD25+ regulatory T-cells (Treg) also have been shown to express LAG-3 upon activation and antibodies to LAG-3 inhibit suppression by induced Treg cells, both in vitro and in vivo, suggesting that LAG-3 contributes to the suppressor activity of Treg cells (Huang et al., Immunity, 21: 503-513 (2004)). Furthermore, LAG-3 has been shown to negatively regulate T-cell homeostasis by regulatory T cell-dependent and -independent mechanisms (Workman, C. J. and Vignali, D. A., J. Immunol, 174: 688-695 (2005)).

Subsets of conventional T-cells that are anergic or display impaired functions express LAG-3, and LAG-3+ T-cells are enriched at tumor sites and during chronic viral infections. However, while LAG-3 knockout mice have been shown to mount normal virus-specific CD4+ and CD8 T-cell responses, blockade of the PD-1/PD-L1 pathway combined with LAG-3 blockade improved viral control as compared with PD-L1 blockade alone (Blackburn et al., Nat. Immunol., 10: 29-37 (2009); and Riehter et al., Int. Immunol., 22: 13-2 (2010)). In a self-tolerance/tumor mouse model where transgenic CD8+ T-cells were rendered unresponsive/anergic in vivo, LAG-3 blockade or deficiency in CD8+ T-cells enhanced T-cell proliferation, T-cell recruitment and effector functions at the tumor site (Grosso et al., J. Clin. Invest., 117: 3383-92 (2007)).

In addition, the interaction between LAG-3 and its major ligand, MHC class II, may play a role in modulating dendritic cell function (Andreae et al., J Immunol., 168:3874-3880, 2002). Recent preclinical studies have documented a role for LAG-3 in CD8+ T cell exhaustion (Blackburn et al., Nat Immunol., 10: 29-37, 2009), and blockade of the LAG-3/MHC class II interaction using a LAG-3Ig fusion protein may be useful for cancer therapy.

In one aspect, the invention features a method of inducing an immune response in a subject, the method comprising: measuring a level of PD-L1 expression in a sample obtained from the subject; and administering to the subject based on the level of PD-L1 expression a therapeutically effective dose of an immune checkpoint inhibitor. In another aspect, the invention features a method of enhancing an immune response or increasing the activity of an immune cell in a subject, the method comprising: measuring a level of PD-L1 expression in a sample obtained from the subject; and administering to the subject based on the level of PD-L1 expression a therapeutically effective dose of an immune checkpoint inhibitor. In still another aspect, the invention features a method of treating a subject, the method comprising: measuring a level of PD-L1 expression in a sample obtained from the subject; and administering to the subject based on the level of PD-L1 expression a therapeutically effective dose of an immune checkpoint inhibitor.

In another aspect, the invention features a method of inducing an immune response in a subject, the method comprising: selecting a subject based a level of PD-L1 expression in a sample obtained from the subject as compared to a reference level; and administering to the selected subject a therapeutically effective dose of an immune checkpoint inhibitor. In another aspect, the invention features a method of enhancing an immune response or increasing the activity of an immune cell in a subject, the method comprising: selecting a subject based a level of PD-L1 expression in a sample obtained from the subject as compared to a reference level; and administering to the selected subject a therapeutically effective dose of an immune checkpoint inhibitor. In still another aspect, the invention features a method of treating a subject, the method comprising: selecting a subject based a level of PD-L1 expression in a sample obtained from the subject as compared to a reference level; and administering to the selected subject a therapeutically effective dose of an immune checkpoint inhibitor.

In embodiments of methods described herein, a subject has a disorder that is responsive to Lymphocyte Activation Gene-3 (LAG-3) inhibition. In embodiments of methods described herein, a subject has a disorder that is responsive to Lymphocyte Activation Gene-3 (LAG-3) inhibition and characterized by PD-L1 expression. In some embodiments, such a method comprises administering an effective amount of an agent that is capable of inhibiting Lymphocyte Activation Gene-3 (LAG-3) signaling (LAG-3 agent). In some embodiments, such a method comprises administering an effective amount of an agent that is capable of inhibiting Lymphocyte Activation Gene-3 (LAG-3) signaling (LAG-3 agent) and an effective amount of a second immune checkpoint inhibitor (e.g., an effective amount of an agent that is capable of inhibiting programmed death-1 protein (PD-1) signaling (PD-1 agent) or an effective amount of an agent that is capable of inhibiting T cell immunoglobulin and mucin protein 3 (TIM-3) signaling (TIM-3 agent)). In some embodiments, such a method comprises administering an effective amount of an agent that is capable of inhibiting Lymphocyte Activation Gene-3 (LAG-3) signaling (LAG-3 agent) and an effective amount of an agent that is capable of inhibiting programmed death-1 protein (PD-1) signaling (PD-1 agent). In some embodiments, such a method comprises administering an effective amount of an agent that is capable of inhibiting Lymphocyte Activation Gene-3 (LAG-3) signaling (LAG-3 agent) and an effective amount of an agent that is capable of inhibiting T cell immunoglobulin and mucin protein 3 (TIM-3) signaling (TIM-3 agent). In some embodiments, such a method comprises administering an effective amount of an agent that is capable of inhibiting Lymphocyte Activation Gene-3 (LAG-3) signaling (LAG-3 agent), an effective amount of an agent that is capable of inhibiting programmed death-1 protein (PD-1) signaling (PD-1 agent), and an effective amount of an agent that is capable of inhibiting T cell immunoglobulin and mucin protein 3 (TIM-3) signaling (TIM-3 agent). In some embodiments, such a method comprises administering an effective amount of a polypeptide that is capable of binding LAG-3. In some embodiments, such a method comprises administering an effective amount of an isolated nucleic acid encoding polypeptide that is capable of binding LAG-3. In some embodiments, such a method comprises administering an effective amount of a vector that encodes a polypeptide that is capable of binding LAG-3. In some embodiments, such a method comprises administering an effective amount of an isolated cell comprising a nucleic acid or a vector encoding polypeptide that is capable of binding LAG-3. In some embodiments, such a method comprises administering an effective amount of a composition comprising a polypeptide, nucleic acid, vector or cell as described herein. In some embodiments, upon administration of a polypeptide, nucleic acid, vector, cell or composition of the present disclosure, an immune response is induced in the mammal. In some embodiments, the immune response is a humoral or cell mediated immune response. In some embodiments, the immune response is a CD4 or CD8 T cell response. In some embodiments, the immune response is a B cell response. In embodiments, a LAG-3 agent is TSR-033. In embodiments, a PD-1 agent is TSR-042. In embodiments, a TIM-3 agent is TSR-022. In embodiments, a disorder is cancer.

Exemplary LAG-3 agents are described in FIG. 1C.

In embodiments, a LAG-3 agent is any of LAG-3 agent nos. 1-24 of FIG. 1C.

In embodiments, an anti-LAG-3 agent is an antibody, an antibody conjugate, or an antigen-binding fragment thereof. In embodiments, an anti-LAG-3 agent is a small molecule, a nucleic acid, a polypeptide (e.g., an antibody), a carbohydrate, a lipid, a metal, or a toxin.

In embodiments, an anti-LAG-3 agent is a small molecule. In embodiments, an anti-LAG-3 agent is a LAG-3 binding agent.

In embodiments, an anti-LAG-3 agent is an antibody, an antibody conjugate, or an antigen-binding fragment thereof.

In embodiments, an anti-LAG-3 agent is IMP321, relatlimab (BMS-986016), BI 754111, GSK2831781 (IMP-731), Novartis LAG525 (IMP701), REGN3767, MK-4280, MGD-013, GSK-2831781, FS-118, XmAb22841, INCAGN-2385, FS-18, ENUM-006, AVA-017, AM-0003, Avacta PD-L1/LAG-3 bispecific affamer, iOnctura anti-LAG-3 antibody, Arcus anti-LAG-3 antibody, or Sym022, or a LAG-3 inhibitor described in WO 2016/126858, WO 2017/019894, or WO 2015/138920, each of which is hereby incorporated by reference in its entirety.

In some embodiments, a LAG-3 antibody agent is as disclosed in International Patent Application Publication WO2016/126858, the entirety of which is incorporated herein. In some embodiments, a LAG-3 antibody agent comprises one or more CDR sequences as disclosed in International Patent Application Publication WO2016/126858, the entirety of which is incorporated herein. In some embodiments, a LAG-3 antibody agent comprises one or more CDR sequences as disclosed in International Patent Application Publication WO2016/126858, the entirety of which is incorporated herein. In some embodiments, a LAG-3 antibody agent comprises a light chain variable domain as disclosed in International Patent Application Publication WO2016/126858, the entirety of which is incorporated herein. In some embodiments, a LAG-3 antibody agent comprises a heavy chain variable domain as disclosed in International Patent Application Publication WO2016/126858, the entirety of which is incorporated herein. In some embodiments, a LAG-3 antibody agent comprises a light chain polypeptide as disclosed in International Patent Application Publication WO2016/126858, the entirety of which is incorporated herein. In some embodiments, a LAG-3 antibody agent comprises a heavy chain polypeptide as disclosed in International Patent Application Publication WO2016/126858, the entirety of which is incorporated herein.

In embodiments, a LAG-3 antibody agent is as disclosed in International Patent Application No. PCT/US18/30027, the entirety of which is incorporated herein. In some embodiments, a LAG-3 antibody agent comprises one or more CDR sequences as disclosed in International Patent Application No. PCT/US18/30027, the entirety of which is incorporated herein. In some embodiments, a LAG-3 antibody agent comprises a light chain variable domain as disclosed in International Patent Application No. PCT/US18/30027, the entirety of which is incorporated herein. In some embodiments, a LAG-3 antibody agent comprises a heavy chain variable domain as disclosed in International Patent Application No. PCT/US18/30027, the entirety of which is incorporated herein. In some embodiments, a LAG-3 antibody agent comprises a light chain polypeptide as disclosed in International Patent Application No. PCT/US18/30027, the entirety of which is incorporated herein. In some embodiments, a LAG-3 antibody agent comprises a heavy chain polypeptide as disclosed in International Patent Application No. PCT/US18/30027, the entirety of which is incorporated herein.

In embodiments, a LAG-3 inhibitor is TSR-033.

In some embodiments, a LAG-3 antibody agent comprises one or more CDR sequences that is 90%, 95%, 97%, 98%, 99% or 100% identical to SEQ ID NOs: 23-28.

In some embodiments, a LAG-3 antibody agent comprises one, two or three heavy chain CDR sequences that is 90%, 95%, 97%, 98%, 99% or 100% identical to CDR sequences of SEQ ID NOs: 23-25.

In some embodiments, a LAG-3 antibody agent comprises one, two or three light chain CDR sequences that is 90%, 95%, 97%, 98%, 99% or 100% identical to CDR sequences of SEQ ID NOs: 26-28.

In some embodiments, a LAG-3 antibody agent comprises one, two or three heavy chain CDR sequences that is 90%, 95%, 97%, 98%, 99% or 100% identical to CDR sequences of SEQ ID NOs: 23-25 and one, two or three light chain CDR sequences that is 90%, 95%, 97%, 98%, 99% or 100% identical to CDR sequences of SEQ ID NOs: 26-28.

In some embodiments, a LAG-3 antibody agent comprises six CDR sequences of SEQ ID NOs: 23-28.

In some embodiments, a LAG-3 antibody agent comprises a heavy chain variable domain that is 90%, 95%, 97%, 98%, 99% or 100% identical to SEQ ID NO:29.

In some embodiments, a LAG-3 antibody agent comprises a light chain variable domain that is 90%, 95%, 97%, 98%, 99% or 100% identical to SEQ ID NO:30.

In some embodiments, a LAG-3 antibody agent comprises a heavy chain variable domain that is 90%, 95%, 97%, 98%, 99% or 100% identical to SEQ ID NO:29 and a light chain variable domain that is 90%, 95%, 97%, 98%, 99% or 100% identical to SEQ ID NO:30.

In some embodiments, a LAG-3 antibody agent comprises a heavy chain polypeptide that is 90%, 95%, 97%, 98%, 99% or 100% identical to SEQ ID NO:31.

In some embodiments, a LAG-3 antibody agent comprises a light chain polypeptide that is 90%, 95%, 97%, 98%, 99% or 100% identical to SEQ ID NO:32.

In some embodiments, a LAG-3 antibody agent comprises a heavy chain polypeptide that is 90%, 95%, 97%, 98%, 99% or 100% identical to SEQ ID NO:31, and a light chain polypeptide that is 90%, 95%, 97%, 98%, 99% or 100% identical to SEQ ID NO:32.

In some embodiments, a provided anti-LAG-3 antibody agent has a structure that includes one or more disulfide bonds. In some embodiments, the one or more disulfide bonds are or include a disulfide bond at the expected position for an IgG4 immunoglobulin. In some embodiments, a disulfide bond is present at one or more residues corresponding to positions selected from residue 22, 96, 128, 141, 197, 220, 223, 255, 315, 361 and 419 of SEQ ID NO: 21. In some embodiments, a disulfide bond is present at one or more residues corresponding to positions selected from residue 23, 93, 139, 199 and 219 of SEQ ID NO: 22. The light chain variable region can be aligned with the variable region of the heavy chain, and the light chain constant region can be aligned with the first constant region of the heavy chain. The remaining constant regions of the heavy chains can be aligned with each other.

Inter- and intra-disulfide bonds were identified, and exemplary disulfide bond assignments are shown in Table 10 for an exemplary anti-LAG-3 antibody agent TSR-033 comprising a heavy chain of SEQ ID NO: 21 and a light chain of SEQ ID NO: 22. The amino acid residues mentioned in each example were numbered according to SEQ ID NO: 21 and SEQ ID NO: 22.

TABLE
Exemplary disulfide bond assignments for an anti-LAG-3 antibody

| | | | Linkage Sites on |
|---|---|---|---|
| | | | HC without |
| | | | signal sequence |
| | | | (SEQ ID NO: 21) |
| | | | and |
| | | | LC without |
| Disulfide | Disulfide- | SEQ ID | signal sequence |
| bond NO. | containing peptides | NOS | (SEQ ID NO: 22) |
| DS1 | DIVMTQTPLSLSVTPGQP | 53 & 54 | LC23-LC93 |
| | ASISCR=VEAEDVGVYFC | | |
| | GQSTHVPYAFGGGTK | | |
| DS2 | SGTASVVCLLNNFYPR=V | 37 & 38 | LC139-LC199 |
| | YACEVTHQGLSSPVTK | | |
| DS3 | GEC=GPSVFPLAPCSR | 41 | LC219-HC128 |
| DS4 | ISCK=SEDTAVYYCTYAF | 55 & 56 | HC22-HC96 |
| | GGYWGQGTTVTVSSAST | | |
| | K | | |
| DS5a | STSESTAALGCLVK=TYT | 44 & 57 | HC141-HC197 |
| | CNVDHK | | |
| DS5b | STSESTAALGCLVK=TYT | 44 & 46 | HC141-HC197 |
| | CNVDHKPSNTK | | |
| DS6 | YGPPCPPCPPAPEFLGGPS | 58 & 47 | HC220-HC220 |
| | VFLFPPK=YGPPCPPCPAP | | and |
| | EFLGGPSVFLFPPK | | HC223-HC223 |
| DS7 | TPEVTCVVVDVSQEDPEV | 50 | HC255-HC315 |
| | QFNWYVDGVEVHNAK= | | |
| | CK | | |
| DS8 | NQVSLTCLVK=WQEGNV | 51 & 52 | HC361-HC419 |
| | FSCSVMHEALHNHYTQK | | |

LC: light chain; HC: heavy chain

An anti-LAG-3 antibody can also be described using N-glycan analysis. This example describes N-glycan profiling of characterization of an exemplary anti-LAG-3 antibody agent The relative abundance of glycan species from a preparation of this exemplary anti-LAG-3 antibody cultured in Chinese Hamster Ovary (CHO) cells was determined. This exemplary anti-LAG-3 antibody exhibits an occupied N-glycosylation site, and the expressed N-glycosylation at this site is a mixture of oligosaccharide species typically observed on IgGs expressed in mammalian cell culture.

For example, N-glycans were released by PNGase F and labeled with 2-AB followed by HILIC separation and fluorescence detection (FLD).

The glycosylation site of the anti-LAG-3 antibody is on the heavy chain N291.

Exemplary N-glycan analyses for two exemplary lots of an anti-LAG-3 antibody agent TSR-033 (comprising heavy chain of SEQ ID NO: 21 and light chain of SEQ ID NO: 22). are shown in Table 11. Detected glycans include GOF, G1F, G2F, and Man-5, as well as other oligosaccharide species.

TABLE
N-Glycan analysis of exemplary lots
of the anti-LAG-3 antibody agent

| Species | Lot 1* | Lot 2* |
|---|---|---|
| G0F | 62.3% | 70.2% |
| G1F | 21.6% | 14.3% |
| G2F | 4.0% | 2.1% |
| Man-5 | 3.7% | 4.5% |
| other species | 8.4% | 8.9% |

*Amounts refer to percentage of total N-linked oligosaccharides

### Exemplary Dosage Regimens

Table 12 also provides exemplary doses of LAG-3 agents (e.g., TSR-033) as administered in exemplary Q2W and Q3W schedules. The exemplary doses of Table 12 are also suitable as doses of an anti-LAG-3 agent (e.g., TSR-033) in combination therapies (e.g., dual or triple blockade therapy).

TABLE
Dosing Schedules for an Exemplary LAG-3 Agent TSR-033

| Q2W Administration | | Q3W Administration | |
|---|---|---|---|
| (once every two weeks) | | (once every three weeks) | |
| | Weight-based | | Weight-based |
| Flat Dose | Dose | Flat Dose | Dose |
| about 240 mg | about 3 mg/kg | about 500 mg | about 10 mg/kg |
| about 500 mg | about 10 mg/kg | about 720 mg | about 12 mg/kg |
| about 720 mg | about 12 mg/kg | about 900 mg | about 15 mg/kg |
| about 900 mg | about 15 mg/kg | about 1000 mg | about 20 mg/kg |
| about 1000 mg | — | about 1500 mg | about 25 mg/kg |
| about 1000 mg | — | about 1800 mg | — |
| — | — | about 2100 mg | — |
| — | — | about 2200 mg | — |
| — | — | about 2500 mg | — |

For example, an anti-LAG-3 agent (e.g., TSR-033) can be administered as: a flat dose of about 240 mg once every two weeks (Q2W), a flat dose of about 500 mg once every two weeks (Q2W), a flat dose of about 720 mg once every two weeks (Q2W), a flat dose of about 900 mg once every two weeks (Q2W), a flat dose of about 1000 mg once every two weeks (Q2W), a flat dose of about 1500 mg once every two weeks (Q2W), a weight-based dose of about 3 mg/kg once every two weeks (Q2W), a weight-based dose of about 10 mg/kg once every two weeks (Q2W), a weight-based dose of about 12 mg/kg once every two weeks (Q2W), a weight-based dose of about 15 mg/kg once every two weeks (Q2W), a flat dose of about 500 mg once every three weeks (Q3W), a flat dose of about 720 mg once every three weeks (Q3W), a flat dose of about 900 mg once every three weeks (Q3W), a flat dose of about 1000 mg once every three weeks (Q3W), a flat dose of about 1500 mg once every three weeks (Q3W), a flat dose of about 1800 mg once every three weeks (Q3W), a flat dose of about 2100 mg once every three weeks (Q3W), a flat dose of about 2200 mg once every three weeks (Q3W), a flat dose of about 2500 mg once every three weeks (Q3W), a weight-based dose of about 10 mg/kg once every three weeks (Q3W), a weight-based dose of about 12 mg/kg once every three weeks (Q3W), a weight-based dose of about 15 mg/kg once every three weeks (Q3W), a weight-based dose of about 20 mg/kg once every three weeks (Q3W), or a weight-based dose of about 25 mg/kg once every three weeks (Q3W).

TSR-033 also can be administered at a dose of: 20 mg/patient, 80 mg/patient, 240 mg/patient, 720 mg/patient, and intermediate doses between 240-720 mg/patient. TSR-033 also can be administered at a dose of up to about 1000 mg/patient (e.g., a dose of about 20, 80, 240, 500, 720, 900, or 1000 mg/patient). A dose of TSR-033 can be less than a dose used for TSR-033 monotherapy. Such a dose can be administered once every two weeks (Q2W) or once every three weeks (Q3W).

### Articles of Manufacture

In one aspect of the present disclosure, an article of manufacture containing materials useful for the treatment, prevention and/or diagnosis of the disorders described above is provided. The article of manufacture can comprise a container and a label or package insert on or associated with the container. Suitable containers can include, for example, bottles, vials, syringes, IV solution bags, etc. The containers may be formed from a variety of materials such as glass or plastic. The container can hold a composition which is by itself or combined with another composition effective for treating, preventing and/or diagnosing the condition and may have a sterile access port (for example, the container may be an intravenous solution bag or a vial having a stopper pierceable by a hypodermic injection needle). At least one active agent in the composition can be an antibody of the present disclosure. The label or package insert can indicate that the composition is used for treating the condition of choice. Moreover, the article of manufacture may comprise (a) a first container with a composition contained therein, wherein the composition comprises an antibody of the disclosure; and (b) a second container with a composition contained therein, wherein the composition comprises a further cytotoxic or otherwise therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

SEQUENCE LISTING
Exemplary Anti-PD-1 Antibody Agent TSR-042 Sequences
Anti-PD-1 antibody agent CDR sequences

| | | |
|---|---|---|
| HC-CDR1 | GFTFSSYD | SEQ ID NO: 1 |
| HC-CDR2 | ISGGGSYT | SEQ ID NO: 2 |
| HC-CDR3 | ASPYYAMDY | SEQ ID NO: 3 |
| LC-CDR1 | QDVGTA | SEQ ID NO: 4 |
| LC-CDR2 | WAS | SEQ ID NO: 5 |
| LC-CDR3 | QHYSSYPWT | SEQ ID NO: 6 |

An anti-PD-1 antibody agent heavy chain variable domain SEQ ID NO: 7-
EVQLLESGGGLVQPGGSLRLSCAASGFTFSSYDMSWVRQAPGKGLEWVSTISGGGSYTY
YQDSVKGRFTISRDNSKNTLYLQMNSLRAEDTAVYYCASPYYAMDYWGQGTTVTVSS
An anti-PD-1 antibody agent light chain variable domain SEQ ID NO: 8-
DIQLTQSPSFLSAYVGDRVTITCKASQDVGTAVAWYQQKPGKAPKLLIYWASTLHTGVP
SRFSGSGSGTEFTLTISSLQPEDFATYYCQHYSSYPWTFGQGTKLEIKR
An anti-PD-1 antibody heavy chain polypeptide SEQ ID NO: 9
EVQLLESGGGLVQPGGSLRLSCAASGFTFSSYDMSWVRQAPGKGLEWVSTISGGGSYTY
YQDSVKGRFTISRDNSKNTLYLQMNSLRAEDTAVYYCASPYYAMDYWGQGTTVTVSS
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIA VEWESNGQPENNYKTTPPVLDSDGSFFLYSRLTVDKSRW
QEGNVFSCSVMHEALHNHYTQKSLSLSLGK
An anti-PD-1 antibody light chain polypeptide SEQ ID NO: 10
DIQLTQSPSFLSAYVGDRVTITCKASQDVGTAVAWYQQKPGKAPKLLIYWASTLHTGVP
SRFSGSGSGTEFTLTISSLOPEDFATYYCQHYSSYPWTFGQGTKLEIKRTVAAPSVFIFPPS
DEQLKSGTASVVCLLNNFYPREAKVQWKVDNALQSGNSQESVTEQDSKDSTYSLSSTLT
LSKADYEKHKVYACEVTHQGLSSPVTKSFNRGEC
Exemplary Anti-TIM-3 Antibody Agent TSR-022 Sequences
Anti-TIM-3 antibody agent CDR sequences

| | | |
|---|---|---|
| HC-CDR1 | GFTFSSYD | SEQ ID NO: 11 |
| HC-CDR2 | ISGGGTYT | SEQ ID NO: 12 |
| HC-CDR3 | ASMDY | SEQ ID NO: 13 |
| LC-CDR1 | QSIRRY | SEQ ID NO: 14 |
| LC-CDR2 | GAS | SEQ ID NO: 15 |
| LC-CDR3 | QQSHSAPLT | SEQ ID NO: 16 |

A TIM-3 binding agent heavy chain variable domain SEQ ID NO: 17-
EVQLLESGGGLVQPGGSLRLSCAAASGFTFSSYDMSWVRQAPGKGLDWVSTISG
GGTYTYYQDSVKGRFTISRDNSKNTLYLQMNSLRAEDTAVYYCASMDYWGQG
TTVTVSSA
A TIM-3 binding agent heavy chain variable domain SEQ ID NO: 18
EVQLLESGGGLVQPGGSLRLSCAAASGFTFSSYDMSWVRQAPGKGLDWVSTISG
GGTYTYYQDSVKGRFTISRDNSKNTLYLQMNSLRAEDTAVYYCASMDYWGQG
TTVTVSS
A TIM-3 binding agent light chain variable domain SEQ ID NO: 19
DIQMTQSPSSLSASVGDRVTITCRASQSIRRYLNWYHQKPGKAPKLLIYGASTLQS
GVPSRFSGSGSGTDFTLTISSLQPEDFAVYYCQQSHSAPLTFGGGTKVEIKR
A TIM-3 binding agent light chain variable domain SEQ ID NO: 20
DIQMTQSPSSLSASVGDRVTITCRASQSIRRYLNWYHQKPGKAPKLLIYGASTLOS
GVPSRFSGSGSGTDFTLTISSLQPEDFAVYYCQQSHSAPLTFGGGTKVEIK
An anti-TIM-3 antibody heavy chain polypeptide (SEQ ID NO: 21)
EVQLLESGGGLVQPGGSLRLSCAAASGFTFSSYDMSWVRQAPGKGLDWVSTISGGGTY
TYYQDSVKGRFTISRDNSKNTLYLQMNSLRAEDTAVYYCASMDYWGQGTTVTVSSAST
KGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEG
NVFSCSVMHEALHNHYTQKSLSLSLGK
An anti-TIM-3 antibody light chain polypeptide (SEQ ID NO: 22)
DIQMTQSPSSLSASVGDRVTITCRASQSIRRYLNWYHQKPGKAPKLLIYGASTLQSGVPS
RFSGSGSGTDFTLTISSLQPEDFAVYYCQQSHSAPLTFGGGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Exemplary Anti-LAG-3 Antibody Agent TSR-033 Sequences
An anti-LAG-3 antibody CDR sequences

| | | |
|---|---|---|
| HC-CDR1 | DDYIH | SEQ ID NO: 23 |
| HC-CDR2 | WIDAMNDDSQYSSKFQG | SEQ ID NO: 24 |
| HC-CDR3 | AFGGY | SEQ ID NO: 25 |
| LC-CDR1 | RSSQSLVHSDSNTYLH | SEQ ID NO: 26 |
| LC-CDR2 | LVSNRFS | SEQ ID NO: 27 |
| LC-CDR3 | GQSTHVPYA | SEQ ID NO: 28 |

An anti-LAG-3 antibody heavy chain variable region amino acid sequence
SEQ ID NO: 29
EVQLVQSGAEVKKPGATVKISCKASGFSIKDDYIHWVQQAPGKGLEWMGWIDAMNDD
SQYSSKFQGRVTITVDTSTNTAYMKLSSLRSEDTAVYYCTYAFGGYWGQGTTVTVSS
An anti-LAG-3 antibody light chain variable region amino acid sequence
SEQ ID NO: 30
DIVMTQTPLSLSVTPGQPASISCRSSQSLVHSDSNTYLHWYLQKPGQSPQLLIYLVSNRFS
GVPDRFSGSGSGTDFTLKISRVEAEDVGVYFCGQSTHVPYAFGGGTKVEIK
An anti-LAG-3 antibody heavy chain polypeptide SEQ ID NO: 31
EVQLVQSGAEVKKPGATVKISCKASGFSIKDDYIHWVQQAPGKGLEWMGWIDAMNDD
SQYSSKFQGRVTITVDTSTNTAYMKLSSLRSEDTAVYYCTYAFGGYWGQGTTVTVSSAS
TKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQE
GNVFSCSVMHEALHNHYTQKSLSLSLGK
An anti-LAG-3 antibody light chain polypeptide SEQ ID NO: 32
DIVMTQTPLSLSVTPGQPASISCRSSQSLVHSDSNTYLHWYLQKPGQSPQLLIYLVSNRFS
GVPDRFSGSGSGTDFTLKISRVEAEDVGVYFCGQSTHVPYAFGGGTKVEIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Exemplary Anti-PD-1 Antibody Agent Pembrolizumab Sequences
SEQ ID NO: 33-Pembrolizumab heavy chain
QVQLVQSGVEVKKPGASVKVSCKASGYTFTNYYMYWVRQAPGQGLEWMGGIN
PSNGGTNFNEKFKNRVTLTTDSSTTTAYMELKSLQFDDTAVYYCARRDYRFDM
GFDYWGQGTTVTVSSASTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDP
EVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALH
NHYTQKSLSLSLGK
SEQ ID NO: 34-Pembrolizumab light chain
EIVLTQSPATLSLSPGERATLSCRASKGVSTSGYSYLHWYQQKPGQAPRLLIYLAS
YLESGVPARFSGSGSGTDFTLTISSLEPEDFAVYYCQHSRDLPLTFGGGTKVEIKR
TVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC

### EXEMPLARY ASPECTS AND EMBODIMENTS OF THE INVENTION

Exemplary aspects and embodiments of the invention are described herein and include items 1-215.

Item 1. A method of treating a cancer in a subject, the method comprising
measuring a level of PD-L1 expression in a sample obtained from the subject; and
administering to the subject based on the level of PD-L1 expression:
   a therapeutically effective dose of a poly (ADP-ribose) polymerase (PARP) inhibitor; and
   a therapeutically effective dose of an anti-programmed death-1 protein (PD-1) therapy,
wherein the subject has not previously received systemic chemotherapy or any previous anti-PD-1 therapy.

Item 2. A method of treating a cancer in a subject, the method comprising
selecting a subject based on the level of PD-L1 expression in a sample obtained from the subject as compared to a reference level and who has not previously received systemic chemotherapy or any previous anti-PD-1 therapy;
   and
administering to the selected subject:
   a therapeutically effective dose of a poly (ADP-ribose) polymerase (PARP) inhibitor; and
   a therapeutically effective dose of an anti-programmed death-1 protein (PD-1) therapy.

Item 3. The method of item 1 or 2, wherein the anti-PD-1 therapy administered intravenously.

Item 4. The method of any one of items 1-3, wherein the anti-PD-1 therapy administered to the subject is an agent that inhibits PD-1 or PD-L1/L2.

Item 5. The method of any one of items 1-4, wherein the anti-PD-1 therapy administered to the subject is an agent that inhibits PD-1.

Item 6. The method of item 5, wherein the agent that inhibits PD-1 is any one of PD-1 Agent Nos. 1-94.

Item 7. The method of item 5, wherein the agent that inhibits PD-1 is a small molecule, a nucleic acid, a polypeptide (e.g. an antibody, a carbohydrate, a lipid, a metal, a toxin, or a PD-1 binding agent.

Item 8. The method of item 7, wherein the agent that inhibits PD-1 is a PD-1-binding agent.

Item 9. The method of item 8, wherein the PD-1 binding agent is an antibody, an antibody conjugate, or an antigen-binding fragment thereof.

Item 10. The method of item 9, wherein the PD-1 binding agent is selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof.

Item 11. The method of item 9, wherein the PD-1 binding agent comprises:
a HC—CDR1 defined by an amino acid sequence that is identical in sequence or contains between 1-5 amino acid substitutions as compared to SEQ ID NO: 1;
a HC—CDR2 defined by an amino acid sequence that is identical in sequence or contains between 1-5 amino acid substitutions as compared to SEQ ID NO: 2;
a HC—CDR3 defined by an amino acid sequence that is identical in sequence or contains between 1-5 amino acid substitutions as compared to SEQ ID NO: 3;
a LC—CDR1 defined by an amino acid sequence that is identical in sequence or contains between 1-5 amino acid substitutions as compared to SEQ ID NO: 4;
a LC—CDR2 defined by an amino acid sequence that is identical in sequence or contains between 1-5 amino acid substitutions as compared to SEQ ID NO: 5; and
a LC—CDR3 defined by an amino acid sequence that is identical in sequence or contains between 1-5 amino acid substitutions as compared to SEQ ID NO: 6.

Item 12. The method of item 11, wherein the PD-1 binding agent comprises:
a HC—CDR1 defined by SEQ ID NO: 1;
a HC—CDR2 defined by SEQ ID NO: 2;
a HC—CDR3 defined by SEQ ID NO: 3;
a LC—CDR1 defined by SEQ ID NO: 4;
a LC—CDR2 defined by SEQ ID NO: 5; and
a LC—CDR3 defined by SEQ ID NO: 6.

Item 13. The method of any one of items 9-12, wherein the PD-1 binding agent comprises
a heavy chain variable domain having an amino acid sequence at least 80%, 85%, 90% or 95% identical to SEQ ID NO: 7; and
a light chain variable domain having an amino acid sequence at least 80%, 85%, 90% or 95% identical to SEQ ID NO: 8.

Item 14. The method of item 13, wherein the PD-1 binding agent comprises
a heavy chain variable domain having an amino acid sequence defined by SEQ ID NO: 7; and
a light chain variable domain having an amino acid sequence defined by SEQ ID NO: 8.

Item 15. The method of any one of items 9-14, wherein the PD-1 binding agent comprises
a heavy chain polypeptide having an amino acid sequence at least 80%, 85%, 90% or 95% identical to SEQ ID NO: 9; and
a light chain polypeptide having an amino acid sequence at least 80%, 85%, 90% or 95% identical to SEQ ID NO: 10.

Item 16. The method of item 15, wherein the PD-1 binding agent comprises
a heavy chain polypeptide having an amino acid sequence defined by SEQ ID NO: 9; and
a light chain polypeptide having an amino acid sequence defined by SEQ ID NO: 10.

Item 17. The method of any one of items 9-16, wherein the PD-1 binding agent is TSR-042.

Item 18. The method of any one of items 11-17, wherein the PD-1 binding agent is administered intravenously to the patient at a dose that is: a flat dose between about 100-2000 mg; a flat dose about 100 mg; a flat dose about 200 mg; a flat dose about 300 mg; a flat dose about 400 mg; a flat dose about 500 mg; a flat dose about 600 mg; a flat dose about 700 mg; a flat dose about 800 mg; a flat dose about 900 mg; a flat dose about 1000 mg; a flat dose about 1100 mg; a flat dose about 1200 mg; a flat dose about 1300 mg; a flat dose about 1400 mg; a flat dose about 1500 mg; a flat dose about 1600 mg; a flat dose about 1700 mg; a flat dose about 1800 mg; a flat dose about 1900 mg; a flat dose about 2000 mg; about 1 mg/kg; about 3 mg/kg; or about 10 mg/kg.

Item 19. The method of any one of items 11-18, wherein the dose of the PD-1 binding agent is administered to the subject at an administration interval of once a week, once every 2 weeks, once every 3 weeks, once every 4 weeks, once every 5 weeks, once every 6 weeks, or more.

Item 20. The method of item 19, wherein the dose of the PD-1 binding agent is administered at an administration interval of once every 3 weeks or once every 6 weeks.

Item 21. The method of any one of items 11-20, wherein the PD-1 binding agent is administered to the subject periodically at a dose of about 500 mg or 1000 mg.

Item 22. The method of any one of items 11-21, wherein the PD-1 binding agent is administered intravenously to the patient at a dose of about 500 mg once every about 3 weeks.

Item 23. The method of any one of items 11-21, wherein the PD-1 binding agent is administered intravenously to the patient at a dose of about 1000 mg once every about 6 weeks.

Item 24. The method of any one of items 11-23, wherein the PD-1 binding agent is administered at a first dose and first administration interval for 3, 4, or 5 cycles followed by a second dose and second administration interval for each subsequent cycle.

Item 25. The method of item 24, wherein the PD-1 binding agent is administered at a first dose of about 500 mg once every 3 weeks for 3, 4, or 5 cycles followed by a second dose of about 1000 mg once every 6 weeks or more.

Item 26. The method of item 25, wherein the PD-1 binding agent is administered intravenously to the patient at a first dose of about 500 mg once every about 3 weeks for the first four treatment cycles and then at a second dose of about 1000 mg once every about 6 weeks for the fifth and subsequent treatment cycles.

Item 27. The method of item 10, wherein the PD-1 binding agent is pembrolizumab.

Item 28. The method of item 27, wherein pembrolizumab is intravenously administered to the patient at a dose of about 200 mg to the patient once every about 3 weeks (Q3W) or about 2 mg/kg to the patient once about every 3 weeks (Q3W).

Item 29. The method of item 10, wherein the PD-1 binding agent is nivolumab.

Item 30. The method of item 29, wherein nivolumab is intravenously administered to the patient at a dose of about 200 mg to the patient once every about 3 weeks (Q3W), about 240 mg to the patient once every about 2 weeks (Q2W), about 480 mg to the patient once every about 4 weeks (Q4W), about 1 mg/kg to the patient once every about Q3W, or about 3 mg/kg to the patient once every about Q3W.

Item 31. The method of any one of items 9-30, wherein the PD-1 binding agent is administered to the patient intravenously over about 30 minutes.

Item 32. The method of any one of items 1-4, wherein the anti-PD-1 therapy administered to the subject is an anti-PD-L1/L2 agent.

Item 33. The method of item 32, wherein the anti-PD-L1/L2 agent is any of PD-L1 Agent Nos. 1-89.

Item 34. The method of item 32, wherein the anti-PD-L1/L2 agent is an anti-PD-L1 antibody agent.

Item 35. The method of item 34, wherein the anti-PD-L1 antibody agent is atezolizumab, avelumab, CX-072, durvalumab, FAZ053, LY3300054, PD-L1 millamolecule, or derivatives thereof.

Item 36. The method of any one of items 1-35, wherein the PARP inhibitor is a small molecule, a nucleic acid, a polypeptide (e.g., an antibody), a carbohydrate, a lipid, a metal, or a toxin.

Item 37. The method of any one of items 1-36, wherein the PARP inhibitor is selected from the group consisting of: ABT-767, AZD 2461, BGB-290, BGP 15, CEP 8983, CEP 9722, DR 2313, E7016, E7449, fluzoparib (SHR 3162), IMP 4297, INO1001, JPI 289, JPI 547, monoclonal antibody B3-LysPE40 conjugate, MP 124, niraparib (ZEJULA) (MK-4827), NU 1025, NU 1064, NU 1076, NU1085, olaparib (AZD2281), ON02231, PD 128763, R 503, R554, rucaparib (RUBRACA) (AG-014699, PF-01367338), SBP 101, SC 101914, simmiparib, talazoparib (BMN-673), veliparib (ABT-888), WW 46, 2-(4-(trifluoromethyl)phenyl)-7,8-dihydro-5H-thiopyrano[4,3-d]pyrimidin-4-ol, and salts or derivatives thereof.

Item 38. The method of item 37, wherein the PARP inhibitor is niraparib.

Item 39. The method of item 38, wherein niraparib is orally administered at a daily dose equivalent to about 100 mg of niraparib free base.

Item 40. The method of item 38, wherein niraparib is orally administered at a daily dose equivalent to about 200 mg of niraparib free base.

Item 41. The method of item 38, wherein niraparib is orally administered at a daily dose equivalent to about 300 mg of niraparib free base.

Item 42. The method of any one of items 1-41, wherein the PARP inhibitor is administered as part of a treatment cycle that is about 3, 4, 5, or 6 weeks.

Item 43. The method of item 42, wherein the PARP inhibitor is administered as part of a treatment cycle that is about 3 weeks or about 6 weeks.

Item 44. The method of any one of items 1-4, wherein the PD-1 therapy administered to the subject is TSR-042 intravenously administered to the patient at a dose of about 500 mg once every about 3 weeks; and the PARP inhibitor is niraparib orally administered at a dose equivalent to about 100 mg, about 200 mg, or about 300 mg of niraparib free base once daily.

Item 45. The method of any one of items 1-4, wherein
the PD-1 therapy administered to the subject is TSR-042 intravenously administered to the patient at a dose of about 1000 mg once every about 6 weeks; and
the PARP inhibitor is niraparib orally administered at a dose equivalent to about 100 mg, about 200 mg, or about 300 mg of niraparib free base once daily.

Item 46. The method of any one of items 1-4, wherein
the PD-1 therapy administered to the subject is TSR-042 intravenously administered to the patient at a first dose of 500 mg once every about 3 weeks for three, four, or five cycles, and a second dose of about 1000 mg once every about 6 weeks for subsequent cycles; and
the PARP inhibitor is niraparib orally administered at a dose equivalent to about 100 mg, about 200 mg, or about 300 mg of niraparib free base once daily.

Item 47. The method of any one of items 1-4, wherein
the PD-1 therapy administered to the subject is pembrolizumab intravenously administered to the patient at a dose of about 200 mg once every about 3 weeks; and
the PARP inhibitor is niraparib orally administered at a dose equivalent to about 100 mg, about 200 mg, or about 300 mg of niraparib free base once daily.

Item 48. The method of any one of items 1-4, wherein
the PD-1 therapy administered to the subject is nivolumab intravenously administered to the patient at a dose of about 200 mg once every about 3 weeks; and
the PARP inhibitor is niraparib orally administered at a dose equivalent to about 100 mg, about 200 mg, or about 300 mg of niraparib free base once daily.

Item 49. The method of any one of items 1-48, wherein the PARP inhibitor is administered at a dose that is less than the FDA-approved dose.

Item 50. The method of any one of items 1-49, wherein the initial dose of a PARP inhibitor is a dose equivalent to about 200 mg of niraparib free base once daily.

Item 51. The method of any one of items 1-48, wherein the initial dose of a PARP inhibitor is a dose equivalent to about 300 mg of niraparib free base once daily.

Item 52. The method of any one of items 1-51, comprising at least three treatment cycles.

Item 53. The method of any one of items 1-52, wherein the dose of the PARP inhibitor is increased if the subject's hemoglobin ≥9 g/dL, platelets ≥100,000/μL and neutrophils ≥1500/μL for all labs performed during one or more treatment cycles.

Item 54. The method of item 53, wherein the dose of the PARP inhibitor is increased after two treatment cycles.

Item 55. The method of item 54, wherein the PARP inhibitor is niraparib, and the dose is increased from a dose equivalent to about 200 mg of niraparib free base once daily to a dose equivalent to about 300 mg of niraparib free base once daily.

Item 56. The method of any one of items 1-55, wherein the anti-PD-1 therapy and the PARP inhibitor are administered according to a treatment regimen that includes at least one 2-12 week treatment cycle.

Item 57. The method of any one of items 1-56, wherein the anti-PD-1 therapy and the PARP inhibitor are administered in repeating cycles of 21 days or 3 weeks.

Item 58. The method of any one of items 1-56, wherein the anti-PD-1 therapy and the PARP inhibitor are administered in repeating cycles of 42 days or 6 weeks.

Item 59. The method of any one of items 56-58, wherein the anti-PD-1 therapy is administered on day one of cycle one.

Item 60. The method of item 59, wherein the anti-PD-1 therapy is administered on day one of a subsequent cycle.

Item 61. The method of item 59, wherein the anti-PD-1 therapy is administered between one to three days before or after day one of a subsequent cycle.

Item 62. The method of any one of items 1-48, wherein the sample obtained from the subject is a skin tissue, liver tissue, kidney tissue, lung tissue, cerebrospinal fluid (CSF), blood, amniotic fluid, sera, urine, feces, epidermal sample, skin sample, cheek swab, sperm, amniotic fluid, cultured cells, bone marrow sample and/or chorionic villi.

Item 63. The method of any one of items 1-62, wherein the sample obtained from the subject is a tissue sample or blood.

Item 64. The method of item 63, wherein the sample obtained from the subject is blood.

Item 65. The method of item 64, wherein circulating tumor cells are detected.

Item 66. The method of item 63, wherein the sample obtained from the subject is a tumor tissue sample or a cancer tissue sample.

Item 67. The method of any one of items 1-63, wherein the sample comprises a tumor cell or a cancer cell.

Item 68. The method of any one of items 1-67, wherein the level of PD-L1 expression is at least about 1% as measured by an assay.

Item 69. The method of any one of items 1-67, wherein the level of PD-L1 expression is at least about 5% as measured by an assay.

Item 70. The method of any one of items 1-67, wherein the level of PD-L1 expression is at least about 10% as measured by an assay.

Item 71. The method of any one of items 1-67, wherein the level of PD-L1 expression is at least about 25% as measured by an assay.

Item 72. The method of any one of items 1-67, wherein the level of PD-L1 expression is at least about 50% as measured by an assay.

Item 73. The method of any one of items 1-72, wherein the level of PD-L1 expression is based on PD-L1 expression in tumor cells (TC) or tumor infiltrating immune cells (IC).

Item 74. The method of any one of items 1-72, wherein the level of PD-L1 expression is measured by a tumor proportion score (TPS) or a combined positive score (CPS).

Item 75. The method of any one of items 1-74, wherein the assay is an immunohistochemical (IHC) assay, flow cytometry, imaging, PET imaging, immunofluorescence, or western blot.

Item 76. The method of item 75, wherein the assay is an immunohistochemical (IHC) assay.

Item 77. The method of any one of items 1-76, wherein the sample obtained from the subject is characterized by higher than or equal PD-L1 expression than the reference level.

Item 78. The method of item 77, wherein said sample obtained from the subject is characterized by ≥1% PD-L1 expression as measured by an assay.

Item 79. The method of item 77, wherein said sample obtained from the subject is characterized by ≥5% PD-L1 expression as measured by an assay.

Item 80. The method of item 77, wherein said sample obtained from the subject is characterized by ≥10% PD-L1 expression as measured by an assay.

Item 81. The method of item 77, wherein said sample obtained from the subject is characterized by ≥25% PD-L1 expression as measured by an assay.

Item 82. The method of any one of items 1-81, wherein said sample obtained from the subject is characterized by high PD-L1 expression.

Item 83. The method of item 82, wherein the sample obtained from the subject is characterized by ≥50% PD-L1 expression as measured by an assay.

Item 84. The method of item 83, wherein the sample obtained from the subject is characterized by ≥60%, 65%, 70%, 75%, 80%, 85%, or 90% PD-L1 expression as measured by an assay.

Item 85. The method of any one of items 1-84, wherein the level of PD-L1 in the sample obtained from the subject is measured by a tumor proportion score (TPS).

Item 86. The method of any one of items 1-85, wherein the level of PD-L1 in the sample obtained from the subject is measured by a combined positive score (CPS).

Item 87. The method of any one of items 83-86, wherein the assay is an immunohistochemical (IHC) assay, flow cytometry, imaging, PET imaging, immunofluorescence, or western blot.

Item 88. The method of item 87, wherein the assay is an immunohistochemical (IHC) assay.

Item 89. A method of treating a cancer in a subject, the method comprising
measuring a level of PD-L1 expression in a sample obtained from the subject;
determining that said sample is characterized by a tumor proportion score (TPS) of at least about 1%; and
administering to the subject a therapeutically effective dose of a poly (ADP-ribose) polymerase (PARP) inhibitor that is niraparib and a therapeutically effective dose of an anti-PD-1 therapy.

Item 90. A method of treating a cancer in a subject, the method comprising
selecting a subject based a level of PD-L1 expression in a sample obtained from the subject that is equal to or higher as compared to a reference level, wherein the reference level is a tumor proportion score (TPS) of at least about 1%; and
administering to the subject a therapeutically effective dose of a poly (ADP-ribose) polymerase (PARP) inhibitor that is niraparib and a therapeutically effective dose of an anti-PD-1 therapy.

Item 91. A method of treating a cancer in a subject, the method comprising
measuring a level of PD-L1 expression in a sample obtained from the subject;
determining that said sample is characterized by a tumor proportion score (TPS) of at least about 50%; and
administering to the subject a therapeutically effective dose of a poly (ADP-ribose) polymerase (PARP) inhibitor that is niraparib and a therapeutically effective dose of an anti-PD-1 therapy.

Item 92. A method of treating a cancer in a subject, the method comprising
selecting a subject based a level of PD-L1 expression in a sample obtained from the subject that is equal to or higher as compared to a reference level, wherein the reference level is a tumor proportion score (TPS) of at least about 50%; and
administering to the subject a therapeutically effective dose of a poly (ADP-ribose) polymerase (PARP) inhibitor that is niraparib and a therapeutically effective dose of an anti-PD-1 therapy.

Item 93. The method of any one of items 89-92, wherein the TPS is ≥60%, 65%, 70%, 75%, 80%, 85%, or 90%.

Item 94. The method of any one of items 89-93, wherein the TPS is measured by an immunohistochemical (IHC) assay.

Item 95. The method of any one of items 89-94, wherein the anti-PD-1 therapy is
i) an agent that inhibits PD-1;
ii) an agent that inhibits PD-L1/L2;
iii) a small molecule, a nucleic acid, a polypeptide (e.g. an antibody, a carbohydrate, a lipid, a metal, a toxin, or a PD-1 binding agent that inhibits PD-1;
iv) a PD-1 binding agent;
v) a PD-1 binding agent that is an antibody, an antibody conjugate, or an antigen-binding fragment thereof;
vi) a PD-1 binding agent selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof;
vii) any one of PD-1 Agent Nos. 1-94;
viii) any of PD-L1 Agent Nos. 1-89;
ix) a small molecule, a nucleic acid, a polypeptide (e.g. an antibody, a carbohydrate, a lipid, a metal, a toxin, or a PD-L1 binding agent that inhibits PD-1;
x) a PD-L1 binding agent;
xi) a PD-L1 binding agent that is an antibody, an antibody conjugate, or an antigen-binding fragment thereof;
xii) a PD-L1 agent selected from the group consisting of: atezolizumab, avelumab, CX-072, durvalumab, FAZ053, LY3300054, PD-L1 millamolecule, and derivatives thereof;
xiii) TSR-042, pembrolizumab, or nivolumab; or
xiv) TSR-042.

Item 96. The method of any one of items 89-95, wherein the anti-PD-1 therapy is TSR-042, pembrolizumab, or nivolumab.

Item 97. The method of any one of items 89-96, wherein the subject has not previously received systemic chemotherapy.

Item 98. The method any one of items 89-97, wherein the subject has not previously received platinum-based chemotherapy.

Item 99. The method of any one of items 89-98, wherein the subject has not previously received any anti-PD-1 therapy.

Item 100. The method of any one of items 89-99, wherein the subject has previously been treated with one or more cancer treatment modalities.

Item 101. The method of item 100, wherein the subject has previously been treated with one or more of surgery or radiotherapy.

Item 102. The method of item 100 or 101, wherein the subject has previously been treated with chemotherapy or immunotherapy.

Item 103. The method of any one of items 100 102, wherein the subject has been treated with one, two, three, four, or five lines of prior therapy.

Item 104. The method of item 103, wherein the subject has been treated with one or two lines of prior therapy.

Item 105. The method of item 103, wherein the subject has been treated with one line of prior therapy.

Item 106. The method of item 103, wherein the subject has been treated with two lines of prior therapy.

Item 107. The method of any one of items 100-106, wherein the subject has previously received immunotherapy.

Item 108. The method of any one of items 100-107, wherein the cancer is recurrent cancer and/or advanced cancer.

Item 109. The method of item 108, wherein the cancer is refractory to a previously received cancer treatment.

Item 110. The method of item 109, wherein the cancer was refractory to a previously received cancer treatment at the beginning of treatment.

Item 111. The method of item 109, wherein the cancer became refractory to a previously received cancer treatment during the treatment.

Item 112. The method of any one of items 100-111, wherein the subject has previously received immunotherapy, wherein the immunotherapy is not an anti-PD-1 therapy.

Item 113. The method of any one of items 100-111, wherein the subject has previously received immunotherapy that is an anti-PD-1 therapy.

Item 114. The method of item 113, wherein the anti-PD-1 therapy is a PD-1 binding agent.

Item 115. The method of item 113 or 114, wherein the cancer is recurrent and/or advanced cancer.

Item 116. The method of any one of items 114-115, wherein the cancer is refractory to the previously received anti-PD-1 therapy.

Item 117. The method of item 117, wherein the cancer was refractory to a previously received anti-PD-1 therapy at the beginning of treatment.

Item 118. The method of item 117, wherein the cancer became refractory to a previously received anti-PD-1 therapy during the treatment.

Item 119. The method of any one of items 116-118, wherein the anti-PD-1 therapy is a PD-1 binding agent.

Item 120. The method of one of items 116-118, wherein the anti-PD-1 therapy is a PD-L1 binding agent.

Item 121. The method of any one of items 100-120, wherein the subject has previously been treated with chemotherapy.

Item 122. The method of item 121, wherein the chemotherapy is platinum-based chemotherapy.

Item 123. The method of item 122, wherein the chemotherapy is platinum-based doublet chemotherapy.

Item 124. The method of item 122 or 123, wherein the chemotherapy comprises administration of cisplatin, carboplatin, oxaliplatin, nedaplatin, triplatin tetranitrate, phenanthriplatin, picoplatin, and/or satraplatin.

Item 125. The method of any one of items 121-124, wherein the cancer is recurrent and/or advanced cancer.

Item 126. The method of any one of items 121-125, wherein the cancer is refractory to the chemotherapy.

Item 127. The method of item 126, wherein the cancer was refractory to a chemotherapy at the beginning of treatment.

Item 128. The method of item 126, wherein the cancer became refractory to a chemotherapy during the treatment.

Item 129. The method of any one of items 89-128, wherein the anti-PD-1 therapy is TSR-042.

Item 130. The method of item 129, wherein TSR-042 is intravenously administered to the subject at a dose of about 500 mg once every about 3 weeks.

Item 131. The method of item 129, wherein TSR-042 is intravenously administered to the subject at a dose of about 1000 mg once every about 6 weeks.

Item 132. The method of item 129, wherein TSR-042 is intravenously administered to the subject at a first dose of about 500 mg once every about 3 weeks for the first three, four, or five treatment cycles and then at a second dose of about 1000 mg once every about 6 weeks for subsequent treatment cycles.

Item 133. The method of any one of items 89-126, wherein the anti-PD-1 therapy is pembrolizumab.

Item 134. The method of item 133, wherein pembrolizumab is intravenously administered to the subject at a dose of about 200 mg once every about 3 weeks or about 2 mg/kg to the patient once about every Q3W.

Item 135. The method of any one of items 89-126, wherein the anti-PD-1 therapy is nivolumab.

Item 136. The method of item 133, wherein nivolumab is intravenously administered to the subject at a dose of about 200 mg once every about 3 weeks, about 240 mg to the patient once every about 2 weeks (Q2W), about 480 mg to the patient once every about 4 weeks (Q4W), about 1 mg/kg to the patient once every about Q3W, or about 3 mg/kg to the patient once every about Q3W.

Item 137. The method of any one of items 89-136, wherein the PARP inhibitor is administered at an initial dose that is less than the FDA-approved dose.

Item 138. The method of any one of items 89-137, wherein the initial dose of a PARP inhibitor is a dose equivalent to about 200 mg of niraparib free base once daily.

Item 139. The method of any one of items 89-136, wherein the initial dose of a PARP inhibitor is a dose equivalent to about 300 mg of niraparib free base once daily.

Item 140. The method of any one of items 89-139, comprising at least three treatment cycles.

Item 141. The method of any one of items 89-140, wherein the PARP inhibitor is increased if the subject's hemoglobin ≥9 g/dL, platelets ≥100,000/μL and neutrophils ≥1500/μL for all labs performed during one or more treatment cycles.

Item 142. The method of item 141, wherein the dose of the PARP inhibitor is increased after two treatment cycles.

Item 143. The method of item 142, wherein the PARP inhibitor is niraparib, and the dose is increased from a dose equivalent to about 200 mg of niraparib free base once daily to a dose equivalent to about 300 mg of niraparib free base once daily.

Item 144. The method of any one of items 1-143, wherein the cancer is MSS or MSI-L, is characterized by microsatellite instability, is MSI-H, has high TMB, has high TMB and is MSS or MSI-L, has high TMB and is MSI-H, has a defective DNA mismatch repair system, has a defect in a DNA mismatch repair gene, is a hypermutated cancer, is an HRD or HRR cancer, comprises a mutation in polymerase delta (POLD), or comprises a mutation in polymerase epsilon (POLE).

Item 145. The method of any one of items 1-144, wherein the cancer is adenocarcinoma, endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anus, squamous cell carcinoma of the penis, squamous cell carcinoma of the cervix, squamous cell carcinoma of the vagina, squamous cell carcinoma of the vulva, soft tissue sarcoma, melanoma, renal cell carcinoma, lung cancer, non-small cell lung cancer, adenocarcinoma of the lung, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, a hematological cancer, multiple myeloma, B-cell lymphoma, T-cell lymphoma, Hodgkin's lymphoma/primary mediastinal B-cell lymphoma, chronic myelogenous leukemia, acute myeloid leukemia, acute lymphoblastic leukemia, non-Hodgkin's lymphoma, neuroblastoma, a CNS tumor, diffuse intrinsic pontine glioma (DIPG), Ewing's sarcoma, embryonal rhabdomyosarcoma, osteosarcoma, or Wilms tumor.

Item 146. The method of item 145, wherein the cancer is melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, endometrial cancer, ovarian cancer, or Merkel cell carcinoma.

Item 147. The method of any one of items 1-146, wherein the cancer is a solid tumor.

Item 148. The method of item 147, wherein the cancer is lung cancer.

Item 149. The method of item 148, wherein the lung cancer is non-small cell lung cancer (NSCLC).

Item 150. The method of item 149, wherein the lung cancer is squamous non-small cell lung cancer (sqNSCLC).

Item 151. The method of item 149, wherein the lung cancer is adenocarcinoma.

Item 152. The method of item 149, wherein the lung cancer is large-cell carcinoma.

Item 153. The method of any one of items 148-152, wherein the lung cancer is characterized by an ALK translocation.

Item 154. The method of any one of items 148-152, wherein the lung cancer does not have an ALK translocation.

Item 155. The method of any one of items 148-154, wherein the lung cancer is characterized by an EGFR mutation.

Item 156. The method of any one of items 148-154, wherein the lung cancer does not have an EGFR mutation.

Item 157. The method of any one of items 148-156, wherein the cancer is characterized by a gene amplification.

Item 158. The method of item 157, wherein the cancer is characterized by a gene amplification in mesenchymal epithelial transition factor (MET).

Item 159. The method of any one of items 148-156, wherein the lung cancer is stage III or stage IV cancer.

Item 160. The method of any one of items 148-159, wherein the lung cancer is locally advanced.

Item 161. The method of any one of items 148-159, wherein the lung cancer is metastatic.

Item 162. A method of treating non-small cell lung cancer (NSCLC) in a subject, the method comprising
measuring a level of PD-L1 expression in a sample obtained from the subject, wherein the subject has not previously received systemic chemotherapy or any previous anti-PD-1 therapy;
determining that said sample is characterized by a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of TSR-042 in an amount that is about 500 mg once every about 3 weeks.

Item 163. A method of treating a non-small cell lung cancer (NSCLC) in a subject, the method comprising
selecting a subject based a level of PD-L1 expression in a sample obtained from the subject that is equal to or higher as compared to a reference level, wherein the reference level is a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of TSR-042 in an amount that is about 500 mg once every about 3 weeks; and
wherein the subject has not previously received systemic chemotherapy or any previous anti-PD-1 therapy.

Item 164. A method of treating non-small cell lung cancer (NSCLC) in a subject, the method comprising
measuring a level of PD-L1 expression in a sample obtained from the subject, wherein the subject has not previously received systemic chemotherapy or any previous anti-PD-1 therapy;
determining that said sample is characterized by a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of TSR-042 in an amount that is about 1000 mg once every about 6 weeks.

Item 165. A method of treating a non-small cell lung cancer (NSCLC) in a subject, the method comprising
selecting a subject based a level of PD-L1 expression in a sample obtained from the subject that is equal to or higher as compared to a reference level, wherein the reference level is a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of TSR-042 in an amount that is about 1000 mg once every about 6 weeks; and
wherein the subject has not previously received systemic chemotherapy or any previous anti-PD-1 therapy.

Item 166. A method of treating non-small cell lung cancer (NSCLC) in a subject, the method comprising
measuring a level of PD-L1 expression in a sample obtained from the subject, wherein the subject has not previously received systemic chemotherapy or any previous anti-PD-1 therapy;
determining that said sample is characterized by a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of TSR-042 as a first dose of about 500 mg TSR-042 once every three weeks for three, four, or five treatment cycles and then as a second dose of about 1000 mg TSR-042 once every about 6 weeks for each subsequent treatment cycle.

Item 167. A method of treating a non-small cell lung cancer (NSCLC) in a subject, the method comprising
selecting a subject based a level of PD-L1 expression in a sample obtained from the subject that is equal to or higher as compared to a reference level, wherein the reference level is a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of TSR-042 as a first dose of about 500 mg TSR-042 once every three weeks for three, four, or five treatment cycles and then as a second dose of about 1000 mg TSR-042 once every about 6 weeks for each subsequent treatment cycle; and
wherein the subject has not previously received systemic chemotherapy or any previous anti-PD-1 therapy.

Item 168. A method of treating non-small cell lung cancer (NSCLC) in a subject, the method comprising
measuring a level of PD-L1 expression in a sample obtained from the subject, wherein the subject has not previously received systemic chemotherapy or any previous anti-PD-1 therapy;
determining that said sample is characterized by a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of pembrolizumab in an amount that is about 200 mg once every about 3 weeks or about 2 mg/kg to the patient once about every 3 weeks.

Item 169. A method of treating a non-small cell lung cancer (NSCLC) in a subject, the method comprising
selecting a subject based a level of PD-L1 expression in a sample obtained from the subject that is equal to or higher as compared to a reference level, wherein the reference level is a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of pembrolizumab in an amount that is about 200 mg once every about 3 weeks or about 2 mg/kg to the patient once about every 3 weeks; and
wherein the subject has not previously received systemic chemotherapy or any previous anti-PD-1 therapy.

Item 170. A method of treating non-small cell lung cancer (NSCLC) in a subject, the method comprising
measuring a level of PD-L1 expression in a sample obtained from the subject, wherein the subject has not previously received systemic chemotherapy or any previous anti-PD-1 therapy;
determining that said sample is characterized by a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of nivolumab in an amount that is about 200 mg once every about 3 weeks, about 240 mg to the patient once every about 2 weeks, about 480 mg to the patient once every about 4 weeks, about 1 mg/kg to the patient once every about 3 weeks, or about 3 mg/kg to the patient once every about 3 weeks.

Item 171. A method of treating a non-small cell lung cancer (NSCLC) in a subject, the method comprising
selecting a subject based a level of PD-L1 expression in a sample obtained from the subject that is equal to or higher as compared to a reference level, wherein the reference level is a tumor proportion score (TPS) of at least about 50%; and
orally administering to the subject a therapeutically effective dose of niraparib in an amount that is equivalent to about 200 mg or 300 mg of niraparib free base once daily, and intravenously administering a therapeutically effective dose of nivolumab in an amount that is about 200 mg once every about 3 weeks, about 240 mg to the patient once every about 2 weeks, about 480 mg to the patient once every about 4 weeks, about 1 mg/kg to the patient once every about 3 weeks, or about 3 mg/kg to the patient once every about 3 weeks; and
wherein the subject has not previously received systemic chemotherapy or any previous anti-PD-1 therapy.

Item 172. The method of any one of items 162-171, wherein the TPS is ≥60%, 65%, 70%, 75%, 80%, 85%, or 90%.

Item 173. The method of any one of items 162-172, wherein the TPS is measured by an immunohistochemical assay.

Item 174. The method of any one of items 147-173, wherein the method inhibits tumor growth or reduces tumor size.

Item 175. The method of any one of items 1-174, wherein the method further comprises administering another therapeutic agent or treatment.

Item 176. The method of item 175, wherein the method further comprises administering one or more of surgery, a radiotherapy, a chemotherapy, an immunotherapy, an anti-angiogenic agent, or an anti-inflammatory agent.

Item 177. The method of item 176, wherein the method further comprises administering an immune checkpoint inhibitor.

Item 178. The method of item 177, comprising further administering one, two, or three immune checkpoint inhibitors.

Item 179. The method of item 177 or 178, wherein an immune checkpoint inhibitor is an inhibitor of PD-1, TIM-3, LAG-3, CTLA-4, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R.

Item 180. The method of item 178 or 179, wherein the immune checkpoint inhibitor is an agent that inhibits programmed death-1 protein (PD-1) signaling, T cell immunoglobulin and mucin protein 3 (TIM-3), lymphocyte activation gene-3 (LAG-3), cytotoxic T-lymphocyte-associated protein 4 (CTLA-4), T cell immunoglobulin and ITIM domain (TIGIT), indoleamine 2,3-dioxygenase (IDO), or colony-stimulating factor 1 receptor (CSF1R).

Item 181. The method of item 178 or 179, further comprising administering a therapeutically effective dose of an anti-Lymphocyte Activation Gene-3 (LAG-3) therapy and/or a therapeutically effective dose of an anti-T Cell Immunoglobulin and Mucin Domain-3 (TIM-3) therapy.

Item 182. The method of item 181, comprising administering a therapeutically effective dose of an anti-T Cell Immunoglobulin and Mucin Domain-3 (TIM-3) therapy.

Item 183. The method of item 182, wherein the anti-TIM-3 therapy is any of TIM-3 Agent Nos. 1-21.

Item 184. The method of item 182, wherein the anti-TIM-3 therapy is an agent that inhibits TIM-3.

Item 185. The method of item 184, wherein the anti-TIM-3 therapy is a small molecule, a nucleic acid, a polypeptide (e.g. an antibody), a carbohydrate, a lipid, a metal, a toxin or a TIM-3 binding agent.

Item 186. The method of item 185, wherein the anti-TIM-3 therapy is a TIM-3 binding agent.

Item 187. The method of item 186, wherein the TIM-3 binding agent is an antibody, an antibody conjugate, or an antigen-binding fragment thereof.

Item 188. The method of item 187, wherein the TIM-3 binding agent is MBG453, LY3321367, Sym023, TSR-022 or a derivative thereof.

Item 189. The method of item 187, wherein the TIM-3 binding agent comprises:
a HC—CDR1 defined by an amino acid sequence that is identical in sequence or contains between 1-5 amino acid substitutions as compared to SEQ ID NO: 11;
a HC—CDR2 defined by an amino acid sequence that is identical in sequence or contains between 1-5 amino acid substitutions as compared to SEQ ID NO: 12;
a HC—CDR3 defined by an amino acid sequence that is identical in sequence or contains between 1-5 amino acid substitutions as compared to SEQ ID NO: 13;
a LC—CDR1 defined by an amino acid sequence that is identical in sequence or contains between 1-5 amino acid substitutions as compared to SEQ ID NO: 14;
a LC—CDR2 defined by an amino acid sequence that is identical in sequence or contains between 1-5 amino acid substitutions as compared to SEQ ID NO: 15; and
a LC—CDR3 defined by an amino acid sequence that is identical in sequence or contains between 1-5 amino acid substitutions as compared to SEQ ID NO: 16.

Item 190. The method of item 189, wherein the TIM-3 binding agent comprises:
a HC—CDR1 defined by SEQ ID NO: 11;
a HC—CDR2 defined by SEQ ID NO: 12;
a HC—CDR3 defined by SEQ ID NO: 13;
a LC—CDR1 defined by SEQ ID NO: 14;
a LC—CDR2 defined by SEQ ID NO: 15; and
a LC—CDR3 defined by SEQ ID NO: 16.

Item 191. The method of any one of items 187 and 189-190, wherein the TIM-3 binding agent comprises
a heavy chain variable domain having an amino acid sequence at least 80%, 85%, 90% or 95% identical to SEQ ID NO: 17 or 18; and
a light chain variable domain having an amino acid sequence at least 80%, 85%, 90% or 95% identical to SEQ ID NO: 19 or 20.

Item 192. The method of item 191, wherein the TIM-3 binding agent comprises
a heavy chain variable domain having an amino acid sequence defined by SEQ ID NO: 17 or 18; and
a light chain variable domain having an amino acid sequence defined by SEQ ID NO: 19 or 20.

Item 193. The method of any one of items 187 and 189-192, wherein the TIM-3 binding agent comprises
a heavy chain polypeptide having an amino acid sequence at least 80%, 85%, 90% or 95% identical to SEQ ID NO: 21; and
a light chain polypeptide having an amino acid sequence at least 80%, 85%, 90% or 95% identical to SEQ ID NO: 22.

Item 194. The method of item 193, wherein the TIM-3 binding agent comprises
a heavy chain polypeptide having an amino acid sequence defined by SEQ ID NO: 21; and
a light chain polypeptide having an amino acid sequence defined by SEQ ID NO: 22.

Item 195. The method of item 188, wherein the TIM-3 binding agent is TSR-022 or a derivative thereof.

Item 196. The method of any one of items 181-195, wherein the therapeutically effective dose of the anti-TIM-3 therapy is a flat dose of about 100 mg, about 300 mg, about 500 mg, about 900 mg, or about 1200 mg or a weight-based dose of about 1 mg/kg, about 3 mg/kg, or about 10 mg/kg.

Item 197. The method of item 196, wherein the therapeutically effective dose of the anti-TIM-3 therapy is a flat dose of about 100 mg.

Item 198. The method of item 196, wherein the therapeutically effective dose of the anti-TIM-3 therapy is a flat dose of about 300 mg.

Item 199. The method of item 196, wherein the therapeutically effective dose of the anti-TIM-3 therapy is a flat dose of about 900 mg.

Item 200. The method of any one of items 181-199, wherein the anti-TIM-3 therapy is administered intravenously once every three weeks.

Item 201. The method of any one of items 181-200, comprising administering a therapeutically effective dose of an anti-LAG-3 agent that inhibits LAG-3.

Item 202. The method of item 201, wherein the agent that inhibits LAG-3 is any one of LAG-3 Agent Nos. 1-24.

Item 203. The method of item 201, wherein the agent that inhibits LAG-3 is a small molecule, a nucleic acid, a polypeptide (e.g. an antibody, a carbohydrate, a lipid, a metal, a toxin, or a LAG-3 binding agent.

Item 204. The method of item 203, wherein the agent that inhibits LAG-3 is a LAG-3-binding agent.

Item 205. The method of item 204, wherein the LAG-3 binding agent is an antibody, an antibody conjugate, or an antigen-binding fragment thereof.

Item 206. The method of item 205, wherein the LAG-3 binding agent is IMP321, relatlimab (BMS-986016), BI 754111, GSK2831781 (IMP-731), Novartis LAG525 (IMP701), REGN3767, MK-4280, MGD-013, GSK-2831781, FS-118, XmAb22841, INCAGN-2385, FS-18, ENUM-006, AVA-017, AM-0003, Avacta PD-L1/LAG-3 bispecific affamer, iOnctura anti-LAG-3 antibody, Arcus anti-LAG-3 antibody, or Sym022, and derivatives thereof.

Item 207. The method of item 205, wherein the LAG-3 binding agent is TSR-033.

Item 208. The method of item 205, wherein the LAG-3 binding agent comprises:
a HC—CDR1 defined by an amino acid sequence that is identical in sequence or contains between 1-5 amino acid substitutions as compared to SEQ ID NO: 23;
a HC—CDR2 defined by an amino acid sequence that is identical in sequence or contains between 1-5 amino acid substitutions as compared to SEQ ID NO: 24;
a HC—CDR3 defined by an amino acid sequence that is identical in sequence or contains between 1-5 amino acid substitutions as compared to SEQ ID NO: 25;
a LC—CDR1 defined by an amino acid sequence that is identical in sequence or contains between 1-5 amino acid substitutions as compared to SEQ ID NO: 26;
a LC—CDR2 defined by an amino acid sequence that is identical in sequence or contains between 1-5 amino acid substitutions as compared to SEQ ID NO: 27; and
a LC—CDR3 defined by an amino acid sequence that is identical in sequence or contains between 1-5 amino acid substitutions as compared to SEQ ID NO: 28.

Item 209. The method of item 208, wherein the LAG-3 binding agent comprises:
a HC—CDR1 defined by SEQ ID NO: 23;
a HC—CDR2 defined by SEQ ID NO: 24;
a HC—CDR3 defined by SEQ ID NO: 25;
a LC—CDR1 defined by SEQ ID NO: 26;
a LC—CDR2 defined by SEQ ID NO: 27; and
a LC—CDR3 defined by SEQ ID NO: 28.

Item 210. The method of any one of items 204-209, wherein the LAG-3 binding agent comprises
a heavy chain variable domain having an amino acid sequence at least 80%, 85%, 90% or 95% identical to SEQ ID NO: 29; and
a light chain variable domain having an amino acid sequence at least 80%, 85%, 90% or 95% identical to SEQ ID NO: 30.

Item 211. The method of item 210, wherein the LAG-3 binding agent comprises
a heavy chain variable domain having an amino acid sequence defined by SEQ ID NO: 29; and
a light chain variable domain having an amino acid sequence defined by SEQ ID NO: 30.

Item 212. The method of any one of items 204-211, wherein the LAG-3 binding agent comprises
a heavy chain polypeptide having an amino acid sequence at least 80%, 85%, 90% or 95% identical to SEQ ID NO: 31; and
a light chain polypeptide having an amino acid sequence at least 80%, 85%, 90% or 95% identical to SEQ ID NO: 32.

Item 213. The method of item 212, wherein the LAG-3 binding agent comprises
a heavy chain polypeptide having an amino acid sequence defined by SEQ ID NO: 31; and
a light chain polypeptide having an amino acid sequence defined by SEQ ID NO: 32.

Item 214. The method of any one of items 181-213, wherein the anti-LAG-3 therapy is administered as a flat dose of about 240 mg once every two weeks (Q2W), a flat dose of about 500 mg once every two weeks (Q2W), a flat dose of about 720 mg once every two weeks (Q2W), a flat dose of about 900 mg once every two weeks (Q2W), a flat dose of about 1000 mg once every two weeks (Q2W), a flat dose of about 1500 mg once every two weeks (Q2W), a weight-based dose of about 3 mg/kg once every two weeks (Q2W), a weight-based dose of about 10 mg/kg once every two weeks (Q2W), a weight-based dose of about 12 mg/kg once every two weeks (Q2W), a weight-based dose of about 15 mg/kg once every two weeks (Q2W), a flat dose of about 500 mg once every three weeks (Q3W), a flat dose of about 720 mg once every three weeks (Q3W), a flat dose of about 900 mg once every three weeks (Q3W), a flat dose of about 1000 mg once every three weeks (Q3W), a flat dose of about 1500 mg once every three weeks (Q3W), a flat dose of about 1800 mg once every three weeks (Q3W), a flat dose of about 2100 mg once every three weeks (Q3W), a flat dose of about 2200 mg once every three weeks (Q3W), a flat dose of about 2500 mg once every three weeks (Q3W), a weight-based dose of about 10 mg/kg once every three weeks (Q3W), a weight-based dose of about 12 mg/kg once every three weeks (Q3W), a weight-based dose of about 15 mg/kg once every three weeks (Q3W), a weight-based dose of about 20 mg/kg once every three weeks (Q3W), or a weight-based dose of about 25 mg/kg once every three weeks (Q3W).

Item 215. The method of any one of items 1-214, wherein the method provides a clinical benefit to the subject that is a complete response (“CR”), a partial response (“PR”) or stable disease (“SD”).

### EXAMPLES

The following examples are provided to illustrate, but not limit the claimed invention.

### Example—Treatment of Cancer with a PARP Inhibitor in Combination with an Anti-PD-1 Agent

This example describes a multicenter, open-label, multi-arm Phase 2 study to evaluate the efficacy and safety of a combination of a PARP inhibitor (niraparib) and an anti-PD-1 agent (pembrolizumab or TSR-042) in locally advanced and metastatic NSCLC (all histologies) patients.

### Inclusion Criteria: Cohorts 1 and 1A

Eligible patients were those having locally advanced and metastatic NSCLC patients (all histologies) with no prior systemic chemotherapy or PD-1/PD-L1 inhibitor treatment, whose tumors have high PD-L1 expression (TPS≥50%), and no known epidermal growth factor receptor (EGFR) sensitizing mutation and/or ROS-1 or anaplastic lymphoma kinase (ALK) translocations will receive combination of niraparib and a PD-1 inhibitor such as pembrolizumab (Cohort 1) or TSR-042 (Cohort 1A). Completion of treatment with chemotherapy and/or radiation as part of neoadjuvant/adjuvant therapy is allowed as long as therapy was completed at least 6 months prior to the diagnosis of metastatic disease.

### Biomarkers

Blood and tumor-based biomarkers can be used to predict sensitivity or resistance to the exemplary treatment methods described herein.

Biomarker analysis was carried out on tumor tissue and blood to include but not be limited to circulating tumor DNA (ctDNA) or circulating tumor cells (CTC) to identify prognostic or predictive biomarkers and to explore potential mechanisms of either de novo or treatment-emergent resistance. Biomarkers can be evaluated in archival or fresh tumor samples obtained during screening to confirm histology morphology, presence of tumor, and to conduct biomarker analysis. An archival FFPE sample should be submitted within 30 days of patient's first dose. Blood samples for the analysis of tumor-related circulating biomarkers such as CTC can be collected at Cycle 1/Day 1 predose. Blood samples for the analysis of ctDNA will be obtained at Screening, Cycle 2/Day 1 predose, as well as EOT.

For example, PD-L1 status can be determined either prospectively or retrospectively using an FDA-approved in vitro companion diagnostic indicated as an aid in identifying NSCLC patients for treatment with a PD-1 inhibitor.

Administration of an Exemplary Anti-PD-1 Agent (Pembrolizumab or TSR-042) in Combination with an Exemplary PARP Inhibitor (Niraparib)

Table 13 provides an overview of exemplary protocols for the administration of a PARP inhibitor with an anti-PD-1 agent.

TABLE
Administration of a PARP inhibitor with an Anti-PD-1 Agent

| | PARP Inhibitor | Anti-PD-1 Agent | |
|---|---|---|---|
| Product Name: | Niraparib | Pembrolizumab | TSR-042 |
| Dosage Form: | 100 mg capsule | infusion | infusion |
| Unit Dose | 2 capsules of | 200 mg infusion | 500 mg or 1,000 |
| | 100 mg each | Administered | mg infusion |
| | (200 mg/day) | over 30 minutes | Administered over |
| | Administered | Day 1 of each | 30 minutes |
| | once daily | 21-day cycle | 500 mg: Day 1 of |
| | 21-day cycle | | each 21-day cycle |
| | | | (Q3W) for the |
| | | | first 4 cycles |
| | | | 1,000 mg: Day 1 |
| | | | of every other |
| | | | 21-day cycle |
| | | | (Q6W) for the |
| | | | rest of treatment, |
| | | | beginning on |
| | | | Cycle 5 |
| Route of | PO | IV | IV |
| Administration | | | |

### Pembrolizumab and TSR-042

For patients of Cohort 1, pembrolizumab was administered at a dose of 200 mg IV using a 30-minute infusion. The target infusion timing was as close to 30 minutes as possible, although some variation was permitted (e.g., a window between −5 minutes and +10 minutes was permitted). Pembrolizumab infusion was administered before niraparib dose at the study site on Day 1 of each 21-day treatment cycle. Pembrolizumab may be administered up to 3 days before or after the scheduled Day 1 of each cycle after Cycle 1. Dose interruption will be allowed for no longer than 28 days.

A patient in Cohort 1A can alternatively receive a TSR-042 infusion instead of pembrolizumab. The TSR-042 infusion will be administered before niraparib dose at the study site on Day 1 of each 21-day treatment cycle (Q3W) in Cycles 1 through 4 and on Day 1 of every other cycle (Q6W) thereafter, beginning on Cycle 5 Day 1 (e.g., Cycle 5, Cycle 7, Cycle 9, etc.). TSR-042 may be administered up to 3 days before or after the scheduled Day 1 of each cycle after Cycle 1 due to administrative reasons. TSR-042 can be administered at a dose of 500 mg IV Q3W in Cycles 1 through 4 and at a dose of 1,000 mg IV Q6W thereafter, beginning on Cycle 5 Day 1, for the rest of the treatment using a 30-minute infusion. The target infusion timing is as close to 30 minutes as possible, although some variation is permitted (e.g., a window between −5 minutes and +15 minutes is permitted). Dose interruption to manage adverse reactions will be allowed for no longer than 28 days.

### Niraparib

An exemplary protocol for administration of niraparib in combination with administration of pembrolizumab is provided (e.g., as administered for the patients of Cohort 1). This protocol can be used for administration in combination with other anti-PD-1 agents such as TSR-042 for the patients of, e.g., Cohort 1A, using the exemplary dosages of TSR 042 described herein.

Niraparib was administered orally once a day, continuously throughout the 21-day cycle. Two capsules of 100 mg strength (200 mg/day) were taken at each dose administration. Niraparib was dispensed to patients on Day 1 of every cycle (every 21 days) thereafter until the patient discontinues study treatment.

Patients were instructed to take their niraparib dose in the morning at approximately the same time each day. Niraparib may be taken with or without food or water. Patients must swallow and not chew the capsules. For certain patients experiencing nausea, bedtime administration may be a potential method for managing nausea.

The niraparib dose may be escalated on or after Cycle 3/Day 1 from 200 mg daily (2 capsules) to 300 mg daily (3 capsules) if platelets ≥100,000/μL, hemoglobin ≥9 g/dL, and neutrophils ≥1,500/μL for all laboratory tests performed during the first 2 cycles. In addition, dose reduction will be allowed based on treatment side effects. Dose reductions to 100 mg daily (1 capsule) will be allowed. No further dose reductions will be allowed. Dose interruption will be allowed for no longer than 28 days.

### Measures of Efficacy

Efficacy of methods described herein can be evaluated by assessment of tumor response to treatment according to RECIST v1.1. Clinical benefits can be assessed by the objective response rate (ORR), duration of response (DOR), disease control rate (DCR), progression-free survival (PFS), or overall survival (OS).

The primary efficacy endpoint is ORR, defined as the proportion of patients with a confirmed best overall response of CR or PR in the analysis population. Tumor assessments after the initiation of further anticancer therapy are excluded for the assessment of best overall response.

DOR will be evaluated as a secondary endpoint and is defined as the time from first documented CR or PR until the subsequently documented disease progression or death, whichever occurs earlier.

DCR will be evaluated as a secondary endpoint and is defined as the proportion of patients with a best overall response of CR, PR or SD.

PFS will be assessed as a secondary endpoint and is defined as the time from the date of first dose to the date of disease progression or death due to any cause, whichever occurs earlier.

OS will be assessed as a secondary endpoint and is defined as the time from date of first dose to the date of death due to any cause. Subjects without documented death at the time of the final analysis will be censored at the last known alive date.

### Results

In Cohort 1, a total of sixteen patients have been evaluated to date, with results shown in Table 14. Two patients have achieved a complete response (CR), which was confirmed by second subsequent tumor scans. Nine patients have achieved a partial response (PR) as evidenced by at least one tumor scan. Of the these nine patients, seven PRs have confirmed by a subsequent scan.

TABLE
Cohort 1 Tumor Response Summary

| | Cohort 1 |
|---|---|
| RECIST response | (N = 16)* |
| No. of patients with | 14 |
| at least 1 scan | |
| CR | 2*** |
| PR | 9** |
| SD | 3 |
| PD | 0 |
| Discontinued | 2 |
| before scan | |
| On study, no | 0 |
| scan yet | |

*1 patient withdrew from study before 1^{st} tumor assessment (not part of ITT per protocol)
**7 patients with confirmed PR
***2 patients with confirmed CR

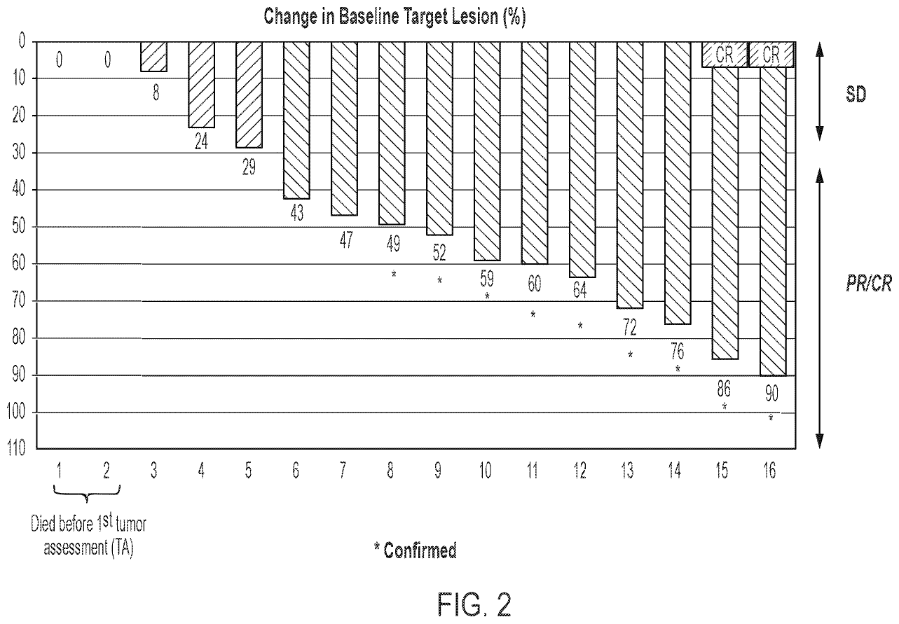

FIG. 2 depicts percent tumor shrinkage observed in the patients of Cohort 1: of sixteen patients, two died before the first tumor assessment (TA). Of the fourteen patients with at least one tumor assessment scan, two patients showed complete response (CR) and nine patients showed partial response (PR) with tumor shrinkage of 30% or more. Moreover, two patients with stable disease (SD) had shrinkage of more than 20%.

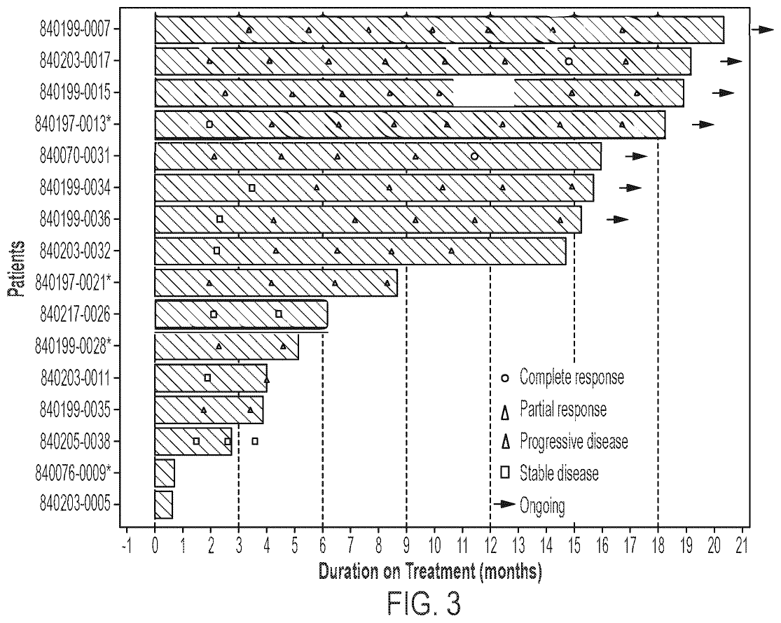

FIG. 3 further illustrates the duration of treatment and tumor response according to RECIST v1.1 for the patients of Cohort 1 who received at least one dose. Of these patients, seven patients have received treatment for at least fifteen months. Each of these seven patients is also continuing to receive treatment as each has a last assessment of either a partial response or a complete response.

These clinical data demonstrate that a PARP inhibitor such as niraparib can be effective in combination with an anti-PD-1 agent such as pembrolizumab. In particular, patients with NSCLC characterized by high PD-L1 (e.g., TPS≥50%) can particularly benefit from these methods: of the fourteen patients of Cohort 1 having at least one tumor assessment scan, 11/14 (79%) showed a complete response (CR) or partial response (PR) to treatment. The unexpectedly high response rate is significant, particularly when compared to other therapies.

### EQUIVALENTS

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

What is claimed is:

1. A method of treating ovarian cancer the method comprising a treatment period and a maintenance period wherein: (i) the treatment period comprises administering 500 mg of dostarlimab every three weeks (Q3W) for two to six doses; and, (ii) the maintenance period comprises administering 1000 mg of dostarlimab every six weeks (Q6W) and a daily dose of from 100 mg to 300 mg of niraparib.

2. The method of claim 1 wherein the maintenance period daily dose of niraparib is 200 mg.

3. The method of claim 1 wherein the maintenance period daily dose of niraparib is 300 mg.

4. The method of any one of claims 1 to 3 wherein the ovarian cancer is advanced ovarian cancer (aOC), metastatic ovarian cancer (mOC), or nonmucinous epithelial ovarian cancer.

5. The method of claim 1 wherein the ovarian cancer is characterized by PD-L1 expression of greater than 5% as determined by an FDA-approved assay.

6. The method of claim 1 wherein the ovarian cancer is characterized as a BRCA mutant ovarian cancer.

7. The method of claim 1 wherein the ovarian cancer is a primary fallopian tube ovarian cancer.

8. The method of claim 1 wherein the ovarian cancer is characterized as homologous recombination deficient (HRD).

9. The method of claim 1 wherein the ovarian cancer is stage III or stage IV metastatic ovarian cancer.

10. A method of treating PD-L1 positive advanced ovarian cancer the method comprising (i) a treatment phase comprising administering from two to six 500 mg doses of dostarlimab every three weeks (Q3W); and; (ii) a maintenance phase comprising administering 200 mg or 300 mg of niraparib daily (QD) and 1000 mg of dostarlimab every six weeks (Q6W).


SEQUENCE LISTING (58 sequences)

SEQ ID NO: 1 - protein, 8 aa, synthetic construct
GFTFSSYD

SEQ ID NO: 2 - protein, 8 aa, synthetic construct
ISGGGSYT

SEQ ID NO: 3 - protein, 9 aa, synthetic construct
ASPYYAMDY

SEQ ID NO: 4 - protein, 6 aa, synthetic construct
QDVGTA

SEQ ID NO: 5 - skipped

SEQ ID NO: 6 - protein, 9 aa, synthetic construct
QHYSSYPWT

SEQ ID NO: 7 - protein, 117 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSA

SEQ ID NO: 8 - protein, 108 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKR

SEQ ID NO: 9 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
GFTFSSYD

SEQ ID NO: 12 - protein, 8 aa, synthetic construct
ISGGGTYT

SEQ ID NO: 13 - protein, 5 aa, synthetic construct
ASMDY

SEQ ID NO: 14 - protein, 6 aa, synthetic construct
QSIRRY

SEQ ID NO: 15 - skipped

SEQ ID NO: 16 - protein, 9 aa, synthetic construct
QQSHSAPLT

SEQ ID NO: 17 - protein, 114 aa, synthetic construct
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 18 - protein, 113 aa, synthetic construct
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSS

SEQ ID NO: 19 - protein, 108 aa, synthetic construct
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 20 - protein, 107 aa, synthetic construct
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIK

SEQ ID NO: 21 - protein, 440 aa, synthetic construct
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 214 aa, synthetic construct
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 5 aa, synthetic construct
DDYIH

SEQ ID NO: 24 - protein, 17 aa, synthetic construct
WIDAMNDDSQYSSKFQG

SEQ ID NO: 25 - protein, 5 aa, synthetic construct
AFGGY

SEQ ID NO: 26 - protein, 16 aa, synthetic construct
RSSQSLVHSDSNTYLH

SEQ ID NO: 27 - protein, 7 aa, synthetic construct
LVSNRFS

SEQ ID NO: 28 - protein, 9 aa, synthetic construct
GQSTHVPYA

SEQ ID NO: 29 - protein, 114 aa, synthetic construct
EVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSS

SEQ ID NO: 30 - protein, 112 aa, synthetic construct
DIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIK

SEQ ID NO: 31 - protein, 441 aa, synthetic construct
EVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 219 aa, synthetic construct
DIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 6 aa, synthetic construct
VTITCR

SEQ ID NO: 36 - protein, 42 aa, synthetic construct
FSGSGSGTDFTLTISSLQPEDFAVYYCQQSHSAPLTFGGGTK

SEQ ID NO: 37 - protein, 16 aa, synthetic construct
SGTASVVCLLNNFYPR

SEQ ID NO: 38 - protein, 17 aa, synthetic construct
VYACEVTHQGLSSPVTK

SEQ ID NO: 39 - protein, 7 aa, synthetic construct
SFNRGEC

SEQ ID NO: 40 - protein, 15 aa, synthetic construct
GPSVFPLAPCSRGEC

SEQ ID NO: 41 - protein, 12 aa, synthetic construct
GPSVFPLAPCSR

SEQ ID NO: 42 - protein, 20 aa, synthetic construct
LSCAAASGFTFSSYDMSWVR

SEQ ID NO: 43 - protein, 29 aa, synthetic construct
AEDTAVYYCASMDYWGQGTTVTVSSASTK

SEQ ID NO: 44 - protein, 14 aa, synthetic construct
STSESTAALGCLVK

SEQ ID NO: 45 - protein, 23 aa, synthetic construct
TYTCNVDHKSTSESTAALGCLVK

SEQ ID NO: 46 - protein, 14 aa, synthetic construct
TYTCNVDHKPSNTK

SEQ ID NO: 47 - protein, 25 aa, synthetic construct
YGPPCPPCPAPEFLGGPSVFLFPPK

SEQ ID NO: 48 - protein, 50 aa, synthetic construct
YGPPCPPCPAPEFLGGPSVFLFPPKYGPPCPPCPAPEFLGGPSVFLFPPK

SEQ ID NO: 49 - protein, 27 aa, synthetic construct
YGPPCPPCPAPEFLGGPSVFLFPPKPK

SEQ ID NO: 50 - protein, 33 aa, synthetic construct
TPEVTCVVVDVSQEDPEVQFNWYVDGVEVHNAK

SEQ ID NO: 51 - protein, 10 aa, synthetic construct
NQVSLTCLVK

SEQ ID NO: 52 - protein, 23 aa, synthetic construct
WQEGNVFSCSVMHEALHNHYTQK

SEQ ID NO: 53 - protein, 24 aa, synthetic construct
DIVMTQTPLSLSVTPGQPASISCR

SEQ ID NO: 54 - protein, 26 aa, synthetic construct
VEAEDVGVYFCGQSTHVPYAFGGGTK

SEQ ID NO: 55 - protein, 4 aa, synthetic construct
ISCK

SEQ ID NO: 56 - protein, 31 aa, synthetic construct
SEDTAVYYCTYAFGGYWGQGTTVTVSSASTK

SEQ ID NO: 57 - protein, 9 aa, synthetic construct
TYTCNVDHK

SEQ ID NO: 58 - protein, 26 aa, synthetic construct
YGPPCPPCPPAPEFLGGPSVFLFPPK